(19) United States
(12) Patent Application Publication
(10) Pub. No.: US 2020/0113927 A1
(43) Pub. Date: Apr. 16, 2020

(54) LACTATE DEHYDROGENASE A (LDHA) iRNA COMPOSITIONS AND METHODS OF USE THEREOF

(71) Applicants: Alnylam Pharmaceuticals, Inc., Cambridge, MA (US); The UAB Research Foundation, Birmingham, AL (US)

(72) Inventors: David Erbe, Arlington, MA (US); Abigail Liebow, Somerville, MA (US); Kevin Fitzgerald, Brookline, MA (US); Gregory Hinkle, Plymouth, MA (US); Kyle David Wood, Homewood, AL (US); Ross Philip Holmes, Birmingham, AL (US); John Knight, Homewood, AL (US)

(21) Appl. No.: 16/716,705

(22) Filed: Dec. 17, 2019

Related U.S. Application Data
(63) Continuation of application No. PCT/US2018/041977, filed on Jul. 13, 2018.
(60) Provisional application No. 62/576,783, filed on Oct. 25, 2017.
(60) Provisional application No. 62/532,020, filed on Jul. 13, 2017.

(51) Int. Cl. A61K 31/7105; C12N 15/113

(52) U.S. Cl. CPC A61K 31/7105; C12N 15/113; C12Y 101/01027; C12N 2310/3525; C12N 2310/3231; C12N 2310/11; C12N 2310/3521; C12N 2310/3523; C12N 2310/313; C12N 2310/3125; C12N 2310/321

(57) ABSTRACT

The invention relates to double-stranded ribonucleic acid (dsRNA) compositions targeting the LDHA gene, as well as methods of inhibiting epression of LDHA, methods of inhibiting LDHA and HAO1, and methods of treating subjects that would benefit from reduction in expression of LDHA, such as subjects having an oxalate pathway-associated disease, disorder, or condition, using such dsRNA compositions.

### RELATED APPLICATIONS

The present application is a 35 § U.S.C. 111(a) continuation application which claims the benefit of priority to PCT/US2018/041977, filed on Jul. 13, 2018, U.S. Provisional Application No. 62/576,783, filed on Oct. 25, 2017 and U.S. Provisional Application No. 62/532,020, filed on Jul. 13, 2017. The entire contents of each of the foregoing applications are incorporated herein by reference.

### SEQUENCE LISTING

The instant application contains a Sequence Listing which has been submitted electronically in ASCII format and is hereby incorporated by reference in its entirety. Said ASCII copy, created on Dec. 17, 2019, is named 121301-07503_SL.TXT and is 1,154,854 bytes in size.

### BACKGROUND OF THE INVENTION

Oxalate (C₂O₄²⁻) is the salt-forming ion of oxalic acid (C₂H₂O₄) that is widely distributed in both plants and animals. It is an unavoidable component of the human diet and a ubiquitous component of plants and plant-derived foods. Oxalate can also be synthesized endogenously via the metabolic pathways that occur in the liver. Dietary and endogenous contributions to urinary oxalate excretion are equal. Glyoxylate is an immediate precursor to oxalate and is derived from the oxidation of glycolate by the enzyme glycolate oxidase (GO), also known, and referred to herein, as hydroxyacid oxidase (HAO1), or by catabolism of hydroxyproline, a component of collagen. Transamination of glyoxylate with alanine by the enzyme alanine/glyoxylate aminotransferase (AGT) results in the formation of pyruvate and glycine. Excess glyoxylate is converted to oxalate by lactate dehydrogenase A (referred to herein as LDHA). The endogenous pathway for oxalate metabolism is illustrated in FIG. 1A.

Lactate dehydrogenase is a protein found in all tissues. It is composed of four subunits with the two most common subunits being the LDH-M and LDH-H proteins. These proteins are encoded by the LDHA and LDHB genes, respectively. Various combinations of the LDH-M and LDH-H proteins result in five distinct isoforms of LDH. LDHA is the most important gene involved in the liver lactate dehydrogenase isoform. Specifically, within the liver, LDHA is important as the final step in the endogenous production of oxalate, by converting the precursor glyoxylate to oxalate. It also serves an important role in the Cori Cycle and in the anaerobic phase of glycolysis where it converts lactate to pyruvate and vice versa.

Oxalic acid may form oxalate salts with various cations, such as sodium, potassium, magnesium, and calcium. Although sodium oxalate, potassium oxalate, and magnesium oxalate are water soluble, calcium oxalate (CaOx) is nearly insoluble. Excretion of oxalate occurs primarily by the kidneys via glomerular filtration and tubular secretion.

Since oxalate binds with calcium in the kidney, urinary CaOx supersaturation may occur, resulting in the formation and deposition of CaOx crystals in renal tissue or collecting system. These CaOx crystals contribute to the formation of diffuse renal calcifications (nephrocalcinosis) and stones (nephrolithiasis). Subjects having diffuse renal calcifications or nonobstructing stones typically have no symptoms. However, obstructing stones can cause severe pain. Moreover, over time, these CaOx crystals cause injury and progressive inflammation to the kidney and, when secondary complications such as obstruction are present, these CaOx crystals may lead to decreased renal function and in severe cases even to end-stage renal failure and the need for dialysis. Furthermore, systemic deposition of CaOx (systemic oxalosis) may occur in extrarenal tissues, including soft tissues (such as thyroid and breast), heart, nerves, joints, skin, and retina, which can lead to early death if left untreated.

Among the most well-known oxalate pathway-associated diseases, e.g., kidney stone formation diseases, are the primary hyperoxalurias which are inherited diseases characterized by increased endogenous oxalate synthesis with variable clinical phenotypes. Therapies that modulate oxalate synthesis are currently not available and there are only a few treatment options that exist for subjects having a hereditary hyperoxaluria. Ultimatly, some subjects with hereditary hyperoxaluria require kidney/liver transplants. Other oxalate pathway-associated diseases, disorders, and conditions include calcium oxalate tissue deposition diseases, disorders, and conditions.

Currently, the primary treatment for many of these oxalate pathway-associated diseases, disorders, and conditions (e.g., with kidney stone disease) is increased fluid intake and dietary alterations (e.g., decreased protein intake, decreased sodium intake, decreased ascorbic acid intake, moderate calcium intake, phosphate or magnesium supplementation, and pyridoxine treatment). However, subjects often fail to adhere to such life-style changes or experience no significant benefit. Treatment for some of the other oxalate pathway-associated diseases, disorders, and conditions, such as chronic kidney disease, include the use of ACE inhibitors (angiotensin converting enzyme inhibitors) and ARBs (angiotensin II antagonists) which may slow the progression of disease. Nonetheless, subjects having chronic kidney disease progressively lose kidney function and progress to the need for dialysis or a kidney transplant. Most of these oxalate pathway-associated diseases are without treatments, and none currently have oxalate reduction treatments available.

Further, there are oxalate pathway-associated diseases, disorders, and conditions include lactate dehydrogenase-associated diseases, disorders, and conditions. For example, the role of lactate dehydrogenase is well known in cancer (hepatocellular), and inhibition has been shown to reduce cancer growth. Other lactate dehydrogenase-associated diseases, disorders and conditions include fatty liver (steatosis), nonalcoholic steatohepatitis (NASH), cirrhosis of the liver, accumulation of fat in the liver, inflammation of the liver, hepatocellular necrosis, liver fibrosis, and nonalcoholic fatty liver disease (NAFLD). Given the essential role of LDH in glycolysis, however, treatment options have been limited.

Accordingly, there is a need in the art for alternative treatments for subjects having an oxalate pathway-associated disease, disorder, and condition.

### SUMMARY OF THE INVENTION

The present invention is based, at least in part, on the discovery that, by targeting LDHA with the iRNA agents, compositions comprising such agents, and methods disclosed herein, a liver specific and superior LDHA and urinary oxalate lowering effect is achieved.

Accordingly, the present invention provides iRNA compositions which effect the RNA-induced silencing complex (RISC)-mediated cleavage of RNA transcripts of an LDHA gene. The LDHA gene may be within a cell, e.g., a cell within a subject, such as a human. The present invention also provides methods of using the iRNA compositions of the invention for inhibiting the expression of an LDHA gene for treating a subject who would benefit from inhibiting or reducing the expression of an LDHA gene, e.g., a subject that would benefit from a reduction or inhibition in urinary oxalate production, e.g., a subject suffering or prone to suffering from an oxalate pathway-associated disease disorder, or condition, such as a subject suffering or prone to suffering from an oxalate-associated disease, disorder, or condition, e.g., a kidney stone formation disease, disorder, or condition or a calcium oxalate tissue deposition disease, disorder, or condition; or an LDHA-associated disease, disorder, or condition.

The present invention also provides iRNA compositions which effect the RNA-induced silencing complex (RISC)-mediated cleavage of RNA transcripts of an LDHA gene and an HAO1 gene. The LDHA gene and the HAO1 gene may be within a cell, e.g., a cell within a subject, such as a human. The present invention also provides methods of using the iRNA compositions of the invention for inhibiting the expression of an LDHA gene and an HAO1 gene for treating a subject who would benefit from inhibiting or reducing the expression of an LDHA gene and an HAO1 gene, e.g., a subject that would benefit from a reduction or inhibition in urinary oxalate production, e.g., a subject suffering or prone to suffering from an an oxalate-associated disease, disorder, or condition, e.g., a kidney stone formation disease, disorder, or condition or a calcium oxalate tissue deposition disease, disorder, or condition; or an LDH-associated disease, disorder, or condition.

Accordingly, in one aspect, the present invention provides a double stranded ribonucleic acid (dsRNA) agent for inhibiting expression of lactic acid dehydrogenase A (LDHA) in a cell, wherein said dsRNA agent comprises a sense strand and an antisense strand, the antisense strand comprising a region of complementarity which comprises at least 15 contiguous nucleotides differing by no more than 3 nucleotides from any one of the antisense sequences listed in any one of Tables 2-5.

In one embodiment, the dsRNA agent comprises at least one modified nucleotide.

In other embodiments, substantially all of the nucleotides of the sense strand comprise a modification; substantially all of the nucleotides of the antisense strand comprise a modification; or substantially all of the nucleotides of the sense strand and substantially all of the nucleotides of the antisense strand comprise a modification.

In yet other embodiments, all of the nucleotides of the sense strand comprise a modification; all of the nucleotides of the antisense strand comprise a modification; or all of the nucleotides of the sense strand and all of the nucleotides of the antisense strand comprise a modification.

In one embodiment, at least one of said modified nucleotides is selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modifice nucleotide, and a 2-O—(N-methylacetamide) modified nucleotide, and combinations thereof.

The region of complementarity may be at least 17 nucleotides in length; 19 to 30 nucleotides in length; 19-25 nucleotides in length; or 21 to 23 nucleotides in length.

Each strand of the dsRNA agent may be no more than 30 nucleotides in length. Each strand of the dsRNA agent may be independently 19-30 nucleotides in length; independently 19-25 nucleotides in length; or independently 21-23 nucleotides in length.

At least one strand of the dsRNA agent may comprise a 3′ overhang of at least 1 nucleotide; or at least one strand may comprise a 3′ overhang of at least 2 nucleotides.

In one embodiment, the dsRNA agent further comprises at least one phosphorothioate or methylphosphonate internucleotide linkage.

The phosphorothioate or methylphosphonate internucleotide linkage may be at the 3′-terminus of one strand (e.g., the antisense strand; or the sense strand); or the phosphorothioate or methylphosphonate internucleotide linkage may be at the 5′-terminus of one strand (e.g., the antisense strand; or the sense strand); or the phosphorothioate or methylphosphonate internucleotide linkage may be at the both the 5′- and 3′-terminus of one strand.

The dsRNA agent may further comprise a ligand.

In one embodiment, the ligand is conjugated to the 3′ end of the sense strand of the dsRNA agent.

In one embodiment, the ligand is one or more N-acetylgalactosamine (GalNAc) derivatives attached through a monovalent, bivalent, or trivalent branched linker.

In another embodiment, the ligand is

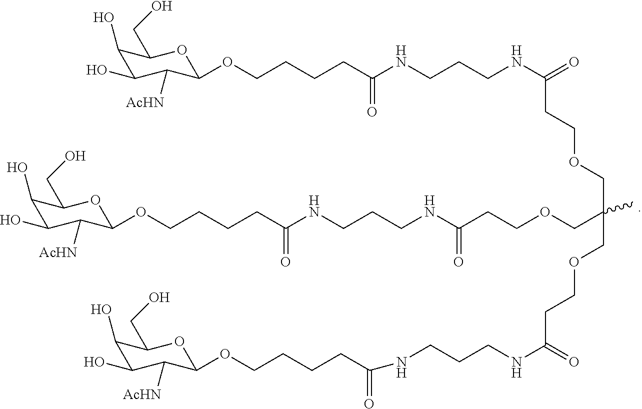 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the dsRNA agent is conjugated to the ligand as shown in the following schematic

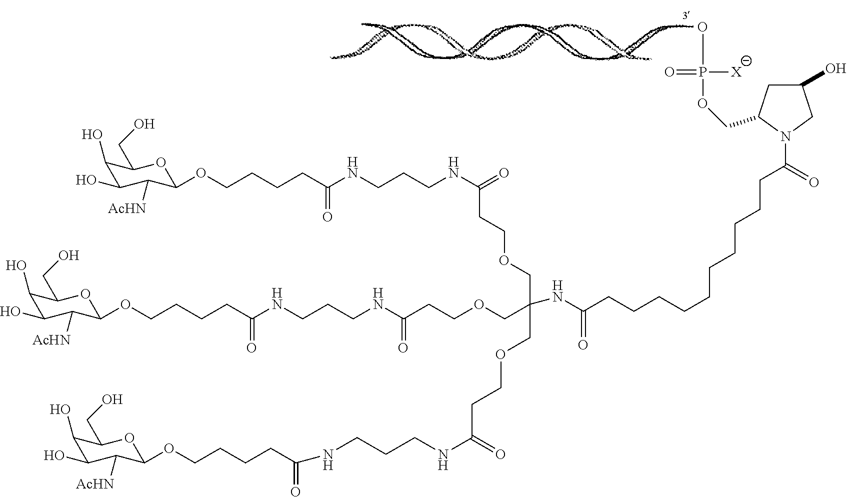

and, wherein X is O or S.

In one embodiment, the X is O.

In one embodiment, the region of complementarity consists of one of the antisense sequences listed in any one of Tables 2-5.

In one embodiment, the sense strand and the antisense strand comprise nucleotide sequences selected from the group consisting of the nucleotide sequences of any one of the agents listed Many one of Tables 2-5.

In another aspect, the present invention provides a dual targeting RNAi agent, comprising a first double stranded ribonucleic acid (dsRNA) agent that inhibits expression of lactic dehydrogenase A (LDHA) comprising a sense strand and an antisense strand; and a second double stranded ribonucleic acid (dsRNA) agent that inhibits expression of hydroxyacid oxidase 1 (glycolate oxidase) (HAO1) comprising a sense strand and an antisense strand, wherein the first dsRNA agent and the second dsRNA agent are covalently attached.

In one embodiment, the sense strand of the first dsRNA agent comprises at least 15 contiguous nucleotides differing by no more than 3 nucleotides from the nucleotide sequence of SEQ ID NO:1, and the antisense strand of the first dsRNA agent comprises at least 15 contiguous nucleotides differing by no more than 3 nucleotides from the nucleotide sequence of SEQ ID NO:2.

In another embodiment, the antisense strand of the first dsRNA agent comprises a region of complementarity which comprises at least 15 contiguous nucleotides differing by no more than 3 nucleotides from any one of the antisense sequences listed in any one of Tables 2-5.

In one embodiment, the sense strand of the second dsRNA agent comprises at least 15 contiguous nucleotides differing by no more than 3 nucleotides from the nucleotide sequence of SEQ ID NO:21, and said antisense strand of the second dsRNA agent comprises at least 15 contiguous nucleotides differing by no more than 3 nucleotides from the nucleotide sequence of SEQ ID NO:22.

In another embodiment, the antisense strand of the second dsRNA agent comprises a region of complementarity which comprises at least 15 contiguous nucleotides differing by no more than 3 nucleotides from any one of the antisense sequences listed in any one of Tables 7-14.

In one embodiment, the first dsRNA agent and the second dsRNA agent each independently comprise at least one modified nucleotide.

In another embodiment, substantially all of the nucleotides of the sense strand and substantially all of the nucleotides of the antisense strand of the first dsRNA agent and substantially all of the nucleotides of the sense strand and substantially all of the nucleotides of the antisense strand of the second dsRNA agent are modified nucleotides.

In one embodiment, at least one of the modified nucleotides of the first dsRNA agent and at least one of the modified nucleotides of the second dsRNA agent are each independently selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, and a nucleotide comprising a 5′-phosphate mimic.

In another embodiment, at least one of the modified nucleotides of the first dsRNA agent and at least one of the modified nucleotides of the second dsRNA agent are each independently selected from the group consisting of 2′-O-methyl and 2′fluoro modifications.

The region of complementarity of the first dsRNA agent and/or the region of complementarity of the second dsRNA agent may each independently be 19 to 30 nucleotides in length.

Each strand of the first dsRNA agent and each strand of the second dsRNA agent may each independently be 19-30 nucleotides in length.

In one embodiment, at least one strand of the first dsRNA agent and/or at least one strand of the second dsRNA agent each independently comprise a 3′ overhang of at least 1 nucleotide. In one embodiment, the first dsRNA agent and/or the second dsRNA agent each independently further comprise at least one phosphorothioate or methylphosphonate internucleotide linkage.

In one embodiment, the first dsRNA agent and/or the second dsRNA agent each independently further comprise at least one ligand.

In another embodiment, the at least one ligand is conjugated to the sense strand of the first dsRNA agent and/or the second dsRNA agent.

In one embodiment, the at least one ligand is conjugated to the 3′-end, 5′-end, or an internal position of one of the sense strands.

In another embodiment, the at least one ligand is conjugated to the antisense strand of the first dsRNA agent and/or the second dsRNA agent.

In one embodiment, the at least one ligand is conjugated to the 3′-end, 5′-end, or an internal position of one of the antisense strands.

In one embodiment, the ligand is an N-acetylgalactosamine (GalNAc) derivative.

In one embodiment, the ligand is one or more GalNAc derivatives attached through a monovalent, a bivalent, or a trivalent branched linker.

In one embodiment, the ligand is

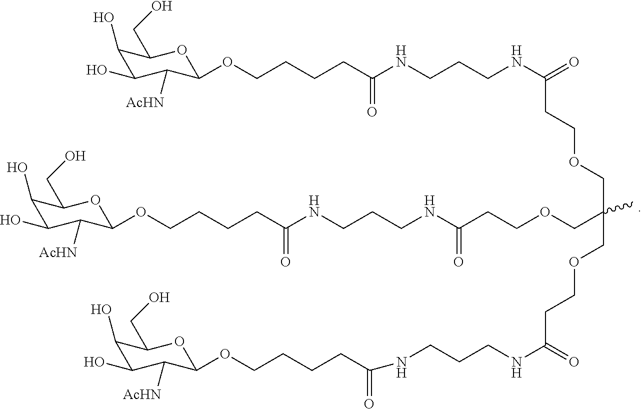 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the first dsRNA agent and the second dsRNA agent are each independently conjugated to the ligand as shown in the following schematic

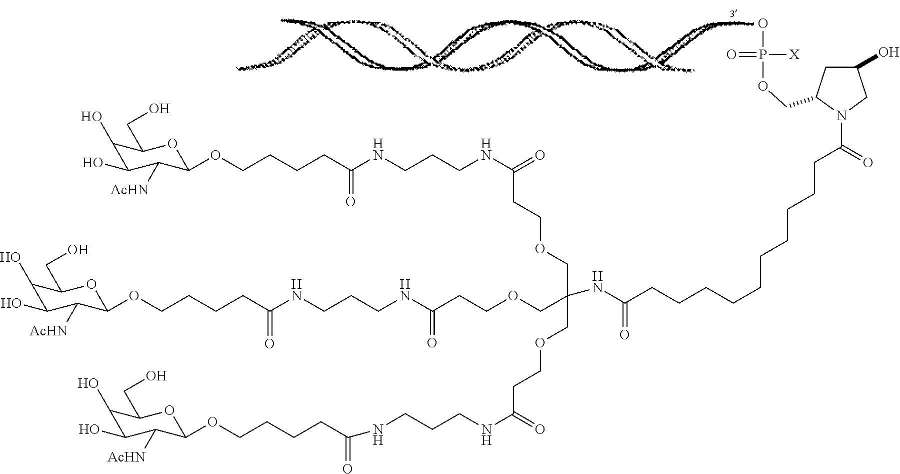

and, wherein X is O or S.

In one embodiment, the X is O.

In one embodiment, the first dsRNA agent and the second dsRNA agent are covalently attached via a covalent linker.

In one embodiment, the covalent linker is selected from the group consisting of a single stranded nucleic acid linker, a double stranded nucleic acid linker, a partially single stranded nucleic acid linker, a partially double stranded nucleic acid linker, a carbohydrate moiety linker, and a peptide linker. In another embodiment, the covalent linker is a cleavable linker or a non-cleavable linker. In one embodiment, the covalent linker attaches the sense strand of the first dsRNA agent to the sense strand of the second dsRNA agent. In another embodiment, the covalent linker attaches the antisense strand of the first dsRNA agent to the antisense strand of the second dsRNA agent.

In one embodiment, the covalent linker further comprises at least one ligand.

In one embodiment, contacting a cell with the dual targeting RNAi agent of the inventioninhibits expression of the LDHA gene and the HAO1 gene to a level substantially the same as the level of inhibition of expression obtained by the contacting of a cell with both dsRNA agents individually. In another embodiment, contacting a cell with the dual targeting RNAi agent inhibits expression of the LDHA gene and the HAO1 gene to a level higher than the level of inhibition of expression obtained by the contacting of a cell with both dsRNA agents individually.

In one embodiment, the level of inhibition of LDHA expression is at least about 5%, about 10%, about 15%, about 20%, about 25%, about 30%, about 40%, about 50%, about 60%, about 70%, about 80%, about 90%, about 95%, about 98% or about 100% higher than the level of inhibition of expression obtained by the contacting of a cell with both dsRNA agents individually.

In one embodiment, the level of inhibition of HAO1 expression is at least about 5%, about 10%, about 15%, about 20%, about 25%, about 30%, about 40%, about 50%, about 60%, about 70%, about 80%, about 90%, about 95%, about 98% or about 100% higher than the level of inhibition of expression obtained by the contacting of a cell with both dsRNA agents individually.

In one embodiment, contacting a cell with the dual targeting RNAi agent inhibits oxalate and/or glyoxylate protein production to a level lower than the level of protein production obtained by the contacting of a cell with both dsRNA agents individually. In another embodiment, contacting a cell with the dual targeting RNAi agent inhibits oxalate and/or glyoxylate protein production to a level lower than the level of protein production obtained by the contacting of a cell with both dsRNA agents individually.

The present invention also provides cells containing a dsRNA agent or a dual targeting RNAi agent of the invention; and vectors encoding at least one strand of a dsRNA agent or a dual targeting RNAi agent of the invention.

Further, the present invention provides a pharmaceutical composition for inhibiting expression of a lactic acid dehydrogenase A (LDHA) gene comprising a dsRNA agent of the invention; or a pharmaceutical composition for inhibiting expression of a lactic acid dehydrogenase A (LDHA) gene and an hydroxyacid oxidase 1 (glycolate oxidase) (HAO1) gene comprising a dual targeting RNAi agent of the invention.

In one aspect, the present invention provides a pharmaceutical composition, comprising a first double stranded ribonucleic acid (dsRNA) agent that inhibits expression of lactic acid dehydrogenase A (LDHA) comprising a sense strand and an antisense strand, wherein said sense strand comprises at least 15 contiguous nucleotides differing by no more than 3 nucleotides from the nucleotide sequence of SEQ ID NO:1, and said antisense strand comprises at least 15 contiguous nucleotides differing by no more than 3 nucleotides from the nucleotide sequence of SEQ ID NO:2; and a second double stranded ribonucleic acid (dsRNA) agent that inhibits expression of hydroxyacid oxidase 1 (glycolate oxidase) (HAO1) comprising a sense strand and an antisense strand, wherein said sense strand comprises at least 15 contiguous nucleotides differing by no more than 3 nucleotides from the nucleotide sequence of SEQ ID NO:21, and said antisense strand comprises at least 15 contiguous nucleotides differing by no more than 3 nucleotides from the nucleotide sequence of SEQ ID NO:22.

In another aspect, the present invention provides a pharmaceutical composition, comprising a first double stranded ribonucleic acid (dsRNA) agent that inhibits expression of lactic acid dehydrogenase A (LDHA) comprising a sense strand and an antisense strand, the antisense strand comprising a region of complementarity which comprises at least 15 contiguous nucleotides differing by no more than 3 nucleotides from any one of the antisense sequences listed in any one of Tables 2-5; and a second double stranded ribonucleic acid (dsRNA) agent that inhibits expression of hydroxyacid oxidase 1 (glycolate oxidase) (HAO1) comprising a sense strand and an antisense strand, the antisense strand comprising a region of complementarity which comprises at least 15 contiguous nucleotides differing by no more than 3 nucleotides from any one of the antisense sequences listed in any one of Tables 7-14.

The agent may be formulated in an unbuffered solution, such as saline or water; or the agent may be formulated with a buffered solution, such as a solution comprising acetate, citrate, prolamine, carbonate, or phosphate or any combination thereof; or phosphate buffered saline (PBS).

The present invention provides a method of inhibiting lactic acid dehydrogenase A (LDHA) expression in a cell. The methods include contacting the cell with an agent or a pharmaceutical composition of the invention, thereby inhibiting expression of LDHA in the cell.

The present invention also provides a method of inhibiting lactic acid dehydrogenase A (LDHA) expression and hydroxyacid oxidase 1 (glycolate oxidase) (HAO1) expression in a cell. The method includes contacting the cell with a dual targeting RNAi agent of the invention or a pharmaceutical composition comprising a dual targeting agent of the invention, thereby inhibiting expression of LDHA and HAO1 in the cell.

In one embodiment, the cell is within a subject, such as a human.

In one embodiment, the LDHA expression is inhibited by at least 30%, 40%, 50%, 60%, 70%, 80%, 90%, 95%, or to below the level of detection of LDHA expression.

In one embodiment, the HAO1 expression is inhibited by at least 30%, 40%, 50%, 60%, 70%, 80%, 90%, 95%, or to below the level of detection of HAO1 expression.

In one embodiment, the human subject suffers from an oxalate pathway-associated disease, disorder, or condition.

In one embodiment, the oxalate pathway-associated disease, disorder, or condition is an oxalate-associated disease, disorder, or condition, or a lactate dehydrogenase-associated disease, disorder, or condition.

In one embodiment, the oxalate-associated disease, disorder, or condition is a kidney stone formation disease, disorder, or condition, or a calcium oxalate tissue deposition disease, disorder, or condition.

In one embodiment, the kidney stone formation disease, disorder, or condition is a calcium oxalate stone formation disease, disorder, or condition or a non-calcium oxalate stone formation disease, disorder, or condition.

In one embodiment, the calcium oxalate stone formation disease, disorder, or condition is a hyperoxaluria disease, disorder, or condition or a non-hyperoxaluria disease, disorder, or condition.

In one embodiment, the hyperoxaluria disease, disorder, or condition is selected from the group consisting of primary hyperoxaluria, enteric hyperoxaluria, dietary hyperoxaluria, and idiopathic hyperoxaluria.

In one embodiment, the non-hyperoxaluria stone formation disease, disorder, or condition is hypercalciuria and/or hypocitraturia.

In one embodiment, the non-hyperoxaluria stone formation disease, disorder, or condition is calcium oxalate or non-calcium oxalate kidney stone formation disease.

In one embodiment, the calcium oxalate tissue deposition disease, disorder, or condition is selected from the group consisting of systemic calcium oxalate tissue deposition disease, disorder, or condition or tissue specific calcium oxalate tissue deposition disease, disorder, or condition.

In one embodiment, the lactate dehydrogenase-associated disease, disorder, or condition is selected from the group consisting of cancer, fatty liver (steatosis), nonalcoholic steatohepatitis (NASH), cirrhosis of the liver, accumulation of fat in the liver, inflammation of the liver, hepatocellular necrosis, liver fibrosis, and nonalcoholic fatty liver disease (NAFLD).

In one embodiment, the cell is a liver cell.

In one aspect, the present invention provides a method of inhibiting the epression of LDHA in a subject. The method includes administering to the subject a therapeutically effective amount of the agent or a pharmaceutical composition of the invention, thereby inhibiting the expression of LDHA in the subject.

In another aspect, the present invention provides a method of inhibiting lactic acid dehydrogenase A (LDHA) expression and hydroxyacid oxidase 1 (glycolate oxidase) (HAO1) expression in a subject. The methods include administering to the subject a therapeutically effective amount of dual targeting RNAi agent of the invention, or a pharmaceutical composition comprising a dual targeting RNAi agent of the invention, thereby inhibiting expression of LDHA and HAO1 in the subject.

In one aspect, the present invention provides a method of treating a subject having a disorder that would benefit from a reduction in LDHA expression. The method includes administering to the subject a therapeutically effective amount of the agent or a pharmaceutical composition of the invention, thereby treating said subject.

In another aspect, the present invention provides a method of preventing at least one symptom in a subject having a disease or disorder that would benefit from reduction in expression of an LDHA gene. The methods include administering to the subject a prophylactically effective amount of an agent or a pharmaceutical composition of the invention, thereby preventing at least one symptom in the subject.

In one embodiment, the disorder is an oxalate pathway-associated disease, disorder, or condition.

In one aspect, the present invention provides a method of treating a subject having an oxalate pathway-associated disease, disorder, or condition. The method includes administering to the subject a therapeutically effective amount of an agent or a pharmaceutical composition of the invention, thereby treating the subject.

In another aspect, the present invention provides a method of preventing at least one symptom in a subject having an oxalate pathway-associated disease, disorder, or condition. The methods includes administering to the subject a prophylactically effective amount of the agent or a pharmaceutical composition of the invention, thereby preventing at least one symptom in the subject.

In one embodiment, the administration of the dsRNA agent or the pharmaceutical composition to the subject causes a decrease in one or urinary oxalate, tissue oxalate, plasma oxalate, a decrease in LDHA enzymatic activity, a decrease in LDHA protein accumulation, and/or a decrease in HAO1 protein accumulation.

In one embodiment, the oxalate pathway-associated disease, disorder, or condition is an oxalate-associated disease, disorder, or condition, or a lactate dehydrogenase-associated disease, disorder, or condition.

In one embodiment, the oxalate-associated disease, disorder, or condition is a kidney stone formation disease, disorder, or condition, or a calcium oxalate tissue deposition disease, disorder, or condition.

In one embodiment, the kidney stone formation disease, disorder, or condition is a calcium oxalate stone formation disease, disorder, or condition or a non-calcium oxalate stone formation disease, disorder, or condition.

In one embodiment, the calcium oxalate stone formation disease, disorder, or condition is a hyperoxaluria disease, disorder, or condition or a non-hyperoxaluria disease, disorder, or condition.

In one embodiment, the hyperoxaluria disease, disorder, or condition is selected from the group consisting of primary hyperoxaluria, enteric hyperoxaluria, dietary hyperoxaluria, and idiopathic hyperoxaluria.

In one embodiment, the non-hyperoxaluria stone formation disease, disorder, or condition is hypercalciuria and/or hypocitraturia.

In one embodiment, the non-hyperoxaluria stone formation disease, disorder, or condition is calcium oxalate or non-calcium oxalate kidney stone formation disease.

In one embodiment, the calcium oxalate tissue deposition disease, disorder, or condition is selected from the group consisting of systemic calcium oxalate tissue deposition disease, disorder, or condition or tissue specific calcium oxalate tissue deposition disease, disorder, or condition.

In one embodiment, the lactate dehydrogenase-associated disease, disorder, or condition is selected from the group consisting of cancer, fatty liver (steatosis), nonalcoholic steatohepatitis (NASH), cirrhosis of the liver, accumulation of fat in the liver, inflammation of the liver, hepatocellular necrosis, liver fibrosis, and nonalcoholic fatty liver disease (NAFLD). In one embodiment, the disease, disorder or condition is primary hyperoxaluria 2 (PH2).

In one embodiment, the method further comprises altering the diet of the subject (e.g., decreasing protein intake, decreasing sodium intake, decreasing ascorbic acid intake, moderatating calcium intake, supplementing phosphate, supplementing magnesium, and pyridoxine treatment; and a combination of any of the foregoing).

In one embodiment, the subject further receives a kidney transplant.

In one embodiment, the subject is human.

In one embodiment, the methods further include administering an additional therapeutic to the subject.

In one embodiment, the RNAi agent is administered to the subject at a dose of about 0.01 mg/kg to about 10 mg/kg or about 0.5 mg/kg to about 50 mg/kg.

In one embodiment, the agent is administered to the subject subcutaneously.

In one embodiment, the agent does not substantially inhibit expression and/or activity of lactate dehydrogenase B (LDHB).

### BRIEF DESCRIPTION OF THE DRAWINGS

FIG. 1A is a schematic of the endogenous pathways for oxalate synthesis.

FIG. 1B is a schematic of the metabolic pathways associated with LDHA.

FIG. 2 is a graph showing the level of Ldha mRNA remaining in wild-type C57BL/6J mice at 10 days post-dose of a single 0.1 mg/kg, 0.3 mg/kg, 1.0 mg/kg, 3.0 mg/kg, or 10 mg/kg dose of AD-84788.

FIG. 3 is a graph showing hepatic LDHA activity in adult male Agxt knockout mice 4 weeks after subcutaneous administration of a single 0.3 mg/kg, 1 mg/kg, 3 mg/kg, or 10 mg/kg dose of AD-84788. Agxt knockout mice administered 0 mg/kg of AD-84788 served as untreated controls.

FIG. 4 is a schematic of the study protocol described in Example 3 and referred to in FIGS. 6-17B.

FIG. 5 is a graph showing the amount of urinary oxalate (mg per g of creatinine) excreted by Agxt knockout mice over a twenty-four hour period at weeks 0, 1, 2, 3, 4, 6, 8, 9, and 10 following subcutaneous administration of a single 0.3 mg/kg, 1 mg/kg, 3 mg/kg, or 10 mg/kg dose of AD-84788. Agxt knockout mice administered 0 mg/kg of AD-84788 served as untreated controls.

FIG. 6 is a graph showing the amount of oxalate (mg per g of creatinine) excreted in the urine of Agxt knockout mice, wild-type mice, and Grhpr (glyoxylate reductase/hydroxypyruvate reductase) knockout mice 4 weeks after a single 10 mg/kg dose of AD-84788.

FIG. 7 is a graph showing the amount of oxalate (mg per g of creatinine) excreted in the urine of Agxt deficient mice administered the dsRNA agent AD-84788 at Day 0 pre-dose (baseline, i.e., at days −6, −5, −4, and −3); at days 7-10 after a single 10 mg/kg dose of AD-84788; and at days 28-31 following the last administration of four 10/mg/kg doses of AD-84788 on days 0, 11, 18, and 25 (see, FIG. 4).

FIG. 8A is a graph showing the enzymatic activity of LdhA in wild-type liver homogenates of untreated control mice and mice administered four 10 mg/kg doses of AD-84788 (see, FIG. 4) using lactic acid as a substrate. Absorbance increases as NAD is reduced to NADH via LDH enzymatic activity. The initial linear range was selected, and absorbances at 1 and 6 minutes were utilized in specific activity calculations as Δ_{abs} across a Δₜᵢₘₑ of 5 minutes.

FIG. 8B is a graph showing the mean specific activity of LdhA in wild-type liver homogenates of untreated control mice and mice administered four 10 mg/kg doses of AD-84788 (see, FIG. 4) using lactic acid as a substrate. Specific activity is expressed as μmol NADH formed/min/g protein. Calculations were performed for all animals individually, and a t-test was conducted comparing all specific activity data from both treatment groups. Mean specific activity of both treatment groups is presented. (p<0.001).

FIG. 9A is a graph showing the enzymatic activity of LdhA in wild-type liver homogenates of untreated control mice and mice administered four 10 mg/kg doses of AD-84788 (see, FIG. 4) using glyoxylate as a substrate. Absorbance increases as NAD is reduced to NADH via LDH enzymatic activity. The initial linear range was selected, and absorbances at 0 and 4 minutes were utilized in specific activity calculations as Δ_{abs} across a Δₜᵢₘₑ of 4 minutes.

FIG. 9B is a graph showing the mean specific activity of LdhA in wild-type liver homogenates of untreated control mice and mice administered four 10 mg/kg doses of AD-84788 (see, FIG. 4) using glyoxylate as a substrate. Specific activity is expressed as μmol NADH formed/min/g protein. Calculations were performed for all animals individually, and a t-test was conducted comparing all specific activity data from both treatment groups. Mean specific activity of both treatment groups is presented. (p<0.001).

FIG. 10A is a graph showing the enzymatic activity of LdhA in Agxt deficient liver homogenates of untreated control mice and mice administered four 10 mg/kg doses of AD-84788 (see, FIG. 4) using lactic acid as a substrate. Absorbance increases as NAD is reduced to NADH via LDH enzymatic activity. The initial linear range was selected, and absorbances at 0 and 4 minutes were utilized in specific activity calculations as Δ_{abs} across a Δₜᵢₘₑ of 4 minutes. SD is too small to be visualized in the mean treated group.

FIG. 10B is a graph showing the mean specific activity of LdhA in Agxt deficient liver homogenates of untreated control mice and mice administered four 10 mg/kg doses of AD-84788 (see, FIG. 4) using lactic acid as a substrate. Specific activity is expressed as μmol NADH formed/min/g protein. Calculations were performed for all animals individually, and a t-test was conducted comparing all specific activity data from both treatment groups. Mean specific activity of both treatment groups is presented. (p<0.001).

FIG. 11A is a graph showing the enzymatic activity of LdhA in Agxt deficient liver homogenates of untreated control mice and mice administered four 10 mg/kg doses of AD-84788 (see, FIG. 4) using glyoxylate as a substrate. Absorbance increases as NAD is reduced to NADH via LDH enzymatic activity. The initial linear range was selected, and absorbances at 0 and 4 minutes were utilized in specific activity calculations as Δ_{abs} across a Δₜᵢₘₑ of 4 minutes.

FIG. 11B is a graph showing the mean specific activity of LdhA in Agxt deficient liver homogenates of untreated control mice and mice administered four 10 mg/kg doses of AD-84788 (see, FIG. 4) using glyoxylate as a substrate. Specific activity is expressed as μmol NADH formed/min/g protein. Calculations were performed for all animals individually, and a t-test was conducted comparing all specific activity data from both treatment groups. Mean specific activity of both treatment groups is presented. (p<0.001).

FIG. 12A is a graph showing the enzymatic activity of LdhA in wild-type heart homogenates of untreated control mice and mice administered four 10 mg/kg doses of AD-84788 (see, FIG. 4) using lactic acid as a substrate. Absorbance for both the control group and the treatment group increases as NAD is reduced to NADH via LDH enzymatic activity. The initial linear range was selected, and absorbances at 0 and 4 minutes were utilized in specific activity calculations as Δ_{abs} across a Δₜᵢₘₑ of 4 minutes.

FIG. 12B is a graph showing the mean specific activity of LdhA in wild-type heart homogenates of untreated control mice and mice administered four 10 mg/kg doses of AD-84788 (see, FIG. 4) using lactic acid as a substrate. Specific activity is expressed as μmol NADH formed/min/g protein. Calculations were performed for all animals individually, and a t-test was conducted comparing all specific activity data from both treatment groups. Mean specific activity of both treatment groups is presented. There is no significant difference.

FIG. 12C is a graph showing the enzymatic activity of LdhA in wild-type thigh muscle homogenates of untreated control mice and mice administered four 10 mg/kg doses of AD-84788 (see, FIG. 4) using lactic acid as a substrate. Absorbance for both the control group and the treatment group increases as NAD is reduced to NADH via LDH enzymatic activity. The initial linear range was selected, and absorbances at 0 and 4 minutes were utilized in specific activity calculations as Δ_{abs} across a Δₜᵢₘₑ of 4 minutes.

FIG. 12D is a graph showing the mean specific activity of LdhA in wild-type thigh muscle homogenates of untreated control mice and mice administered four 10 mg/kg doses of AD-84788 (see, FIG. 4) using lactic acid as a substrate. Specific activity is expressed as μmol NADH formed/min/g protein. Calculations were performed for all animals individually, and a t-test was conducted comparing all specific activity data from both treatment groups. Mean specific activity of both treatment groups is presented. There is no significant difference.

FIG. 13A is a graph showing the mean amount of lactate in wild-type liver homogenates of wild-type mice prior to the administration of four 10 mg/kg doses of AD-84788 (baseline) and the mean amount of lactate in wild-type liver homogenates of wild-type mice four weeks after the administration of four 10 mg/kg doses of AD-84788 (see, FIG. 4).

FIG. 13B is a graph showing the mean amount of pyruvate in wild-type liver homogenates of wild-type mice prior to the administration of four 10 mg/kg doses of AD-84788 (baseline) and the mean amount of pyruvate in wild-type liver homogenates of wild-type mice four weeks after the administration of four 10 mg/kg doses of AD-84788 (see, FIG. 4).

FIG. 14A is a graph showing the mean amount of lactate in Agxt deficient liver homogenates of Agxt deficient mice prior to the administration of four 10 mg/kg doses of AD-84788 (baseline) and the mean amount of lactate in Agxt deficient liver homogenates of Agxt deficient mice four weeks after the administration of four 10 mg/kg doses of AD-84788 (see, FIG. 4).

FIG. 14B is a graph showing the mean amount of pyruvate in Agxt deficient liver homogenates of Agxt deficient mice prior to the administration of four 10 mg/kg doses of AD-84788 (baseline) and the mean amount of pyruvate in Agxt deficient liver homogenates of Agxt deficient mice four weeks after the administration of four 10 mg/kg doses of AD-84788 (see, FIG. 4)

FIG. 15A is a graph showing the mean amount of glyoxylate in wild-type liver homogenates of wild-type mice prior to the administration of four 10 mg/kg doses of AD-84788 (baseline) and the mean amount of glyoxylate in wild-type liver homogenates of wild-type mice four weeks after the administration of four 10 mg/kg doses of AD-84788 (see, FIG. 4).

FIG. 15B is a graph showing the mean amount of glyoxylate in Agxt deficient liver homogenates of Agxt deficient mice prior to the administration of four 10 mg/kg doses of AD-84788 (baseline) and the mean amount of glyoxylate in Agxt deficient liver homogenates of Agxt deficient mice four weeks after the administration of four 10 mg/kg doses of AD-84788 (see, FIG. 4).

FIG. 16A is a graph showing the mean body weights of wild-type mice prior to the administration of four 10 mg/kg doses of AD-84788 (baseline) and the mean body weights of wild-type mice four weeks after the administration of four 10 mg/kg doses of AD-84788 (see, FIG. 4).

FIG. 16B is a graph showing the mean body weights of Agxt deficient mice prior to the administration of four 10 mg/kg doses of AD-84788 (baseline) and the mean body weights of Agxt deficient mice four weeks after the administration of four 10 mg/kg doses of AD-84788 (see, FIG. 4).

FIG. 17A is a graph showing the mean plasma lactate levels of wild-type mice prior to the administration of four 10 mg/kg doses of AD-84788 (baseline) and the mean plasma lactate levels of wild-type mice four weeks after the administration of four 10 mg/kg doses of AD-84788 (see, FIG. 4).

FIG. 17B is a graph showing the mean plasma lactate levels of Agxt deficient mice prior to the administration of four 10 mg/kg doses of AD-84788 (baseline) and the mean plasma lactate levels of Agxt deficient mice four weeks after the administration of four 10 mg/kg doses of AD-84788 (see, FIG. 4).

FIGS. 18A-18O depict exemplary dual targeting agents of the invention.

FIG. 18A depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (S) and a second antisense strand, wherein the 3′end of the first sense strand is covalently attached to the 5′ end of the second sense strand with a nucleotide linker comprising 2′OMe modified nucleotides (uuu), wherein the 3′ end of the second sense strand comprises a GalNAc ligand, and wherein the two 5′-most nucleotides of the first sense strand each independently comprise a phosphorothioate linkage.

FIG. 18B depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (S) and a second antisense strand (AS). wherein the 3′end of the first sense strand is covalently attached to the 5′ end of the second sense strand with a nucleotide linker comprising 2′ Fluoro modified nucleotides (GfAfAf), wherein the 3′ end of the second sense strand comprises a GalNAc ligand, and wherein the two 5′-most nucleotides of the first sense strand, the 3′-most nucleotide of the first sense strand, and the 5′-most nucleotide of the second sense strand each independently comprise a phosphorothioate linkage.

FIG. 18C depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (S) and a second antisense strand (AS), wherein the 3′end of the first sense strand is covalently attached to the 5′ end of the second sense strand with a nucleotide linker comprising 2′Fluoro modified nucleotides (GfAffUf), wherein the 3′ end of the second sense strand comprises a GalNAc ligand, and wherein the two 5′-most nucleotides of the first sense strand, the 3′-most nucleotide of the first sense strand, and the 5′-most nucleotide of the second sense strand each independently comprise a phosphorothioate linkage.

FIG. 18D depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (S) and a second antisense strand (AS), wherein the 3′end of the first sense strand is covalently attached to the 5′ end of the second sense strand with a nucleotide linker comprising deoxynucleotides (dgdada), wherein the 3′ end of the second sense strand comprises a GalNAc ligand, and wherein the two 5′-most nucleotides of the first sense strand, the 3′-most nucleotide of the first sense strand, and the 5′-most nucleotide of the second sense strand each independently comprise a phosphorothioate linkage.

FIG. 18E depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (5) and a second antisense strand (AS), wherein the 3′end of the first sense strand is covalently attached to the 5′ end of the second sense strand with a nucleotide linker comprising deoxynucleotides (dgda), wherein the 3′ end of the second sense strand comprises a GalNAc ligand, and wherein the two 5′-most nucleotides of the first sense strand, the 3′-most nucleotide of the first sense strand, and the 5′-most nucleotide of the second sense strand each independently comprise a phosphorothioate linkage.

FIG. 18F depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (S) and a second antisense strand (AS), and the 3′end of the first sense strand is directly attached (no linker) to the 5′ end of the second sense strand, wherein the two 5′-most nucleotides of the first sense strand and the two 3′-most nucleotides of the second sense strand each independently comprise a phosphorothioate linkage, and wherein the 3′ end of the first sense strand comprises a GalNAc ligand.

FIG. 18G depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (S) and a second antisense strand (AS), wherein the 5′end of the first antisense strand is covalently attached to the 3′ end of the second antisense strand with a nucleotide linker comprising 2′OMe modified nucleotides (acu), wherein the 3′ end of the second sense strand comprises a GalNAc ligand, and wherein the two 3′-most nucleotides of the first antisense strand and the two 5′-most nucleotides of the second antisense strand each independently comprise a phosphorothioate linkage.

FIG. 18H depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (5) and a second antisense strand (AS), wherein the 5′end of the first antisense strand is covalently attached to the 3′ end of the second antisense strand with a nucleotide linker comprising 2′Flouro modified nucleotides (AfAfGf), wherein the 3′ end of the second sense strand comprises a GalNAc ligand, and wherein the two 3′-most nucleotides of the first antisense strand, the 5′ nucleotide of the first antisense strand, the 3′ nucleotide of the second antisense strand, and the two 5′-most nucleotides of the second antisense strand each independently comprise a phosphorothioate linkage.

FIG. 18I depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (S) and a second antisense strand (AS), wherein the 5′end of the first antisense strand is directly attached (no linker) to the 3′ end of the second antisense strand, wherein the 3′ end of the second sense strand comprises a GalNAc ligand, and wherein the two 3′-most nucleotides of the first antisense strand and the two 5′-most nucleotides of the second antisense strand each independently comprise a phosphorothioate linkage.

FIG. 18J depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (5) and a second antisense strand (AS), wherein the 3′end of the first sense strand is covalently attached to the 5′ end of the second sense strand with a nucleotide linker comprising 2′ OMe modified nucleotides (uuu), wherein the 5′ end of the first sense strand and the 3′ end of the second sense strand each independently comprise a GAlNAc ligand, and wherein the 5′ nucleotide of the first sense strand comprises a phosphorothioate linkage.

FIG. 18K depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (S) and a second antisense strand (AS), wherein the 3′end of the first sense strand is covalently attached to the 5′ end of the second sense strand with a nucleotide linker comprising 2′Fluoro modified nucleotides (GfAfAt), wherein the 5′ end of the first sense strand and the 3′ end of the second sense strand each independently comprise a GalNAc ligand, and wherein the 5′ nucleotide of the first sense strand, the 3′ nucleotide of the first sense strand, and the 5′ nucleotide of the second sense strand each independently comprise a phosphorothioate linkage.

FIG. 18L depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (S) and a second antisense strand (AS), wherein the 3′end of the first sense strand is directly attached (no linker) to the 5′ end of the second sense strand, wherein the 3′ end of the first sense strand and the 3′ end of the second sense strand each independently comprise a GalNAc ligand, and wherein the two 5′-most nucleotides of the first sense strand each independently comprise a phosphorothioate linkage.

FIG. 18M depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (S) and a second antisense strand (AS), wherein the 5′end of the first antisense strand is covalently attached to the 3′ end of the second antisense strand with a nucleotide linker comprising 2′-O-Me modified nucleotides (acu), wherein the 3′ end of the first antisense strand and the 3′ end of the second sense strand each independently comprise a GalNAc ligand, and wherein the two most 5′ nucleotides of the second antisense strand each independently comprise a phosphorothioate linkage.

FIG. 18N depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (S) and a second antisense strand (AS), wherein the 5′end of the first antisense strand is covalently attached to the 3′ end of the second antisense strand with a nucleotide linker comprising 2′Fluoro modified nucleotides (AfAfGf), wherein the 3′ end of the first antisense strand and the 3′ end of the second sense strand each independently comprise a GalNAc ligand, and wherein the 5′ nucleotide of the first antisense strand, the 3′ nucleotide of the second antisense strand, and the two 5′-most nucleotides of the second antisense strand each independently comprise a phosphorothioate linkage.

FIG. 18O depicts an exemplary dual targeting agent of the invention comprising a first dsRNA agent targeting LDHA and a second dsRNA agent targeting HAO1, wherein the first dsRNA agent comprises a first sense strand (S) and a first antisense strand (AS), wherein the second dsRNA agent comprises a second sense strand (S) and a second antisense strand (AS), wherein the 5′end of the first antisense strand is directly attached (no linker) to the 3′ end of the second antisense strand, wherein the 3′ end of the first antisense strand and the 3′ end of the second sense strand each independently comprise a GalNAc ligand, and wherein the two most 5′ nucleotides of the second antisense strand each independently comprise a phosphorothioate linkage.

### DETAILED DESCRIPTION OF THE INVENTION

The present invention provides iRNA compositions, which effect the RNA-induced silencing complex (RISC)-mediated cleavage of RNA transcripts of an LDHA gene. The LDHA gene may be within a cell, e.g., a cell within a subject, such as a human. The present invention also provides methods of using the iRNA compositions of the invention for inhibiting the expression of an LDHA gene, and for treating a subject who would benefit from inhibiting or reducing the expression of an LDHA gene, e.g., a subject that would benefit from a reduction or inhibition in urinary oxalate production, e.g., a subject suffering or prone to suffering from an oxalate pathway-associated disease disorder, or condition, such as a subject suffering or prone to suffering from an oxalate-associated disease, disorder, or condition, e.g., a kidney stone formation disease, disorder, or condition or a calcium oxalate tissue deposition disease, disorder, or condition; or an LDH-associated disease, disorder, or condition.

The present invention also provides methods of using the iRNA compositions of the invention for inhibiting the expression of an LDHA gene and an HAO1 gene for treating a subject who would benefit from inhibiting or reducing the expression of an LDHA gene and an HAO1 gene, e.g., a subject that would benefit from a reduction or inhibition in urinary oxalate production, e.g., a subject suffering or prone to suffering from an oxalate pathway-associated disease disorder, or condition, such as a subject suffering or prone to suffering from an oxalate-associated disease, disorder, or condition, e.g., a kidney stone formation disease, disorder, or condition or a calcium oxalate tissue deposition disease, disorder, or condition; or an LDH-associated disease, disorder, or condition.

The iRNAs of the invention targeting LDHA may include an RNA strand (the antisense strand) having a region which is about 30 nucleotides or less in length, e.g., 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, which region is substantially complementary to at least part of an mRNA transcript of an LDHA gene.

The iRNAs of the invention targeting HAO1 may include an RNA strand (the antisense strand) having a region which is about 30 nucleotides or less in length, e.g., 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, which region is substantially complementary to at least part of an mRNA transcript of an HAO1 gene.

When the RNAi agent is a dual targeting RNAi agent, as described herein, the agent targeting LDHA may include an antisense strand comprising a region of complementarity to LDHA which is the same length or a different length from the region of complementarity of the antisense strand of the agent targeting HAO1.

In some embodiments, one or both of the strands of the double stranded RNAi agents of the invention is up to 66 nucleotides in length, e.g., 36-66, 26-36, 25-36, 31-60, 22-43, 27-53 nucleotides in length, with a region of at least 19 contiguous nucleotides that is substantially complementary to at least a part of an mRNA transcript of an LDHA gene. In some embodiments, such iRNA agents having longer length antisense strands may include a second RNA strand (the sense strand) of 20-60 nucleotides in length wherein the sense and antisense strands form a duplex of 18-30 contiguous nucleotides.

In other embodiments, one or both of the strands of the double stranded RNAi agents of the invention is up to 66 nucleotides in length, e.g., 36-66, 26-36, 25-36, 31-60, 22-43, 27-53 nucleotides in length, with a region of at least 19 contiguous nucleotides that is substantially complementary to at least a part of an mRNA transcript of an HAO1 gene. In some embodiments, such iRNA agents having longer length antisense strands may include a second RNA strand (the sense strand) of 20-60 nucleotides in length wherein the sense and antisense strands form a duplex of 18-30 contiguous nucleotides.

In embodiments in which a first dsRNA agent targeting LDHA and a second dsRNA agent targeting HAO1 are covalently attached, the duplex lengths of the first agent and the second agent may be the same or different.

The use of these iRNA agents described herein enables the targeted degradation of mRNAs of an LDHA gene in mammals or the targeted degradation of an LDHA gene and an HAO1 gene in mammals.

Very low dosages of the iRNAs, in particular, can specifically and efficiently mediate RNA interference (RNAi), resulting in significant inhibition of expression of an LDHA gene or an LDHA gene and an HAO1 gene. Using cell-based and in vivo assays, the present inventors have demonstrated that iRNAs targeting LDHA can mediate RNAi, resulting in significant inhibition of expression of an LDHA gene and significant inhibition of oxalate production. Thus, methods and compositions including these iRNAs are useful for treating a subject who would benefit by a reduction or inhibition in LDHA expression or LDHA expression and HAO1 expression, e.g., a subject suffering or prone to suffering from an oxalate pathway-associated disease, disorder, or condition.

The following detailed description discloses how to make and use compositions containing iRNAs to inhibit the expression of an LDHA gene, an HAO1 gene, and both an LDHA gene and an HAO1 gene, as well as compositions and methods for treating subjects having diseases and disorders that would benefit from inhibition and/or reduction of the expression of these genes.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”. The term “or” is used herein to mean, and is used interchangeably with, the term “and/or,” unless context clearly indicates otherwise.

The term “LDHA” (used interchangeable herein with the term “Ldha”), also known as Cell Proliferation-Inducing Gene 19 Protein, Renal Carcinoma Antigen NY-REN-59, LDH Muscle Subunit, EC 1.1.1.27 4 61, LDH-A, LDH-M, Epididymis Secretory Sperm Binding Protein Li 133P, L-Lactate Dehydrogenase A Chain, Proliferation-Inducing Gene 19, Lactate Dehydrogenase M, HEL-S-133P, EC 1.1.1, GSD11, PIG19, and LDHM, refers to the well known gene encoding a lactate dehydrogenase A from any vertebrate or mammalian source, including, but not limited to, human, bovine, chicken, rodent, mouse, rat, porcine, ovine, primate, monkey, and guinea pig, unless specified otherwise.

The term also refers to fragments and variants of native LDHA that maintain at least one in vivo or in vitro activity of a native LDHA. The term encompasses full-length unprocessed precursor forms of LDHA as well as mature forms resulting from post-translational cleavage of the signal peptide and forms resulting from proteolytic processing.

The sequence of a human LDHA mRNA transcript can be found at, for example, GenBank Accession No. GI: 207028493 (NM_001135239.1; SEQ ID NO:1), GenBank Accession No. GI: 260099722 (NM_001165414.1; SEQ ID NO:3), GenBank Accession No. GI: 260099724 (NM_001165415.1; SEQ ID NO:5), GenBank Accession No. GI: 260099726 (NM_001165416.1; SEQ ID NO:7), GenBank Accession No. GI: 207028465 (NM_005566.3; SEQ ID NO:9); the sequence of a mouse LDHA mRNA transcript can be found at, for example, GenBank Accession No. GI: 257743038 (NM_001136069.2; SEQ ID NO:11), GenBank Accession No. GI: 257743036(NM_010699.2; SEQ ID NO:13); the sequence of a rat LDHA mRNA transcript can be found at, for example, GenBank Accession No. GI: 8393705 (NM_017025.1; SEQ ID NO:15); and the sequence of a monkey LDHA mRNA transcript can be found at, for example, GenBank Accession No. GI: 402766306 (NM_001257735.2; SEQ ID NO:17), GenBank Accession No. GI: 545687102 (NM_001283551.1; SEQ ID NO:19).

Additional examples of LDHA mRNA sequences are readily available using publicly available databases, e.g., GenBank, UniProt, and OMIM.

The term“LDHA” as used herein also refers to a particular polypeptide expressed in a cell by naturally occurring DNA sequence variations of the LDHA gene, such as a single nucleotide polymorphism in the LDHA gene. Numerous SNPs within the LDHA gene have been identified and may be found at, for example, NCBI dbSNP (see, e.g., www.ncbi.nlm nih.gov/snp).

As used herein, the term “HAO1” refers to the well known gene encoding the enzyme hydroxyacid oxidase 1 from any vertebrate or mammalian source, including, but not limited to, human, bovine, chicken, rodent, mouse, rat, porcine, ovine, primate, monkey, and guinea pig, unless specified otherwise. Other gene names include GO, GOX, GOX1, HAO, and HAOX1. The protein is also known as glycolate oxidase and (S)-2-hydroxy-acid oxidase.

The term also refers to fragments and variants of native HAO1 that maintain at least one in vivo or in vitro activity of a native HAO1. The term encompasses full-length unprocessed precursor forms of HAO1 as well as mature forms resulting from post-translational cleavage of the signal peptide and forms resulting from proteolytic processing. The sequence of a human HAO1 mRNA transcript can be found at, for example, GenBank Accession No. GI:11184232 (NM_017545.2; SEQ ID NO:21); the sequence of a monkey HAO1 mRNA transcript can be found at, for example, GenBank Accession No. GI:544464345 (XM_005568381.1; SEQ I DNO:23); the sequence of a mouse HAO1 mRNA transcript can be found at, for example, GenBank Accession No. GI:133893166 (NM_010403.2; SEQ ID NO:25); and the sequence of a rat HAO1 mRNA transcript can be found at, for example, GenBank Accession No. GI: 166157785 (NM_001107780.2; SEQ ID NO:27).

The term“HAO1,” as used herein, also refers to naturally occurring DNA sequence variations of the HAO1 gene, such as a single nucleotide polymorphism (SNP) in the HAO1 gene. Exemplary SNPs may be found in the NCBI dbSNP Short Genetic Variations database available at www.ncbi.nlm.nih.gov/projects/SNP.

As used herein, “target sequence” refers to a contiguous portion of the nucleotide sequence of an mRNA molecule formed during the transcription of an LDHA gene or an HAO1 gene, including mRNA that is a product of RNA processing of a primary transcription product. In one embodment, the target portion of the sequence will be at least long enough to serve as a substrate for iRNA-directed cleavage at or near that portion of the nucleotide sequence of an mRNA molecule formed during the transcription of an LDHA gene. In another embodment, the target portion of the sequence will be at least long enough to serve as a substrate for iRNA-directed cleavage at or near that portion of the nucleotide sequence of an mRNA molecule formed during the transcription of an HAO1 gene.

The target sequence of an LDHA gene may be from about 9-36 nucleotides in length, e.g., about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the invention.

The target sequence of an HAO1 gene may be from about 9-36 nucleotides in length, e.g., about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the invention.

In aspects in which a first dsRNA agent targeting LDHA and a second dsRNA agent targeting HAO1 are covalently attached (i.e., a dual targeting RNAi agent), the length of the LDHA target sequence may be the same as the HAO1 target sequence or different.

As used herein, the term “strand comprising a sequence” refers to an oligonucleotide comprising a chain of nucleotides that is described by the sequence referred to using the standard nucleotide nomenclature.

“G,” “C,” “A,” “T” and “U” each generally stand for a nucleotide that contains guanine, cytosine, adenine, thymidine and uracil as a base, respectively. However, it will be understood that the term “ribonucleotide” or “nucleotide” can also refer to a modified nucleotide, as further detailed below, or a surrogate replacement moiety (see, e.g., Table 1). The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

The terms “iRNA”, “RNAi agent,” “iRNA agent,”, “RNA interference agent” as used interchangeably herein, refer to an agent that contains RNA as that term is defined herein, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of LDHA and/or HAO1 gene in a cell, e.g., a cell within a subject, such as a mammalian subject.

In one embodiment, an RNAi agent of the invention includes a single stranded RNA that interacts with a target RNA sequence, e.g., an LDHA target mRNA sequence and/or an HAO1 target mRNA seuqnce, to direct the cleavage of the target RNA. Without wishing to be bound by theory it is believed that long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the invention relates to a single stranded RNA (sssiRNA) generated within a cell and which promotes the formation of a RISC complex to effect silencing of the target gene, i.e., an LDHA gene and/or an HAO1 gene. Accordingly, the term “siRNA” is also used herein to refer to an RNAi as described above.

In another embodiment, the RNAi agent may be a single-stranded RNAi agent that is introduced into a cell or organism to inhibit a target mRNA. Single-stranded RNAi agents (ssRNAi) bind to the RISC endonuclease, Argonaute 2, which then cleaves the target mRNA. The single-stranded siRNAs are generally 15-30 nucleotides and are chemically modified. The design and testing of single-stranded RNAi agents are described in U.S. Pat. No. 8,101,348 and in Lima et al., (2012) Cell 150: 883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In another embodiment, an “iRNA” for use in the compositions and methods of the invention is a double-stranded RNA and is referred to herein as a “double stranded RNAi agent,” “double-stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, i.e., an LDHA gene and/or an HAO1 gene. In some embodiments of the invention, a double-stranded RNA (dsRNA) triggers the degradation of a target RNA, e.g., an mRNA, through a post-transcriptional gene-silencing mechanism referred to herein as RNA interference or RNAi.

In yet another embodiment, an “iRNA” for use in the compositions and methods of the invention is a “dual targeting RNAi agent.” The term “dual targeting RNAi agent” refers to a molecule comprising a first dsRNA agent comprising a complex of ribonucleic acid molecules, having a duplex structure comprising two anti-parallel and substantially complementary nucleic acid strands, referred to as having “sense” and “antisense” orientations with respect to a first target RNA, i.e., an LDHA gene, covalently attached to a molecule comprising a second dsRNA agent comprising a complex of ribonucleic acid molecules, having a duplex structure comprising two anti-parallel and substantially complementary nucleic acid strands, referred to as having “sense” and “antisense” orientations with respect to a second target RNA, i.e., an HAO1 gene. In some embodiments of the invention, a dual targeting RNAi agent triggers the degradation of the first and the second target RNAs, e.g., mRNAs, through a post-transcriptional gene-silencing mechanism referred to herein as RNA interference or RNAi.

In general, the majority of nucleotides of each strand of a dsRNA molecule are ribonucleotides, but as described in detail herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. As used herein, the term “modified nucleotide” refers to a nucleotide having, independently, a modified sugar moiety, a modified internucleotide linkage, and/or a modified nucleobase. Thus, the term modified nucleotide encompasses substitutions, additions or removal of, e.g., a functional group or atom, to internucleoside linkages, sugar moieties, or nucleobases. The modifications suitable for use in the agents of the invention include all types of modifications disclosed herein or known in the art. Any such modifications, as used in a siRNA type molecule, are encompassed by “RNAi agent” for the purposes of this specification and claims.

The duplex region may be of any length that permits specific degradation of a desired target RNA through a RISC pathway, and may range from about 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

In embodiments in which a first dsRNA agent targeting LDHA and a second dsRNA agent targeting HAO1 are covalently attached (i.e., a dual targeting RNAi agent), the length of the duplex region of the first agent and the second agent may be the same or different.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 2, at least 3, at least 4, at least 5, at least 6, at least 7, at least 8, at least 9, at least 10, at least 20, at least 23 or more unpaired nucleotides.

In embodiments in which a first dsRNA agent targeting LDHA and a second dsRNA agent targeting HAO1 are covalently attached (i.e., a dual targeting RNAi agent), the first dsRNA agent may comprise a harpin loop, the second dsRNA agent may comprise a hairpin loop, or both the first and the second dsRNA agents may independently comprise a hairpin loop.

In addition, in embodiments in which a first dsRNA agent targeting LDHA and a second dsRNA agent targeting HAO1 are covalently attached (i.e., a dual targeting RNAi agent), the first dsRNA agent may comprise unpaired nucleotides, the second dsRNA agent may comprise unpaired nucleotides, or both the first and the second dsRNA agents may independently comprise unpaired nucleotides. When both the first and the second dsRNA agents independently comprise unpaired nucleotides, the first dsRNA agent and the second dsRNA agent may comprise the same or a different number of unpaired nucleotides.

Where the two substantially complementary strands of a dsRNA are comprised by separate RNA molecules, those molecules need not, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs.

In one embodiment, an RNAi agent of the invention is a dsRNA, each strand of which comprises 19-23 nucleotides, that interacts with a target RNA sequence, e.g., an LDHA target mRNA sequence, to direct the cleavage of the target RNA. In another embodiment, an RNAi agent of the invention is a dsRNA, each strand of which comprises 19-23 nucleotides, that interacts with a target RNA sequence, e.g., an HAO1 target mRNA sequence, to direct the cleavage of the target RNA. In yet other embodiments an RNAi agent of the invention comprises a first dsRNA agent, each strand of which comprises 19-23 nucleotides, that interacts with a target RNA sequence, e.g., an LDHA target mRNA sequence, to direct the cleavage of the target RNA, and a second dsRNA agent, each strand of which independently comprises 19-23 nucleotides, that interacts with a target RNA sequence, e.g., an HAO1 target mRNA sequence, to direct the cleavage of the target RNA, wherein the first and second dsRNA agents are covalently attached.

In embodiments in which a first dsRNA agent targeting LDHA and a second dsRNA agent targeting HAO1 are covalently attached (i.e., a dual targeting RNAi agent), the two strands of the first dsRNA agent may be connected covalently by means other than an uninterrupted chain of nucleotides between the 3′-end of one strand and the 5′-end of the respective other strand forming the duplex structure, the two strands of the second dsRNA agent may be connected covalently by means other than an uninterrupted chain of nucleotides between the 3′-end of one strand and the 5′-end of the respective other strand forming the duplex structure, or the two strands of the first dsRNA agent and the two strands of the second dsRNA agent may independently be connected covalently by means other than an uninterrupted chain of nucleotides between the 3′-end of one strand and the 5′-end of the respective other strand forming the duplex structure.

As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

In embodiments in which a first dsRNA agent targeting LDHA and a second dsRNA agent targeting HAO1 are covalently attached (i.e., a dual targeting RNAi agent), the first agent may comprise a nucleotide overhang, the second agent may comprise a nucleotide overhang, or both the first and the second agent may independently comprise a nucleotide overhang, e.g., the 5′ end of the sense strand of the first agent may comprise an overhang, the 3′ end of the sense strand of the first agent may comprise an overhang, the 5′ end of the antisense strand of the first agent may comprise an overhang, the 3′ end of the antisense strand of the first agent may comprise an overhang, the 5′ end and the 3′ end of the sense stand of the first agent may comprise an overhang, the 5′ end and the 3′ end of the antisense stand of the first agent may comprise an overhang, the 5′ end of the sense strand of the second agent may comprise an overhang, the 3′ end of the sense strand of the second agent may comprise an overhang, the 5′ end of the antisense strand of the second agent may comprise an overhang, the 3′ end of the antisense strand of the second agent may comprise an overhang, the 5′ end and the 3′ end of the sense stand of the second agent may comprise an overhang, the 5′ end and the 3′ end of the antisense stand of the second agent may comprise an overhang, or any combination of the foregoing.

In embodiments in which a first dsRNA agent targeting LDHA and a second dsRNA agent targeting HAO1 are covalently attached (i.e., a dual targeting RNAi agent), the length of an overhang of the first agent and the second agent may be the same or different.

In one embodiment, the antisense strand of a dsRNA has a 1-10 nucleotide, e.g., a 1, 2, 3, 4, 5, 6, 7, 8, 9, or 10 nucleotide, overhang at the 3′-end and/or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end and/or the 5′-end. In another embodiment, one or more of the nucleotides in the overhang is replaced with a nucleoside thiophosphate.

In certain embodiments, the overhang on the sense strand or the antisense strand, or both, can include extended lengths longer than 10 nucleotides, e.g., 10-30 nucleotides, 10-25 nucleotides, 10-20 nucleotides or 10-15 nucleotides in length. In certain embodiments, an extended overhang is on the sense strand of the duplex. In certain embodiments, an extended overhang is present on the 3′end of the sense strand of the duplex. In certain embodiments, an extended overhang is present on the 5′end of the sense strand of the duplex. In certain embodiments, an extended overhang is on the antisense strand of the duplex. In certain embodiments, an extended overhang is present on the 3′end of the antisense strand of the duplex. In certain embodiments, an extended overhang is present on the 5′end of the antisense strand of the duplex. In certain embodiments, one or more of the nucleotides in the extended overhang is replaced with a nucleoside thiophosphate.

In embodiments in which a first dsRNA agent targeting LDHA and a second dsRNA agent targeting HAO1 are covalently attached (i.e., a dual targeting RNAi agent), and one and/or both strands of both the first and the second dsRNA agent independently comprise an overhang, e.g., an extended overhang, the length of the overhang may be the same or different, and/or, in some embodiments, one or more of the nucleotides in the overhang in the first dsRNA agent and one or more nucleotides in the overhang of the second dsRNA agent may be independently replaced with a nucleoside thiophosphate.

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double-stranded over its entire length.

In embodiments in which a first dsRNA agent targeting LDHA and a second dsRNA agent targeting HAO1 are covalently attached (i.e., a dual targetingRNAi agent), one or both of the dsRNA agents may independently comprise a blunt end.

The term “antisense strand” or “guide strand” refers to the strand of an iRNA, e.g., a dsRNA, which includes a region that is substantially complementary to a target sequence, e.g., an LDHA mRNA or an HAO1 mRNA.

As used herein, the term “region of complementarity” refers to the region on the antisense strand that is substantially complementary to a sequence, for example a target sequence, e.g., an LDHA nucleotide sequence or an HAO1 nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the iRNA.

In embodiments in which a first dsRNA agent targeting LDHA and a second dsRNA agent targeting HAO1 are covalently attached (i.e., a dual targetingRNAi agent), one or both of the dsRNA agents may independently comprise a mismatch.

The term “sense strand” or “passenger strand” as used herein, refers to the strand of an iRNA that includes a region that is substantially complementary to a region of the antisense strand as that term is defined herein.

As used herein, the term “cleavage region” refers to a region that is located immediately adjacent to the cleavage site. The cleavage site is the site on the target at which cleavage occurs.

In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs and/or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary,” “fully complementary” and “substantially complementary” herein can be used with respect to the base matching between the sense strand and the antisense strand of a dsRNA, or between the antisense strand of an iRNA agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding LDHA or an mRNA encoding HAO1). For example, a polynucleotide is complementary to at least a part of an LDHA mRNA if the sequence is substantially complementary to a non-interrupted portion of an mRNA encoding LDHA.

Accordingly, in some embodiments, the antisense strand polynucleotides disclosed herein are fully complementary to the target LDHA sequence. In other embodiments, the antisense strand polynucleotides disclosed herein are substantially complementary to the target LDHA sequence and comprise a contiguous nucleotide sequence which is at least about 80% complementary over its entire length to the equivalent region of the nucleotide sequence of SEQ ID NO:1, or a fragment of SEQ ID NO:1, such as about 85%, about 86%, about 87%, about 88%, about 89%, about 90%, about % 91%, about 92%, about 93%, about 94%, about 95%, about 96%, about 97%, about 98%, or about 99% complementary.

In one embodiment, an RNAi agent of the invention includes a sense strand that is substantially complementary to an antisense polynucleotide which, in turn, is complementary to a target LDHA sequence, and wherein the sense strand polynucleotide comprises a contiguous nucleotide sequence which is at least about 80% complementary over its entire length to the equivalent region of the nucleotide sequence of SEQ ID NO:2, or a fragment of any one of SEQ ID NO:2, such as about 85%, about 86%, about 87%, about 88%, about 89%, about 90%, about 91%, about 92%, about 93%, about 94%, about 95%, about 96%, about 97%, about 98%, or about 99% complementary.

In some embodiments, an iRNA of the invention includes an antisense strand that is substantially complementary to the target LDHA sequence and comprises a contiguous nucleotide sequence which is at least about 80% complementary over its entire length to the equivalent region of the nucleotide sequence of any one of the sense strands in any one of Tables 2-5, or a fragment of any one of the sense strands in any one of Tables 2-5, such as about 85%, 86%, 87%, 88%, 89%, 90%, 91%, 92%, 93%, 94%, 95%, 96%, 97%, 98%, or 99% complementary, or 100% complementary.

Accordingly, in some embodiments, the antisense strand polynucleotides disclosed herein are fully complementary to the target HAO1 sequence. In other embodiments, the antisense strand polynucleotides disclosed herein are substantially complementary to the target HAO1 sequence and comprise a contiguous nucleotide sequence which is at least about 80% complementary over its entire length to the equivalent region of the nucleotide sequence of SEQ ID NO:21, or a fragment of SEQ ID NO:21, such as about 85%, about 86%, about 87%, about 88%, about 89%, about 90%, about % 91%, about 92%, about 93%, about 94%, about 95%, about 96%, about 97%, about 98%, or about 99% complementary.

In one embodiment, an RNAi agent of the invention includes a sense strand that is substantially complementary to an antisense polynucleotide which, in turn, is complementary to a target HAO1 sequence, and wherein the sense strand polynucleotide comprises a contiguous nucleotide sequence which is at least about 80% complementary over its entire length to the equivalent region of the nucleotide sequence of SEQ ID NO:22, or a fragment of any one of SEQ ID NO:22, such as about 85%, about 86%, about 87%, about 88%, about 89%, about 90%, about % 91%, about 92%, about 93%, about 94%, about 95%, about 96%, about 97%, about 98%, or about 99% complementary.

In some embodiments, an iRNA of the invention includes an antisense strand that is substantially complementary to the target HAO1 sequence and comprises a contiguous nucleotide sequence which is at least about 80% complementary over its entire length to the equivalent region of the nucleotide sequence of any one of the sense strands in any one of Tables 7-14, or a fragment of any one of the sense strands in any one of Tables 7-14, such as about about 85%, 86%, 87%, 88%, 89%, 90%, 91%, 92%, 93%, 94%, 95%, 96%, 97%, 98%, or 99% complementary, or 100% complementary.

The term “inhibiting,” as used herein, is used interchangeably with “reducing,” “silencing,” “downregulating,” “suppressing” and other similar terms, and includes any level of inhibition.

The phrase “inhibiting expression of an LDHA gene,” as used herein, includes inhibition of expression of any LDHA gene (such as, e.g., a mouse LDHA gene, a rat LDHA gene, a monkey LDHA gene, or a human LDHA gene) as well as variants or mutants of an LDHA gene that encode an LDHA protein.

“Inhibiting expression of an LDHA gene” includes any level of inhibition of an LDHA gene, e.g., at least partial suppression of the expression of an LDHA gene, such as an inhibition by at least about 20%. In certain embodiments, inhibition is by at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

The phrase “inhibiting expression of an HAO1 gene,” as used herein, includes inhibition of expression of any HAO1 gene (such as, e.g., a mouse HAO1 gene, a rat HAO1 gene, a monkey HAO1 gene, or a human HAO1 gene) as well as variants or mutants of an HAO1 gene that encode an HAO1 protein.

“Inhibiting expression of an HAO1 gene” includes any level of inhibition of an HAO1 gene, e.g., at least partial suppression of the expression of an HAO1 gene, such as an inhibition by at least about 20%. In certain embodiments, inhibition is by at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

In embodiments in which a first dsRNA agent targeting LDHA and a second dsRNA agent targeting HAO1 are covalently attached, the inhibition of expression of LDHA may be the same or different than the inhibition of HAO1 expression.

The expression of an LDHA gene and/or an HAO1 gene may be assessed based on the level of any variable associated with LDHA gene expression and/or HAO1 gene expression, e.g., LDHA and/or HAO1 mRNA level or LDHA and/or HAO1 protein level. The expression of an LDHA gene and/or an HAO1 gene may also be assessed indirectly based on the levels of oxalate or glycolate in a urine, a plasma, or a tissue sample, or the enzymatic activity of LDHA in a tissue sample, such as a liver sample, a skeletal muscle sample, and/or a heart sample. Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In one embodiment, at least partial suppression of the expression of an LDHA gene, is assessed by a reduction of the amount of LDHA mRNA which can be isolated from, or detected, in a first cell or group of cells in which an LDHA gene is transcribed and which has or have been treated such that the expression of an LDHA gene is inhibited, as compared to a second cell or group of cells substantially identical to the first cell or group of cells but which has or have not been so treated (control cells).

In one embodiment, at least partial suppression of the expression of an HAO1 gene, is assessed by a reduction of the amount of HAO1 mRNA which can be isolated from or detected in a first cell or group of cells in which an HAO1 gene is transcribed and which has or have been treated such that the expression of an HAO1 gene is inhibited, as compared to a second cell or group of cells substantially identical to the first cell or group of cells but which has or have not been so treated (control cells).

In one embodiment, at least partial suppression of the expression of an LDHA gene and an HAO1 gene, is assessed by a reduction of the amount of LDHA mRNA and HAO1 mRNA which can be isolated from or detected in a first cell or group of cells in which an LDHA gene and an HAO1 gene are transcribed and which has or have been treated such that the expression of an LDHA gene and an HAO1 gene is inhibited, as compared to a second cell or group of cells substantially identical to the first cell or group of cells but which has or have not been so treated (control cells).

The degree of inhibition may be expressed in terms of:

$$\frac{( mRNA in control cells ) - ( mRNA in treated cells )}{( mRNA in control cells )} \cdot 100 \%$$

The phrase “contacting a cell with an RNAi agent,” such as a dsRNA, as used herein, includes contacting a cell by any possible means. Contacting a cell with an RNAi agent includes contacting a cell in vitro with the iRNA or contacting a cell in vivo with the iRNA. The contacting may be done directly or indirectly. Thus, for example, the RNAi agent may be put into physical contact with the cell by the individual performing the method, or alternatively, the RNAi agent may be put into a situation that will permit or cause it to subsequently come into contact with the cell.

In the methods of the invention in which a first dsRNA agent targeting LDHA and a second dsRNA agent targeting HAO1 are covalently attached (i.e., a dual targetingRNAi agent), contacting a cell may include contacting the cell with the first agent at the same time or at a different time than contacting the cell with the second agent.

Contacting a cell in vitro may be done, for example, by incubating the cell with the RNAi agent. Contacting a cell in vivo may be done, for example, by injecting the RNAi agent into or near the tissue where the cell is located, or by injecting the RNAi agent into another area, e.g., the bloodstream or the subcutaneous space, such that the agent will subsequently reach the tissue where the cell to be contacted is located. For example, the RNAi agent may contain and/or be coupled to a ligand, e.g., GalNAc3, that directs the RNAi agent to a site of interest, e.g., the liver. Combinations of in vitro and in vivo methods of contacting are also possible. For example, a cell may also be contacted in vitro with an RNAi agent and subsequently transplanted into a subject.

In one embodiment, contacting a cell with an iRNA includes “introducing” or “delivering the iRNA into the cell” by facilitating or effecting uptake or absorption into the cell. Absorption or uptake of an iRNA can occur through unaided diffusive or active cellular processes, or by auxiliary agents or devices. Introducing an iRNA into a cell may be in vitro and/or in vivo. For example, for in vivo introduction, iRNA can be injected into a tissue site or administered systemically. In vivo delivery can also be done by a beta-glucan delivery system, such as those described in U.S. Pat. Nos. 5,032,401 and 5,607,677, and U.S. Publication No. 2005/0281781, the entire contents of which are hereby incorporated herein by reference. In vitro introduction into a cell includes methods known in the art such as electroporation and lipofection. Further approaches are described herein below and/or are known in the art.

The term “lipid nanoparticle” or “LNP” is a vesicle comprising a lipid layer encapsulating a pharmaceutically active molecule, such as a nucleic acid molecule, e.g., an iRNA or a plasmid from which an iRNA is transcribed. LNPs are described in, for example, U.S. Pat. Nos. 6,858,225, 6,815,432, 8,158,601, and 8,058,069, the entire contents of which are hereby incorporated herein by reference.

As used herein, a “subject” is an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose).

In an embodiment, the subject is a human, such as a human being treated or assessed for a disease, disorder or condition that would benefit from reduction in LDHA expression; a human at risk for a disease, disorder or condition that would benefit from reduction in LDHA expression; a human having a disease, disorder or condition that would benefit from reduction in LDHA expression; and/or human being treated for a disease, disorder or condition that would benefit from reduction in LDHA expression as described herein.

It is to be understood that a human being treated or assessed for a disease, disorder or condition that would benefit from reduction in LDHA expression includes a a human being treated or assessed for a disease, disorder or condition that would benefit from reduction in LDHA and HAO1 expression; that a human at risk for a disease, disorder or condition that would benefit from reduction in LDHA expression includes a human at risk for a disease, disorder or condition that would benefit from reduction in LDHA and HAO1 expression; that a human having a disease, disorder or condition that would benefit from reduction in LDHA expression includes a human at risk for a disease, disorder or condition that would benefit from reduction in LDHA and HAO1 expression; and that a human being treated for a disease, disorder or condition that would benefit from reduction in LDHA expression includes a human being treated for a disease, disorder or condition that would benefit from reduction in LDHA and HAO1 expression as described herein.

As used herein, the terms “treating” or “treatment” refer to a beneficial or desired result, such as lowering urinary excretion levels of oxalate in a subject. The terms “treating” or “treatment” also include, but are not limited to, alleviation or amelioration of one or more symptoms of an oxalate pathway-associated disease disorder, or condition, such as, e.g., slowing the course of the disease; reducing the severity of later-developing disease; reduction in edema of the extremities, face, larynx, upper respiratory tract, abdomen, trunk, and/or genitals, prodrome, laryngeal swelling, nonpruritic rash, nausea, vomiting, and/or abdominal pain; decreasing progression of liver disease to cirrhosis or hepatocellular carcinoma; stabilizing current stone burden; decreasing recurrence of stones formed; and/or preventing further oxalate tissue deposition. “Treatment” can also mean prolonging survival as compared to expected survival in the absence of treatment.

The term “lower” in the context of a disease marker or symptom refers to a statistically significant decrease in such level. The decrease can be, for example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more and is preferably down to a level accepted as within the range of normal for an individual without such disorder.

As used herein, “prevention” or “preventing,” when used in reference to a disease, disorder or condition thereof, that would benefit from a reduction in expression of an LDHA gene, refers to a reduction in the likelihood that a subject will develop a symptom associated with such disease, disorder, or condition, e.g., stone formation. The likelihood of, e.g., stone formation, is reduced, for example, when an individual having one or more risk factors for stone formation either fails to develop stones or develops stones with less severity relative to a population having the same risk factors and not receiving treatment as described herein. The failure to develop a disease, disorder or condition, or the reduction in the development of a symptom associated with such a disease, disorder or condition (e.g., by at least about 10% on a clinically accepted scale for that disease or disorder), or the exhibition of delayed symptoms delayed (e.g., by days, weeks, months or years) is considered effective prevention.

There are numerous disorders that would benefit from reduction in expression of an LDHA gene, such as an oxalate pathway-associated disease disorder, or condition.

As used herein, the term “oxalate pathway-associated disease, disorder, or condition” refers to a disease, disorder or condition thereof, in which lactate dehydrogenase knockdown is known or predicted to be therapeutic or otherwise advantageous, e.g., associated with or caused by a disturbance in lactate dehydrogenase production and/or urinary oxalate production.

In one embodiment, an “oxalate pathway-associated disease, disorder, or condition” is a “lactate dehydrogenase-associated disease, disorder, or condition.” As used herein, a “lactate dehydrogenase-associated disease, disorder, or condition” includes any disease, disorder or condition that would benefit from a decrease in lactate dehydrogenase gene expression, replication, or protein activity. Exemplary lactate dehydrogenase-associated disease, disorders, and conditions include, for example, fatty liver (steatosis), nonalcoholic steatohepatitis (NASH), cirrhosis of the liver, accumulation of fat in the liver, inflammation of the liver, hepatocellular necrosis, liver fibrosis, obesity, nonalcoholic fatty liver disease (NAFLD), and cancer, e.g., hepatocellular carcinoma.

In another embodiment, an “oxalate pathway-associated disease, disorder, or condition” is “an oxalate-associated disease, disorder, or condition.” As used herein, “an oxalate-associated disease, disorder, or condition” includes any disease, disorder or condition that would benefit from a decrease in lactate dehydrogenase gene expression, replication, or protein activity. The term “oxalate-associated disease, disorder, or condition” refers to inherited disorders, or induced or acquired disorders. Exemplary “oxalate-associated diseases, disorders, or conditions” include “kidney stone formation diseases, disorders, and conditions” and “calcium oxalate tissue deposition diseases, disorders, and conditions.”

Exemplary kidney stone formation diseases, disorders, and conditions include “calcium oxalate stone formation diseases, disorders, and conditions” and “non-calcium oxalate stone formation diseases, disorders, and conditions.”

Non-limiting examples of “calcium oxalate stone formation diseases, disorders, and conditions” include a hyperoxaluria (e.g., a. primary hyperoxaluria, such as primary hyperoxaluria 1 (PH1), primary hyperoxaluria 2 (PH2), primary hyperoxaluria 3 (PH3) and nonPH1/PH2/PH3; enteric hyperoxaluria; dietary hyperoxaluria; and idiopathic hyperoxaluria) and a non-hyperoxaluria disorder (e.g., a hypercalciuria, such as primary hyperparathyroid, Dent's disease, absorptive hypercalciuria, and renal hypercalciuria; and hypocitraturia).

Non-limiting examples of “non-calcium oxalate stone formation diseases, disorders, and conditions” include subjects having kidney stones that are comprised of less than about 50%, less than about 45%, less than about 40%, less than about 35%, less than about 30%, less than about 25% less than about 20%, less than about 15%, or less than about 10% oxalate, and more than about 50% non-oxalate, e.g. calcium phosphate, uric acid, struvite, cystinuria, or other component.

Exemplary “calcium oxalate tissue deposition diseases, disorders, and conditions” include systemic calcium oxalate tissue deposition diseases, disorders, and conditions, such as calcium oxalate tissue deposition due to end-stage renal disease, sarcoidosis, or arthritis; and tissue specific calcium oxalate deposition diseases, disorders, and conditions, e.g., in the kidney (e.g., due to nephrocalcinosis, or medullary sponge kidney), in the thyroid, in the breast, in the bone, in the heart, in the vasculature, or in any soft tissue due to an organ transplant, such as a kidney transplant.

“Therapeutically effective amount,” as used herein, is intended to include the amount of an RNAi agent that, when administered to a subject having an oxalate pathway-associated disease, disorder, or condition, is sufficient to effect treatment of the disease (e.g., by diminishing, ameliorating or maintaining the existing disease or one or more symptoms of disease). The “therapeutically effective amount” may vary depending on the RNAi agent, how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of an iRNA that, when administered to a subject having an oxalate pathway-associated disease, disorder, or condition, is sufficient to prevent or ameliorate the disease or one or more symptoms of the disease. Ameliorating the disease includes slowing the course of the disease or reducing the severity of later-developing disease. The “prophylactically effective amount” may vary depending on the iRNA, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylacticaly effective amount” also includes an amount of an RNAi agent that produces some desired local or systemic effect at a reasonable benefit/risk ratio applicable to any treatment. iRNA employed in the methods of the present invention may be administered in a sufficient amount to produce a reasonable benefit/risk ratio applicable to such treatment.

In the methods of the invention which include administering to a subject a pharmaceutical composition comprising a first dsRNA agent targeting LDHA and a second dsRNA agent targeting HAO1, the therapeutically effective amountof the first dsRNA agent may be the same or different than the therapeutically effective amount of the second dsRNA agent. Similarly, in the methods of the invention which include administering to a subject a pharmaceutical composition comprising a first dsRNA agent targeting LDHA and a second dsRNA agent targeting HAO1, the prophylacticly effective amountof the first dsRNA agent may be the same or different than the prophylacticaly effective amount of the second dsRNA agent.

The phrase “pharmaceutically acceptable” is employed herein to refer to those compounds, materials, compositions, and/or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs or localized regions. For example, samples may be derived from particular organs, parts of organs, or fluids or cells within those organs. In certain embodiments, samples may be derived from the liver (e.g., whole liver or certain segments of liver or certain types of cells in the liver, such as, e.g., hepatocytes). In some embodiments, a “sample derived from a subject” refers to blood or plasma drawn from the subject.

### II. iRNAs of the Invention

Described herein are iRNAs which inhibit the expression of a target gene. In one embodiment, the iRNAs inhibit the expression of an LDHA gene. In one embodiment, the iRNA agent includes double stranded ribonucleic acid (dsRNA) molecules for inhibiting the expression of an LDHA gene in a cell, such as a liver cell, such as a liver cell within a subject, e.g., a mammal, such as a human having an oxalate pathway-associated disease, disorder, or condition, e.g., a stone formation disease, disorder, or condition. In another embodiment, the iRNAs inhibit the expression of an HAO1 gene. In one embodiment, the iRNA agent includes double stranded ribonucleic acid (dsRNA) molecules for inhibiting the expression of an HAO1 gene in a cell, such as a liver cell, such as a liver cell within a subject, e.g., a mammal, such as a human having a an oxalate pathway-associated disease, disorder, or condition, e.g., an oxalate-associated disease, disorder, or condition, e.g., a kidney stone formation disease, disorder, or condition or a calcium oxalate tissue deposition disease, disorder, or condition; or an LDH-associated disease, disorder, or condition.

Also provided herein are iRNAs which inhibit the expression of two target genes, referred to as dual targeting RNAi agents. In one embodiment, the dual targeting RNAi agent includes a first double stranded ribonucleic acid (dsRNA) agent for inhibiting the expression of an LDHA gene in a cell (such as a liver cell, e.g., a liver cell within a subject) covalently attached to a second double stranded ribonucleic acid (dsRNA) agent for inhibiting the expression of an HAO1 gene in a cell (such as a liver cell, e.g., a liver cell within a subject), such as a cell within a subject, e.g., a mammal, such as a human having an oxalate pathway-associated disease, disorder, or condition, e.g., an oxalate-associated disease, disorder, or condition, e.g., a kidney stone formation disease, disorder, or condition or a calcium oxalate tissue deposition disease, disorder, or condition; or an LDH-associated disease, disorder, or condition.

The dsRNA includes an antisense strand having a region of complementarity which is complementary to at least a part of an mRNA formed in the expression of an LDHA gene or an HAO1 gene, The region of complementarity is about 30 nucleotides or less in length (e.g., about 30, 29, 28, 27, 26, 25, 24, 23, 22, 21, 20, 19, or 18 nucleotides or less in length). Upon contact with a cell expressing the target gene, the iRNA inhibits the expression of the target gene (e.g., a human, a primate, a non-primate, or a bird target gene) by at least about 10% as assayed by, for example, a PCR or branched DNA (bDNA)-based method, or by a protein-based method, such as by immunofluorescence analysis, using, for example, Western Blotting or flowcytometric techniques.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of an LDHA gene or an HAO1 gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is between 15 and 30 base pairs in length, e.g., between, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

Similarly, the region of complementarity to the target sequence is between 15 and 30 nucleotides in length, e.g., between 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the invention.

In some embodiments, the dsRNA is between about 15 and about 23 nucleotides in length, or between about 25 and about 30 nucleotides in length. In general, the dsRNA is long enough to serve as a substrate for the Dicer enzyme. For example, it is well known in the art that dsRNAs longer than about 21-23 nucleotides can serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 9 to 36 base pairs, e.g., about 10-36, 11-36, 12-36, 13-36, 14-36, 15-36, 9-35, 10-35, 11-35, 12-35, 13-35, 14-35, 15-35, 9-34, 10-34, 11-34, 12-34, 13-34, 14-34, 15-34, 9-33, 10-33, 11-33, 12-33, 13-33, 14-33, 15-33, 9-32, 10-32, 11-32, 12-32, 13-32, 14-32, 15-32, 9-31, 10-31, 11-31, 12-31, 13-32, 14-31, 15-31, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an iRNA agent useful to target LDHA expression or LDHA and HAO1 expression is not generated in the target cell by cleavage of a larger dsRNA.

A dsRNA as described herein can further include one or more single-stranded nucleotide overhangs e.g., 1, 2, 3, or 4 nucleotides. dsRNAs having at least one nucleotide overhang can have unexpectedly superior inhibitory properties relative to their blunt-ended counterparts. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

A dsRNA can be synthesized by standard methods known in the art as further discussed below, e.g., by use of an automated DNA synthesizer, such as are commercially available from, for example, Biosearch, Applied Biosystems, Inc.

iRNA compounds of the invention may be prepared using a two-step procedure. First, the individual strands of the double-stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Single-stranded oligonucleotides of the invention can be prepared using solution-phase or solid-phase organic synthesis or both.

In one aspect, a dsRNA of the invention includes at least two nucleotide sequences, a sense sequence and an anti-sense sequence. The sense strand sequence is selected from the group of sequences provided in any one of Tables 2-5 and the corresponding nucleotide sequence of the antisense strand of the sense strand is selected from the group of sequences of any one of Tables 2-5. In this aspect, one of the two sequences is complementary to the other of the two sequences, with one of the sequences being substantially complementary to a sequence of an mRNA generated in the expression of an LDHA gene. As such, in this aspect, a dsRNA will include two oligonucleotides, where one oligonucleotide is described as the sense strand (passenger strand) in any one of Tables 2-5 and the second oligonucleotide is described as the corresponding antisense strand (guide strand) of the sense strand in any one of Tables 2-5. In one embodiment, the substantially complementary sequences of the dsRNA are contained on separate oligonucleotides. In another embodiment, the substantially complementary sequences of the dsRNA are contained on a single oligonucleotide.

In another aspect, a dsRNA of the invention targets an HAO1 gene and includes at least two nucleotide sequences, a sense sequence and an anti-sense sequence. The sense strand sequence is selected from the group of sequences provided in any one of Tables 7-14 and the corresponding nucleotide sequence of the antisense strand of the sense strand is selected from the group of sequences of any one of Tables 7-14. In this aspect, one of the two sequences is complementary to the other of the two sequences, with one of the sequences being substantially complementary to a sequence of an mRNA generated in the expression of an HAO1 gene. As such, in this aspect, a dsRNA will include two oligonucleotides, where one oligonucleotide is described as the sense strand (passenger strand) in any one of Tables 7-14 and the second oligonucleotide is described as the corresponding antisense strand (guide strand) of the sense strand in any one of Tables 7-14. In one embodiment, the substantially complementary sequences of the dsRNA are contained on separate oligonucleotides. In another embodiment, the substantially complementary sequences of the dsRNA are contained on a single oligonucleotide.

It will be understood that, although the sequences in Tables 2-5 and 7-14 are described as modified, unmodified, unconjugated. and/or conjugated sequences, the RNA of the iRNA of the invention e.g., a dsRNA of the invention, may comprise any one of the sequences set forth in any one of Table 2-5 and 7-14 that is un-modified, un-conjugated, and/or modified and/or conjugated differently than described therein.

The skilled person is well aware that dsRNAs having a duplex structure of between about 20 and 23 base pairs, e.g., 21, base pairs have been hailed as particularly effective in inducing RNA interference (Elbashir et al., (2001) EMBO J., 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, by virtue of the nature of the oligonucleotide sequences provided herein, dsRNAs described herein can include at least one strand of a length of minimally 21 nucleotides. It can be reasonably expected that shorter duplexes minus only a few nucleotides on one or both ends can be similarly effective as compared to the dsRNAs described above. Hence, dsRNAs having a sequence of at least 15, 16, 17, 18, 19, 20, or more contiguous nucleotides derived from one of the sequences provided herein, and differing in their ability to inhibit the expression of an LDHA gene or an HAO1 gene by not more than about 5, 10, 15, 20, 25, or 30% inhibition from a dsRNA comprising the full sequence, are contemplated to be within the scope of the present invention.

In addition, the RNAs described in any one of Tables 2-5 identify a site(s) in an LDHA transcript that is susceptible to RISC-mediated cleavage and those RNAs described in any one of Tables 7-14 identify a site(s) in an HAO1 transcript that is susceptible to RISC-mediated cleavage. As such, the present invention further features iRNAs that target within this site(s). As used herein, an iRNA is said to target within a particular site of an RNA transcript if the iRNA promotes cleavage of the transcript anywhere within that particular site. Such an iRNA will generally include at least about 15 contiguous nucleotides from one of the sequences provided herein coupled to additional nucleotide sequences taken from the region contiguous to the selected sequence in the gene.

While a target sequence is generally about 15-30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an iRNA agent, mediate the best inhibition of target gene expression. Thus, while the sequences identified herein represent effective target sequences, it is contemplated that further optimization of inhibition efficiency can be achieved by progressively “walking the window” one nucleotide upstream or downstream of the given sequences to identify sequences with equal or better inhibition characteristics.

Further, it is contemplated that for any sequence identified herein, further optimization could be achieved by systematically either adding or removing nucleotides to generate longer or shorter sequences and testing those sequences generated by walking a window of the longer or shorter size up or down the target RNA from that point. Again, coupling this approach to generating new candidate targets with testing for effectiveness of iRNAs based on those target sequences in an inhibition assay as known in the art and/or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes) as an expression inhibitor.

An iRNA agent as described herein can contain one or more mismatches to the target sequence. In one embodiment, an iRNA as described herein contains no more than 3 mismatches. If the antisense strand of the iRNA contains mismatches to a target sequence, it is preferable that the area of mismatch is not located in the center of the region of complementarity. If the antisense strand of the iRNA contains mismatches to the target sequence, it is preferable that the mismatch be restricted to be within the last 5 nucleotides from either the 5′- or 3′-end of the region of complementarity. For example, for a 23 nucleotide iRNA agent the strand which is complementary to a region of an LDHA gene or an HAO1 gene, generally does not contain any mismatch within the central 13 nucleotides. The methods described herein or methods known in the art can be used to determine whether an iRNA containing a mismatch to a target sequence is effective in inhibiting the expression of an LDHA gene and/or an HAO1 gene. Consideration of the efficacy of iRNAs with mismatches in inhibiting expression of an LDHA gene and/or an HAO1 gene is important, especially if the particular region of complementarity in an LDHA gene and/or HAO1 gene is known to have polymorphic sequence variation within the population.

The dual targeting RNAi agents of the invention, which include two dsRNA agents, are covalently attached via, e.g., a covalent linker. Covalent linkers are well known in the art and include, e.g., nucleic acid linkers, peptide linkers, carbohydrate linkers, and the like. The covalent linker can include RNA and/or DNA and/or a peptide. The linker can be single stranded, double stranded, partially single strands, or partially double stranded. Modified nucleotides or a mixture of nucleotides can also be present in a nucleic acid linker.

Suitable linkers for use in the dual targeting agent of the invention include those described in U.S. Pat. No. 9,187,746, the entire contents of which are incorporated herein by reference.

In some embodiments the linker includes a disulfide bond. The linker can be cleavable or non-cleavable.

The linker can be, e.g., dTsdTuu=(5′-2′deoxythymidyl-3′-thiophosphate-5′-2/deoxythymidyl-3′-phosphate-5′-uridyl-3′-phosphate-5′-uridyl-3′-phosphate); rUsrU (a thiophosphate linker: 5′-uridyl-3′-thiophosphate-5′-uridyl-3′-phosphate); an rUrU linker; dTsdTaa (aadTsdT, 5′-2′deoxythymidyl-3′-thiophosphate-5′-2′deoxythymidyl-3′-phosphate-5′-adenyl-3′-phosphate-5′-adenyl-3′-phosphate); dTsdT (5′-2′deoxythymidyl-3′-thiophosphate-5′-2′ deoxythymidyl-3′-phosphate); dTsdTuu=uudTsdT=5′-2′deoxythymidyl-3′-thiophosphate-5′-2′deoxythymidyl-3′-phosphate-5′-uridyl-3′-phosphate-5′-uridyl-3′-phosphate.

The linker can be a polyRNA, such as poly(5′-adenyl-3′-phosphate-AAAAAAAA) or poly(5′-cytidyl-3′-phosphate-5′-uridyl-3′-phosphate-CUCUCUCU)), e.g., Xn single stranded poly RNA linker wherein n is an integer from 2-50 inclusive, preferable 4-15 inclusive, most preferably 7-8 inclusive. Modified nucleotides or a mixture of nucleotides can also be present in said polyRNA linker. The covalent linker can be a polyDNA, such as poly(5′-2′deoxythymidyl-3′-phosphate-TTTTTTTT), e.g., wherein n is an integer from 2-50 inclusive, preferable 4-15 inclusive, most preferably 7-8 inclusive. Modified nucleotides or a mixture of nucleotides can also be present in said polyDNA linker, a single stranded polyDNA linker wherein n is an integer from 2-50 inclusive, preferable 4-inclusive, most preferably 7-8 inclusive. Modified nucleotides or a mixture of nucleotides can also be present in said polyDNA linker.

The linker can include a disulfide bond, optionally a bis-hexyl-disulfide linker. In one embodiment, the disulfide linker is

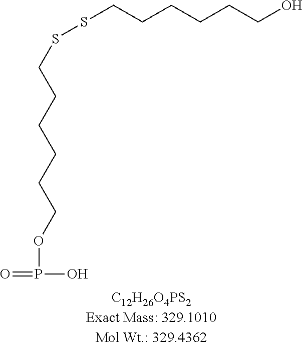

The linker can include a peptide bond, e.g., include amino acids. In one embodiment, the covalent linker is a 1-10 amino acid long linker, preferably comprising 4-5 amino acids, optionally X-Gly-Phe-Gly-Y wherein X and Y represent any amino acid.

The linker can include HEG, a hexaethylenglycol linker.

The covalent linker can attach the sense strand of the first dsRNA agent to the sense strand of the second dsRNA agent; the antisense strand of the first dsRNA agent to the antisense strand of the second dsRNA agent; the sense strand of the first dsRNA agent to the antisense strand of the second dsRNA agent; or the antisense strand of the first dsRNA agent to the sense strand of the second dsRNA agent.

In some embodiments, the covalent linker further comprises at least one ligand, described below.

### III. Modified iRNAs of the Invention

In one embodiment, the RNA of the iRNA of the invention e.g., a dsRNA, is un-modified, and does not comprise, e.g., chemical modifications and/or conjugations known in the art and described herein. In another embodiment, the RNA of an iRNA of the invention, e.g., a dsRNA, is chemically modified to enhance stability or other beneficial characteristics. In certain embodiments of the invention, substantially all of the nucleotides of an iRNA of the invention are modified. In other embodiments of the invention, all of the nucleotides of an iRNA of the invention are modified. iRNAs of the invention in which “substantially all of the nucleotides are modified” are largely but not wholly modified and can include not more than 5, 4, 3, 2, or 1 unmodified nucleotides.

In embodiments in which a first dsRNA agent targeting LDHA and a second dsRNA agent targeting HAO1 are covalently attached (i.e., a dual targeting RNAi agent), substantially all of the nucleotides of the first agent and substantially all of the nucleotides of the second agent may be independently modified; all of the nucleotides of the first agent may be modified and all of the nucleotides of the second agent may be independently modified; substantially all of the nucleotides of the first agent and all of the nucleotides of the second agent may be independently modified; or all of the nucleotides of the first agent may be modified and substantially all of the nucleotides of the second agent may be independently modified.

In some aspects of the invention, substantially all of the nucleotides of an iRNA of the invention are modified and the iRNA agents comprise no more than 10 nucleotides comprising 2′-fluoro modifications (e.g., no more than 9 2′-fluoro modifications, no more than 8 2′-fluoro modifications, no more than 7 2′-fluoro modifications, no more than 6 2′-fluoro modifications, no more than 5 2′-fluoro modifications, no more than 4 2′-fluoro modifications, no more than 5 2′-fluoro modifications, no more than 4 2′-fluoro modifications, no more than 3 2′-fluoro modifications, or no more than 2 2′-fluoro modifications). For example, in some embodiments, the sense strand comprises no more than 4 nucleotides comprising 2′-fluoro modifications (e.g., no more than 3 2′-fluoro modifications, or no more than 2 2′-fluoro modifications). In other embodiments, the antisense strand comprises no more than 6 nucleotides comprising 2′-fluoro modifications (e.g., no more than 5 2′-fluoro modifications, no more than 4 2′-fluoro modifications, no more than 4 2′-fluoro modifications, or no more than 2 2′-fluoro modifications).

In embodiments in which a first dsRNA agent targeting LDHA and a second dsRNA agent targeting HAO1 are covalently attached (i.e., a dual targeting RNAi agent), substantially all of the nucleotides of the first agent and/or substantially all of the nucleotides of the second agent may be independently modified and the first and second agents may independently comprise no more than 10 nucleotides comprising 2′-fluoro modifications.

In other aspects of the invention, all of the nucleotides of an iRNA of the invention are modified and the iRNA agents comprise no more than 10 nucleotides comprising 2′-fluoro modifications (e.g., no more than 9 2′-fluoro modifications, no more than 8 2′-fluoro modifications, no more than 7 2′-fluoro modifications, no more than 6 2′-fluoro modifications, no more than 5 2′-fluoro modifications, no more than 4 2′-fluoro modifications, no more than 5 2′-fluoro modifications, no more than 4 2′-fluoro modifications, no more than 3 2′-fluoro modifications, or no more than 2 2′-fluoro modifications).

In embodiments in which a first dsRNA agent targeting LDHA and a second dsRNA agent targeting HAO1 are covalently attached (i.e., a dual targeting RNAi agent), all of the nucleotides of the first agent and/or all of the nucleotides of the second agent may be independently modified and the first and second agents may independently comprise no more than 10 nucleotides comprising 2′-fluoro modifications.

In one embodiment, the double stranded RNAi agent of the invention further comprises a 5′-phosphate or a 5′-phosphate mimic at the 5′ nucleotide of the antisense strand. In another embodiment, the double stranded RNAi agent further comprises a 5′-phosphate mimic at the 5′ nucleotide of the antisense strand. In a specific embodiment, the 5′-phosphate mimic is a 5′-vinyl phosphate (5′-VP).

In embodiments in which a first dsRNA agent targeting LDHA and a second dsRNA agent targeting HAO1 are covalently attached (i.e., a dual targeting RNAi agent), the first agent may further comprise a 5′-phosphate or a 5′-phosphate mimic at the 5′ nucleotide of the antisense strand; the second agent may further comprise a 5′-phosphate or a 5′-phosphate mimic at the 5′ nucleotide of the antisense strand; or the first agent and the second agent may further independently comprise a 5′-phosphate or a 5′-phosphate mimic at the 5′ nucleotide of the antisense strand.

The nucleic acids featured in the invention can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; and/or backbone modifications, including modification or replacement of the phosphodiester linkages. Specific examples of iRNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified iRNA will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other embodiments, suitable RNA mimetics are contemplated for use in iRNAs, in which both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the iRNAs of the invention are described in, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

An iRNA of the invention can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

An iRNA of the invention can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

An iRNA of the invention can also be modified to include one or more bicyclic sugar moities. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the invention may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH2-O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Examples of bicyclic nucleosides for use in the polynucleotides of the invention include without limitation nucleosides comprising a bridge between the 4′ and the 2′ ribosyl ring atoms. In certain embodiments, the antisense polynucleotide agents of the invention include one or more bicyclic nucleosides comprising a 4′ to 2′ bridge. Examples of such 4′ to 2′ bridged bicyclic nucleosides, include but are not limited to 4′-(CH2)-O-2′ (LNA); 4′-(CH2)-S-2′; 4′-(CH2)2-O-2′ (ENA); 4′-CH(CH3)-O-2′ (also referred to as “constrained ethyl” or “cEt”) and 4′-CH(CH2OCH3)-O-2′ (and analogs thereof; see, e.g., U.S. Pat. No. 7,399,845); 4′-C(CH3)(CH3)-O-2′ (and analogs thereof; see e.g., U.S. Pat. No. 8,278,283); 4′-CH2-N(OCH3)-2′ (and analogs thereof; see e.g., U.S. Pat. No. 8,278,425); 4′-CH2-O—N(CH3)-2′ (see, e.g., U.S. Patent Publication No. 2004/0171570); 4′-CH2-N(R)—O-2′, wherein R is H, C1-C12 alkyl, or a protecting group (see, e.g., U.S. Pat. No. 7,427,672); 4′-CH2—C(H)(CH3)-2′ (see, e.g., Chattopadhyaya et al., J. Org. Chem., 2009, 74, 118-134); and 4′-CH2-C(═CH2)-2′ (and analogs thereof; see, e.g., U.S. Pat. No. 8,278,426). The entire contents of each of the foregoing are hereby incorporated herein by reference.

Additional representative U.S. patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see WO 99/14226).

An iRNA of the invention can also be modified to include one or more constrained ethyl nucleotides. As used herein, a “constrained ethyl nucleotide” or “cEt” is a locked nucleic acid comprising a bicyclic sugar moiety comprising a 4′-CH(CH3)-O-2′ bridge. In one embodiment, a constrained ethyl nucleotide is in the S conformation referred to herein as “S-cEt.”

An iRNA of the invention may also include one or more “conformationally restricted nucleotides” (“CRN”). CRN are nucleotide analogs with a linker connecting the C2′ and C4′ carbons of ribose or the C3 and −05′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some embodiments, an iRNA of the invention comprises one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-0-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other modifications of an iRNA of the invention include a 5′ phosphate or 5′ phosphate mimic, e.g., a 5′-terminal phosphate or phosphate mimic on the antisense strand of an RNAi agent. Suitable phosphate mimics are disclosed in, for example US Patent Publication No. 2012/0157511, the entire contents of which are incorporated herein by reference.

In certain specific embodiments, an RNAi agent of the present invention is an agent that inhibits the expression of an LDHA gene which is selected from the group of agents listed in any one of Tables 2-5. In other embodiments, an RNAi agent of the present invention is an dual targeting iRNA agent that inhibits the expression of an LDHA gene and an HAO1, wherein the first dsRNA inhibits expression of an LDHA gene and is selected from the group of agents listed in any one of Tables 2-5, and and the first dsRNA inhibits expression of an HAO1 gene and is selected from the group of agents listed in any one of Tables 7-14. Any of these agents may further comprise a ligand.

A. Modified iRNAs Comprising Motifs of the Invention

In certain aspects of the invention, the double stranded RNAi agents of the invention include agents with chemical modifications as disclosed, for example, in WO 2013/075035, filed on Nov. 16, 2012, the entire contents of which are incorporated herein by reference.

It is to be understood that, in embodiments in which a first dsRNA agent targeting LDHA and a second dsRNA agent targeting HAO1 are covalently attached (i.e., a dual targeting RNAi agent), the first agent may comprise any one or more of the motifs described below, the second agent may comprise any one or more of the motifs described below, or both the first agent and the second agent may independently comprise any one or more of the motifs described below.

Accordingly, the invention provides double stranded RNAi agents capable of inhibiting the expression of a target gene (i.e., an LDHA gene or an LDHA gene and an HAO1 gene) in vivo. The RNAi agent comprises a sense strand and an antisense strand. Each strand of the RNAi agent may range from 12-30 nucleotides in length. For example, each strand may be between 14-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length.

The sense strand and antisense strand typically form a duplex double stranded RNA (“dsRNA”), also referred to herein as an “RNAi agent.” The duplex region of an RNAi agent may be 12-30 nucleotide pairs in length. For example, the duplex region can be between 14-30 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length.

In one embodiment, the RNAi agent may contain one or more overhang regions and/or capping groups at the 3′-end, 5′-end, or both ends of one or both strands. The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one embodiment, the nucleotides in the overhang region of the RNAi agent can each independently be a modified or unmodified nucleotide including, but no limited to 2′-sugar modified, such as, 2-F, 2′-Omethyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand or both strands of the RNAi agent may be phosphorylated. In some embodiments, the overhang region(s) contains two nucleotides having a phosphorothioate between the two nucleotides, where the two nucleotides can be the same or different. In one embodiment, the overhang is present at the 3′-end of the sense strand, antisense strand, or both strands. In one embodiment, this 3′-overhang is present in the antisense strand. In one embodiment, this 3′-overhang is present in the sense strand.

The RNAi agent may contain only a single overhang, which can strengthen the interference activity of the RNAi, without affecting its overall stability. For example, the single-stranded overhang may be located at the 3′-terminal end of the sense strand or, alternatively, at the 3′-terminal end of the antisense strand. The RNAi may also have a blunt end, located at the 5′-end of the antisense strand (or the 3′-end of the sense strand) or vice versa. Generally, the antisense strand of the RNAi has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In one embodiment, the RNAi agent is a double ended bluntmer of 19 nucleotides in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In another embodiment, the RNAi agent is a double ended bluntmer of 20 nucleotides in length, wherein the sense strand contains at least one motif of three 2′-F modifications on three consecutive nucleotides at positions 8, 9, 10 from the 5′end. The antisense strand contains at least one motif of three 2′-O-methyl modifications on three consecutive nucleotides at positions 11, 12, 13 from the 5′end.

In yet another embodiment, the RNAi agent is a double ended bluntmer of 21 nucleotides in length, wherein the sense strand contains at least one motif of three 2′-F modifications on three consecutive nucleotides at positions 9, 10, 11 from the 5′end. The antisense strand contains at least one motif of three 2′-O-methyl modifications on three consecutive nucleotides at positions 11, 12, 13 from the 5′end.

In one embodiment, the RNAi agent comprises a 21 nucleotide sense strand and a 23 nucleotide antisense strand, wherein the sense strand contains at least one motif of three 2′-F modifications on three consecutive nucleotides at positions 9, 10, 11 from the 5′end; the antisense strand contains at least one motif of three 2′-O-methyl modifications on three consecutive nucleotides at positions 11, 12, 13 from the 5′end, wherein one end of the RNAi agent is blunt, while the other end comprises a 2 nucleotide overhang. Preferably, the 2 nucleotide overhang is at the 3′-end of the antisense strand.

When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In one embodiment, every nucleotide in the sense strand and the antisense strand of the RNAi agent, including the nucleotides that are part of the motifs are modified nucleotides. In one embodiment each residue is independently modified with a 2′-O-methyl or 3′-fluoro, e.g., in an alternating motif. Optionally, the RNAi agent further comprises a ligand (preferably GalNAc₃).

In one embodiment, the RNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3 ‘ terminal nucleotide of antisense strand is unpaired with sense strand, and up to 6 consecutive 3’ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when the double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the RNAi agent comprises sense and antisense strands, wherein the RNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein the 3′ end of the first strand and the 5′ end of the second strand form a blunt end and the second strand is 1-4 nucleotides longer at its 3′ end than the first strand, wherein the duplex region region which is at least 25 nucleotides in length, and the second strand is sufficiently complemenatary to a target mRNA along at least 19 nucleotide of the second strand length to reduce target gene expression when the RNAi agent is introduced into a mammalian cell, and wherein dicer cleavage of the RNAi agent preferentially results in an siRNA comprising the 3′ end of the second strand, thereby reducing expression of the target gene in the mammal. Optionally, the RNAi agent further comprises a ligand.

In one embodiment, the sense strand of the RNAi agent contains at least one motif of three identical modifications on three consecutive nucleotides, where one of the motifs occurs at the cleavage site in the sense strand.

In one embodiment, the antisense strand of the RNAi agent can also contain at least one motif of three identical modifications on three consecutive nucleotides, where one of the motifs occurs at or near the cleavage site in the antisense strand.

For an RNAi agent having a duplex region of 17-23 nucleotide in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the RNAi from the 5′-end.

The sense strand of the RNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand. Alternatively, at least two nucleotides may overlap, or all three nucleotides may overlap.

In one embodiment, the sense strand of the RNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adajacent to the first motif or is separated by at least one or more nucleotides. When the motifs are immediately adjacent to each other then the chemistry of the motifs are distinct from each other and when the motifs are separated by one or more nucleotide than the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the RNAi agent may contain more than one motifs of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In one embodiment, the wing modification on the sense strand or antisense strand of the RNAi agent typically does not include the first one or two terminal nucleotides at the 3′-end, 5′-end or both ends of the strand.

In another embodiment, the wing modification on the sense strand or antisense strand of the RNAi agent typically does not include the first one or two paired nucleotides within the duplex region at the 3′-end, 5′-end or both ends of the strand.

When the sense strand and the antisense strand of the RNAi agent each contain at least one wing modification, the wing modifications may fall on the same end of the duplex region, and have an overlap of one, two or three nucleotides.

When the sense strand and the antisense strand of the RNAi agent each contain at least two wing modifications, the sense strand and the antisense strand can be so aligned that two modifications each from one strand fall on one end of the duplex region, having an overlap of one, two or three nucleotides; two modifications each from one strand fall on the other end of the duplex region, having an overlap of one, two or three nucleotides; two modifications one strand fall on each side of the lead motif, having an overlap of one, two or three nucleotides in the duplex region.

In one embodiment, every nucleotide in the sense strand and antisense strand of the RNAi agent, including the nucleotides that are part of the motifs, may be modified. Each nucleotide may be modified with the same or different modification which can include one or more alteration of one or both of the non-linking phosphate oxygens and/or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases the modification will occur at all of the subject positions in the nucleic acid but in many cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of a RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. For example, it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In one embodiment, each residue of the sense strand and antisense strand is independently modified with LNA, CRN, cET, UNA, HNA, CeNA, 2′-methoxyethyl, 2′-O-methyl, 2′-O-allyl, 2′-C-allyl, 2′-deoxy, 2′-hydroxyl, or 2′-fluoro. The strands can contain more than one modification. In one embodiment, each residue of the sense strand and antisense strand is independently modified with 2′-O-methyl or 2′-fluoro.

At least two different modifications are typically present on the sense strand and antisense strand. Those two modifications may be the 2′-O-methyl or 2′-fluoro modifications, or others.

In one embodiment, the Nₐ and/or N_{b} comprise modifications of an alternating pattern. The term “alternating motif” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In one embodiment, the RNAi agent of the invention comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 5′-3′ of the strand within the duplex region. As another example, the alternating motif in the sense strand may start with “AABBAABB” from 5′-3′ of the strand and the alternating motif in the antisenese strand may start with “BBAABBAA” from 5′-3′ of the strand within the duplex region, so that there is a complete or partial shift of the modification patterns between the sense strand and the antisense strand.

In one embodiment, the RNAi agent comprises the pattern of the alternating motif of 2′-O-methyl modification and 2′-F modification on the sense strand initially has a shift relative to the pattern of the alternating motif of 2′-O-methyl modification and 2′-F modification on the antisense strand initially, i.e., the 2′-O-methyl modified nucleotide on the sense strand base pairs with a 2′-F modified nucleotide on the antisense strand and vice versa. The 1 position of the sense strand may start with the 2′-F modification, and the 1 position of the antisense strand may start with the 2′-O-methyl modification.

The introduction of one or more motifs of three identical modifications on three consecutive nucleotides to the sense strand and/or antisense strand interrupts the initial modification pattern present in the sense strand and/or antisense strand. This interruption of the modification pattern of the sense and/or antisense strand by introducing one or more motifs of three identical modifications on three consecutive nucleotides to the sense and/or antisense strand surprisingly enhances the gene silencing acitivty to the target gene.

In one embodiment, when the motif of three identical modifications on three consecutive nucleotides is introduced to any of the strands, the modification of the nucleotide next to the motif is a different modification than the modification of the motif. For example, the portion of the sequence containing the motif is “ . . . NₐYYYN_{b} . . . ,” where “Y” represents the modification of the motif of three identical modifications on three consecutive nucleotide, and “Nₐ” and “N_{b}” represent a modification to the nucleotide next to the motif “YYY” that is different than the modification of Y, and where Nₐ and N_{b} can be the same or different modifications. Alternatively, Nₐ and/or N_{b} may be present or absent when there is a wing modification present.

The RNAi agent may further comprise at least one phosphorothioate or methylphosphonate internucleotide linkage. The phosphorothioate or methylphosphonate internucleotide linkage modification may occur on any nucleotide of the sense strand or antisense strand or both strands in any position of the strand. For instance, the internucleotide linkage modification may occur on every nucleotide on the sense strand and/or antisense strand; each internucleotide linkage modification may occur in an alternating pattern on the sense strand and/or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand. In one embodiment, a double-standed RNAi agent comprises 6-8phosphorothioate internucleotide linkages. In one embodiment, the antisense strand comprises two phosphorothioate internucleotide linkages at the 5′-terminus and two phosphorothioate internucleotide linkages at the 3′-terminus, and the sense strand comprises at least two phosphorothioate internucleotide linkages at either the 5′-terminus or the 3′-terminus.

In one embodiment, the RNAi comprises a phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within the duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. These terminal three nucleotides may be at the 3′-end of the antisense strand, the 3′-end of the sense strand, the 5′-end of the antisense strand, and/or the 5′end of the antisense strand.

In one embodiment, the 2 nucleotide overhang is at the 3′-end of the antisense strand, and there are two phosphorothioate internucleotide linkages between the terminal three nucleotides, wherein two of the three nucleotides are the overhang nucleotides, and the third nucleotide is a paired nucleotide next to the overhang nucleotide. Optionally, the RNAi agent may additionally have two phosphorothioate internucleotide linkages between the terminal three nucleotides at both the 5′-end of the sense strand and at the 5′-end of the antisense strand.

In one embodiment, the RNAi agent comprises mismatch(es) with the target, within the duplex, or combinations thereof. The mistmatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the RNAi agent comprises at least one of the first 1, 2, 3, 4, or 5 base pairs within the duplex regions from the 5′-end of the antisense strand independently selected from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In another embodiment, the nucleotide at the 3′-end of the sense strand is deoxy-thymine (dT). In another embodiment, the nucleotide at the 3′-end of the antisense strand is deoxy-thymine (dT). In one embodiment, there is a short sequence of deoxy-thymine nucleotides, for example, two dT nucleotides on the 3′-end of the sense and/or antisense strand.

In one embodiment, the sense strand sequence may be represented by formula (I):

(I)
5′ nₚ-Nₐ-(X X X)ᵢ-N_{b}-Y Y Y-N_{b}-(Z Z Z)ⱼ-Nₐ-n_{q} 3′

wherein:

i and j are each independently 0 or 1;

p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 0-10 modified nucleotides;
each nₚ and n_{q} independently represent an overhang nucleotide;
wherein Nb and Y do not have the same modification; and
XXX, YYY and ZZZ each independently represent one motif of three identical modifications on three consecutive nucleotides. Preferably YYY is all 2′-F modified nucleotides.

In one embodiment, the Nₐ and/or N_{b} comprise modifications of alternating pattern.

In one embodiment, the YYY motif occurs at or near the cleavage site of the sense strand. For example, when the RNAi agent has a duplex region of 17-23 nucleotides in length, the YYY motif can occur at or the vicinity of the cleavage site (e.g.: can occur at positions 6, 7, 8, 7, 8, 9, 8, 9, 10, 9, 10, 11, 10, 11, 12 or 11, 12, 13) of—the sense strand, the count starting from the 1^{st} nucleotide, from the 5′-end; or optionally, the count starting at the 1^{st} paired nucleotide within the duplex region, from the 5′-end.

In one embodiment, i is 1 and j is 0, or i is 0 and j is 1, or both i and j are 1. The sense strand can therefore be represented by the following formulas:

(Ib)

5′ nₚ-Nₐ-YYY-N_{b}-ZZZ-Nₐ-n_{q} 3′;

(Ic)

5′ nₚ-Nₐ-XXX-N_{b}-YYY-Nₐ-n_{q} 3′;
or

(Id)

5′ nₚ-Nₐ-XXX-N_{b}-YYY-N_{b}-ZZZ-Nₐ-n_{q} 3′.

When the sense strand is represented by formula (Ib), N_{b} represents an oligonucleotide sequence comprising 0-10, 0-7, 0-5, 0-4, 0-2 or 0 modified nucleotides. Each Nₐ independently can represent an oligonucleotide sequence comprising 2-20, 2-15, or 2-10 modified nucleotides.

When the sense strand is represented as formula (Ic), N_{b} represents an oligonucleotide sequence comprising 0-10, 0-7, 0-10, 0-7, 0-5, 0-4, 0-2 or 0 modified nucleotides. Each Nₐ can independently represent an oligonucleotide sequence comprising 2-20, 2-15, or 2-10 modified nucleotides.

When the sense strand is represented as formula (Id), each N_{b} independently represents an oligonucleotide sequence comprising 0-10, 0-7, 0-5, 0-4, 0-2 or 0 modified nucleotides. Preferably, N_{b} is 0, 1, 2, 3, 4, 5 or 6. Each Nₐ can independently represent an oligonucleotide sequence comprising 2-20, 2-15, or 2-10 modified nucleotides.

Each of X, Y and Z may be the same or different from each other.

In other embodiments, i is 0 and j is 0, and the sense strand may be represented by the formula:

(Ia)

5′ nₚ-Nₐ-YYY-Nₐ-n_{q} 3′.

When the sense strand is represented by formula (Ia), each Nₐ independently can represent an oligonucleotide sequence comprising 2-20, 2-15, or 2-10 modified nucleotides.

In one embodiment, the antisense strand sequence of the RNAi may be represented by formula (II):

(II)
5′ n_{q′}-Nₐ′-(Z′Z′Z′)ₖ-N_{b}′-Y′Y′Y′-N_{b}′-(X′X′X′)₁-Nₐ′-
nₚ′ 3′

wherein:

k and l are each independently 0 or 1;

p′ and q′ are each independently 0-6;
each Nₐ′ independently represents an oligonucleotide sequence comprising 0-25 modified nucleotides, each sequence comprising at least two differently modified nucleotides;
each N_{b}′ independently represents an oligonucleotide sequence comprising 0-10 modified nucleotides;
each nₚ′ and n_{q}′ independently represent an overhang nucleotide;
wherein N_{b}′ and Y′ do not have the same modification; and
X′X′X′, Y′Y′Y′ and Z′Z′Z′ each independently represent one motif of three identical modifications on three consecutive nucleotides.

In one embodiment, the Nₐ′ and/or N_{b}′ comprise modifications of alternating pattern.

The Y′Y′Y′ motif occurs at or near the cleavage site of the antisense strand. For example, when the RNAi agent has a duplex region of 17-23nucleotidein length, the Y′Y′Y′ motif can occur at positions 9, 10, 11; 10, 11, 12; 11, 12, 13; 12, 13, 14; or 13, 14, 15 of the antisense strand, with the count starting from the 1^{st} nucleotide, from the 5′-end; or optionally, the count starting at the 1^{st} paired nucleotide within the duplex region, from the 5′-end. Preferably, the Y′Y′Y′ motif occurs at positions 11, 12, 13.

In one embodiment, Y′Y′Y′ motif is all 2′-OMe modified nucleotides.

In one embodiment, k is 1 and 1 is 0, or k is 0 and 1 is 1, or both k and 1 are 1.

The antisense strand can therefore be represented by the following formulas:

(IIb)
5′ n_{q′}-Nₐ′-Z′Z′Z′-N_{b}′-Y′Y′Y′-Nₐ′-n_{p′} 3′;
(IIc)
5′ n_{q′}-Nₐ′-Y′Y′Y′-N_{b}′-X′X′X′-n_{p′} 3′;
or
(IId)
5′ n_{q′}-Nₐ′-Z′Z′Z′-N_{b}′-Y′Y′Y′-N_{b}′-X′X′X′-Nₐ′-
n_{p′} 3′.

When the antisense strand is represented by formula (IIb), N_{b}′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IIc), N_{b}′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IId), each N_{b}′ independently represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides. Preferably, N_{b} is 0, 1, 2, 3, 4, 5 or 6.

In other embodiments, k is 0 and 1 is 0 and the antisense strand may be represented by the formula:

(Ia)

5′ n_{p′}-N_{a′}-Y′Y′Y′-N_{a′}-n_{q′} 3′.

When the antisense strand is represented as formula (IIa), each Nₐ′ independently represents an oligonucleotide sequence comprising 2-20, 2-15, or 2-10 modified nucleotides.

Each of X′, Y′ and Z′ may be the same or different from each other.

Each nucleotide of the sense strand and antisense strand may be independently modified with LNA, CRN, UNA, cEt, HNA, CeNA, 2′-methoxyethyl, 2′-O-methyl, 2′-O-allyl, 2′-C-allyl, 2′-hydroxyl, or 2′-fluoro. For example, each nucleotide of the sense strand and antisense strand is independently modified with 2′-O-methyl or 2′-fluoro. Each X, Y, Z, X′, Y′ and Z′, in particular, may represent a 2′-O-methyl modification or a 2′-fluoro modification.

In one embodiment, the sense strand of the RNAi agent may contain YYY motif occurring at 9, 10 and 11 positions of the strand when the duplex region is 21 nt, the count starting from the 1^{st} nucleotide from the 5′-end, or optionally, the count starting at the 1^{st} paired nucleotide within the duplex region, from the 5′-end; and Y represents 2′-F modification. The sense strand may additionally contain XXX motif or ZZZ motifs as wing modifications at the opposite end of the duplex region; and XXX and ZZZ each independently represents a 2′-OMe modification or 2′-F modification.

In one embodiment the antisense strand may contain Y′Y′Y′ motif occurring at positions 11, 12, 13 of the strand, the count starting from the 1st nucleotide from the 5′ end, or optionally, the count starting at the 1st paired nucleotide within the duplex region, from the 5′-end; and Y′ represents 2′-O-methyl modification. The antisense strand may additionally contain X′X′X′ motif or Z′Z′Z′ motifs as wing modifications at the opposite end of the duplex region; and X′X′X′ and Z′Z′Z′ each independently represents a 2′-OMe modification or 2′-F modification.

The sense strand represented by any one of the above formulas (Ia), (Ib), (Ic), and (Id) forms a duplex with a antisense strand being represented by any one of formulas (IIa), (IIb), (IIc), and (IId), respectively.

Accordingly, the RNAi agents for use in the methods of the invention may comprise a sense strand and an antisense strand, each strand having 14 to 30 nucleotides, the RNAi duplex represented by formula (III).

sense:
5′ np-Na-(X X X)i-Nb-Y Y Y-Nb-(Z Z Z)j-Na-nq 3′
antisense:
(III)
3′ np′-Na′-(X′X′X′)k-Nb′-Y′Y′Y′-Nb′-(Z′Z′Z′)1-Na′-
nq′ 5′

wherein:

i, j, k, and 1 are each independently 0 or 1;

p, p′, q, and q′ are each independently 0-6;
each Na and Na′ independently represents an oligonucleotide sequence comprising 0-25 modified nucleotides, each sequence comprising at least two differently modified nucleotides;
each Nb and Nb′ independently represents an oligonucleotide sequence comprising 0-10 modified nucleotides;
wherein each np′, np, nq′, and nq, each of which may or may not be present, independently represents an overhang nucleotide; and
XXX, YYY, ZZZ, X′X′X′, Y′Y′Y′, and Z′Z′Z′ each independently represent one motif of three identical modifications on three consecutive nucleotides.

In one embodiment, i is 0 and j is 0; or i is 1 and j is 0; or i is 0 and j is 1; or both i and j are 0; or both i and j are 1. In another embodiment, k is 0 and l is 0; or k is 1 and l is 0; k is 0 and l is 1; or both k and 1 are 0; or both k and l are 1.

Exemplary combinations of the sense strand and antisense strand forming a RNAi duplex include the formulas below:

(IIIa)
5′ np-Na-Y Y Y-Na-nq 3′
3′ np′-Na′-Y′Y′Y′-Na′nq′ 5′
(IIIb)
5′ np-Na -Y Y Y-Nb-Z Z Z-Na-nq 3′
3′ np′-Na′-Y′Y′Y′-Nb′-Z′Z′Z′-Na′nq′ 5′
(IIIc)
5′ np-Na-X X X-Nb-Y Y Y-Na-nq 3′
3′ np′-Na′-X′X′X′-Nb′-Y′Y′Y′-Na′-nq′ 5′
(IIId)
5′ np-Na-X X X-Nb-Y Y Y-Nb-Z Z Z-Na-nq 3′
3′ np′-Na′-X′X′X′-Nb′-Y′Y′Y′-Nb′-Z′Z′Z′-Na-nq′ 5′

When the RNAi agent is represented by formula (IIIa), each Na independently represents an oligonucleotide sequence comprising 2-20, 2-15, or 2-10 modified nucleotides.

When the RNAi agent is represented by formula (IIIB), each Nb independently represents an oligonucleotide sequence comprising 1-10, 1-7, 1-5 or 1-4 modified nucleotides. Each Na independently represents an oligonucleotide sequence comprising 2-20, 2-15, or 2-10 modified nucleotides.

When the RNAi agent is represented as formula (Inc), each Nb, Nb′ independently represents an oligonucleotide sequence comprising 0-10, 0-7, 0-10, 0-7, 0-5, 0-4, 0-2 or 0 modified nucleotides. Each Na independently represents an oligonucleotide sequence comprising 2-20, 2-15, or 2-10 modified nucleotides.

When the RNAi agent is represented as formula (Ind), each Nb, Nb′ independently represents an oligonucleotide sequence comprising 0-10, 0-7, 0-10, 0-7, 0-5, 0-4, 0-2 or Omodified nucleotides. Each Na, Na′ independently represents an oligonucleotide sequence comprising 2-20, 2-15, or 2-10 modified nucleotides. Each of Na, Na′, Nb and Nb′ independently comprises modifications of alternating pattern.

Each of X, Y and Z in formulas (III), (IIIa), (IIIb), (IIIc), and (IIId) may be the same or different from each other.

When the RNAi agent is represented by formula (III), (IIIa), (IIIb), (IIIc), and (IIId), at least one of the Y nucleotides may form a base pair with one of the Y′ nucleotides. Alternatively, at least two of the Y nucleotides form base pairs with the corresponding Y′ nucleotides; or all three of the Y nucleotides all form base pairs with the corresponding Y′ nucleotides.

When the RNAi agent is represented by formula (IIIb) or (IIId), at least one of the Z nucleotides may form a base pair with one of the Z′ nucleotides. Alternatively, at least two of the Z nucleotides form base pairs with the corresponding Z′ nucleotides; or all three of the Z nucleotides all form base pairs with the corresponding Z′ nucleotides.

When the RNAi agent is represented as formula (Inc) or (IIkt), at least one of the X nucleotides may form a base pair with one of the X′ nucleotides. Alternatively, at least two of the X nucleotides form base pairs with the corresponding X′ nucleotides; or all three of the X nucleotides all form base pairs with the corresponding X′ nucleotides.

In one embodiment, the modification on the Y nucleotide is different than the modification on the Y′ nucleotide, the modification on the Z nucleotide is different than the modification on the Z′ nucleotide, and/or the modification on the X nucleotide is different than the modification on the X′ nucleotide.

In one embodiment, when the RNAi agent is represented by formula (IIkt), the Na modifications are 2′-O-methyl or 2′-fluoro modifications. In another embodiment, when the RNAi agent is represented by formula (IIkt), the Na modifications are 2′-O-methyl or 2′-fluoro modifications and np′>0 and at least one np′ is linked to a neighboring nucleotide a via phosphorothioate linkage. In yet another embodiment, when the RNAi agent is represented by formula (IIkt), the Na modifications are 2′-O-methyl or 2′-fluoro modifications, np′>0 and at least one np′ is linked to a neighboring nucleotide via phosphorothioate linkage, and the sense strand is conjugated to one or more GalNAc derivatives attached through a bivalent or trivalent branched linker (described below). In another embodiment, when the RNAi agent is represented by formula (IIId), the Na modifications are 2′-O-methyl or 2′-fluoro modifications, np′>0 and at least one np′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In one embodiment, when the RNAi agent is represented by formula (IIIa), the Na modifications are 2′-O-methyl or 2′-fluoro modifications, np′>0 and at least one np′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In one embodiment, the RNAi agent is a multimer containing at least two duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, the RNAi agent is a multimer containing three, four, five, six or more duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, two RNAi agents represented by formula (III), (IIIa), (IIIB), (IIIc), and (IIId) are linked to each other at the 5′ end, and one or both of the 3′ ends and are optionally conjugated to a ligand. Each of the agents can target the same gene or two different genes; or each of the agents can target same gene at two different target sites.

In certain embodiments, an RNAi agent of the invention may contain a low number of nucleotides containing a 2′-fluoro modification, e.g., 10 or fewer nucleotides with 2′-fluoro modification. For example, the RNAi agent may contain 10, 9, 8, 7, 6, 5, 4, 3, 2, 1 or 0 nucleotides with a 2′-fluoro modification. In a specific embodiment, the RNAi agent of the invention contains 10 nucleotides with a 2′-fluoro modification, e.g., 4 nucleotides with a 2′-fluoro modification in the sense strand and 6 nucleotides with a 2′-fluoro modification in the antisense strand. In another specific embodiment, the RNAi agent of the invention contains 6 nucleotides with a 2′-fluoro modification, e.g., 4 nucleotides with a 2′-fluoro modification in the sense strand and 2 nucleotides with a 2′-fluoro modification in the antisense strand.

In other embodiments, an RNAi agent of the invention may contain an ultra low number of nucleotides containing a 2′-fluoro modification, e.g., 2 or fewer nucleotides containing a 2′-fluoro modification. For example, the RNAi agent may contain 2, 1 of 0 nucleotides with a 2′-fluoro modification. In a specific embodiment, the RNAi agent may contain 2 nucleotides with a 2′-fluoro modification, e.g., 0 nucleotides with a 2-fluoro modification in the sense strand and 2 nucleotides with a 2′-fluoro modification in the antisense strand.

Various publications describe multimeric RNAi agents that can be used in the methods of the invention. Such publications include WO2007/091269, U.S. Pat. No. 7,858,769, WO2010/141511, WO2007/117686, WO2009/014887 and WO2011/031520 the entire contents of each of which are hereby incorporated herein by reference.

As described in more detail below, the RNAi agent that contains conjugations of one or more carbohydrate moieties to a RNAi agent can optimize one or more properties of the RNAi agent. In many cases, the carbohydrate moiety will be attached to a modified subunit of the RNAi agent. For example, the ribose sugar of one or more ribonucleotide subunits of a dsRNA agent can be replaced with another moiety, e.g., a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the polynucleotide via a carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The RNAi agents may be conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and and decalin; preferably, the acyclic group is selected from serinol backbone or diethanolamine backbone.

In another embodiment of the invention, an iRNA agent comprises a sense strand and an antisense strand, each strand having 14 to 40 nucleotides. The RNAi agent may be represented by formula (L):

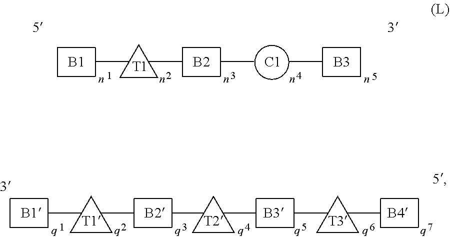

In formula (L), B1, B2, B3, B1′, B2′, B3′, and B4′ each are independently a nucleotide containing a modification selected from the group consisting of 2′-O-alkyl, 2′-substituted alkoxy, 2′-substituted alkyl, 2′-halo, ENA, and BNA/LNA. In one embodiment, B1, B2, B3, B1′, B2′, B3′, and B4′ each contain 2′-OMe modifications. In one embodiment, B1, B2, B3, B1′, B2′, B3′, and B4′ each contain 2′-OMe or 2′-F modifications. In one embodiment, at least one of B1, B2, B3, B1′, B2′, B3′, and B4′ contain 2′-O—N-methylacetamido (2′-O-NMA) modification.

C1 is a thermally destabilizing nucleotide placed at a site opposite to the seed region of the antisense strand (i.e., at positions 2-8 of the 5′-end of the antisense strand). For example, C1 is at a position of the sense strand that pairs with a nucleotide at positions 2-8 of the 5′-end of the antisense strand. In one example, C1 is at position 15 from the 5′-end of the sense strand. C1 nucleotide bears the thermally destabilizing modification which can include abasic modification; mismatch with the opposing nucleotide in the duplex; and sugar modification such as 2′-deoxy modification or acyclic nucleotide e.g., unlocked nucleic acids (UNA) or glycerol nucleic acid (GNA). In one embodiment, C1 has thermally destabilizing modification selected from the group consisting of: i) mismatch with the opposing nucleotide in the antisense strand; ii) abasic modification selected from the group consisting of:

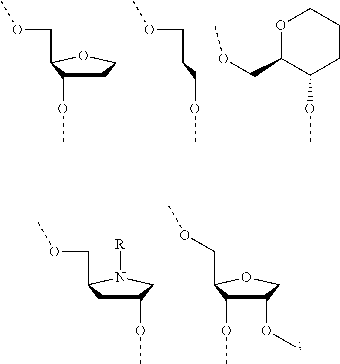

and iii) sugar modification selected from the group consisting of:

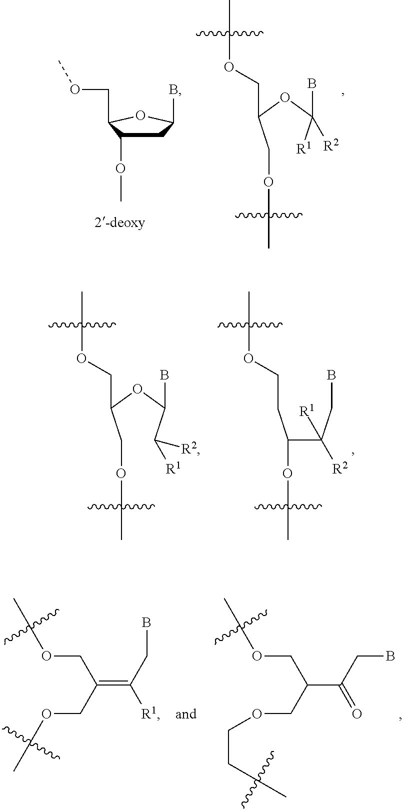

wherein B is a modified or unmodified nucleobase, R¹ and R² independently are H, halogen, OR₃, or alkyl; and R₃ is H, alkyl, cycloalkyl, aryl, aralkyl, heteroaryl or sugar. In one embodiment, the thermally destabilizing modification in C1 is a mismatch selected from the group consisting of G:G, G:A, G:U, G:T, A:A, A:C, C:C, C:U, C:T, U:U, T:T, and U:T; and optionally, at least one nucleobase in the mismatch pair is a 2′-deoxy nucleobase. In one example, the thermally destabilizing modification in C1 is GNA or

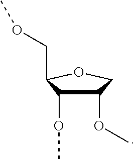

T1, T1′, T2′, and T3′ each independently represent a nucleotide comprising a modification providing the nucleotide a steric bulk that is less or equal to the steric bulk of a 2′-OMe modification. A steric bulk refers to the sum of steric effects of a modification. Methods for determining steric effects of a modification of a nucleotide are known to one skilled in the art. The modification can be at the 2′ position of a ribose sugar of the nucleotide, or a modification to a non-ribose nucleotide, acyclic nucleotide, or the backbone of the nucleotide that is similar or equivalent to the 2′ position of the ribose sugar, and provides the nucleotide a steric bulk that is less than or equal to the steric bulk of a 2′-OMe modification. For example, T1, T1′, T2′, and T3′ are each independently selected from DNA, RNA, LNA, 2′-F, and 2′-F-5′-methyl. In one embodiment, T1 is DNA. In one embodiment, T1′ is DNA, RNA or LNA. In one embodiment, T2′ is DNA or RNA. In one embodiment, T3′ is DNA or RNA.

n¹, n³, and q¹ are independently 4 to 15 nucleotides in length.

n⁵, q³, and q⁷ are independently 1-6 nucleotide(s) in length.

n⁴, q², and q⁶ are independently 1-3 nucleotide(s) in length; alternatively, n⁴ is 0.

q⁵ is independently 0-10 nucleotide(s) in length.

n² and q⁴ are independently 0-3 nucleotide(s) in length.

Alternatively, n⁴ is 0-3 nucleotide(s) in length.

In one embodiment, n⁴ can be 0. In one example, n⁴ is 0, and q² and q⁶ are 1. In another example, n⁴ is 0, and q² and q⁶ are 1, with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴, q², and q⁶ are each 1.

In one embodiment, n², n⁴, q², q⁴, and q⁶ are each 1.

In one embodiment, C1 is at position 14-17 of the 5′-end of the sense strand, when the sense strand is 19-22 nucleotides in length, and n⁴ is 1. In one embodiment, C1 is at position 15 of the 5′-end of the sense strand

In one embodiment, T3′ starts at position 2 from the 5′ end of the antisense strand. In one example, T3′ is at position 2 from the 5′ end of the antisense strand and q⁶ is equal to 1.

In one embodiment, T1′ starts at position 14 from the 5′ end of the antisense strand. In one example, T1′ is at position 14 from the 5′ end of the antisense strand and q² is equal to 1.

In an exemplary embodiment, T3′ starts from position 2 from the 5′ end of the antisense strand and T1′ starts from position 14 from the 5′ end of the antisense strand. In one example, T3′ starts from position 2 from the 5′ end of the antisense strand and q⁶ is equal to 1 and T1′ starts from position 14 from the 5′ end of the antisense strand and q² is equal to 1.

In one embodiment, T1′ and T3′ are separated by 11 nucleotides in length (i.e. not counting the T1′ and T3′ nucleotides).

In one embodiment, T1′ is at position 14 from the 5′ end of the antisense strand. In one example, T1′ is at position 14 from the 5′ end of the antisense strand and q² is equal to 1, and the modification at the 2′ position or positions in a non-ribose, acyclic or backbone that provide less steric bulk than a 2′-OMe ribose.

In one embodiment, T3′ is at position 2 from the 5′ end of the antisense strand. In one example, T3′ is at position 2 from the 5′ end of the antisense strand and q⁶ is equal to 1, and the modification at the 2′ position or positions in a non-ribose, acyclic or backbone that provide less than or equal to steric bulk than a 2′-OMe ribose.

In one embodiment, T1 is at the cleavage site of the sense strand. In one example, T1 is at position 11 from the 5′ end of the sense strand, when the sense strand is 19-22 nucleotides in length, and n² is 1. In an exemplary embodiment, T1 is at the cleavage site of the sense strand at position 11 from the 5′ end of the sense strand, when the sense strand is 19-22 nucleotides in length, and n² is 1,

In one embodiment, T2′ starts at position 6 from the 5′ end of the antisense strand. In one example, T2′ is at positions 6-10 from the 5′ end of the antisense strand, and q⁴ is 1.

In an exemplary embodiment, T1 is at the cleavage site of the sense strand, for instance, at position 11 from the 5′ end of the sense strand, when the sense strand is 19-22 nucleotides in length, and n² is 1; T1′ is at position 14 from the 5′ end of the antisense strand, and q² is equal to 1, and the modification to T1′ is at the 2′ position of a ribose sugar or at positions in a non-ribose, acyclic or backbone that provide less steric bulk than a 2′-OMe ribose; T2′ is at positions 6-10 from the 5′ end of the antisense strand, and q⁴ is 1; and T3′ is at position 2 from the 5′ end of the antisense strand, and q⁶ is equal to 1, and the modification to T3′ is at the 2′ position or at positions in a non-ribose, acyclic or backbone that provide less than or equal to steric bulk than a 2′-OMe ribose.

In one embodiment, T2′ starts at position 8 from the 5′ end of the antisense strand. In one example, T2′ starts at position 8 from the 5′ end of the antisense strand, and q⁴ is 2.

In one embodiment, T2′ starts at position 9 from the 5′ end of the antisense strand. In one example, T2′ is at position 9 from the 5′ end of the antisense strand, and q⁴ is 1.

In one embodiment, BF is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 6, T1 is 2′F, n² is 3, B2 is 2′-OMe, n³ is 7, n⁴ is 0, B3 is 2′OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 6, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1; with two phosphorothioate internucleotide linkage modifications within positions 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

The RNAi agent can comprise a phosphorus-containing group at the 5′-end of the sense strand or antisense strand. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos), or 5′-deoxy-5′-C-malonyl

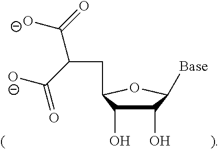

When the 5′-end phosphorus-containing group is 5′-end vinylphosphonate (5′-VP), the 5′-VP can be either 5′-E-VP isomer (i.e., trans-vinylphosphate,

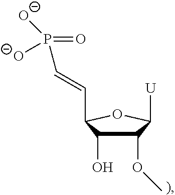

5′-Z-VP isomer (i.e., cis-vinylphosphate,

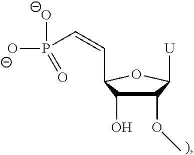

or mixtures thereof.

In one embodiment, the RNAi agent comprises a phosphorus-containing group at the 5′-end of the sense strand. In one embodiment, the RNAi agent comprises a phosphorus-containing group at the 5′-end of the antisense strand.

In one embodiment, the RNAi agent comprises a 5′-P. In one embodiment, the RNAi agent comprises a 5′-P in the antisense strand.

In one embodiment, the RNAi agent comprises a 5′-PS. In one embodiment, the RNAi agent comprises a 5′-PS in the antisense strand.

In one embodiment, the RNAi agent comprises a 5′-VP. In one embodiment, the RNAi agent comprises a 5′-VP in the antisense strand. In one embodiment, the RNAi agent comprises a 5′-E-VP in the antisense strand. In one embodiment, the RNAi agent comprises a 5′-Z-VP in the antisense strand.

In one embodiment, the RNAi agent comprises a 5′-PS₂. In one embodiment, the RNAi agent comprises a 5′-PS₂ in the antisense strand.

In one embodiment, the RNAi agent comprises a 5′-PS₂. In one embodiment, the RNAi agent comprises a 5′-deoxy-5′-C-malonyl in the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNAi RNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P. In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof), and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS₂ and a targeting ligand. In one embodiment, the 5′-PS₂ is at the 5′-end of the antisense strand, and the targeting ligand is at the 3′-end of the sense strand. In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′- OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In a particular embodiment, an RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker; and
   (iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
      and
(b) an antisense strand having:
   (i) a length of 23 nucleotides;
   (ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
   (iii) phosphorothioate internucleotide linkages between nucleotide positions 21 and 22, and between nucleotide positions 22 and 23 (counting from the 5′ end);
      wherein the dsRNA agents have a two nucleotide overhang at the 3′-end of the antisense strand, and a blunt end at the 5′-end of the antisense strand.

In another particular embodiment, an RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, 13, 15, 17, 19, and 21, and 2′-OMe modifications at positions 2, 4, 6, 8, 12, 14, 16, 18, and 20 (counting from the 5′ end); and
   (iv) phosphorothioate internucleotide linkages between nucleotide positions 1 and 2, and between nucleotide positions 2 and 3 (counting from the 5′ end);
      and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, 10, and 12 to 21, 2′-F modifications at positions 7, and 9, and a desoxy-nucleotide (e.g. dT) at position 11 (counting from the 5′ end); and
   (iv) phosphorothioate internucleotide linkages between nucleotide positions 1 and 2, and between nucleotide positions 2 and 3 (counting from the 5′ end);
      and
(b) an antisense strand having:
   (i) a length of 23 nucleotides;
   (ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the RNAi agents have a two nucleotide overhang at the 3′-end of the antisense strand, and a blunt end at the 5′-end of the antisense strand.

In another particular embodiment, aRNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, 10, 12, 14, and 16 to 21, and 2′-F modifications at positions 7, 9, 11, 13, and 15; and
   (iv) phosphorothioate internucleotide linkages between nucleotide positions 1 and 2, and between nucleotide positions 2 and 3 (counting from the 5′ end);
      and
(b) an antisense strand having:
   (i) a length of 23 nucleotides;
   (ii) 2′-OMe modifications at positions 1, 5, 7, 9, 11, 13, 15, 17, 19, and 21 to 23, and 2′-F modifications at positions 2 to 4, 6, 8, 10, 12, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 9, and 12 to 21, and 2′-F modifications at positions 10, and 11; and
   (iv) phosphorothioate internucleotide linkages between nucleotide positions 1 and 2, and between nucleotide positions 2 and 3 (counting from the 5′ end);
      and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′-F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, and 13, and 2′-OMe modifications at positions 2, 4, 6, 8, 12, and 14 to 21; and
   (iv) phosphorothioate internucleotide linkages between nucleotide positions 1 and 2, and between nucleotide positions 2 and 3 (counting from the 5′ end);
      and
(b) an antisense strand having:
   (i) a length of 23 nucleotides;
   (ii) 2′-OMe modifications at positions 1, 3, 5 to 7, 9, 11 to 13, 15, 17 to 19, and 21 to 23, and 2′-F modifications at positions 2, 4, 8, 10, 14, 16, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the RNAi agents have a two nucleotide overhang at the 3′-end of the antisense strand, and a blunt end at the 5′-end of the antisense strand.

In another particular embodiment, a RNAi agentsof the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1, 2, 4, 6, 8, 12, 14, 15, 17, and 19 to 21, and 2′-F modifications at positions 3, 5, 7, 9 to 11, 13, 16, and 18; and
   (iv) phosphorothioate internucleotide linkages between nucleotide positions 1 and 2, and between nucleotide positions 2 and 3 (counting from the 5′ end);
      and
(b) an antisense strand having:
   (i) a length of 25 nucleotides;
   (ii) 2′-OMe modifications at positions 1, 4, 6, 7, 9, 11 to 13, 15, 17, and 19 to 23, 2′-F modifications at positions 2, 3, 5, 8, 10, 14, 16, and 18, and desoxy-nucleotides (e.g. dT) at positions 24 and 25 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the RNAi agents have a four nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
      and
(b) an antisense strand having:
   (i) a length of 23 nucleotides;
   (ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
      and
(b) an antisense strand having:
   (i) a length of 23 nucleotides;
   (ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 19 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 4, 6, and 10 to 19, and 2′-F modifications at positions 5, and 7 to 9; and
   (iv) phosphorothioate internucleotide linkages between nucleotide positions 1 and 2, and between nucleotide positions 2 and 3 (counting from the 5′ end);
      and
(b) an antisense strand having:
   (i) a length of 21 nucleotides;
   (ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 19 and 20, and between nucleotide positions 20 and 21 (counting from the 5′ end);
   wherein the RNAi agents have a two nucleotide overhang at the 3′-end of the antisense strand, and a blunt end at the 5′-end of the antisense strand.

### IV. iRNAs Conjugated to Ligands

Another modification of the RNA of an iRNA of the invention involves chemically linking to the RNA one or more ligands, moieties or conjugates that enhance the activity, cellular distribution or cellular uptake of the iRNA. Such moieties include but are not limited to lipid moieties such as a cholesterol moiety (Letsinger et al., (1989) Proc. Natl. Acid. Sci. USA, 86: 6553-6556), cholic acid (Manoharan et al., (1994) Biorg. Med. Chem. Let., 4:1053-1060), a thioether, e.g., beryl-S-tritylthiol (Manoharan et al., (1992) Ann. N.Y. Acad. Sci., 660:306-309; Manoharan et al., (1993) Biorg. Med. Chem. Let., 3:2765-2770), a thiocholesterol (Oberhauser et al., (1992) Nucl. Acids Res., 20:533-538), an aliphatic chain, e.g., dodecandiol or undecyl residues (Saison-Behmoaras et al., (1991) EMBO J, 10:1111-1118; Kabanov et al., (1990) FEBS Lett., 259:327-330; Svinarchuk et al., (1993) Biochimie, 75:49-54), a phospholipid, e.g., di-hexadecyl-rac-glycerol or triethyl-ammonium 1,2-di-O-hexadecyl-rac-glycero-3-phosphonate (Manoharan et al., (1995) Tetrahedron Lett., 36:3651-3654; Shea et al., (1990) Nucl. Acids Res., 18:3777-3783), a polyamine or a polyethylene glycol chain (Manoharan et al., (1995) Nucleosides & Nucleotides, 14:969-973), or adamantane acetic acid (Manoharan et al., (1995) Tetrahedron Lett., 36:3651-3654), a palmityl moiety (Mishra et al., (1995) Biochim. Biophys. Acta, 1264:229-237), or an octadecylamine or hexylamino-carbonyloxycholesterol moiety (Crooke et al., (1996) J. Pharmacol. Exp. Ther., 277:923-937).

In embodiments in which a first dsRNA agent targeting LDHA and a second dsRNA agent targeting HAO1 are covalently attached (i.e., a dual targeting RNAi agent described herein), one or both of the dsRNA agents may independently comprise one or more ligands.

In one embodiment, a ligand alters the distribution, targeting or lifetime of an iRNA agent into which it is incorporated. In preferred embodiments a ligand provides an enhanced affinity for a selected target, e.g., molecule, cell or cell type, compartment, e.g., a cellular or organ compartment, tissue, organ or region of the body, as, e.g., compared to a species absent such a ligand. Preferred ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralen, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an iRNA as described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated oligonucleotides of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides, such as the phosphorothioates and alkylated derivatives.

In the ligand-conjugated oligonucleotides and ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

A. Lipid Conujugates

In one embodiment, the ligand or conjugate is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In a preferred embodiment, the lipid based ligand binds HSA. Preferably, it binds HSA with a sufficient affinity such that the conjugate will be preferably distributed to a non-kidney tissue. However, it is preferred that the affinity not be so strong that the HSA-ligand binding cannot be reversed.

In another preferred embodiment, the lipid based ligand binds HSA weakly or not at all, such that the conjugate will be preferably distributed to the kidney. Other moieties that target to kidney cells can also be used in place of or in addition to the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by target cells such as liver cells. Also included are HSA and low density lipoprotein (LDL).

B. Cell Permeation Agents

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 2986). An RFGF analogue (e.g., amino acid sequence AALLPVLLAAP (SEQ ID NO: 2987) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO: 2988) and the Drosophila Antennapedia protein (RQIKIWFQNRRMKWKK (SEQ ID NO: 2989) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to a dsRNA agent via an incorporated monomer unit for cell targeting purposes is an arginine-glycine-aspartic acid (RGD)-peptide, or RGD mimic A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glyciosylated or methylated, to facilitate targeting to a specific tissue(s). RGD-containing peptides and peptidiomimemtics may include D-amino acids, as well as synthetic RGD mimics. In addition to RGD, one can use other moieties that target the integrin ligand. Preferred conjugates of this ligand target PECAM-1 or VEGF.

A “cell permeation peptide” is capable of permeating a cell, e.g., a microbial cell, such as a bacterial or fungal cell, or a mammalian cell, such as a human cell. A microbial cell-permeating peptide can be, for example, a α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

C. Carbohydrate Conjugates

In some embodiments of the compositions and methods of the invention, an iRNA oligonucleotide further comprises a carbohydrate. The carbohydrate conjugated iRNA are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In embodiments in which a first dsRNA agent targeting LDHA and a second dsRNA agent targeting HAO1 are covalently attached (i.e., a dual targetingRNAi agent), one or both of the dsRNA agents may independently comprise one or more carbohydrate ligands.

In one embodiment, a carbohydrate conjugate for use in the compositions and methods of the invention is selected from the group consisting of:

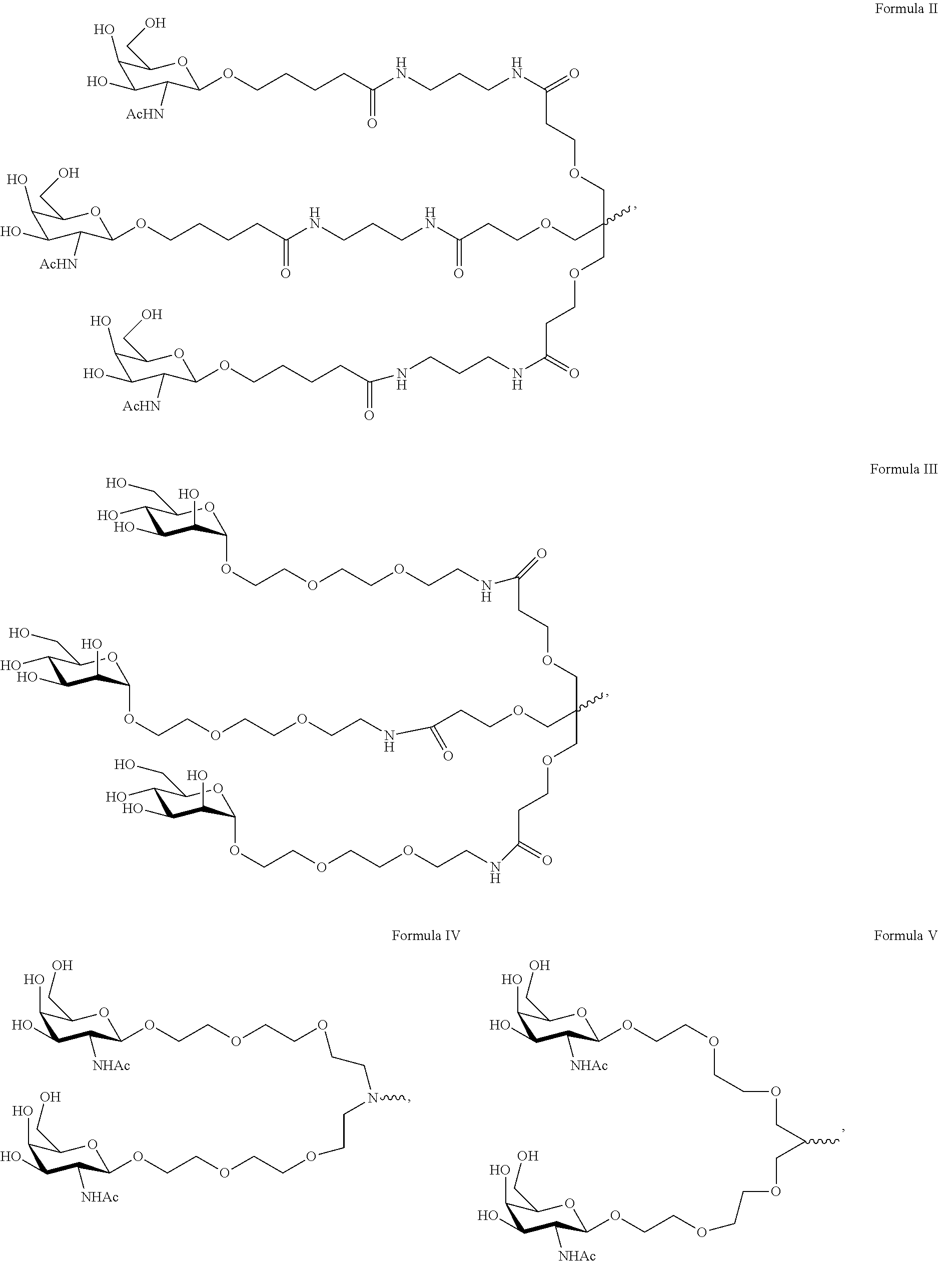

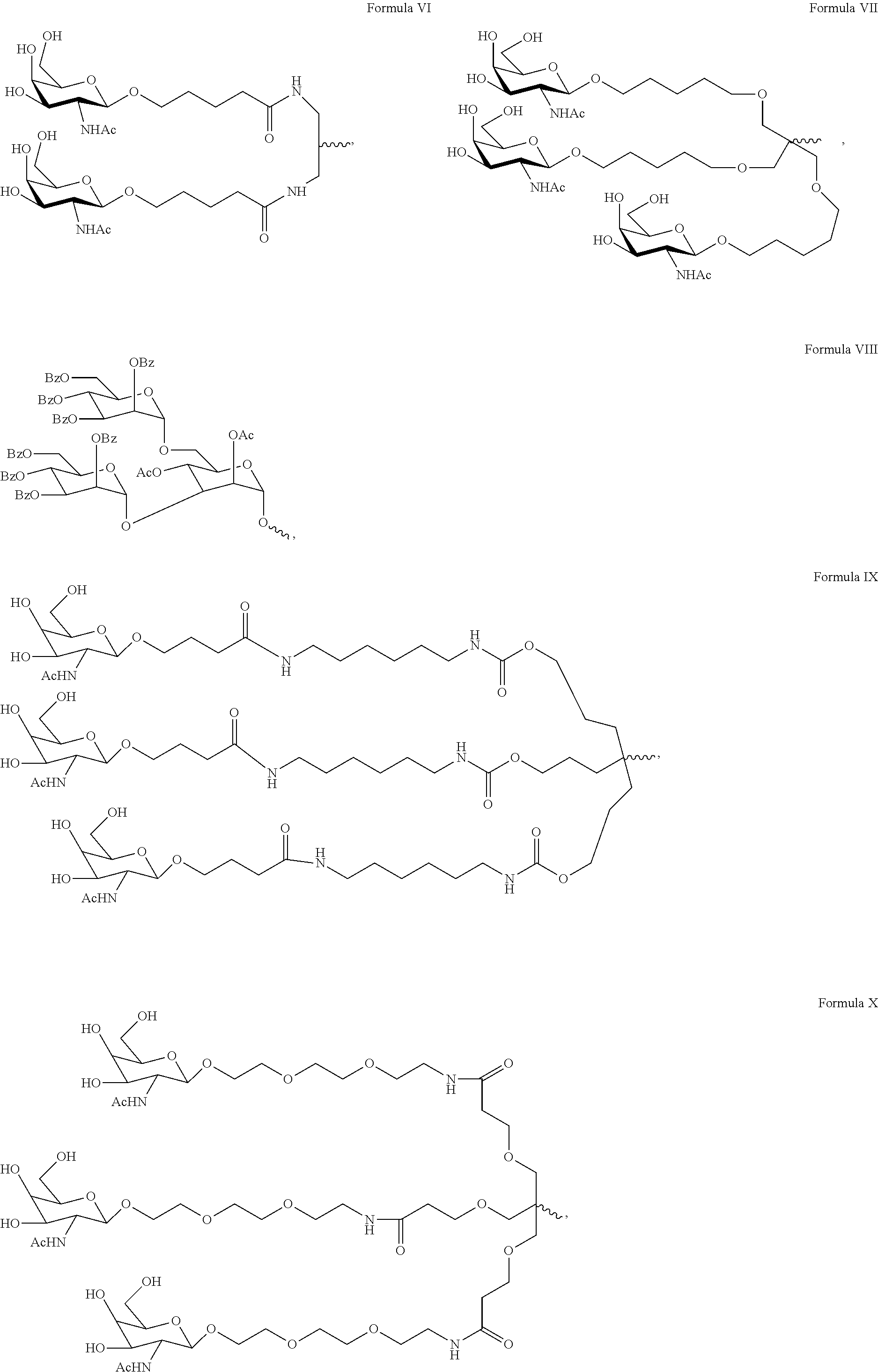

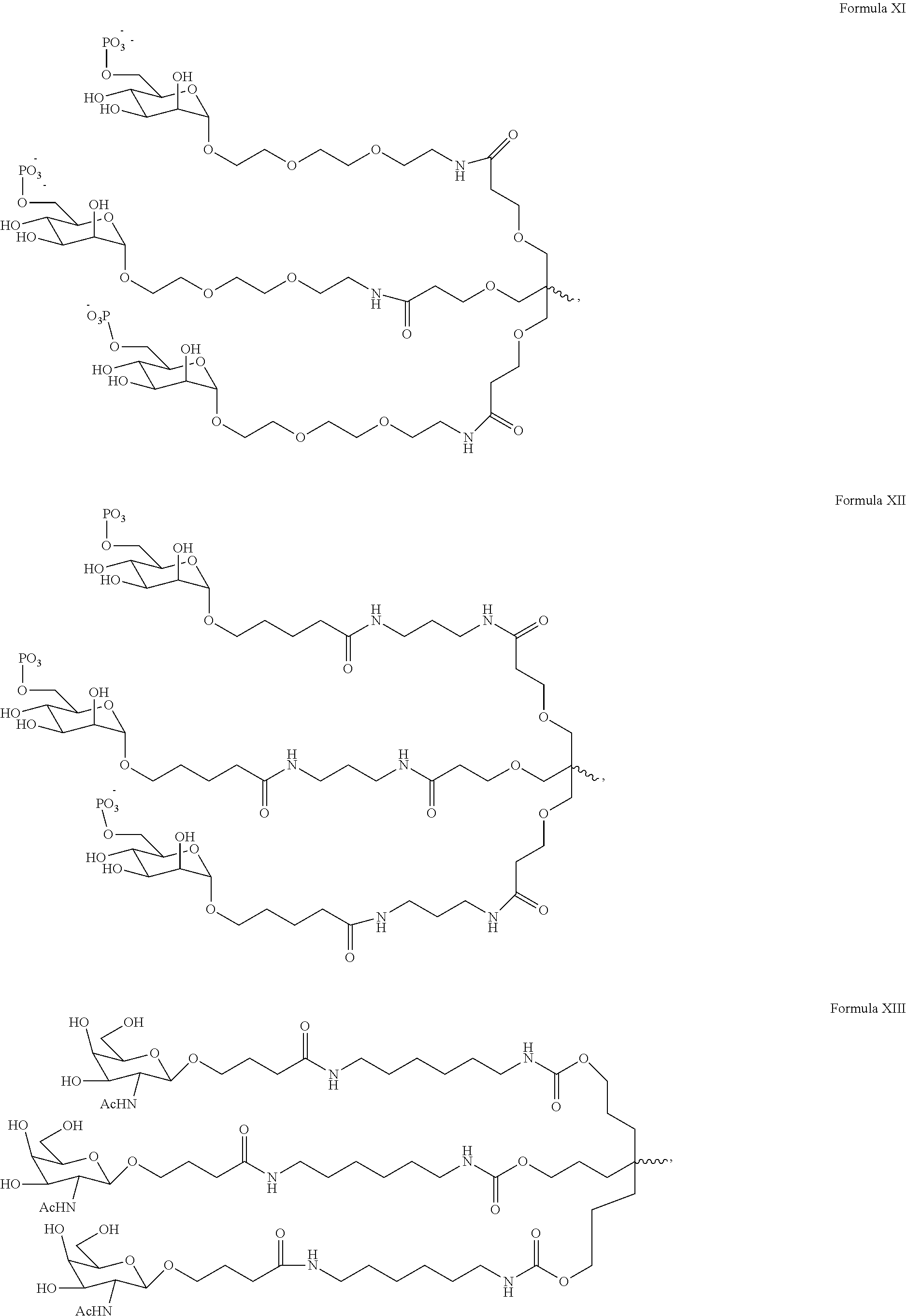

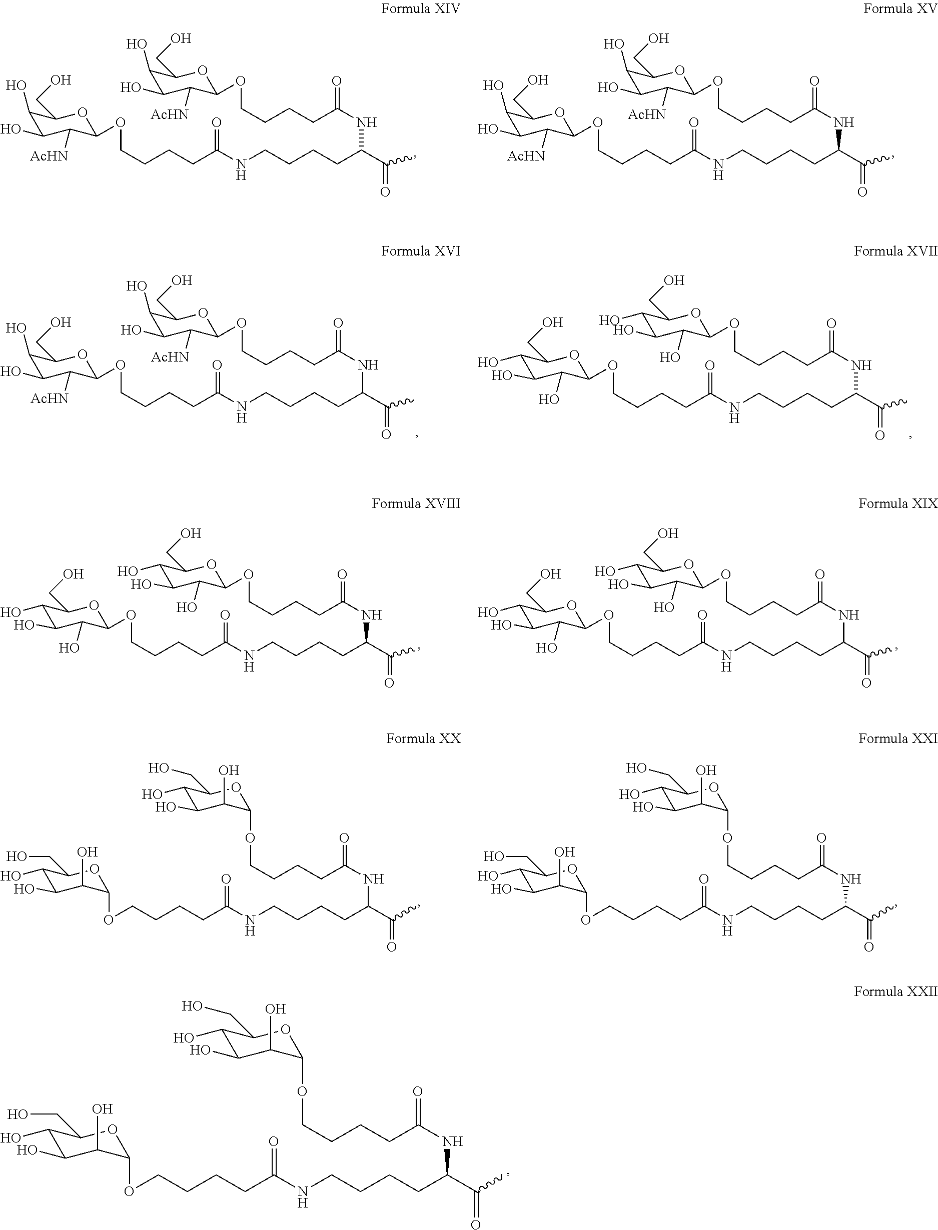

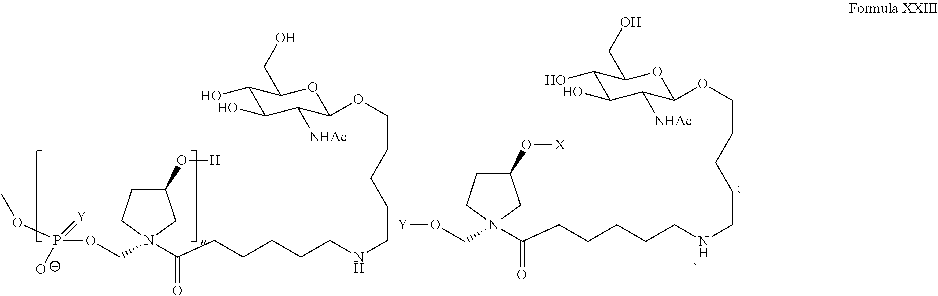

wherein Y is O or S and n is 3-6 (Formula XXIV);

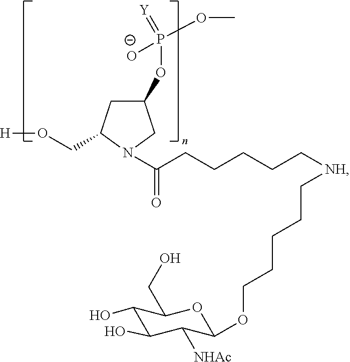

wherein Y is O or S and n is 3-6 (Formula XXV);

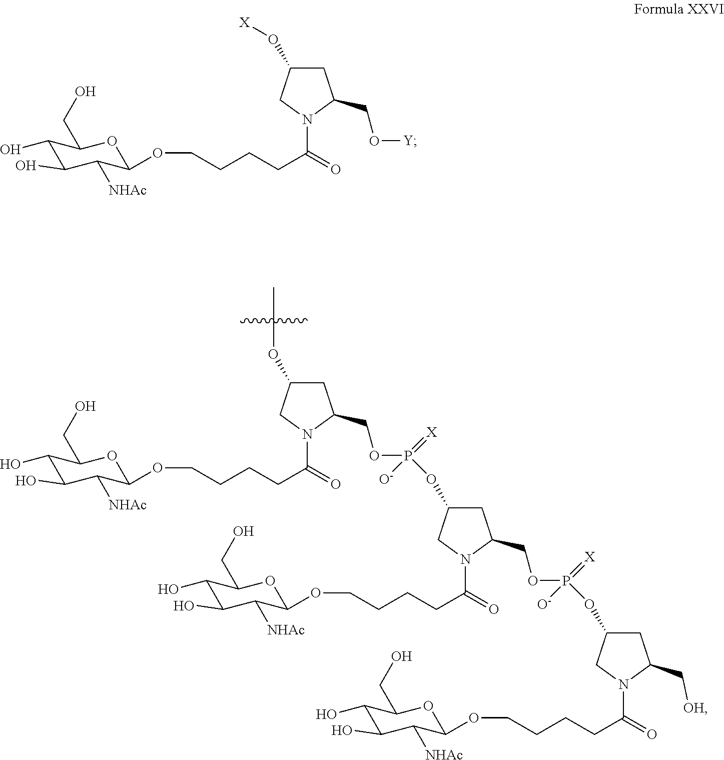

wherein X is O or S (Formula XXVII);

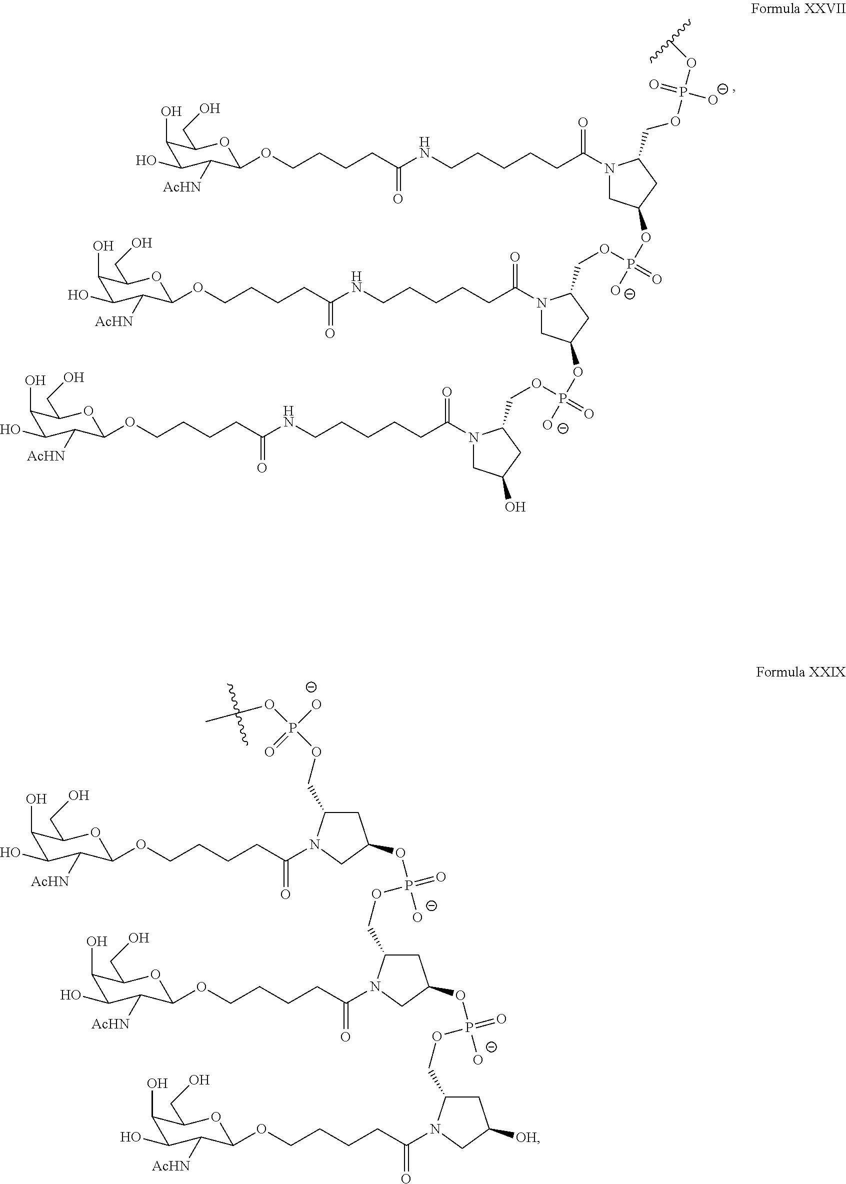 [SMILES: *OP(=O)([O-])OC[C@@H]1C[C@@H](OP(=O)([O-])OC[C@@H]2C[C@@H](OP(=O)([O-])OC[C@@H]3C[C@@H](O)CN3C(=O)CCCCCNC(=O)CCCCOC3OC(CO)C(O)C(O)C3NC(C)=O)CN2C(=O)CCCCCNC(=O)CCCCOC2OC(CO)C(O)C(O)C2NC(C)=O)CN1C(=O)CCCCCNC(=O)CCCCOC1OC(CO)C(O)C(O)C1NC(C)=O ; *OP(=O)([O-])OC[C@@H]1C[C@@H](OP(=O)([O-])OC[C@@H]2C[C@@H](OP(=O)([O-])OC[C@@H]3C[C@@H](O)CN3C(=O)CCCCOC3OC(CO)C(O)C(O)C3NC(C)=O)CN2C(=O)CCCCOC2OC(CO)C(O)C(O)C2NC(C)=O)CN1C(=O)CCCCOC1OC(CO)C(O)C(O)C1NC(C)=O]

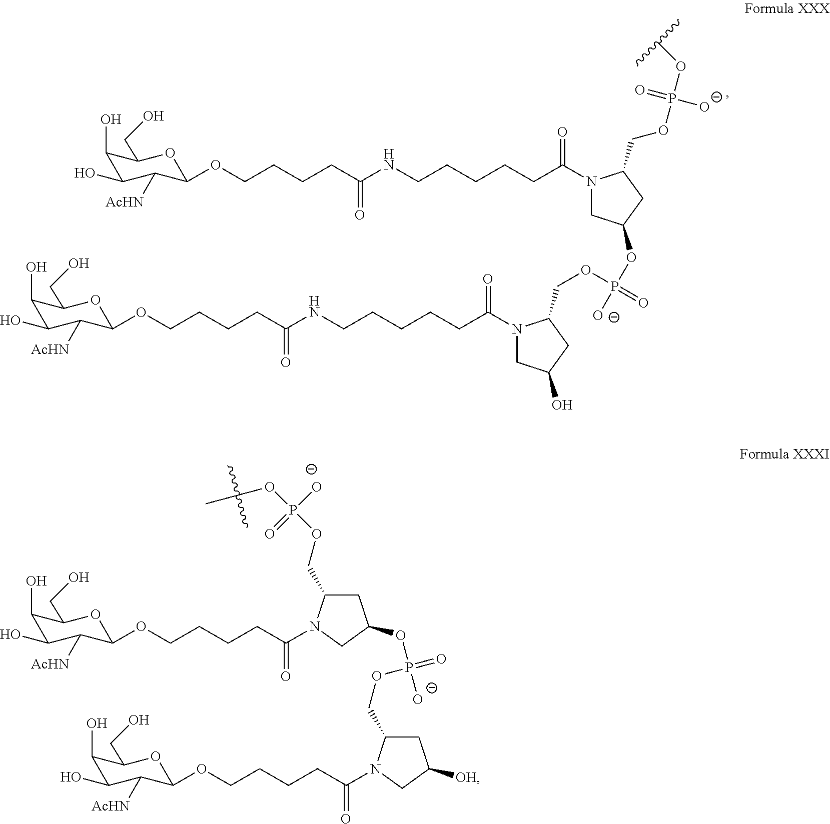 [SMILES: *OP(=O)([O-])OC[C@@H]1C[C@@H](OP(=O)([O-])OC[C@@H]2C[C@@H](O)CN2C(=O)CCCCCNC(=O)CCCCOC2OC(CO)C(O)C(O)C2NC(C)=O)CN1C(=O)CCCCCNC(=O)CCCCOC1OC(CO)C(O)C(O)C1NC(C)=O ; *OP(=O)([O-])OC[C@@H]1C[C@@H](OP(=O)([O-])OC[C@@H]2C[C@@H](O)CN2C(=O)CCCCOC2OC(CO)C(O)C(O)C2NC(C)=O)CN1C(=O)CCCCOC1OC(CO)C(O)C(O)C1NC(C)=O]

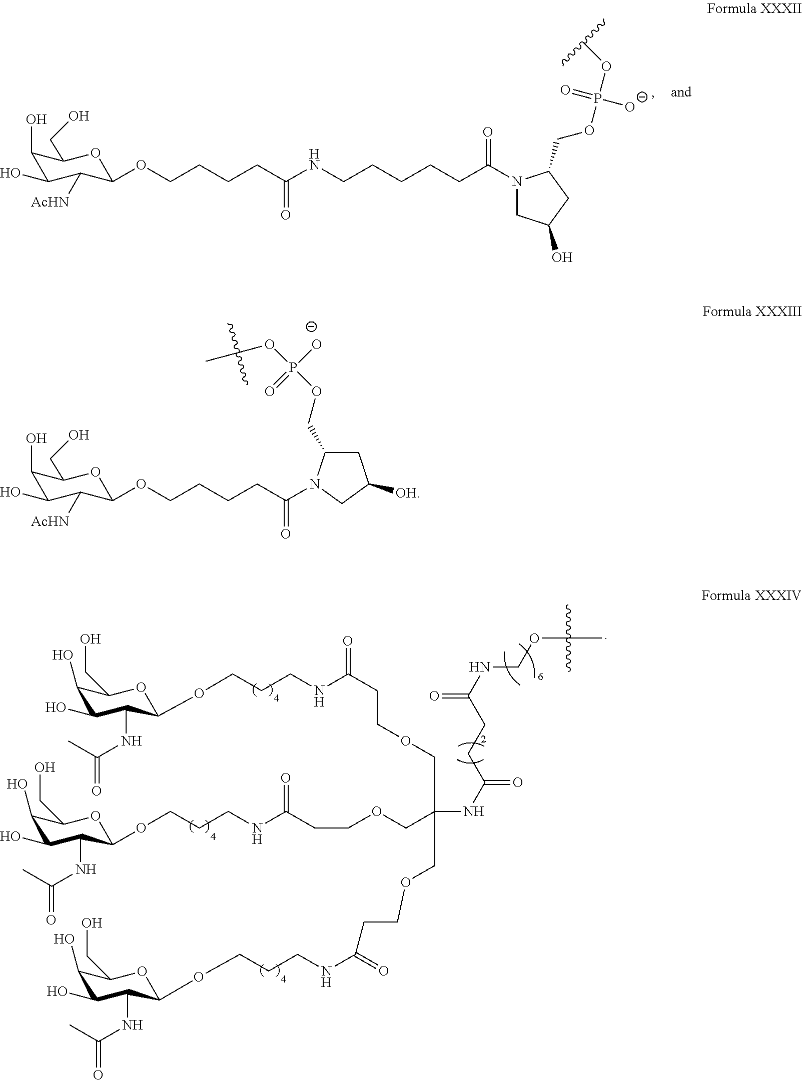

In another embodiment, a carbohydrate conjugate for use in the compositions and methods of the invention is a monosaccharide. In one embodiment, the monosaccharide is an N-acetylgalactosamine, such as

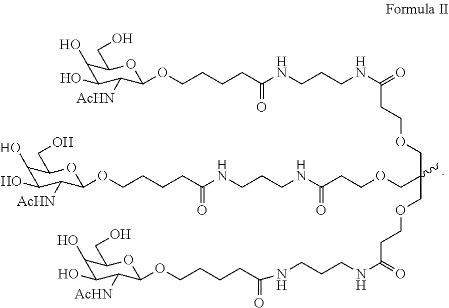 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

Another representative carbohydrate conjugate for use in the embodiments described herein includes, but is not limited to,

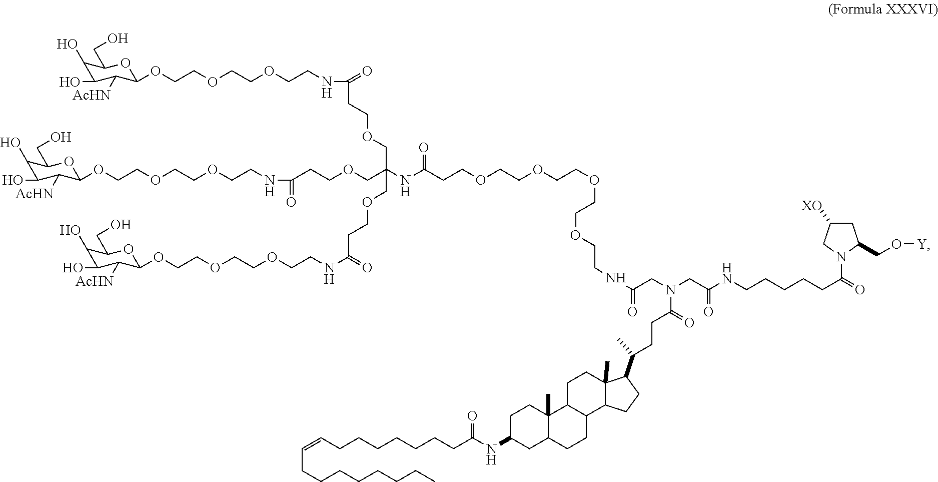

when one of X or Y is an oligonucleotide, the other is a hydrogen.

In embodiments in which a first dsRNA agent targeting LDHA and a second dsRNA agent targeting HAO1 are covalently attached (i.e., a dual targetingRNAi agent), one or both of the dsRNA agents may independently comprise a GalNAc or GalNAc derivative ligand.

In certain embodiments of the invention, the GalNAc or GalNAc derivative is attached to an iRNA agent of the invention via a monovalent linker. In some embodiments, the GalNAc or GalNAc derivative is attached to an iRNA agent of the invention via a bivalent linker. In yet other embodiments of the invention, the GalNAc or GalNAc derivative is attached to an iRNA agent of the invention via a trivalent linker.

In one embodiment, the double stranded RNAi agents of the invention comprise one GalNAc or GalNAc derivative attached to the iRNA agent, e.g., the 5′end of the sense strand of a dsRNA agent, or the 5′ end of one or both sense strands of a dual targeting RNAi agent as described herein. In another embodiment, the double stranded RNAi agents of the invention, or one or both dsRNA agents of a dual targeting RNAi agent as described herein, comprise a plurality (e.g., 2, 3, 4, 5, or 6) GalNAc or GalNAc derivatives, each independently attached to a plurality of nucleotides of the double stranded RNAi agent through a plurality of monovalent linkers.

In some embodiments, for example, when the two strands of an iRNA agent of the invention are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker.

In some embodiments, the carbohydrate conjugate further comprises one or more additional ligands as described above, such as, but not limited to, a PK modulator and/or a cell permeation peptide.

Additional carbohydrate conjugates (and linkers) suitable for use in the present invention include those described in PCT Publication Nos. WO 2014/179620 and WO 2014/179627, the entire contents of each of which are incorporated herein by reference.

D. Linkers

In some embodiments, the conjugate or ligand described herein can be attached to an iRNA oligonucleotide with various linkers that can be cleavable or non cleavable.

The term “linker” or “linking group” means an organic moiety that connects two parts of a compound, e.g., covalently attaches two parts of a compound. Linkers typically comprise a direct bond or an atom such as oxygen or sulfur, a unit such as NRB, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by 0, S, S(O), SO₂, N(R8), C(O), substituted or unsubstituted aryl, substituted or unsubstituted heteroaryl, substituted or unsubstituted heterocyclic; where R8 is hydrogen, acyl, aliphatic or substituted aliphatic. In one embodiment, the linker is between about 1-24 atoms, 2-24, 3-24, 4-24, 5-24, 6-24, 6-18, 7-18, 8-18 atoms, 7-17, 8-17, 6-16, 7-17, or 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

i. Redox Cleavable Linking Groups

In one embodiment, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

ii. Phosphate-Based Cleavable Linking Groups

In another embodiment, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

iii. Acid Cleavable Linking Groups

In another embodiment, a cleavable linker comprises an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

iv. Ester-Based Linking Groups

In another embodiment, a cleavable linker comprises an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

v. Peptide-Based Cleaving Groups

In yet another embodiment, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

In one embodiment, an iRNA of the invention is conjugated to a carbohydrate through a linker. Non-limiting examples of iRNA carbohydrate conjugates with linkers of the compositions and methods of the invention include, but are not limited to,

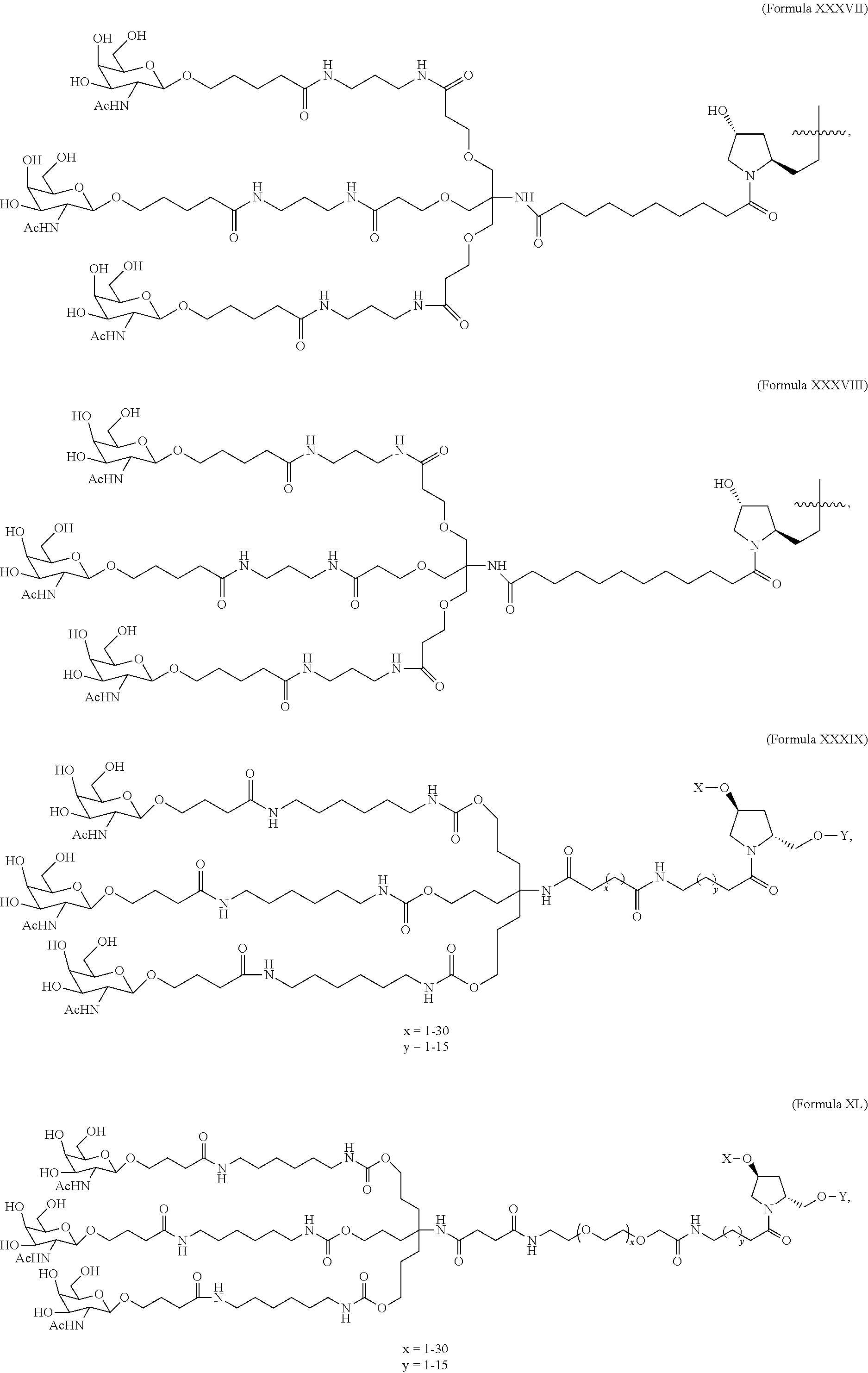

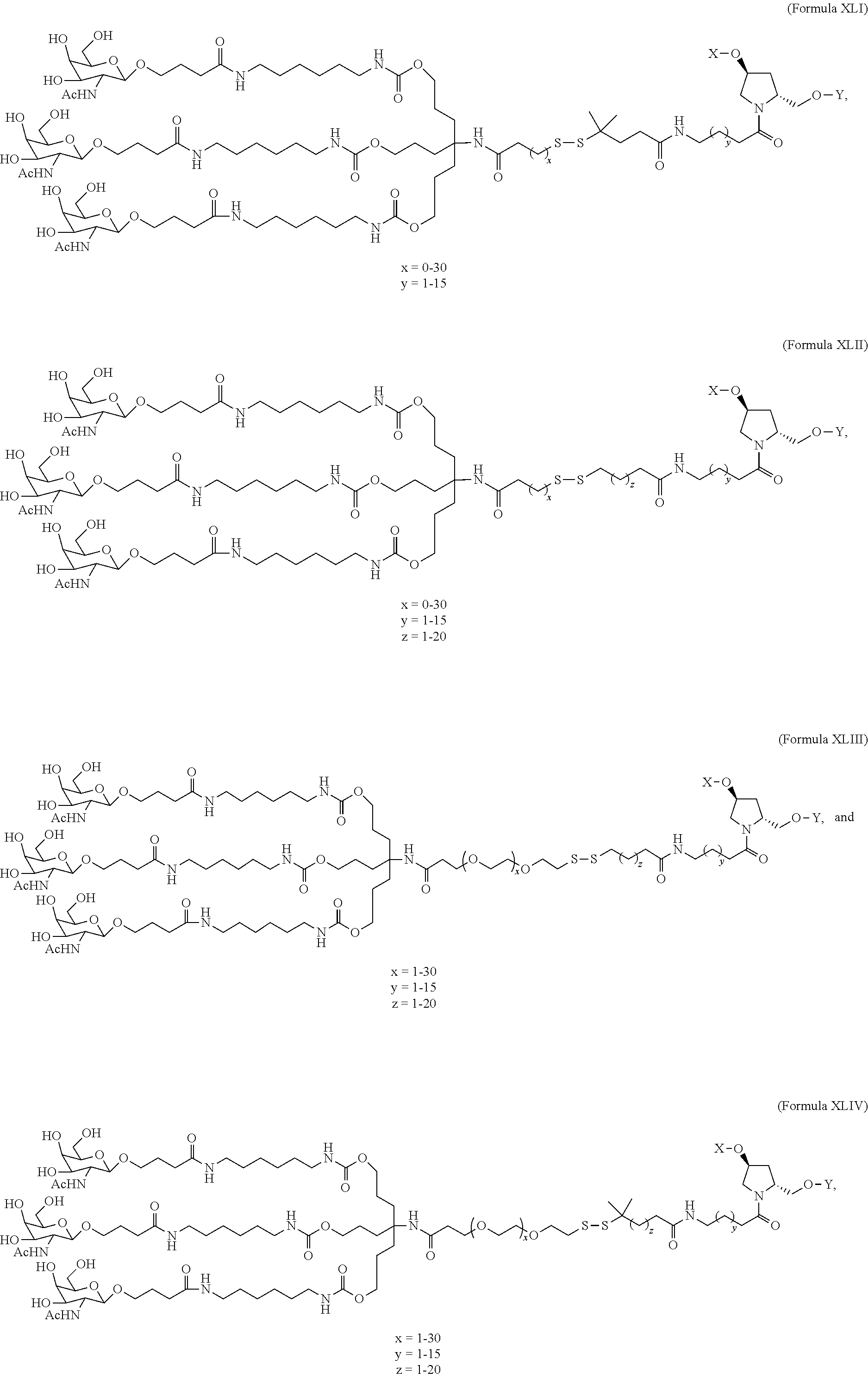

when one of X or Y is an oligonucleotide, the other is a hydrogen.

In certain embodiments of the compositions and methods of the invention, a ligand is one or more GalNAc (N-acetylgalactosamine) derivatives attached through a bivalent or trivalent branched linker.

In embodiments in which a first dsRNA agent targeting LDHA and a second dsRNA agent targeting HAO1 are covalently attached (i.e., a dual targetingRNAi agent), one or both of the dsRNA agents may independently a ligand comprising one or more GalNAc (N-acetylgalactosamine) derivatives attached through a bivalent or trivalent branched linker.

In one embodiment, a dsRNA of the invention is conjugated to a bivalent or trivalent branched linker selected from the group of structures shown in any of formula (XLV)-(XLVI):

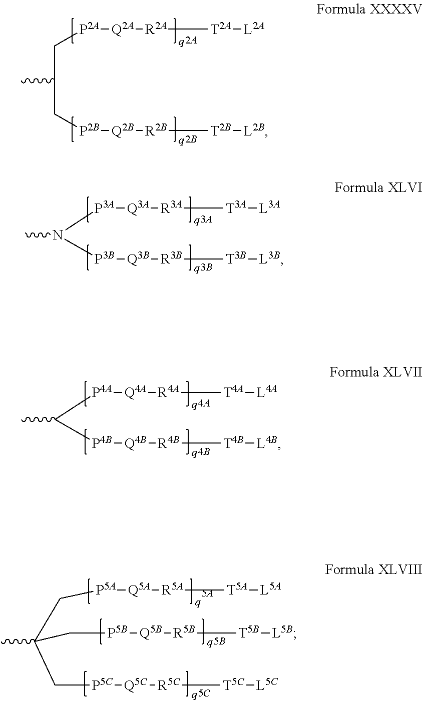

wherein:
q2A, q2B, q3A, q3B, q4A, q4B, q5A, q5B and q5C represent independently for each occurrence 0-20 and wherein the repeating unit can be the same or different;
P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{4A}, T^{5B}, T^{5C} are each independently for each occurrence absent, CO, NH, O, S, OC(O), NHC(O), CH₂, CH₂NH or CH₂O;
Q^{2A}, Q^{2B}, Q_{3A}, Q^{4A}, Q^{4B}, Q^{5A}, Q^{5B}, Q^{5C} are independently for each occurrence absent, alkylene, substituted alkylene wherin one or more methylenes can be interrupted or terminated by one or more of O, S, S(O), SO₂, N(R^{N}), C(R′)═C(R″), CC or C(O);

R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO, CH═N—O,

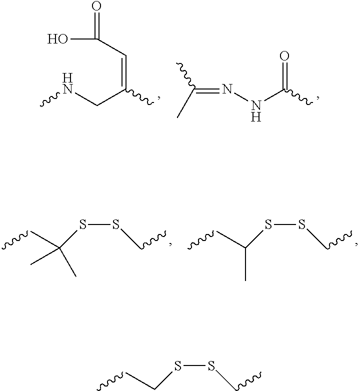

or heterocyclyl;

L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and R^{a} is H or amino acid side chain. Trivalent conjugating GalNAc derivatives are particularly useful for use with RNAi agents for inhibiting the expression of a target gene, such as those of formula (XLIX):

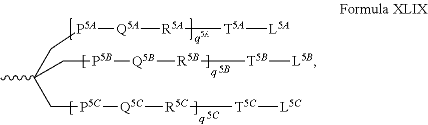

wherein L^{5A}, L^{5B} and L^{5C} represent a monosaccharide, such as GalNAc derivative.

Examples of suitable bivalent and trivalent branched linker groups conjugating GalNAc derivatives include, but are not limited to, the structures recited above as formulas II, VII, XI, X, and XIII.

Representative U.S. patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an iRNA. The present invention also includes iRNA compounds that are chimeric compounds.

“Chimeric” iRNA compounds or “chimeras,” in the context of this invention, are iRNA compounds, preferably dsRNAs, which contain two or more chemically distinct regions, each made up of at least one monomer unit, i.e., a nucleotide in the case of a dsRNA compound. These iRNAs typically contain at least one region wherein the RNA is modified so as to confer upon the iRNA increased resistance to nuclease degradation, increased cellular uptake, and/or increased binding affinity for the target nucleic acid. An additional region of the iRNA can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of an iRNA can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm, 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of an RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

### IV. Delivery of an iRNA of the Invention

The delivery of an iRNA of the invention to a cell e.g., a cell within a subject, such as a human subject (e.g., a subject in need thereof, such as a subject having a disorder of lipid metabolism) can be achieved in a number of different ways. For example, delivery may be performed by contacting a cell with an iRNA of the invention either in vitro or in vivo. In vivo delivery may also be performed directly by administering a composition comprising an iRNA, e.g., a dsRNA, to a subject. Alternatively, in vivo delivery may be performed indirectly by administering one or more vectors that encode and direct the expression of the iRNA. These alternatives are discussed further below.

In the methods of the invention which include a first dsRNA agent targeting LDHA and a second dsRNA agent targeting HAO1 are covalently attached (i.e., a dual targeting RNAi agent), the delivery of the first agent may be the same or different than the delivery of the second agent.

In general, any method of delivering a nucleic acid molecule (in vitro or in vivo) can be adapted for use with an iRNA of the invention (see e.g., Akhtar S. and Julian R L., (1992) Trends Cell. Biol. 2(5):139-144 and WO94/02595, which are incorporated herein by reference in their entireties). For in vivo delivery, factors to consider in order to deliver an iRNA molecule include, for example, biological stability of the delivered molecule, prevention of non-specific effects, and accumulation of the delivered molecule in the target tissue. The non-specific effects of an iRNA can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the iRNA molecule to be administered. Several studies have shown successful knockdown of gene products when an iRNA is administered locally. For example, intraocular delivery of a VEGF dsRNA by intravitreal injection in cynomolgus monkeys (Tolentino, M J. et al., (2004) Retina 24:132-138) and subretinal injections in mice (Reich, S J. et al. (2003) Mol. Vis. 9:210-216) were both shown to prevent neovascularization in an experimental model of age-related macular degeneration. In addition, direct intratumoral injection of a dsRNA in mice reduces tumor volume (Pille, J. et al. (2005) Mol. Ther. 11:267-274) and can prolong survival of tumor-bearing mice (Kim, W J. et al., (2006) Mol. Ther. 14:343-350; Li, S. et al., (2007) Mol. Ther. 15:515-523). RNA interference has also shown success with local delivery to the CNS by direct injection (Dorn, G. et al., (2004) Nucleic Acids 32:e49; Tan, P H. et al. (2005) Gene Ther. 12:59-66; Makimura, H. et a.l (2002) BMC Neurosci. 3:18; Shishkina, G T., et al. (2004) Neuroscience 129:521-528; Thakker, E R., et al. (2004) Proc. Natl. Acad. Sci. U.S.A. 101:17270-17275; Akaneya, Y., et al. (2005) J. Neurophysiol. 93:594-602) and to the lungs by intranasal administration (Howard, K A. et al., (2006) Mol. Ther. 14:476-484; Zhang, X. et al., (2004) J. Biol. Chem. 279:10677-10684; Bitko, V. et al., (2005) Nat. Med. 11:50-55). For administering an iRNA systemically for the treatment of a disease, the RNA can be modified or alternatively delivered using a drug delivery system; both methods act to prevent the rapid degradation of the dsRNA by endo- and exo-nucleases in vivo. Modification of the RNA or the pharmaceutical carrier can also permit targeting of the iRNA composition to the target tissue and avoid undesirable off-target effects. iRNA molecules can be modified by chemical conjugation to lipophilic groups such as cholesterol to enhance cellular uptake and prevent degradation. For example, an iRNA directed against ApoB conjugated to a lipophilic cholesterol moiety was injected systemically into mice and resulted in knockdown of apoB mRNA in both the liver and jejunum (Soutschek, J. et al., (2004) Nature 432:173-178). Conjugation of an iRNA to an aptamer has been shown to inhibit tumor growth and mediate tumor regression in a mouse model of prostate cancer (McNamara, J O. et al., (2006) Nat. Biotechnol. 24:1005-1015). In an alternative embodiment, the iRNA can be delivered using drug delivery systems such as a nanoparticle, a dendrimer, a polymer, liposomes, or a cationic delivery system. Positively charged cationic delivery systems facilitate binding of an iRNA molecule (negatively charged) and also enhance interactions at the negatively charged cell membrane to permit efficient uptake of an iRNA by the cell. Cationic lipids, dendrimers, or polymers can either be bound to an iRNA, or induced to form a vesicle or micelle (see e.g., Kim S H. et al., (2008) Journal of Controlled Release 129(2):107-116) that encases an iRNA. The formation of vesicles or micelles further prevents degradation of the iRNA when administered systemically. Methods for making and administering cationic-iRNA complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al., (2007) J. Hypertens. 25:197-205, which are incorporated herein by reference in their entirety). Some non-limiting examples of drug delivery systems useful for systemic delivery of iRNAs include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, an iRNA forms a complex with cyclodextrin for systemic administration. Methods for administration and pharmaceutical compositions of iRNAs and cyclodextrins can be found in U.S. Pat. No. 7,427,605, which is herein incorporated by reference in its entirety.

A. Vector Encoded iRNAs of the Invention

iRNA targeting the LDHA gene and iRNA targeting LDHA and HAO1 can be expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92:1292).

The individual strand or strands of an iRNA can be transcribed from a promoter on an expression vector. Where two separate strands are to be expressed to generate, for example, a dsRNA, two separate expression vectors can be co-introduced (e.g., by transfection or infection) into a target cell. Alternatively each individual strand of a dsRNA can be transcribed by promoters both of which are located on the same expression plasmid. In one embodiment, a dsRNA is expressed as inverted repeat polynucleotides joined by a linker polynucleotide sequence such that the dsRNA has a stem and loop structure.

iRNA expression vectors are generally DNA plasmids or viral vectors. Expression vectors compatible with eukaryotic cells, preferably those compatible with vertebrate cells, can be used to produce recombinant constructs for the expression of an iRNA as described herein. Eukaryotic cell expression vectors are well known in the art and are available from a number of commercial sources. Typically, such vectors are provided containing convenient restriction sites for insertion of the desired nucleic acid segment. Delivery of iRNA expressing vectors can be systemic, such as by intravenous or intramuscular administration, by administration to target cells ex-planted from the patient followed by reintroduction into the patient, or by any other means that allows for introduction into a desired target cell.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of an iRNA will generally require regulatory elements, e.g., promoters, enhancers, etc., to ensure the expression of the iRNA in target cells. Other aspects to consider for vectors and constructs are known in the art.

### V. Pharmaceutical Compositions of the Invention

The present invention also includes pharmaceutical compositions and formulations which include the iRNAs of the invention. Accordingly, in one embodiment, provided herein are pharmaceutical compositions comprising a double stranded ribonucleic acid (dsRNA) agent that inhibits expression of lactic acid dehydrogenase A (LDHA) in a cell, such as a liver cell, wherein the dsRNA agent comprises a sense strand and an antisense strand, wherein the sense strand comprises at least 15 contiguous nucleotides differing by no more than 3 nucleotides from the nucleotide sequence of SEQ ID NO:1, and said antisense strand comprises at least 15 contiguous nucleotides differing by no more than 3 nucleotides from the nucleotide sequence of SEQ ID NO:2; and a pharmaceutically acceptable carrier.

In another embodiment, provided herein are pharmaceutical compositions comprising a dsRNA agent that inhibits expression of lactic acid dehydrogenase A (LDHA) in a cell, such as a liver cell, wherein the dsRNA agent comprises a sense strand and an antisense strand, the antisense strand comprising a region of complementarity which comprises at least 15 contiguous nucleotides differing by no more than 3 nucleotides from any one of the antisense sequences listed in any one of Tables 2-5; and a pharmaceutically acceptable carrier.

In one embodiment, provided herein are pharmaceutical compositions comprising a first double stranded ribonucleic acid (dsRNA) agent that inhibits expression of lactic acid dehydrogenase A (LDHA) in a cell, such as a liver cell, comprising a sense strand and an antisense strand, wherein the sense strand comprises at least 15 contiguous nucleotides differing by no more than 3 nucleotides from the nucleotide sequence of SEQ ID NO:1, and the antisense strand comprises at least 15 contiguous nucleotides differing by no more than 3 nucleotides from the nucleotide sequence of SEQ ID NO:2; and a second double stranded ribonucleic acid (dsRNA) agent that inhibits expression of hydroxyacid oxidase 1 (glycolate oxidase) (HAO1) in a cell, such as a liver cell, comprising a sense strand and an antisense strand, wherein the sense strand comprises at least 15 contiguous nucleotides differing by no more than 3 nucleotides from the nucleotide sequence of SEQ ID NO:21, and the antisense strand comprises at least 15 contiguous nucleotides differing by no more than 3 nucleotides from the nucleotide sequence of SEQ ID NO:22; and a pharmaceutically acceptable carrier.

In another embodiment, provided herein are pharmaceutical compositions a first double stranded ribonucleic acid (dsRNA) agent that inhibits expression of lactic acid dehydrogenase A (LDHA) in a cell, such as a liver cell, comprising a sense strand and an antisense strand, the antisense strand comprising a region of complementarity which comprises at least 15 contiguous nucleotides differing by no more than 3 nucleotides from any one of the antisense sequences listed in any one of Tables 2-5; and a second double stranded ribonucleic acid (dsRNA) agent that inhibits expression of hydroxyacid oxidase 1 (glycolate oxidase) (HAO1) in a cell, such as a liver cell, comprising a sense strand and an antisense strand, the antisense strand comprising a region of complementarity which comprises at least 15 contiguous nucleotides differing by no more than 3 nucleotides from any one of the antisense sequences listed in any one of Tables 7-14.

In yet another embodiment, the present invention provides pharmaceutical compositions and formulations comprising a dual targeting RNAi agent of the invention, and a pharmaceutically acceptable carrier.

The pharmaceutical compositions containing the iRNA of the invention are useful for treating a disease or disorder associated with the expression or activity of an LDHA gene or an LDHA gene and an HAO1 gene, e.g., an oxalate pathway-associated disease, disorder, or condition.

Such pharmaceutical compositions are formulated based on the mode of delivery. One example is compositions that are formulated for systemic administration via parenteral delivery, e.g., by intravenous (IV) or for subcutaneous delivery. Another example is compositions that are formulated for direct delivery into the liver, e.g., by infusion into the liver, such as by continuous pump infusion.

The pharmaceutical compositions of the invention may be administered in dosages sufficient to inhibit expression of an LDHA gene or an LDHA gene and an HAO1 gene. In general, a suitable dose of an iRNA of the invention will be in the range of about 0.001 to about 200.0 milligrams per kilogram body weight of the recipient per day, generally in the range of about 1 to 50 mg per kilogram body weight per day. Typically, a suitable dose of an iRNA of the invention will be in the range of about 0.1 mg/kg to about 5.0 mg/kg, preferably about 0.3 mg/kg and about 3.0 mg/kg.

In the methods of the invention which include a first dsRNA agent targeting LDHA and a second dsRNA agent targeting HAO1, the first agent and the second agent may be present in the same pharmaceutical formulation or separate pharmaceutical formulations.

A repeat-dose regimine may include administration of a therapeutic amount of iRNA on a regular basis, such as every other day to once a year. In certain embodiments, the iRNA is administered about once per month to about once per quarter (i.e., about once every three months).

After an initial treatment regimen, the treatments can be administered on a less frequent basis.

The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual iRNAs encompassed by the invention can be made using conventional methodologies or on the basis of in vivo testing using an appropriate animal model, as described elsewhere herein.

Advances in mouse genetics have generated a number of mouse models for the study of various human diseases, such as an oxalate pathway-associated disease, disorder, or condition that would benefit from reduction in the expression of LDHA and/or LDHA and HAO1. Such models can be used for in vivo testing of iRNA, as well as for determining a therapeutically effective dose. Suitable mouse models are known in the art and include, for example, mouse models which may include mutations or deletions in the AGXT or GRHPR genes (see, e.g., Salido E C, et al. (2006) PNAS 103(48): 18249-18254 and Knight J, et al. (2012) Am. J. Physiol. Renal Physiol. 302: F688-F693); a PH3 mouse model (see, e.g., Li, et al. (2015) biochem Biophys Acta 1852(12):2700); and the ethylene glycol urolithiasis mouse model.

The pharmaceutical compositions of the present invention can be administered in a number of ways depending upon whether local or systemic treatment is desired and upon the area to be treated. Administration can be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration.

The iRNA can be delivered in a manner to target a particular cell or tissue, such as the liver (e.g., the hepatocytes of the liver).

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the iRNAs featured in the invention are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). iRNAs featured in the invention can be encapsulated within liposomes or can form complexes thereto, in particular to cationic liposomes. Alternatively, iRNAs can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In some embodiments, oral formulations are those in which dsRNAs featured in the invention are administered in conjunction with one or more penetration enhancer surfactants and chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether. DsRNAs featured in the invention can be delivered orally, in granular form including sprayed dried particles, or complexed to form micro or nanoparticles. DsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Suitable complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g., p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. Pat. No. 6,887,906, US Publn. No. 20030027780, and U.S. Pat. No. 6,747,014, each of which is incorporated herein by reference.

Compositions and formulations for parenteral, intraparenchymal (into the brain), intrathecal, intraventricular or intrahepatic administration can include sterile aqueous solutions which can also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions can be generated from a variety of components that include, but are not limited to, preformed liquids, self-emulsifying solids and self-emulsifying semisolids. Particularly preferred are formulations that target the liver when treating hepatic disorders such as hepatic carcinoma.

The pharmaceutical formulations of the present invention, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention can be formulated into any of many possible dosage forms such as, but not limited to, tablets, capsules, gel capsules, liquid syrups, soft gels, suppositories, and enemas. The compositions of the present invention can also be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers.

A. Additional Formulations

i. Emulsions

The compositions of the present invention can be prepared and formulated as emulsions. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution in either aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion can be a semisolid or a solid, as is the case of emulsion-style ointment bases and creams. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic and amphoteric (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y. Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

ii. Microemulsions

In one embodiment of the present invention, the compositions of iRNAs and nucleic acids are formulated as microemulsions. A microemulsion can be defined as a system of water, oil and amphiphile which is a single optically isotropic and thermodynamically stable liquid solution (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (M0310), hexaglycerol monooleate (P0310), hexaglycerol pentaoleate (P0500), decaglycerol monocaprate (MCA750), decaglycerol monooleate (M0750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (see e.g., U.S. Pat. Nos. 6,191,105; 7,063,860; 7,070,802; 7,157,099; Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions can form spontaneously when their components are brought together at ambient temperature. This can be particularly advantageous when formulating thermolabile drugs, peptides or iRNAs. Microemulsions have also been effective in the transdermal delivery of active components in both cosmetic and pharmaceutical applications. It is expected that the microemulsion compositions and formulations of the present invention will facilitate the increased systemic absorption of iRNAs and nucleic acids from the gastrointestinal tract, as well as improve the local cellular uptake of iRNAs and nucleic acids.

Microemulsions of the present invention can also contain additional components and additives such as sorbitan monostearate (Grill 3), Labrasol, and penetration enhancers to improve the properties of the formulation and to enhance the absorption of the iRNAs and nucleic acids of the present invention. Penetration enhancers used in the microemulsions of the present invention can be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

iii. Microparticles

an RNAi agent of the invention may be incorporated into a particle, e.g., a microparticle. Microparticles can be produced by spray-drying, but may also be produced by other methods including lyophilization, evaporation, fluid bed drying, vacuum drying, or a combination of these techniques.

iv. Penetration Enhancers

In one embodiment, the present invention employs various penetration enhancers to effect the efficient delivery of nucleic acids, particularly iRNAs, to the skin of animals. Most drugs are present in solution in both ionized and nonionized forms. However, usually only lipid soluble or lipophilic drugs readily cross cell membranes. It has been discovered that even non-lipophilic drugs can cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers can be classified as belonging to one of five broad categories, i.e., surfactants, fatty acids, bile salts, chelating agents, and non-chelating non-surfactants (see e.g., Malmsten, M. Surfactants and polymers in drug delivery, Informa Health Care, New York, N.Y., 2002; Lee et al., Critical Reviews in Therapeutic Drug Carrier Systems, 1991, p. 92). Each of the above mentioned classes of penetration enhancers are described below in greater detail.

Surfactants (or “surface-active agents”) are chemical entities which, when dissolved in an aqueous solution, reduce the surface tension of the solution or the interfacial tension between the aqueous solution and another liquid, with the result that absorption of iRNAs through the mucosa is enhanced. In addition to bile salts and fatty acids, these penetration enhancers include, for example, sodium lauryl sulfate, polyoxyethylene-9-lauryl ether and polyoxyethylene-20-cetyl ether) (see e.g., Malmsten, M. Surfactants and polymers in drug delivery, Informa Health Care, New York, N.Y., 2002; Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₂₀ alkyl esters thereof (e.g., methyl, isopropyl and t-butyl), and mono- and di-glycerides thereof (i.e., oleate, laurate, caprate, myristate, palmitate, stearate, linoleate, etc.) (see e.g., Touitou, E., et al. Enhancement in Drug Delivery, CRC Press, Danvers, Mass., 2006; Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

The physiological role of bile includes the facilitation of dispersion and absorption of lipids and fat-soluble vitamins (see e.g., Malmsten, M. Surfactants and polymers in drug delivery, Informa Health Care, New York, N.Y., 2002; Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (see e.g., Malmsten, M. Surfactants and polymers in drug delivery, Informa Health Care, New York, N.Y., 2002; Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating agents, as used in connection with the present invention, can be defined as compounds that remove metallic ions from solution by forming complexes therewith, with the result that absorption of iRNAs through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(see e.g., Katdare, A. et al., Excipient development for pharmaceutical, biotechnology, and drug delivery, CRC Press, Danvers, Mass., 2006; Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Ra, 1990, 14, 43-51).

As used herein, non-chelating non-surfactant penetration enhancing compounds can be defined as compounds that demonstrate insignificant activity as chelating agents or as surfactants but that nonetheless enhance absorption of iRNAs through the alimentary mucosa (see e.g., Muranishi, Critical Reviews in Therapeutic Drug Carrier Systems, 1990, 7, 1-33). This class of penetration enhancers includes, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of iRNAs at the cellular level can also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of dsRNAs. Examples of commercially available transfection reagents include, for example Lipofectamine™ (Invitrogen; Carlsbad, Calif.), Lipofectamine 2000™ (Invitrogen; Carlsbad, Calif.), 293fectin™ (Invitrogen; Carlsbad, Calif.), Cellfectin™ (Invitrogen; Carlsbad, Calif.), DMRIE-C™ (Invitrogen; Carlsbad, Calif.), FreeStyle™ MAX (Invitrogen; Carlsbad, Calif.), Lipofectamine™ 2000 CD (Invitrogen; Carlsbad, Calif.), Lipofectamine™ (Invitrogen; Carlsbad, Calif.), RNAiMAX (Invitrogen; Carlsbad, Calif.), Oligofectamine™ (Invitrogen; Carlsbad, Calif.), Optifect™ (Invitrogen; Carlsbad, Calif.), X-tremeGENE Q2 Transfection Reagent (Roche; Grenzacherstrasse, Switzerland), DOTAP Liposomal Transfection Reagent (Grenzacherstrasse, Switzerland), DOSPER Liposomal Transfection Reagent (Grenzacherstrasse, Switzerland), or Fugene (Grenzacherstrasse, Switzerland), Transfectam® Reagent (Promega; Madison, Wis.), TransFast™ Transfection Reagent (Promega; Madison, Wis.), Tfx™-20 Reagent (Promega; Madison, Wis.), Tfx™-50 Reagent (Promega; Madison, Wis.), DreamFect™ (OZ Biosciences; Marseille, France), EcoTransfect (OZ Biosciences; Marseille, France), TransPass' D1 Transfection Reagent (New England Biolabs; Ipswich, Mass., USA), LyoVec™/LipoGen™ (Invitrogen; San Diego, Calif., USA), PerFectin Transfection Reagent (Genlantis; San Diego, Calif., USA), NeuroPORTER Transfection Reagent (Genlantis; San Diego, Calif., USA), GenePORTER Transfection reagent (Genlantis; San Diego, Calif., USA), GenePORTER 2 Transfection reagent (Genlantis; San Diego, Calif., USA), Cytofectin Transfection Reagent (Genlantis; San Diego, Calif., USA), BaculoPORTER Transfection Reagent (Genlantis; San Diego, Calif., USA), TroganPORTER™ transfection Reagent (Genlantis; San Diego, Calif., USA), RiboFect (Bioline; Taunton, Mass., USA), PlasFect (Bioline; Taunton, Mass., USA), UniFECTOR (B-Bridge International; Mountain View, Calif., USA), SureFECTOR (B-Bridge International; Mountain View, Calif., USA), or HiFect™ (B-Bridge International, Mountain View, Calif., USA), among others.

Other agents can be utilized to enhance the penetration of the administered nucleic acids, including glycols such as ethylene glycol and propylene glycol, pyrrols such as 2-pyrrol, azones, and terpenes such as limonene and menthone.

v. Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate dsRNA in hepatic tissue can be reduced when it is coadministered with polyinosinic acid, dextran sulfate, polycytidic acid or 4-acetamido-4′isothiocyano-stilbene-2,2′-disulfonic acid (Miyao et al., DsRNA Res. Dev., 1995, 5, 115-121; Takakura et al., DsRNA & Nucl. Acid Drug Dev., 1996, 6, 177-183.

vi. Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipients suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

vii. Other Components

The compositions of the present invention can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions can contain substances which increase the viscosity of the suspension including, for example, sodium carboxymethylcellulose, sorbitol and/or dextran. The suspension can also contain stabilizers.

In some embodiments, pharmaceutical compositions featured in the invention include (a) one or more iRNA compounds and (b) one or more agents which function by a non-RNAi mechanism and which are useful in treating an oxalate pathway-associated disease, disorder, or condition. Examples of such agents include, but are not lmited to pyridoxine, an ACE inhibitor (angiotensin converting enzyme inhibitors), e.g., benazepril (Lotensin); an angiotensin II receptor antagonist (ARB) (e.g., losartan potassium, such as Merck & Co.'s Cozaar®), e.g., Candesartan (Atacand); an HMG-CoA reductase inhibitor (e.g., a statin); dietary oxalate degrading compounds, e.g., Oxalate decarboxylase (Oxazyme); calcium binding agents, e.g., Sodium cellulose phosphate (Calcibind); diuretics, e.g., thiazide diuretics, such as hydrochlorothiazide (Microzide); phosphate binders, e.g., Sevelamer (Renagel); magnesium and Vitamin B6 supplements; potassium citrate; orthophosphates, bisphosphonates; oral phosphate and citrate solutions; high fluid intake, urinary tract endoscopy; extracorporeal shock wave lithotripsy; kidney dialysis; kidney stone removal (e.g., surgery); and kidney/liver transplant; or a combination of any of the foregoing.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred.

The data obtained from cell culture assays and animal studies can be used in formulating a range of dosage for use in humans. The dosage of compositions featured herein in the invention lies generally within a range of circulating concentrations that include the ED₅₀ with little or no toxicity. The dosage can vary within this range depending upon the dosage form employed and the route of administration utilized. For any compound used in the methods featured in the invention, the therapeutically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

In addition to their administration, as discussed above, the iRNAs featured in the invention can be administered in combination with other known agents effective in treatment of pathological processes mediated by LDHA or LDHA and HAO1 expression. In any event, the administering physician can adjust the amount and timing of iRNA administration on the basis of results observed using standard measures of efficacy known in the art or described herein.

### VI. Methods of the Invention

The present invention also provides methods of using an iRNA of the invention and/or a composition of the invention to reduce and/or inhibit LDHA or LDHA and HAO1 expression in a cell, such as a cell in a subject. The methods include contacting the cell with a RNAi agent (or pharmaceutical composition comprising an iRNA agent) or pharmaceutical composition of the invention. In some embodiments, the cell is maintained for a time sufficient to obtain degradation of the mRNA transcript of an LDHA gene. In other embodiments, the cell is maintained for a time sufficient to obtain degradation of the mRNA transcript of an LDHA gene and an HAO1 gene in the cell.

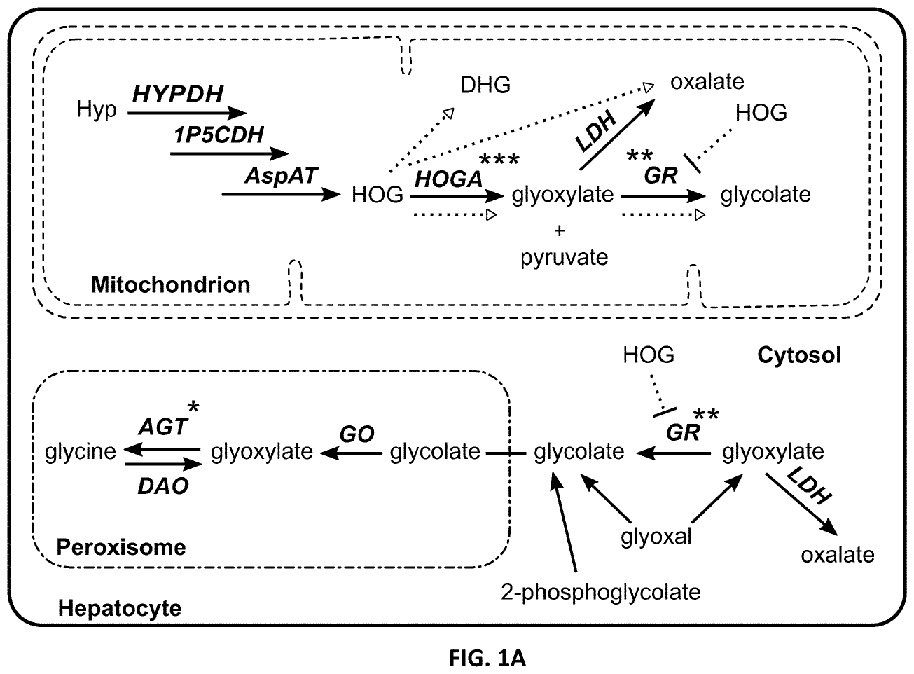

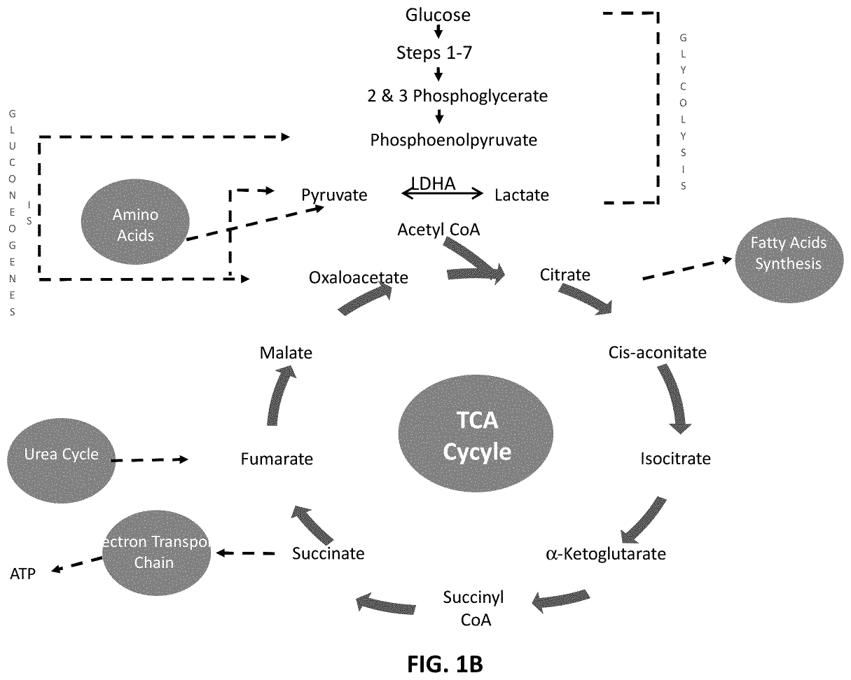

It should be noted that, although the compositions of the invention target LDHA, an enzyme involved in numerous cellular processes (see, e.g., FIGS. 1A and 1B), as demonstrated in the Examples below, contacting a cell with a composition of the invention, or administering a composition of the invention to a subject, does not result in adverse effects in either wild-type or diseased subjects, thereby demonstrating the safety of the compostions of the invention.

Reduction in gene expression can be assessed by any methods known in the art. For example, a reduction in the expression of LDHA, and/or HAO1, and/or glycolate may be determined by determining the mRNA expression level of LDHA, and/or HAO1, and/or glycolate using methods routine to one of ordinary skill in the art, e.g., Northern blotting, qRT-PCR; by determining the protein level of LDHA, and/or HAO1, and/or glycolate using methods routine to one of ordinary skill in the art, such as Western blotting, immunological techniques. A reduction in the expression of LDHA, and/or HAO1, and/or glycolate may also be assessed indirectly by measuring a decrease in biological activity of LDHA, and/or HAO1, and/or glycolate, e.g., a decrease in the enzymatic activity of LDHA and/or a decrease in tissue or plasma oxalate, or urinary oxalate and/or glycolate excretion.

In the methods of the invention the cell may be contacted in vitro or in vivo, i.e., the cell may be within a subject.

A cell suitable for treatment using the methods of the invention may be any cell that expresses an LDHA gene, a cell that expresses an HAO1 gene, a cell that expresses a glycolate gene, a cell that expresses, an LDHA gene and a glycolate gene, a cell that expresses an HAO1 gene and a glycolate gene, a cell that expresses an LDHA gene and an HAO1 gene, or a cell that expresses an LDHA gene, an HAO1 gene, and a glycolate gene. A cell suitable for use in the methods of the invention may be a mammalian cell, e.g., a primate cell (such as a human cell or a non-human primate cell, e.g., a monkey cell or a chimpanzee cell), a non-primate cell (such as a cow cell, a pig cell, a camel cell, a llama cell, a horse cell, a goat cell, a rabbit cell, a sheep cell, a hamster, a guinea pig cell, a cat cell, a dog cell, a rat cell, a mouse cell, a lion cell, a tiger cell, a bear cell, or a buffalo cell), a bird cell (e.g., a duck cell or a goose cell), or a whale cell. In one embodiment, the cell is a human cell, e.g., a human liver cell.

LDHA expression is inhibited in the cell by at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100%. In preferred embodiments, LDHA expression is inhibited by at least 20%.

HAO1 expression may be inhibited in the cell by at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100%. In preferred embodiments, HAO1 expression is inhibited by at least 20%.

In embodiments in which a cell is contacted with a dual targeting RNAi agent of the invention, the level of inhibition of LDHA may be the same or different than the level of HAO1.

In one embodiment, the in vivo methods of the invention may include administering to a subject a composition containing an iRNA, where the iRNA includes a nucleotide sequence that is complementary to at least a part of an RNA transcript of the LDHA gene of the mammal to be treated. In another embodiment, the in vivo methods of the invention may include administering to a subject a composition containing an iRNA, where the iRNA includes a nucleotide sequence that is complementary to at least a part of an RNA transcript of the LDHA gene and a nucleotide sequence that is complementary to at least a part of an RNA transcript of the HAO1 gene of the mammal to be treated.

When the organism to be treated is a mammal such as a human, the composition can be administered by any means known in the art including, but not limited to oral, intraperitoneal, or parenteral routes, including intracranial (e.g., intraventricular, intraparenchymal and intrathecal), intravenous, intramuscular, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection.

In some embodiments, the administration is via a depot injection. A depot injection may release the iRNA in a consistent way over a prolonged time period. Thus, a depot injection may reduce the frequency of dosing needed to obtain a desired effect, e.g., a desired inhibition of LDHA, or a desired inhibition of both LDHA and HAO1, or a therapeutic or prophylactic effect. A depot injection may also provide more consistent serum concentrations. Depot injections may include subcutaneous injections or intramuscular injections. In preferred embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intravenous, subcutaneous, arterial, or epidural infusions. In preferred embodiments, the infusion pump is a subcutaneous infusion pump. In other embodiments, the pump is a surgically implanted pump that delivers the iRNA to the liver.

An iRNA of the invention may be present in a pharmaceutical composition, such as in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the iRNA can be adjusted such that it is suitable for administering to a subject.

Alternatively, an iRNA of the invention may be administered as a pharmaceutical composition, such as a dsRNA liposomal formulation.

The mode of administration may be chosen based upon whether local or systemic treatment is desired and based upon the area to be treated. The route and site of administration may be chosen to enhance targeting.

In one aspect, the present invention also provides methods for inhibiting the expression of an LDHA gene in a mammal. The methods include administering to the mammal a composition comprising a dsRNA that targets an LDHA gene in a cell of the mammal, thereby inhibiting expression of the LDHA gene in the cell.

In another aspect, the present invention also provides methods for inhibiting the expression of an LDHA gene and an HAO1 gene in a mammal. The methods include administering to the mammal a pharmaceutical composition comprising a dsRNA agent that targets an LDHA gene and a dsRNA agent that targets an HAO1 gene in a cell of the mammal, thereby inhibiting expression of the LDHA gene and the HAO1 gene in the mammal. In one aspect, the present invention provides methods for inhibiting the expression of an LDHA gene and an HAO1 gene in a mammal. The methods include administering to the mammal a dual targeting RNAi agent (or pharmaceutical composition comprising a dual targeting agent) that targets an LDHA gene and an HAO1 gene in a cell of the mammal, thereby inhibiting expression of the LDHA gene and the HAO1 gene in the subject.

Reduction in gene expression can be assessed by any methods known it the art and by methods, e.g. qRT-PCR, described herein. Reduction in protein production can be assessed by any methods known it the art and by methods, e.g. ELISA, enzymatic activity, described herein.

The present invention also provides therapeutic and prophylactic methods which include administering to a subject having, or prone to developing an oxalate-associate disease, disorder, or condition, the iRNA agents, pharmaceutical compositions comprising an iRNA agent, or vectors comprising an iRNA of the invention.

In one aspect, the present invention provides methods of treating a subject having a disorder that would benefit from reduction in LDHA expression, e.g., an oxalate pathway-associated disease, disorder, or condition.

The treatment methods (and uses) of the invention include administering to the subject, e.g., a human, a therapeutically effective amount of a dsRNA agent, a dual targeting iRNA agent or a pharmaceutical composition comprising a dsRNA, a pharmaceutical compositions comprising a dual targeting RNAi agent or pharmaceutical composition of the invention comprising a first dsRNA agent that inhibits expression of LDHA and a second dsRNA agent that inhibits expression of HAO1, thereby treating the subject.

In one aspect, the invention provides methods of preventing at least one symptom in a subject having a disorder that would benefit from reduction in LDHA expression, e.g., an oxalate pathway-associated disease, disorder, or condition. The methods include administering to the subject a prophylactically effective amount of dsRNA agent, a dual targeting iRNA agent or a pharmaceutical composition comprising a dsRNA, a pharmaceutical compositions comprising a dual targeting RNAi agent or pharmaceutical composition of the invention comprising a first dsRNA agent that inhibits expression of LDHA and a second dsRNA agent that inhibits expression of HAO1, thereby preventing at least one symptom in the subject.

Subjects that would benefit from a reduction and/or inhibition of an LDHA gene expression include subjects that would benefit from reduction in both LDHA and HAO1 gene expression.

Therefore, in one embodiment, a subject that would benefit from reduction in the expression level of LDHA or a reduction in the expression of LDHA and HAO1, has normal urinary oxalate excretion levels, e.g., less than about 40 mg (440 μmol) in 24 hours (e.g., men have a normal urinary oxalate excretion level of less than about 43 mg/day and women have a normal urinary oxalate excretion level of less than about 32 mg/day). In another embodiment, a subject that would benefit from a reduction in the expression level of LDHA or a reduction in the expression of LDHA and HAO1 has mild hyperoxaluria (a urinary oxalate excretion level of about 40 to about 60 mg/day). In another embodiment, a subject that would benefit from reduction in the expression level of LDHA or a reduction in the expression of LDHA and HAO1 has high hyperoxaluria (a urinary oxalate excretion level of greater than about 60 mg/day).

In one embodiment, a subject that would benefit from reduction in LDHA expression or LDHA and HAO1 expression is a human at risk of developing an oxalate pathway-associated disease, disorder, or condition. In one embodiment, a subject that would benefit from reduction in LDHA expression or LDHA and HAO1 expression is a human having an oxalate pathway-associated disease, disorder, or condition. In yet another embodiment, a subject that would benefit from reduction in LDHA expression or LDHA and HAO1 expression is a human being treated for an oxalate pathway-associated disease, disorder, or condition.

In one embodiment, a subject having an oxalate pathway-associated disease, disorder, or condition has an oxalate-associated disease, disorder, or condition. Non-limiting examples of oxalate-associated disease, disorder, or condition include a kidney stone formation disease, disorder, or condition, or a calcium oxalate tissue deposition disease, disorder, or condition. The kidney stone formation disease, disorder, or condition may be a calcium oxalate stone formation disease, disorder, or condition or a non-calcium oxalate stone formation disease, disorder, or condition. The calcium oxalate stone formation disease, disorder, or condition may be a hyperoxaluria disease, disorder, or condition (e.g., mild hyperoxaluria (a urinary oxalate excretion level of about 40 to about 60 mg/day) or high hyperoxaluria (a urinary oxalate excretion level of greater than about 60 mg/day)); or a non-hyperoxaluria disease, disorder, or condition (i.e., a calcium oxalate stone formation disease without hyperoxaluria, e.g., normal urinary oxalate excretion levels, e.g., less than about 40 mg (440 μmol) in 24 hours (e.g., men have a normal urinary oxalate excretion level of less than about 43 mg/day and women have a normal urinary oxalate excretion level of less than about 32 mg/day).

In one embodiment, the hyperoxaluria disease, disorder, or condition is selected from the group consisting of primary hyperoxaluria, enteric hyperoxaluria, dietary hyperoxaluria, and idiopathic hyperoxaluria.

In one embodiment, the non-hyperoxaluria stone formation disease, disorder, or condition is hypercalciuria and/or hypocitraturia. In another embodiment, the non-hyperoxaluria stone formation disease, disorder, or condition is calcium oxalate or non-calcium oxalate kidney stone formation disease.

In one embodiment, the calcium oxalate stone formation disease, disorder, or condition is an inherited disorder, such as a Primary Hyperoxaluria (PH), e.g., Primary Hyperoxaluria Type 1 (PH1); Primary Hyperoxaluria Type 2 (PH2); Primary Hyperoxaluria Type 3 (PH3); or Primary Hyperoxaluria Non-Type 1, Non-Type 2, Non-Type 3 (PH-Non-Type 1, Non-Type 2, Non-Type 3). PH1 is a hereditary disorder casued by mutations in alanine glyoxylate aminotransferase (AGT), PH2 is due to mutations in glyoxylate reductase/hydroxypyruvate reductase (GRHPR), and PH3 is caused by mutations in HOGA1 (formerly DHDPSL). Subjects having PH-Non-Type 1, Non-Type 2, Non-Type 3 have clinical characteristics indistinguishable from type 1, 2, and 3, but with normal AGT, GRHPR, and HOGA1 liver enzyme activity, yet the etiology of the marked hyperoxaluria in such subjects remains to be elucidated.

A deficiency in either AGT or GRHPR activities results in an excess of glyoxylate and oxalate (see, e.g., Knight et al., (2011) Am J Physiol Renal Physiol 302(6): F688-F693). Therefore, inhibition of LDHA expression and/or activity will decrease the level of excess oxalate. In addition, the inhibition of glycolate oxidase (HAO1) will further reduce the level of glyoxylate. The buildup of oxalate in subjects having PH causes increased excretion of oxalate, which in turn results in renal and bladder stones. Stones cause urinary obstruction (often with severe and acute pain), secondary infection of urine and eventually kidney damage. Oxalate stones tend to be severe, resulting in relatively early kidney damage (e.g., onset in teenage years to early adulthood), which impairs the excretion of oxalate, leading to a further acceleration in accumulation of oxalate in the body. After the development of renal failure, patients may get deposits of oxalate in the bones, joints and bone marrow. Severe cases may develop haematological problems such as anaemia and thrombocytopaenia. The deposition of oxalate in the body is sometimes called “oxalosis” to be distinguished from “oxaluria” which refers to oxalate in the urine. Renal failure is a serious complication requiring treatment in its own right. Dialysis can control renal failure but tends to be inadequate to dispose of excess oxalate. Renal transplant is more effective and this is the primary treatment of severe hyperoxaluria. Liver transplantation (often in addition to renal transplant) may be able to control the disease by correcting the metabolic defect. In a proportion of patients with primary hyperoxaluria type 1, pyridoxine treatment (vitamin B6) may also decrease oxalate excretion and prevent kidney stone formation.

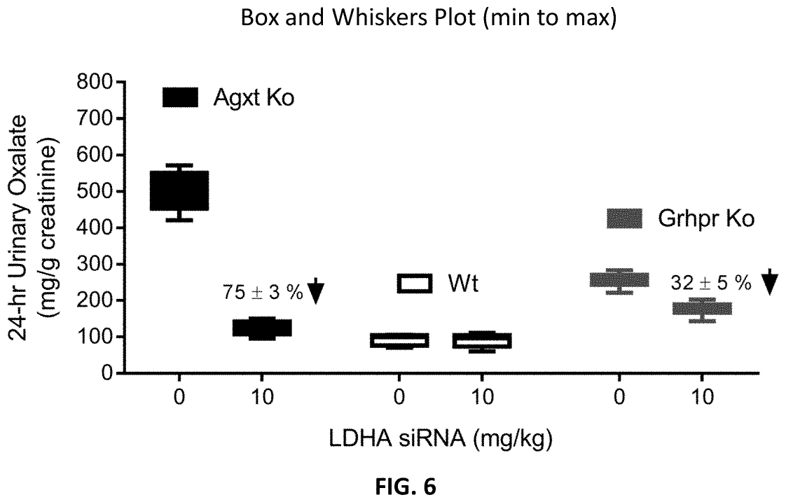

As exemplified in Example 3, the level of endogenous oxalate excreted in the urine of an art recognized animal model of PH1, e.g., an Agxt deficient mouse, was reduced following administration of an LDHA-specific siRNA (see, e.g., FIG. 6). Accordingly, in one aspect, the present invention provides methods for treating a subject having PHE The methods include administering to the subject a therapeutically effective amount of a dsRNA targeting an LDHA gene and/or an HAO1 gene, a pharmaceutical composition comprising a dsRNA agent that targets an LDHA gene and/or a dsRNA agent that targets an HAO1 gene.

As also exemplified in Example 3, the level of endogenous oxalate excreted in the urine of an art recognized animal model of PH2, e.g., a Grhpr deficient mouse, was reduced following administration of an LDHA-specific siRNA (see, e.g., FIG. 6). Accordingly, in one aspect, the present invention provides methods for treating a subject having PH2. The methods include administering to the subject a therapeutically effective amount of a dsRNA targeting an LDHA gene and/or an HAO1 gene, a pharmaceutical composition comprising a dsRNA agent that targets an LDHA gene and/or a dsRNA agent that targets an HAO1 gene in a cell of the subject.

In some embodiment, the methods for treating a subject having PH2 further include altering the diet of the subject (e.g., decreasing protein intake, decreasing sodium intake, decreasing ascorbic acid intake, moderatating calcium intake, supplementing phosphate, supplementing magnesium, or pyridoxine treatment; or a combination of any of the foregoing) and/or transplanting a kidney in the subject

In another embodiment, the calcium oxalate stone formation disease, disorder, or condition is enteric hyperoxaluria. Enteric hyperoxaluria is the formation of calcium oxalate calculi in the urinary tract due to excessive absorption of oxalate from the colon, occurring as a result of intestinal bacterial overgrowth syndromes, fat malabsorption, chronic biliary or pancreatic disease, various intestinal surgical procedures, gastric bypass surgery, inflammatory bowel disease, or any medical condition that causes chronic diarrhea, e.g., Crohn's disease or ulcerative colitis).

In another embodiment, the calcium oxalate stone formation disease, disorder, or condition is dietary hyperoxaluria, e.g., hyperoxaluria as a result of too much oxalate in the diet, e.g., from too much spinach, rhubarb, almonds, bulgur, millet, corn grits, soy flour, cornmeal, navy beans, etc.

In another embodiment, the calcium oxalate stone formation disease, disorder, or condition is idiopathic hyperoxaluria. Subjects having idiopathic hyperoxaluria have above normal levels of urinary oxalate of unknown cause, but still develop stones. Subjects at risk of developing idiopathic hyperoxaluria include diabetics and obese subjects. For example, epidemiological data has demonstrated that as body mass index (BMI) increases, urinary oxalate excretion increases and subjects having diabetes have increases urinary oxalate levels.

In one embodiment, the non-calcium oxalate stone formation disease, disorder, or condition is hypercalciuria (hypercalcinuria). Hypercalciuria is a condition of elevated calcium in the urine. Chronic hypercalcinuria may lead to impairment of renal function, nephrocalcinosis, and renal insufficiency. Subjects at risk of developing hypercalciuria include subjects having Dent's disease, absorptive hypercalciuria, and primary hyperparathyroid.

In another embodiment, the non-calcium oxalate stone formation disease, disorder, or condition is hypocitraturia. In one embodiment, the hypocitraturia is severe hypocitraturia, e.g., citrate excretion of less than 100 mg per day. In another embodiment, the hypocitraturia is mild to moderate hypocitraturi, e.g., citrate excretion of 100-320 mg per day.

In one embodiment, a non-calcium oxalate stone formation disease, disorder, or condition is a disease, disorder, or condition, such as a ureterolithiasis or a nephrocalcinosis, of calcium stones; struvite (magnesium ammonium phosphate) stones; uric acid stones; or cystine stones. Although the primary component of the stones in such diseases, disorders, and conditions is other than oxalate, oxalate may still be present and form a nidus for further growth of the stones. Accordingly, subjects having a disease, disorder, or condition of calcium stones, struvite (magnesium ammonium phosphate) stones, uric acid stones, or cystine stones would benefit from the methods of the invention.

In one embodiment, an oxalate-associated disease, disorder, or condition is a calcium oxalate tissue deposition disease, disorder, or condition. For example, when glomerular filtration rate (GFR) drops below about 30-40 mL/min per 1.73 m², renal capacity to excrete calcium oxalate is significantly impaired. At this stage, calcium oxalate starts to deposit in extrarenal tissues. Calcium oxalate deposits may occur in the thyroid, breasts, kidneys, bones, and bone marrow, myocardium, cardiac conduction system. This leads to cardiomyopathy, heart block and other cardiac conduction defects, vascular disease, retinopathy, synovitis, oxalate osteopathy and anemia that is noted to be resistant to treatment. The deposition of calcium oxalate mat be systemic or tissue specific. For example, subjects having arthritis, sarcoidosis, end-stage renal disease are at risk of developing systemic calcium oxalate tissue deposition disease, disorder, or condition. Subjects at risk of developing tissue specific depositions in the kidney, for example, include subjects having medullary sponge kidney, nephrocalcinosis, renal tubular acidosis (RTA), and transplant recipients, e.g., kidney transplant receipients.

In one embodiment, an oxalate pathway-associated disease, disorder, or condition is a lactate dehydrogenase-associated disease, disorder, or condition. Non-limiting examples of lactate dehydrogenase-associated diseases, disorders, or conditions include cancer, e.g., cancer, e.g., hepatocellular carcinoma, fatty liver (steatosis), nonalcoholic steatohepatitis (NASH), cirrhosis of the liver, accumulation of fat in the liver, inflammation of the liver, hepatocellular necrosis, liver fibrosis, and nonalcoholic fatty liver disease (NAFLD).

A diagnosis of nonalcoholic fatty liver disease (NAFLD) requires that (a) there is evidence of hepatic steatosis, either by imaging or by histology and (b) there are no causes for secondary hepatic fat accumulation such as significant alcohol consumption, use of steatogenic medication or hereditary disorders. In the majority of patients, NAFLD is associated with metabolic risk factors such as obesity, diabetes mellitus, and dyslipidemia. NAFLD is histologically further categorized into nonalcoholic fatty liver (NAFL) and nonalcoholic steatohepatitis (NASH). NAFL is defined as the presence of hepatic steatosis with no evidence of hepatocellular injury in the form of ballooning of the hepatocytes. NASH is defined as the presence of hepatic steatosis and inflammation with hepatocyte injury (ballooning) with or without fibrosis (Chalasani et al., Hepatol. 55:2005-2023, 2012). It is generally agreed that patients with simple steatosis have very slow, if any, histological progression, while patients with NASH can exhibit histological progression to cirrhotic-stage disease. The long term outcomes of patients with NAFLD and NASH have been reported in several studies.

LHDA is required for the initiation, maintenance and progression of tumors (Shi and Pinto, PLOS ONE 2014, 9(1), e86365; Le et al. Proc Natl Acad Sci USA 107: 2037-2042) and up-regulation of LDHA is a characteristic of many cancer types (Goldman R D et al., Cancer Res 24: 389-399.; Koukourakis M I, et al, Br J Cancer 89: 877-885.; Koukourakis M I, et al, L J Clin Oncol 24: 4301-4308.; Kolev Y, et al, Ann Surg Oncol 15: 2336-2344; Zhuang L, et al, Mod Pathol 23: 45-53), including, e.g., breast cancer, lymphoma, renal cancer (including renal cell cancer tumors), hereditary leiomyomatosis, pancreatic cancer, liver cancer (including hepatocellular carcinoma), and other forms of cancer.

In another aspect, the present invention provides uses of a therapeutically effective amount of a dsRNA agent, a dual targeting iRNA agent or a pharmaceutical composition comprising a dsRNA, a pharmaceutical compositions comprising a dual targeting RNAi agent or pharmaceutical composition of the invention comprising a first dsRNA agent that inhibits expression of LDHA and a second dsRNA agent that inhibits expression of HAO1 for treating a subject, e.g., a subject that would benefit from a reduction and/or inhibition of LDHA expression or LDHA and HAO1 expression, e.g., an oxalate pathway-associated disease, disorder, or condition.

In a further aspect, the present invention provides uses of a dual targeting iRNA agent or a pharmaceutical composition comprising of a dsRNA agent, a dual targeting iRNA agent or a pharmaceutical composition comprising a dsRNA, a pharmaceutical composition comprising a dual targeting RNAi agent or pharmaceutical composition of the invention comprising a first dsRNA agent that inhibits expression of LDHA and a second dsRNA agent that inhibits expression of HAO1 in the manufacture of a medicament for treating a subject, e.g., a subject that would benefit from a reduction and/or inhibition of LDHA expression or LDHA and HAO1 expression, e.g., an oxalate pathway-associated disease, disorder, or condition.

In the methods (and uses) of the invention which comprise administering to a subject a first dsRNA agent targeting LDHA and a second dsRNA agent targeting HAO1, the first and second dsRNA agents may be formulated in the same composition or different compositions and may administered to the subject in the same composition or in separate compositions.

The dsRNA agent may be administered to the subject at a dose of about 0.1 mg/kg to about 50 mg/kg. Typically, a suitable dose will be in the range of about 0.1 mg/kg to about 5.0 mg/kg, preferably about 0.3 mg/kg and about 3.0 mg/kg. In addition, the. The dual targeting RNAi agent may be administered to the subject at a dose of about 0.1 mg/kg to about 50 mg/kg. Typically, a suitable dose will be in the range of about 0.1 mg/kg to about 5.0 mg/kg, preferably about 0.3 mg/kg and about 3.0 mg/kg. In addition, the first dsRNA agent and the second dsRNA agent may be each independently administered to the subject at a dose of about 0.5 mg/kg to about 50 mg/kg, e.g., in the range of about 0.1 mg/kg to about 5.0 mg/kg, preferably about 0.3 mg/kg and about 3.0 mg/kg.

In the methods (and uses) of the invention which comprise administering to a subject a first dsRNA agent targeting LDHA and a second dsRNA agent targeting HAO1, the first and second dsRNA agents may be administered to a subject at the same dose or different doses.

The iRNA can be administered by intravenous infusion over a period of time, on a regular basis. In certain embodiments, after an initial treatment regimen, the treatments can be administered on a less frequent basis.

Administration of the iRNA can reduce LDHA levels, e.g., in a cell, tissue, blood, urine or other compartment of the patient by at least about 5%, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 39, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or at least about 99% or more. In a preferred embodiment, administration of the iRNA can reduce LDHA levels, e.g., in a cell, tissue, blood, urine or other compartment of the patient by at least 20%.

Administration of the iRNA can reduce HAO1 levels, e.g., in a cell, tissue, blood, urine or other compartment of the patient by at least about 5%, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 39, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or at least about 99% or more. In a preferred embodiment, administration of the iRNA can reduce HAO1 levels, e.g., in a cell, tissue, blood, urine or other compartment of the patient by at least 20%.

In the methods (and uses) of the invention which comprise administering to a subject a first dsRNA agent targeting LDHA and a second dsRNA agent targeting HAO1, the level of inhibition of LDHA may be the same or different that the level of inhibition of HAO1.

In the methods (and uses) of the invention which comprise administering to a subject a dual targeting RNAi agent, the dual targeting RNAi agent may inhibit expression of the LDHA gene and the HAO1 gene to a level substantially the same as the level of inhibition of expression obtained by the contacting of a cell with both dsRNA agents individually, or the dual targeting RNAi agent may inhibit expression of the LDHA gene and the HAO1 gene to a level higher than the level of inhibition of expression obtained by the contacting of a cell with both dsRNA agents individually.

Before administration of a full dose of the iRNA, patients can be administered a smaller dose, such as a 5% infusion reaction, and monitored for adverse effects, such as an allergic reaction. In another example, the patient can be monitored for unwanted immunostimulatory effects, such as increased cytokine (e.g., TNF-alpha or INF-alpha) levels.

Alternatively, the iRNA can be administered subcutaneously, i.e., by subcutaneous injection. One or more injections may be used to deliver the desired daily dose of iRNA to a subject. The injections may be repeated over a period of time. The administration may be repeated on a regular basis. In certain embodiments, after an initial treatment regimen, the treatments can be administered on a less frequent basis. A repeat-dose regimine may include administration of a therapeutic amount of iRNA on a regular basis, such as every other day or to once a year. In certain embodiments, the iRNA is administered about once per month to about once per quarter (i.e., about once every three months).

In one embodiment, the method includes administering a composition featured herein such that expression of the target LDHA gene and/or the target HAO1 gene is decreased, such as for about 1, 2, 3, 4, 5, 6, 7, 8, 12, 16, 18, 24 hours, 28, 32, or abour 36 hours. In one embodiment, expression of the target LDHA gene and the HAO1 gene is decreased for an extended duration, e.g., at least about two, three, four days or more, e.g., about one week, two weeks, three weeks, or four weeks or longer.

Preferably, the iRNAs useful for the methods and compositions featured herein specifically target RNAs (primary or processed) of the target LDHA and HAO1 genes. Compositions and methods for inhibiting the expression of these genes using iRNAs can be prepared and performed as described herein.

Administration of the dsRNA according to the methods of the invention may result in a reduction of the severity, signs, symptoms, and/or markers of such diseases or disorders in a patient with a disorder of lipid metabolism. By “reduction” in this context is meant a statistically significant decrease in such level. The reduction can be, for example, at least about 5%, 10%, 15%, 20%, 25%, 30%, 35%, 40%, 45%, 50%, 55%, 60%, 65%, 70%, 75%, 80%, 85%, 90%, 95%, or about 100%.

Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. For example, efficacy of treatment of a disorder of lipid metabolism may be assessed, for example, by periodic monitoring of one or more serum lipid levels, e.g., triglyceride levels. Comparisons of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of an iRNA or pharmaceutical composition thereof, “effective against” a disorder of lipid metabolism indicates that administration in a clinically appropriate manner results in a beneficial effect for at least a statistically significant fraction of patients, such as a improvement of symptoms, a cure, a reduction in disease, extension of life, improvement in quality of life, or other effect generally recognized as positive by medical doctors familiar with treating disorder of lipid metabolisms and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given iRNA drug or formulation of that drug can also be judged using an experimental animal model for the given disease as known in the art.

The invention further provides methods for the use of a iRNA agent or a pharmaceutical composition of the invention, e.g., for treating a subject that would benefit from reduction and/or inhibition of LDHA expression or LDHA and HAOlexpression, e.g., a subject having an oxalate pathway-associated disease, disorder, or condition, in combination with other pharmaceuticals and/or other therapeutic methods, e.g., with known pharmaceuticals and/or known therapeutic methods, such as, for example, those which are currently employed for treating these disorders. For example, in certain embodiments, an iRNA agent or pharmaceutical composition of the invention is administered in combination with, e.g., pyridoxine, an ACE inhibitor (angiotensin converting enzyme inhibitors), e.g., benazepril (Lotensin); an angiotensin II receptor antagonist (ARB) (e.g., losartan potassium, such as Merck & Co.'s Cozaar®), e.g., Candesartan (Atacand); an HMG-CoA reductase inhibitor (e.g., a statin); dietary oxalate degrading compounds, e.g., Oxalate decarboxylase (Oxazyme); calcium binding agents, e.g., Sodium cellulose phosphate (Calcibind); diuretics, e.g., thiazide diuretics, such as hydrochlorothiazide (Microzide); phosphate binders, e.g., Sevelamer (Renagel); magnesium and Vitamin B6 supplements; potassium citrate; orthophosphates, bisphosphonates; oral phosphate and citrate solutions; high fluid intake, urinary tract endoscopy; extracorporeal shock wave lithotripsy; kidney dialysis; kidney stone removal (e.g., surgery); and kidney/liver transplant; or a combination of any of the foregoing.

In certain embodiments, an iRNA agent as described herein is administered in combination with an iRNA agent targeting hydroxyproline dehydrogenase (HYPDH; also known as HPDX or PRODH2) (see, e.g., Li, et al. (Biochem Biophys Acta (2016) 1862:233-239) or an inhibitory analog of HYPDH (see, e.g., Summitt, et al. (Biochem J (2015) 466:273-281).

The iRNA agent and an additional therapeutic agent and/or treatment may be administered at the same time and/or in the same combination, e.g., subcutaneously, or the additional therapeutic agent can be administered as part of a separate composition or at separate times and/or by another method known in the art or described herein.

### VII. Kits

The present invention also provides kits for performing any of the methods of the invention. Such kits include one or more RNAi agent(s) and instructions for use, e.g., instructions for inhibiting expression of a LDHA or LDHA and HAO1 in a cell by contacting the cell with an RNAi agent or pharmaceutical composition of the invention in an amount effective to inhibit expression of the LDHA or LDHA and HAO1. The kits may optionally further comprise means for contacting the cell with the RNAi agent (e.g., an injection device), or means for measuring the inhibition of LDHA and/or HAO1 (e.g., means for measuring the inhibition of LDHA and/or HAO1 mRNA and/or LDHA and/or HAO1 protein). Such means for measuring the inhibition of LDHA and/or HAO1 may comprise a means for obtaining a sample from a subject, such as, e.g., a plasma sample. The kits of the invention may optionally further comprise means for administering the RNAi agent(s) to a subject or means for determining the therapeutically effective or prophylactically effective amount.

Unless otherwise defined, all technical and scientific terms used herein have the same meaning as commonly understood by one of ordinary skill in the art to which this invention belongs. Although methods and materials similar or equivalent to those described herein can be used in the practice or testing of the iRNAs and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1. iRNA Design, Synthesis, Selection, and In Vitro Evaluation

Source of Reagents

Where the source of a reagent is not specifically given herein, such reagent can be obtained from any supplier of reagents for molecular biology at a quality/purity standard for application in molecular biology.

Transcripts

A set of iRNAs targeting LDHA that cross-react with mouse and rat Ldha (human NCBI refseqID: NM_010699.2) were designed using custom R and Python scripts. The mouse Ldha, variant 1 REFSEQ mRNA has a length of 1,661 bases.

An additional set of iRNAs targeting LDHA (human: NCBI refseqID NM_005566.3; NCBI GeneID: 3939) as well as toxicology-species LDHA orthologs (cynomolgus monkey: NM_001283551.1) was designed using custom R and Python scripts. The human NM_005566 REFSEQ mRNA, version 3, has a length of 2226 bases.

A detailed list of the unmodified mouse/rat cross-reactive LDHA sense and antisense strand sequences is shown in Table 2. A detailed list of the modified mouse/rat cross-reactive LDHA sense and antisense strand sequences is shown in Table 3.

A detailed list of the unmodified human/Cynomolgus cross-reactive LDHA sense and antisense strand sequences is shown in Table 4. A detailed list of the modified human/Cynomolgus cross-reactive LDHA sense and antisense strand sequences is shown in Table 5.

As described in PCT Publication, WO 2016/057893 (the entire contents of thwich is incorporated herein by reference), a set of iRNAs targeting HAO1 were also designed. Design used the following transcripts from the NCBI RefSeq collection: human (Homo sapiens) HAO1 mRNA is NM_017545.2; cynomolgus monkey (Macaca fascicularis) HAO1 mRNA is XM_005568381.1; Mouse (Mus musculus) HAO1 mRNA is NM_010403.2; Rat (Rattus norvegicus) HAO1 mRNA is XM_006235096.1.

Tables 7 and 8 provide the modified sense and antisense strand sequences of duplexes targeting HAO1. Tables 9, 10, 11, 14, and 15 provide the unmodified sense and antisense strand sequences of duplexes targeting HAO1. Tables 12, 13, and 16 provide the unmodified and modified sense and antisense strand sequences of duplexes targeting HAO1.

When known, the species of HAO1 that is inhibited by the duplex is noted: Hs indicates that the agent inhibits the expression of human HAO1; Mm indicates that the agent inhibits the expression of mouse HAO1; and Hs/Mm indicates that the agent inhibits expression of both human and mouse HAO.

In Vitro Screening:

Cell culture and transfections Primary Mouse Hepatocyte cells (PMH) (MSCP10, Lot # MC613) were transfected by adding 4.9 μl of Opti-MEM plus 0.1 μl of Lipofectamine RNAiMax per well (Invitrogen, Carlsbad Calif. cat #13778-150) to 5 μl of siRNA duplexes per well into a 384-well plate and incubated at room temperature for 15 minutes. Forty μl of DMEM (Hep3b) of William's E Medium (PMH) containing about 5×10³ cells was then added to the siRNA mixture. Cells were incubated for 24 hours prior to RNA purification. Single dose experiments were performed at 10 nM and 0.1 nM final duplex concentration.

Hep3b cells (ATCC) were transfected by adding 4.9 μl of Opti-MEM plus 0.1 μl of Lipofectamine RNAiMax per well (Invitrogen, Carlsbad Calif. cat #13778-150) to 5 μl of siRNA duplexes per well into a 384-well plate and incubated at room temperature for 15 minutes. Forty ul of Eagle's Minimal Essential Medium (Life Tech) containing ˜5×10³ cells were then added to the siRNA mixture. Cells were incubated for 24 hours prior to RNA purification. Single dose experiments were performed at 10 nM.

Total RNA Isolation Using DYNABEADS mRNA Isolation Kit (Invitrogen, Part #: 610-12)

Cells were lysed in 75 μl of Lysis/Binding Buffer containing 3 μL of beads per well and mixed for 10 minutes on an electrostatic shaker. The washing steps were automated on a Biotek EL406, using a magnetic plate support. Beads were washed (90 μL) once in Buffer A, once in Buffer B, and twice in Buffer E, with aspiration steps in between. Following a final aspiration, complete 10 μL RT mixture was added to each well, as described below.

cDNA Synthesis Using ABI High Capacity cDNA Reverse Transcription Kit (Applied Biosystems, Foster City, Calif., Cat #4368813)

A master mix of 1 μl 10× Buffer, 0.4 μl 25×dNTPs, 1 μl Random primers, 0.5 μl Reverse Transcriptase, 0.5 μl RNase inhibitor and 6.6 μl of H₂O per reaction was added per well. Plates were sealed, agitated for 10 minutes on an electrostatic shaker, and then incubated at 37° C. for 2 hours. Following this, the plates were agitated at 80° C. for 8 minutes.

Real Time PCR

Two μ1 of cDNA was added to a master mix containing 0.5 μl of human GAPDH TaqMan Probe (4326317E), 0.5 μl human LDHA, 2 μl nuclease-free water and 50 μl Lightcycler 480 probe master mix (Roche Cat #04887301001) per well in a 384 well plates (Roche cat #04887301001). Real time PCR was performed in a LightCycler480 Real Time PCR system (Roche) using the ΔΔCt(RQ) assay. Each duplex was tested in at least two independent transfections, unless otherwise noted in the summary tables.

To calculate relative fold change, real time data was analyzed using the ΔΔCt method and normalized to assays performed with cells transfected with 10 nM nonspecific siRNA, or mock transfected cells.

Table 6A shows the results of a single dose screen in primary mouse hepatocytes transfected with the indicated GalNAC conjugated modified iRNAs. Data are expressed as percent of message remaining relative to untreated cells.

Table 6B shows the results of a single dose screen in primary mouse hepatocytes transfected with the indicated GalNAC conjugated modified iRNAs. Data are expressed as percent of message remaining relative to untreated cells.

TABLE 1
Abbreviations of nucleotide monomers used in
nucleic acid sequence representation.
It will be understood that these monomers, when present in an
oligonucleotide, are mutually linked by 5′-3′-phosphodiester bonds.

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide (G, A, C, T or U) |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol |
| | Hyp-(GalNAc-alkyl)3 |
| Y34 | 2-hydroxymethyl-tetrahydrofurane-4-methoxy-3-phosphate |
| | (abasic 2′-OMe furanose) |
| Y44 | inverted abasic DNA (2-hydroxymethyl-tetrahydrofurane-5- |
| | phosphate) |
| (Agn) | Adenosine-glycol nucleic acid (GNA) |
| (Cgn) | Cytidine-glycol nucleic acid (GNA) |
| (Ggn) | Guanosine-glycol nucleic acid (GNA) |
| (Tgn) | Thymidine-glycol nucleic acid (GNA) S-Isomer |
| P | Phosphate |
| VP | Vinyl-phosphate |
| (Aam) | 2′-O-(N-methylacetamide)adenosine-3′-phosphate |
| (Aams) | 2′-O-(N-methylacetamide)adenosine-3′-phosphorothioate |
| (Gam) | 2′-O-(N-methylacetamide)guanosine-3′-phosphate |
| (Gams) | 2′-O-(N-methylacetamide)guanosine-3′-phosphorothioate |
| (Tam) | 2′-O-(N-methylacetamide)thymidine-3′-phosphate |
| (Tams) | 2′-O-(N-methylacetamide)thymidine-3′-phosphorothioate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |
| (Aeo) | 2′-O-methoxyethyladenosine-3′-phosphate |
| (Aeos) | 2′-O-methoxyethyladenosine-3′-phosphorothioate |
| (Geo) | 2′-O-methoxyethylguanosine-3′-phosphate |
| (Geos) | 2′-O-methoxyethylguanosine-3′-phosphorothioate |
| (Teo) | 2′-O-methoxyethyl-5-methyluridine-3′-phosphate |
| (Teos) | 2′-O-methoxyethyl-5-methyluridine-3′-phosphorothioate |
| (m5Ceo) | 2′-O-methoxyethyl-5-methylcytidine-3′-phosphate |
| (m5Ceos) | 2′-O-methoxyethyl-5-methylcytidine-3′-phosphorothioate |
| (A3m) | 3′-O-methyladenosine-2′-phosphate |
| (A3mx) | 3′-O-methyl-xylofuranosyladenosine-2′-phosphate |
| (G3m) | 3′-O-methylguanosine-2′-phosphate |
| (G3mx) | 3′-O-methyl-xylofuranosylguanosine-2′-phosphate |
| (C3m) | 3′-O-methylcytidine-2′-phosphate |
| (C3mx) | 3′-O-methyl-xylofuranosylcytidine-2′-phosphate |
| (U3m) | 3′-O-methyluridine-2′-phosphate |
| U3mx) | 3′-O-methyl-xylofuranosyluridine-2′-phosphate |
| (m5Cam) | 2′-O-(N-methylacetamide)-5-methylcytidine-3′-phosphate |
| (m5Cams) | 2′-O-(N-methylacetamide)-5-methylcytidine-3′- |
| | phosphorothioate |
| (Chd) | 2′-O-hexadecyl-cytidine-3′-phosphate |
| (Chds) | 2′-O-hexadecyl-cytidine-3′-phosphorothioate |
| (Uhd) | 2′-O-hexadecyl-uridine-3′-phosphate |
| (Uhds) | 2′-O-hexadecyl-uridine-3′-phosphorothioate |
| (pshe) | Hydroxyethylphosphorothioate |

TABLE 2
UNMODIFIED MOUSE/RAT CROSS-REACTIVE LDHA iRNA SEQUENCES

| | Sense | | SEQ | | Antisense | | SEQ | |
|---|---|---|---|---|---|---|---|---|
| Duplex | Oligo | Sense | ID | Range in | Oligo | Antisense | ID | Range in |
| Name | Name | Sequence 5′ to 3′ | NO | NM_010699.2 | Name | Sequence 5′ to 3′ | NO | NM_010699.2 |
| AD- | A-169171 | AACACCAAAAAUUGUCUCCAA | 2990 | 357-377 | A-169172 | UUGGAGACAAUUUUUGGUGUUUU | 3034 | 355-377 |
| 84747 | | | | | | | | |
| AD- | A-169173 | AAACCGAGUAAUUGGAAGUGA | 2991 | 603-623 | A-169174 | UCACUUCCAAUUACUCGGUUUUU | 3035 | 601-623 |
| 84748 | | | | | | | | |
| AD- | A-169175 | AAAUCAGUGGCUUUCCCAAAA | 2992 | 584-604 | A-169176 | UUUUGGGAAAGCCACUGAUUUUC | 3036 | 582-604 |
| 84749 | | | | | | | | |
| AD- | A-169177 | UCCCAACAUUGUCAAGUACAA | 2993 | 501-521 | A-169178 | UUGUACUUGACAAUGUUGGGAAU | 3037 | 499-521 |
| 84750 | | | | | | | | |
| AD- | A-169179 | UGUGCCAUCAGUAUCUUAAUA | 2994 | 241-261 | A-169180 | UAUUAAGAUACUGAUGGCACAAG | 3038 | 239-261 |
| 84751 | | | | | | | | |
| AD- | A-169181 | AAAUUGUCUCCAGCAAAGACU | 2995 | 365-385 | A-169182 | AGUCUUUGCUGGAGACAAUUUUU | 3039 | 363-385 |
| 84752 | | | | | | | | |
| AD- | A-169183 | ACCUUGAACAGUGAAAAAAAA | 2996 | 1610-1630 | A-169184 | UUUUUUUUCACUGUUCAAGGUUU | 3040 | 1608-1630 |
| 84753 | | | | | | | | |
| AD- | A-169185 | AAAACACCAAAAAUUGUCUCA | 2997 | 355-375 | A-169186 | UGAGACAAUUUUUGGUGUUUUAA | 3041 | 353-375 |
| 84754 | | | | | | | | |
| AD- | A-169187 | ACCAAAAAUUGUCUCCAGCAA | 2998 | 360-380 | A-169188 | UUGCUGGAGACAAUUUUUGGUGU | 3042 | 358-380 |
| 84755 | | | | | | | | |
| AD- | A-169189 | CAAGUUCAUCAUUCCCAACAU | 2999 | 489-509 | A-169190 | AUGUUGGGAAUGAUGAACUUGAA | 3043 | 487-509 |
| 84756 | | | | | | | | |
| AD- | A-169191 | GCAAUAUUAUGUGAGAUGUAA | 3000 | 1538-1558 | A-169192 | UUACAUCUCACAUAAUAUUGCAA | 3044 | 1536-1558 |
| 84757 | | | | | | | | |
| AD- | A-169193 | GUCUCAAAAGAUUCAAAGUCA | 3001 | 115-135 | A-169194 | UGACUUUGAAUCUUUUGAGACCG | 3045 | 113-135 |
| 84758 | | | | | | | | |
| AD- | A-169195 | CAUUCCCAACAUUGUCAAGUA | 3002 | 498-518 | A-169196 | UACUUGACAAUGUUGGGAAUGAU | 3046 | 496-518 |
| 84759 | | | | | | | | |
| AD- | A-169197 | AAACCUUGAACAGUGAAAAAA | 3003 | 1608-1628 | A-169198 | UUUUUUCACUGUUCAAGGUUUUA | 3047 | 1606-1628 |
| 84760 | | | | | | | | |
| AD- | A-169199 | UCAAAAGAUUCAAAGUCCAAA | 3004 | 118-138 | A-169200 | UUUGGACUUUGAAUCUUUUGAGA | 3048 | 116-138 |
| 84761 | | | | | | | | |
| AD- | A-169203 | ACAUCUUCAAGUUCAUCAUUA | 3005 | 482-502 | A-169204 | UAAUGAUGAACUUGAAGAUGUUC | 3049 | 480-502 |
| 84762 | | | | | | | | |
| AD- | A-169205 | CAGCUGAUUGUGAAUCUUCUU | 3006 | 157-177 | A-169206 | AAGAAGAUUCACAAUCAGCUGGU | 3050 | 155-177 |
| 84763 | | | | | | | | |
| AD- | A-169207 | CUCAAAAGAUUCAAAGUCCAA | 3007 | 117-137 | A-169208 | UUGGACUUUGAAUCUUUUGAGAC | 3051 | 115-137 |
| 84764 | | | | | | | | |
| AD- | A-169209 | AAAACCGAGUAAUUGGAAGUA | 3008 | 602-622 | A-169210 | UACUUCCAAUUACUCGGUUUUUG | 3052 | 600-622 |
| 84765 | | | | | | | | |
| AD- | A-169213 | UGAUGCAUAUCUUGUGCAUAA | 3009 | 1469-1489 | A-169214 | UUAUGCACAAGAUAUGCAUCAUG | 3053 | 1467-1489 |
| 84766 | | | | | | | | |
| AD- | A-169215 | CCAUCAGUAUCUUAAUGAAGA | 3010 | 245-265 | A-169216 | UCUUCAUUAAGAUACUGAUGGCA | 3054 | 243-265 |
| 84767 | | | | | | | | |
| AD- | A-169217 | AAUCAGUGGCUUUCCCAAAAA | 3011 | 585-605 | A-169218 | UUUUUGGGAAAGCCACUGAUUUU | 3055 | 583-605 |
| 84768 | | | | | | | | |
| AD- | A-169219 | UUAAAACACCAAAAAUUGUCU | 3012 | 353-373 | A-169220 | AGACAAUUUUUGGUGUUUUAAGG | 3056 | 351-373 |
| 84769 | | | | | | | | |
| AD- | A-169221 | CUGAUUGUGAAUCUUCUUAAA | 3013 | 160-180 | A-169222 | UUUAAGAAGAUUCACAAUCAGCU | 3057 | 158-180 |
| 84770 | | | | | | | | |
| AD- | A-169223 | AUAAAACCUUGAACAGUGAAA | 3014 | 1605-1625 | A-169224 | UUUCACUGUUCAAGGUUUUAUUU | 3058 | 1603-1625 |
| 84771 | | | | | | | | |
| AD- | A-169225 | AGUGUCAUGCCAAAUAAAACA | 3015 | 1592-1612 | A-169226 | UGUUUUAUUUGGCAUGACACUUG | 3059 | 1590-1612 |
| 84772 | | | | | | | | |
| AD- | A-169227 | ACACCAAAAAUUGUCUCCAGA | 3016 | 358-378 | A-169228 | UCUGGAGACAAUUUUUGGUGUUU | 3060 | 356-378 |
| 84773 | | | | | | | | |
| AD- | A-169229 | GCAUUGCAAUAUUAUGUGAGA | 3017 | 1533-1553 | A-169230 | UCUCACAUAAUAUUGCAAUGCAC | 3061 | 1531-1553 |
| 84774 | | | | | | | | |
| AD- | A-169231 | GUCAUGCCAAAUAAAACCUUA | 3018 | 1595-1615 | A-169232 | UAAGGUUUUAUUUGGCAUGACAC | 3062 | 1593-1615 |
| 84775 | | | | | | | | |
| AD- | A-169233 | AUAUCUUGUGCAUAAAUGUUA | 3019 | 1475-1495 | A-169234 | UAACAUUUAUGCACAAGAUAUGC | 3063 | 1473-1495 |
| 84776 | | | | | | | | |
| AD- | A-169235 | AAACACCAAAAAUUGUCUCCA | 3020 | 356-376 | A-169236 | UGGAGACAAUUUUUGGUGUUUUA | 3064 | 354-376 |
| 84777 | | | | | | | | |
| AD- | A-169237 | UAACCUGGCUCCAGUGUGUAA | 3021 | 1443-1463 | A-169238 | UUACACACUGGAGCCAGGUUAUA | 3065 | 1441-1463 |
| 84778 | | | | | | | | |
| AD- | A-169239 | UGCAUAUCUUGUGCAUAAAUA | 3022 | 1472-1492 | A-169240 | UAUUUAUGCACAAGAUAUGCAUC | 3066 | 1470-1492 |
| 84779 | | | | | | | | |
| AD- | A-169241 | ACAUUGUCAAGUACAGUCCAA | 3023 | 506-526 | A-169242 | UUGGACUGUACUUGACAAUGUUG | 3067 | 504-526 |
| 84780 | | | | | | | | |
| AD- | A-169243 | AACCUUGAACAGUGAAAAAAA | 3024 | 1609-1629 | A-169244 | UUUUUUUCACUGUUCAAGGUUUU | 3068 | 1607-1629 |
| 84781 | | | | | | | | |
| AD- | A-169245 | GUGUGCAUUGCAAUAUUAUGU | 3025 | 1529-1549 | A-169246 | ACAUAAUAUUGCAAUGCACACUA | 3069 | 1527-1549 |
| 84782 | | | | | | | | |
| AD- | A-169247 | CCAAAAACCGAGUAAUUGGAA | 3026 | 599-619 | A-169248 | UUCCAAUUACUCGGUUUUUGGGA | 3070 | 597-619 |
| 84783 | | | | | | | | |
| AD- | A-169249 | CAAAAACCGAGUAAUUGGAAA | 3027 | 600-620 | A-169250 | UUUCCAAUUACUCGGUUUUUGGG | 3071 | 598-620 |
| 84784 | | | | | | | | |
| AD- | A-169251 | CCAAGUGGUACUUGUGUAGUA | 3028 | 1285-1305 | A-169252 | UACUACACAAGUACCACUUGGCA | 3072 | 1283-1305 |
| 84785 | | | | | | | | |
| AD- | A-169253 | CAGCGAAACGUGAACAUCUUA | 3029 | 469-489 | A-169254 | UAAGAUGUUCACGUUUCGCUGGA | 3073 | 467-489 |
| 84786 | | | | | | | | |
| AD- | A-169255 | UGAUUGUGAAUCUUCUUAAGA | 3030 | 161-181 | A-169256 | UCUUAAGAAGAUUCACAAUCAGC | 3074 | 159-181 |
| 84787 | | | | | | | | |
| AD- | A-169257 | CUUCAAGUUCAUCAUUCCCAA | 3031 | 486-506 | A-169258 | UUGGGAAUGAUGAACUUGAAGAU | 3075 | 484-506 |
| 84788 | | | | | | | | |
| AD- | A-169259 | GGACCAGCUGAUUGUGAAUCU | 3032 | 153-173 | A-169260 | AGAUUCACAAUCAGCUGGUCCUU | 3076 | 151-173 |
| 84789 | | | | | | | | |
| AD- | A-169261 | AUGCCAAAUAAAACCUUGAAA | 3033 | 1598-1618 | A-169262 | UUUCAAGGUUUUAUUUGGCAUGA | 3077 | 1596-1618 |
| 84790 | | | | | | | | |

TABLE 3
MODIFIED MOUSE/RAT CROSS-REACTIVE LDHA iRNA SEQUENCES

| | | SEQ | |
|---|---|---|---|
| | | ID | |
| Duplex Name | Sense Sequence 5′ to 3′ | NO | Antisense Sequence 5′ to 3′ |
| AD-84747 | asascaccAfaAfAfAfuugucuccaaL96 | 3078 | usUfsggaGfaCfAfauuuUfuGfguguususu |
| AD-84748 | asasaccgAfgUfAfAfuuggaagugaL96 | 3079 | usCfsacuUfcCfAfauuaCfuCfgguuususu |
| AD-84749 | asasaucaGfuGfGfCfuuucccaaaaL96 | 3080 | usUfsuugGfgAfAfagccAfcUfgauuususc |
| AD-84750 | uscsccaaCfaUfUfGfucaaguacaaL96 | 3081 | usUfsguaCfuUfGfacaaUfgUfugggasasu |
| AD-84751 | usgsugccAfuCfAfGfuaucuuaauaL96 | 3082 | usAfsuuaAfgAfUfacugAfuGfgcacasasg |
| AD-84752 | asasauugUfcUfCfCfagcaaagacuL96 | 3083 | asGfsucuUfuGfCfuggaGfaCfaauuususu |
| AD-84753 | ascscuugAfaCfAfGfugaaaaaaaaL96 | 3084 | usUfsuuuUfuUfCfacugUfuCfaaggususu |
| AD-84754 | asasaacaCfcAfAfAfaauugucucaL96 | 3085 | usGfsagaCfaAfUfuuuuGfgUfguuuusasa |
| AD-84755 | ascscaaaAfaUfUfGfucuccagcaaL96 | 3086 | usUfsgcuGfgAfGfacaaUfuUfuuggusgsu |
| AD-84756 | csasaguuCfaUfCfAfuucccaacauL96 | 3087 | asUfsguuGfgGfAfaugaUfgAfacuugsasa |
| AD-84757 | gscsaauaUfuAfUfGfugagauguaaL96 | 3088 | usUfsacaUfcUfCfacauAfaUfauugcsasa |
| AD-84758 | gsuscucaAfaAfGfAfuucaaagucaL96 | 3089 | usGfsacuUfuGfAfaucuUfuUfgagacscsg |
| AD-84759 | csasuuccCfaAfCfAfuugucaaguaL96 | 3090 | usAfscuuGfaCfAfauguUfgGfgaaugsasu |
| AD-84760 | asasaccuUfgAfAfCfagugaaaaaaL96 | 3091 | usUfsuuuUfcAfCfuguuCfaAfgguuususa |
| AD-84761 | uscsaaaaGfaUfUfCfaaaguccaaaL96 | 3092 | usUfsuggAfcUfUfugaaUfcUfuuugasgsa |
| AD-84762 | ascsaucuUfcAfAfGfuucaucauuaL96 | 3093 | usAfsaugAfuGfAfacuuGfaAfgaugususc |
| AD-84763 | csasgcugAfuUfGfUfgaaucuucuuL96 | 3094 | asAfsgaaGfaUfUfcacaAfuCfagcugsgsu |
| AD-84764 | csuscaaaAfgAfUfUfcaaaguccaaL96 | 3095 | usUfsggaCfuUfUfgaauCfuUfuugagsasc |
| AD-84765 | asasaaccGfaGfUfAfauuggaaguaL96 | 3096 | usAfscuuCfcAfAfuuacUfcGfguuuususg |
| AD-84766 | usgsaugcAfuAfUfCfuugugcauaaL96 | 3097 | usUfsaugCfaCfAfagauAfuGfcaucasusg |
| AD-84767 | cscsaucaGfuAfUfCfuuaaugaagaL96 | 3098 | usCfsuucAfuUfAfagauAfcUfgauggscsa |
| AD-84768 | asasucagUfgGfCfUfuucccaaaaaL96 | 3099 | usUfsuuuGfgGfAfaagcCfaCfugauususu |
| AD-84769 | ususaaaaCfaCfCfAfaaaauugucuL96 | 3100 | asGfsacaAfuUfUfuuggUfgUfuuuaasgsg |
| AD-84770 | csusgauuGfuGfAfAfucuucuuaaaL96 | 3101 | usUfsuaaGfaAfGfauucAfcAfaucagscsu |
| AD-84771 | asusaaaaCfcUfUfGfaacagugaaaL96 | 3102 | usUfsucaCfuGfUfucaaGfgUfuuuaususu |
| AD-84772 | asgsugucAfuGfCfCfaaauaaaacaL96 | 3103 | usGfsuuuUfaUfUfuggcAfuGfacacususg |
| AD-84773 | ascsaccaAfaAfAfUfugucuccagaL96 | 3104 | usCfsuggAfgAfCfaauuUfuUfggugususu |
| AD-84774 | gscsauugCfaAfUfAfuuaugugagaL96 | 3105 | usCfsucaCfaUfAfauauUfgCfaaugcsasc |
| AD-84775 | gsuscaugCfcAfAfAfuaaaaccuuaL96 | 3106 | usAfsaggUfuUfUfauuuGfgCfaugacsasc |
| AD-84776 | asusaucuUfgUfGfCfauaaauguuaL96 | 3107 | usAfsacaUfuUfAfugcaCfaAfgauausgsc |
| AD-84777 | asasacacCfaAfAfAfauugucuccaL96 | 3108 | usGfsgagAfcAfAfuuuuUfgGfuguuususa |
| AD-84778 | usasaccuGfgCfUfCfcaguguguaaL96 | 3109 | usUfsacaCfaCfUfggagCfcAfgguuasusa |
| AD-84779 | usgscauaUfcUfUfGfugcauaaauaL96 | 3110 | usAfsuuuAfuGfCfacaaGfaUfaugcasusc |
| AD-84780 | ascsauugUfcAfAfGfuacaguccaaL96 | 3111 | usUfsggaCfuGfUfacuuGfaCfaaugususg |
| AD-84781 | asasccuuGfaAfCfAfgugaaaaaaaL96 | 3112 | usUfsuuuUfuCfAfcuguUfcAfagguususu |
| AD-84782 | gsusgugcAfuUfGfCfaauauuauguL96 | 3113 | asCfsauaAfuAfUfugcaAfuGfcacacsusa |
| AD-84783 | cscsaaaaAfcCfGfAfguaauuggaaL96 | 3114 | usUfsccaAfuUfAfcucgGfuUfuuuggsgsa |
| AD-84784 | csasaaaaCfcGfAfGfuaauuggaaaL96 | 3115 | usUfsuccAfaUfUfacucGfgUfuuuugsgsg |
| AD-84785 | cscsaaguGfgUfAfCfuuguguaguaL96 | 3116 | usAfscuaCfaCfAfaguaCfcAfcuuggscsa |
| AD-84786 | csasgcgaAfaCfGfUfgaacaucuuaL96 | 3117 | usAfsagaUfgUfUfcacgUfuUfcgcugsgsa |
| AD-84787 | usgsauugUfgAfAfUfcuucuuaagaL96 | 3118 | usCfsuuaAfgAfAfgauuCfaCfaaucasgsc |
| AD-84788 | csusucaaGfuUfCfAfucauucccaaL96 | 3119 | usUfsgggAfaUfGfaugaAfcUfugaagsasu |
| AD-84789 | gsgsaccaGfcUfGfAfuugugaaucuL96 | 3120 | asGfsauuCfaCfAfaucaGfcUfgguccsusu |
| AD-84790 | asusgccaAfaUfAfAfaaccuugaaaL96 | 3121 | usUfsucaAfgGfUfuuuaUfuUfggcausgsa |

| Duplex Name | SEQ ID NO | mRNA target sequence | SEQ ID NO |
|---|---|---|---|
| AD-84747 | 3122 | AAAACACCAAAAAUUGUCUCCAG | 3166 |
| AD-84748 | 3123 | AAAAACCGAGUAAUUGGAAGUGG | 3167 |
| AD-84749 | 3124 | GAAAAUCAGUGGCUUUCCCAAAA | 3168 |
| AD-84750 | 3125 | AUUCCCAACAUUGUCAAGUACAG | 3169 |
| AD-84751 | 3126 | CUUGUGCCAUCAGUAUCUUAAUG | 3170 |
| AD-84752 | 3127 | AAAAAUUGUCUCCAGCAAAGACU | 3171 |
| AD-84753 | 3128 | AAACCUUGAACAGUGAAAAAAAA | 3172 |
| AD-84754 | 3129 | UUAAAACACCAAAAAUUGUCUCC | 3173 |
| AD-84755 | 3130 | ACACCAAAAAUUGUCUCCAGCAA | 3174 |
| AD-84756 | 3131 | UUCAAGUUCAUCAUUCCCAACAU | 3175 |
| AD-84757 | 3132 | UUGCAAUAUUAUGUGAGAUGUAA | 3176 |
| AD-84758 | 3133 | CGGUCUCAAAAGAUUCAAAGUCC | 3177 |
| AD-84759 | 3134 | AUCAUUCCCAACAUUGUCAAGUA | 3178 |
| AD-84760 | 3135 | UAAAACCUUGAACAGUGAAAAAA | 3179 |
| AD-84761 | 3136 | UCUCAAAAGAUUCAAAGUCCAAG | 3180 |
| AD-84762 | 3137 | GAACAUCUUCAAGUUCAUCAUUC | 3181 |
| AD-84763 | 3138 | ACCAGCUGAUUGUGAAUCUUCUU | 3182 |
| AD-84764 | 3139 | GUCUCAAAAGAUUCAAAGUCCAA | 3183 |
| AD-84765 | 3140 | CAAAAACCGAGUAAUUGGAAGUG | 3184 |
| AD-84766 | 3141 | CAUGAUGCAUAUCUUGUGCAUAA | 3185 |
| AD-84767 | 3142 | UGCCAUCAGUAUCUUAAUGAAGG | 3186 |
| AD-84768 | 3143 | AAAAUCAGUGGCUUUCCCAAAAA | 3187 |
| AD-84769 | 3144 | CCUUAAAACACCAAAAAUUGUCU | 3188 |
| AD-84770 | 3145 | AGCUGAUUGUGAAUCUUCUUAAG | 3189 |
| AD-84771 | 3146 | AAAUAAAACCUUGAACAGUGAAA | 3190 |
| AD-84772 | 3147 | CAAGUGUCAUGCCAAAUAAAACC | 3191 |
| AD-84773 | 3148 | AAACACCAAAAAUUGUCUCCAGC | 3192 |
| AD-84774 | 3149 | GUGCAUUGCAAUAUUAUGUGAGA | 3193 |
| AD-84775 | 3150 | GUGUCAUGCCAAAUAAAACCUUG | 3194 |
| AD-84776 | 3151 | GCAUAUCUUGUGCAUAAAUGUUG | 3195 |
| AD-84777 | 3152 | UAAAACACCAAAAAUUGUCUCCA | 3196 |
| AD-84778 | 3153 | UAUAACCUGGCUCCAGUGUGUAC | 3197 |
| AD-84779 | 3154 | GAUGCAUAUCUUGUGCAUAAAUG | 3198 |
| AD-84780 | 3155 | CAACAUUGUCAAGUACAGUCCAC | 3199 |
| AD-84781 | 3156 | AAAACCUUGAACAGUGAAAAAAA | 3200 |
| AD-84782 | 3157 | UAGUGUGCAUUGCAAUAUUAUGU | 3201 |
| AD-84783 | 3158 | UCCCAAAAACCGAGUAAUUGGAA | 3202 |
| AD-84784 | 3159 | CCCAAAAACCGAGUAAUUGGAAG | 3203 |
| AD-84785 | 3160 | UGCCAAGUGGUACUUGUGUAGUG | 3204 |
| AD-84786 | 3161 | UCCAGCGAAACGUGAACAUCUUC | 3205 |
| AD-84787 | 3162 | GCUGAUUGUGAAUCUUCUUAAGG | 3206 |
| AD-84788 | 3163 | AUCUUCAAGUUCAUCAUUCCCAA | 3207 |
| AD-84789 | 3164 | AAGGACCAGCUGAUUGUGAAUCU | 3208 |
| AD-84790 | 3165 | UCAUGCCAAAUAAAACCUUGAAC | 3209 |

TABLE 4
UNMODIFIED HUMAN/CYNOMOLGUS CROSS-REACTIVE LDHA iRNA SEQUENCES

| | Sense Oligo | | | Position in | Antisense | | | |
|---|---|---|---|---|---|---|---|---|
| Duplex Name | Name | Sense Sequence 5′ to 3′ | SEQ ID NO | NM_005566.3 | Oligo Name | Antisense Sequence 5′ to 3′ | SEQ ID NO | Position in NM_005566.3 |
| AD-159469 | A-314810 | UUUAUCUGAUCUGUGAUUAAA | 3210 | 1347-1367 | A-314811 | UUUAAUCACAGAUCAGAUAAAAA | 3396 | 1345-1367 |
| AD-159607 | A-315086 | ACUGGUUAGUGUGAAAUAGUU | 3211 | 1489-1509 | A-315087 | AACUAUUUCACACUAACCAGUUG | 3397 | 1487-1509 |
| AD-159713 | A-315298 | AACAUGCCUAGUCCAACAUUU | 3212 | 1615-1635 | A-315299 | AAAUGUUGGACUAGGCAUGUUCA | 3398 | 1613-1635 |
| AD-158504 | A-312881 | CAAGUCCAAUAUGGCAACUCU | 3213 | 263-283 | A-312882 | AGAGUUGCCAUAUUGGACUUGGA | 3399 | 261-283 |
| AD-159233 | A-314338 | UCCACCAUGAUUAAGGGUCUU | 3214 | 1092-1112 | A-314339 | AAGACCCUUAAUCAUGGUGGAAA | 3400 | 1090-1112 |
| AD-159411 | A-314694 | UCAUUUCACUGUCUAGGCUAA | 3215 | 1289-1309 | A-314695 | UUAGCCUAGACAGUGAAAUGAUA | 3401 | 1287-1309 |
| AD-159462 | A-314796 | UGUCCUUUUUAUCUGAUCUGU | 3216 | 1340-1360 | A-314797 | ACAGAUCAGAUAAAAAGGACAAC | 3402 | 1338-1360 |
| AD-159742 | A-315356 | CCAGUGUAUAAAUCCAAUAUA | 3217 | 1662-1682 | A-315357 | UAUAUUGGAUUUAUACACUGGAU | 3403 | 1660-1682 |
| AD-159863 | A-315598 | UCCAAGUGUUAUACCAACUAA | 3218 | 1791-1811 | A-315599 | UUAGUUGGUAUAACACUUGGAUA | 3404 | 1789-1811 |
| AD-158626 | A-313124 | GUCAUCGAAGACAAAUUGAAA | 3219 | 429-449 | A-313125 | UUUCAAUUUGUCUUCGAUGACAU | 3405 | 427-449 |
| AD-158687 | A-313246 | GAACACCAAAGAUUGUCUCUA | 3220 | 490-510 | A-313247 | UAGAGACAAUCUUUGGUGUUCUA | 3406 | 488-510 |
| AD-158688 | A-313248 | AACACCAAAGAUUGUCUCUGA | 3221 | 491-511 | A-313249 | UCAGAGACAAUCUUUGGUGUUCU | 3407 | 489-511 |
| AD-159458 | A-314788 | AUGUUGUCCUUUUUAUCUGAU | 3222 | 1336-1356 | A-314789 | AUCAGAUAAAAAGGACAACAUGC | 3408 | 1334-1356 |
| AD-159519 | A-314910 | UCAACUCCUGAAGUUAGAAAU | 3223 | 1401-1421 | A-314911 | AUUUCUAACUUCAGGAGUUGAUG | 3409 | 1399-1421 |
| AD-159858 | A-315588 | AACUAUCCAAGUGUUAUACCA | 3224 | 1786-1806 | A-315589 | UGGUAUAACACUUGGAUAGUUGG | 3410 | 1784-1806 |
| AD-158681 | A-313234 | UCCUUAGAACACCAAAGAUUA | 3225 | 484-504 | A-313235 | UAAUCUUUGGUGUUCUAAGGAAA | 3411 | 482-504 |
| AD-159583 | A-315038 | GGUAUUAAUCUUGUGUAGUCU | 3226 | 1465-1485 | A-315039 | AGACUACACAAGAUUAAUACCAU | 3412 | 1463-1485 |
| AD-159700 | A-315272 | GGCUCCUUCACUGAACAUGCA | 3227 | 1602-1622 | A-315273 | UGCAUGUUCAGUGAAGGAGCCAG | 3413 | 1600-1622 |
| AD-159807 | A-315486 | UAUCAGUAGUGUACAUUACCA | 3228 | 1728-1748 | A-315487 | UGGUAAUGUACACUACUGAUAUA | 3414 | 1726-1748 |
| AD-158673 | A-313218 | CAGCCUUUUCCUUAGAACACA | 3229 | 476-496 | A-313219 | UGUGUUCUAAGGAAAAGGCUGCC | 3415 | 474-496 |
| AD-159608 | A-315088 | CUGGUUAGUGUGAAAUAGUUA | 3230 | 1490-1510 | A-315089 | UAACUAUUUCACACUAACCAGUU | 3416 | 1488-1510 |
| AD-159803 | A-315478 | ACUAUAUCAGUAGUGUACAUU | 3231 | 1724-1744 | A-315479 | AAUGUACACUACUGAUAUAGUUC | 3417 | 1722-1744 |
| AD-159805 | A-315482 | UAUAUCAGUAGUGUACAUUAA | 3232 | 1726-1746 | A-315483 | UUAAUGUACACUACUGAUAUAGU | 3418 | 1724-1746 |
| AD-159489 | A-314850 | GUAAUAUUUUAAGAUGGACUA | 3233 | 1371-1391 | A-314851 | UAGUCCAUCUUAAAAUAUUACUG | 3419 | 1369-1391 |
| AD-159495 | A-314862 | UUUUAAGAUGGACUGGGAAAA | 3234 | 1377-1397 | A-314863 | UUUUCCCAGUCCAUCUUAAAAUA | 3420 | 1375-1397 |
| AD-159609 | A-315090 | UGGUUAGUGUGAAAUAGUUCU | 3235 | 1491-1511 | A-315091 | AGAACUAUUUCACACUAACCAGU | 3421 | 1489-1511 |
| AD-159706 | A-315284 | UUCACUGAACAUGCCUAGUCA | 3236 | 1608-1628 | A-315285 | UGACUAGGCAUGUUCAGUGAAGG | 3422 | 1606-1628 |
| AD-159855 | A-315582 | ACCAACUAUCCAAGUGUUAUA | 3237 | 1783-1803 | A-315583 | UAUAACACUUGGAUAGUUGGUUG | 3423 | 1781-1803 |
| AD-159864 | A-315600 | CCAAGUGUUAUACCAACUAAA | 3238 | 1792-1812 | A-315601 | UUUAGUUGGUAUAACACUUGGAU | 3424 | 1790-1812 |
| AD-158491 | A-312855 | UUCCUUUUGGUUCCAAGUCCA | 3239 | 250-270 | A-312856 | UGGACUUGGAACCAAAAGGAAUC | 3425 | 248-270 |
| AD-158672 | A-313216 | GCAGCCUUUUCCUUAGAACAA | 3240 | 475-495 | A-313217 | UUGUUCUAAGGAAAAGGCUGCCA | 3426 | 473-495 |
| AD-159488 | A-314848 | AGUAAUAUUUUAAGAUGGACU | 3241 | 1370-1390 | A-314849 | AGUCCAUCUUAAAAUAUUACUGC | 3427 | 1368-1390 |
| AD-159553 | A-314978 | AAAAUCCACAGCUAUAUCCUA | 3242 | 1435-1455 | A-314979 | UAGGAUAUAGCUGUGGAUUUUAC | 3428 | 1433-1455 |
| AD-159703 | A-315278 | UCCUUCACUGAACAUGCCUAA | 3243 | 1605-1625 | A-315279 | UUAGGCAUGUUCAGUGAAGGAGC | 3429 | 1603-1625 |
| AD-159708 | A-315288 | CACUGAACAUGCCUAGUCCAA | 3244 | 1610-1630 | A-315289 | UUGGACUAGGCAUGUUCAGUGAA | 3430 | 1608-1630 |
| AD-159866 | A-315604 | AAGUGUUAUACCAACUAAAAC | 3245 | 1794-1814 | A-315605 | GUUUUAGUUGGUAUAACACUUGG | 3431 | 1792-1814 |
| AD-159232 | A-314336 | UUCCACCAUGAUUAAGGGUCU | 3246 | 1091-1111 | A-314337 | AGACCCUUAAUCAUGGUGGAAAC | 3432 | 1089-1111 |
| AD-159712 | A-315296 | GAACAUGCCUAGUCCAACAUU | 3247 | 1614-1634 | A-315297 | AAUGUUGGACUAGGCAUGUUCAG | 3433 | 1612-1634 |
| AD-159808 | A-315488 | AUCAGUAGUGUACAUUACCAU | 3248 | 1729-1749 | A-315489 | AUGGUAAUGUACACUACUGAUAU | 3434 | 1727-1749 |
| AD-159862 | A-315596 | AUCCAAGUGUUAUACCAACUA | 3249 | 1790-1810 | A-315597 | UAGUUGGUAUAACACUUGGAUAG | 3435 | 1788-1810 |
| AD-158503 | A-312879 | CCAAGUCCAAUAUGGCAACUA | 3250 | 262-282 | A-312880 | UAGUUGCCAUAUUGGACUUGGAA | 3436 | 260-282 |
| AD-159311 | A-314494 | AUCUCAGACCUUGUGAAGGUA | 3251 | 1170-1190 | A-314495 | UACCUUCACAAGGUCUGAGAUUC | 3437 | 1168-1190 |
| AD-159412 | A-314696 | CAUUUCACUGUCUAGGCUACA | 3252 | 1290-1310 | A-314697 | UGUAGCCUAGACAGUGAAAUGAU | 3438 | 1288-1310 |
| AD-159558 | A-314988 | CCACAGCUAUAUCCUGAUGCU | 3253 | 1440-1460 | A-314989 | AGCAUCAGGAUAUAGCUGUGGAU | 3439 | 1438-1460 |
| AD-159705 | A-315282 | CUUCACUGAACAUGCCUAGUA | 3254 | 1607-1627 | A-315283 | UACUAGGCAUGUUCAGUGAAGGA | 3440 | 1605-1627 |
| AD-159113 | A-314098 | GUGGUUGAGAGUGCUUAUGAA | 3255 | 972-992 | A-314099 | UUCAUAAGCACUCUCAACCACCU | 3441 | 970-992 |
| AD-159139 | A-314150 | CAAACUCAAAGGCUACACAUA | 3256 | 998-1018 | A-314151 | UAUGUGUAGCCUUUGAGUUUGAU | 3442 | 996-1018 |
| AD-159806 | A-315484 | AUAUCAGUAGUGUACAUUACA | 3257 | 1727-1747 | A-315485 | UGUAAUGUACACUACUGAUAUAG | 3443 | 1725-1747 |
| AD-159853 | A-315578 | CAACCAACUAUCCAAGUGUUA | 3258 | 1781-1801 | A-315579 | UAACACUUGGAUAGUUGGUUGCA | 3444 | 1779-1801 |
| AD-158627 | A-313126 | UCAUCGAAGACAAAUUGAAGA | 3259 | 430-450 | A-313127 | UCUUCAAUUUGUCUUCGAUGACA | 3445 | 428-450 |
| AD-159182 | A-314236 | GCAGAUUUGGCAGAGAGUAUA | 3260 | 1041-1061 | A-314237 | UAUACUCUCUGCCAAAUCUGCUA | 3446 | 1039-1061 |
| AD-159702 | A-315276 | CUCCUUCACUGAACAUGCCUA | 3261 | 1604-1624 | A-315277 | UAGGCAUGUUCAGUGAAGGAGCC | 3447 | 1602-1624 |
| AD-159715 | A-315302 | CAUGCCUAGUCCAACAUUUUU | 3262 | 1617-1637 | A-315303 | AAAAAUGUUGGACUAGGCAUGUU | 3448 | 1615-1637 |
| AD-158575 | A-313022 | UGCCAUCAGUAUCUUAAUGAA | 3263 | 377-397 | A-313023 | UUCAUUAAGAUACUGAUGGCACA | 3449 | 375-397 |
| AD-158576 | A-313024 | GCCAUCAGUAUCUUAAUGAAA | 3264 | 378-398 | A-313025 | UUUCAUUAAGAUACUGAUGGCAC | 3450 | 376-398 |
| AD-158684 | A-313240 | UUAGAACACCAAAGAUUGUCU | 3265 | 487-507 | A-313241 | AGACAAUCUUUGGUGUUCUAAGG | 3451 | 485-507 |
| AD-159410 | A-314692 | AUCAUUUCACUGUCUAGGCUA | 3266 | 1288-1308 | A-314693 | UAGCCUAGACAGUGAAAUGAUAU | 3452 | 1286-1308 |
| AD-159416 | A-314704 | UCACUGUCUAGGCUACAACAA | 3267 | 1294-1314 | A-314705 | UUGUUGUAGCCUAGACAGUGAAA | 3453 | 1292-1314 |
| AD-159738 | A-315348 | GGAUCCAGUGUAUAAAUCCAA | 3268 | 1658-1678 | A-315349 | UUGGAUUUAUACACUGGAUCCCA | 3454 | 1656-1678 |
| AD-159857 | A-315586 | CAACUAUCCAAGUGUUAUACA | 3269 | 1785-1805 | A-315587 | UGUAUAACACUUGGAUAGUUGGU | 3455 | 1783-1805 |
| AD-158497 | A-312867 | UUGGUUCCAAGUCCAAUAUGA | 3270 | 256-276 | A-312868 | UCAUAUUGGACUUGGAACCAAAA | 3456 | 254-276 |
| AD-159124 | A-314120 | UGCUUAUGAGGUGAUCAAACU | 3271 | 983-1003 | A-314121 | AGUUUGAUCACCUCAUAAGCACU | 3457 | 981-1003 |
| AD-159140 | A-314152 | AAACUCAAAGGCUACACAUCA | 3272 | 999-1019 | A-314153 | UGAUGUGUAGCCUUUGAGUUUGA | 3458 | 997-1019 |
| AD-159312 | A-314496 | UCUCAGACCUUGUGAAGGUGA | 3273 | 1171-1191 | A-314497 | UCACCUUCACAAGGUCUGAGAUU | 3459 | 1169-1191 |
| AD-159552 | A-314976 | UAAAAUCCACAGCUAUAUCCU | 3274 | 1434-1454 | A-314977 | AGGAUAUAGCUGUGGAUUUUACA | 3460 | 1432-1454 |
| AD-159704 | A-315280 | CCUUCACUGAACAUGCCUAGU | 3275 | 1606-1626 | A-315281 | ACUAGGCAUGUUCAGUGAAGGAG | 3461 | 1604-1626 |
| AD-159737 | A-315346 | GGGAUCCAGUGUAUAAAUCCA | 3276 | 1657-1677 | A-315347 | UGGAUUUAUACACUGGAUCCCAG | 3462 | 1655-1677 |
| AD-159869 | A-315610 | CAAUAAACCUUGAACAGUGAA | 3277 | 1818-1838 | A-315611 | UUCACUGUUCAAGGUUUAUUGGG | 3463 | 1816-1838 |
| AD-158570 | A-313012 | GGCCUGUGCCAUCAGUAUCUU | 3278 | 371-391 | A-313013 | AAGAUACUGAUGGCACAGGCCAU | 3464 | 369-391 |
| AD-158618 | A-313108 | UUGUUGAUGUCAUCGAAGACA | 3279 | 421-441 | A-313109 | UGUCUUCGAUGACAUCAACAAGA | 3465 | 419-441 |
| AD-159788 | A-315448 | GGAUCUUAUUUUGUGAACUAU | 3280 | 1708-1728 | A-315449 | AUAGUUCACAAAAUAAGAUCCUU | 3466 | 1706-1728 |
| AD-159786 | A-315444 | AAGGAUCUUAUUUUGUGAACU | 3281 | 1706-1726 | A-315445 | AGUUCACAAAAUAAGAUCCUUUG | 3467 | 1704-1726 |
| AD-159760 | A-315392 | AUCAUGUCUUGUGCAUAAUUA | 3282 | 1680-1700 | A-315393 | UAAUUAUGCACAAGACAUGAUAU | 3468 | 1678-1700 |
| AD-159404 | A-314680 | UGUCAUAUCAUUUCACUGUCU | 3283 | 1282-1302 | A-314681 | AGACAGUGAAAUGAUAUGACAUC | 3469 | 1280-1302 |
| AD-159406 | A-314684 | UCAUAUCAUUUCACUGUCUAA | 3284 | 1284-1304 | A-314685 | UUAGACAGUGAAAUGAUAUGACA | 3470 | 1282-1304 |
| AD-158536 | A-312944 | AUUUAUAAUCUUCUAAAGGAA | 3285 | 297-317 | A-312945 | UUCCUUUAGAAGAUUAUAAAUCA | 3471 | 295-317 |
| AD-159545 | A-314962 | UGGUUUGUAAAAUCCACAGCU | 3286 | 1427-1447 | A-314963 | AGCUGUGGAUUUUACAAACCAUU | 3472 | 1425-1447 |
| AD-159574 | A-315020 | AUGCUGGAUGGUAUUAAUCUU | 3287 | 1456-1476 | A-315021 | AAGAUUAAUACCAUCCAGCAUCA | 3473 | 1454-1476 |
| AD-159802 | A-315476 | AACUAUAUCAGUAGUGUACAU | 3288 | 1723-1743 | A-315477 | AUGUACACUACUGAUAUAGUUCA | 3474 | 1721-1743 |
| AD-159518 | A-314908 | AUCAACUCCUGAAGUUAGAAA | 3289 | 1400-1420 | A-314909 | UUUCUAACUUCAGGAGUUGAUGU | 3475 | 1398-1420 |
| AD-159577 | A-315026 | CUGGAUGGUAUUAAUCUUGUA | 3290 | 1459-1479 | A-315027 | UACAAGAUUAAUACCAUCCAGCA | 3476 | 1457-1479 |
| AD-159409 | A-314690 | UAUCAUUUCACUGUCUAGGCU | 3291 | 1287-1307 | A-314691 | AGCCUAGACAGUGAAAUGAUAUG | 3477 | 1285-1307 |
| AD-159551 | A-314974 | GUAAAAUCCACAGCUAUAUCA | 3292 | 1433-1453 | A-314975 | UGAUAUAGCUGUGGAUUUUACAA | 3478 | 1431-1453 |
| AD-159276 | A-314424 | UCCUUAGUGUUCCUUGCAUUU | 3293 | 1135-1155 | A-314425 | AAAUGCAAGGAACACUAAGGAAG | 3479 | 1133-1155 |
| AD-159407 | A-314686 | CAUAUCAUUUCACUGUCUAGA | 3294 | 1285-1305 | A-314687 | UCUAGACAGUGAAAUGAUAUGAC | 3480 | 1283-1305 |
| AD-159515 | A-314902 | AACAUCAACUCCUGAAGUUAA | 3295 | 1397-1417 | A-314903 | UUAACUUCAGGAGUUGAUGUUUU | 3481 | 1395-1417 |
| AD-159570 | A-315012 | CCUGAUGCUGGAUGGUAUUAA | 3296 | 1452-1472 | A-315013 | UUAAUACCAUCCAGCAUCAGGAU | 3482 | 1450-1472 |
| AD-159849 | A-315570 | AAUGCAACCAACUAUCCAAGU | 3297 | 1777-1797 | A-315571 | ACUUGGAUAGUUGGUUGCAUUGU | 3483 | 1775-1797 |
| AD-159252 | A-314376 | UUUACGGAAUAAAGGAUGAUA | 3298 | 1111-1131 | A-314377 | UAUCAUCCUUUAUUCCGUAAAGA | 3484 | 1109-1131 |
| AD-159275 | A-314422 | UUCCUUAGUGUUCCUUGCAUU | 3299 | 1134-1154 | A-314423 | AAUGCAAGGAACACUAAGGAAGA | 3485 | 1132-1154 |
| AD-159848 | A-315568 | CAAUGCAACCAACUAUCCAAA | 3300 | 1776-1796 | A-315569 | UUUGGAUAGUUGGUUGCAUUGUU | 3486 | 1774-1796 |
| AD-159184 | A-314240 | AGAUUUGGCAGAGAGUAUAAU | 3301 | 1043-1063 | A-314241 | AUUAUACUCUCUGCCAAAUCUGC | 3487 | 1041-1063 |
| AD-159231 | A-314334 | UUUCCACCAUGAUUAAGGGUA | 3302 | 1090-1110 | A-314335 | UACCCUUAAUCAUGGUGGAAACU | 3488 | 1088-1110 |
| AD-159607 | A-315086 | ACUGGUUAGUGUGAAAUAGUU | 3303 | 1489-1509 | A-315087 | AACUAUUUCACACUAACCAGUUG | 3489 | 1487-1509 |
| AD-158504 | A-312881 | CAAGUCCAAUAUGGCAACUCU | 3304 | 263-283 | A-312882 | AGAGUUGCCAUAUUGGACUUGGA | 3490 | 261-283 |
| AD-159233 | A-314338 | UCCACCAUGAUUAAGGGUCUU | 3305 | 1092-1112 | A-314339 | AAGACCCUUAAUCAUGGUGGAAA | 3491 | 1090-1112 |
| AD-159411 | A-314694 | UCAUUUCACUGUCUAGGCUAA | 3306 | 1289-1309 | A-314695 | UUAGCCUAGACAGUGAAAUGAUA | 3492 | 1287-1309 |
| AD-159462 | A-314796 | UGUCCUUUUUAUCUGAUCUGU | 3307 | 1340-1360 | A-314797 | ACAGAUCAGAUAAAAAGGACAAC | 3493 | 1338-1360 |
| AD-159742 | A-315356 | CCAGUGUAUAAAUCCAAUAUA | 3308 | 1662-1682 | A-315357 | UAUAUUGGAUUUAUACACUGGAU | 3494 | 1660-1682 |
| AD-159863 | A-315598 | UCCAAGUGUUAUACCAACUAA | 3309 | 1791-1811 | A-315599 | UUAGUUGGUAUAACACUUGGAUA | 3495 | 1789-1811 |
| AD-158687 | A-313246 | GAACACCAAAGAUUGUCUCUA | 3310 | 490-510 | A-313247 | UAGAGACAAUCUUUGGUGUUCUA | 3496 | 488-510 |
| AD-158688 | A-313248 | AACACCAAAGAUUGUCUCUGA | 3311 | 491-511 | A-313249 | UCAGAGACAAUCUUUGGUGUUCU | 3497 | 489-511 |
| AD-159458 | A-314788 | AUGUUGUCCUUUUUAUCUGAU | 3312 | 1336-1356 | A-314789 | AUCAGAUAAAAAGGACAACAUGC | 3498 | 1334-1356 |
| AD-159519 | A-314910 | UCAACUCCUGAAGUUAGAAAU | 3313 | 1401-1421 | A-314911 | AUUUCUAACUUCAGGAGUUGAUG | 3499 | 1399-1421 |
| AD-159858 | A-315588 | AACUAUCCAAGUGUUAUACCA | 3314 | 1786-1806 | A-315589 | UGGUAUAACACUUGGAUAGUUGG | 3500 | 1784-1806 |
| AD-159583 | A-315038 | GGUAUUAAUCUUGUGUAGUCU | 3315 | 1465-1485 | A-315039 | AGACUACACAAGAUUAAUACCAU | 3501 | 1463-1485 |
| AD-159700 | A-315272 | GGCUCCUUCACUGAACAUGCA | 3316 | 1602-1622 | A-315273 | UGCAUGUUCAGUGAAGGAGCCAG | 3502 | 1600-1622 |
| AD-159807 | A-315486 | UAUCAGUAGUGUACAUUACCA | 3317 | 1728-1748 | A-315487 | UGGUAAUGUACACUACUGAUAUA | 3503 | 1726-1748 |
| AD-158673 | A-313218 | CAGCCUUUUCCUUAGAACACA | 3318 | 476-496 | A-313219 | UGUGUUCUAAGGAAAAGGCUGCC | 3504 | 474-496 |
| AD-159608 | A-315088 | CUGGUUAGUGUGAAAUAGUUA | 3319 | 1490-1510 | A-315089 | UAACUAUUUCACACUAACCAGUU | 3505 | 1488-1510 |
| AD-159803 | A-315478 | ACUAUAUCAGUAGUGUACAUU | 3320 | 1724-1744 | A-315479 | AAUGUACACUACUGAUAUAGUUC | 3506 | 1722-1744 |
| AD-159805 | A-315482 | UAUAUCAGUAGUGUACAUUAA | 3321 | 1726-1746 | A-315483 | UUAAUGUACACUACUGAUAUAGU | 3507 | 1724-1746 |
| AD-159489 | A-314850 | GUAAUAUUUUAAGAUGGACUA | 3322 | 1371-1391 | A-314851 | UAGUCCAUCUUAAAAUAUUACUG | 3508 | 1369-1391 |
| AD-159495 | A-314862 | UUUUAAGAUGGACUGGGAAAA | 3323 | 1377-1397 | A-314863 | UUUUCCCAGUCCAUCUUAAAAUA | 3509 | 1375-1397 |
| AD-159706 | A-315284 | UUCACUGAACAUGCCUAGUCA | 3324 | 1608-1628 | A-315285 | UGACUAGGCAUGUUCAGUGAAGG | 3510 | 1606-1628 |
| AD-159855 | A-315582 | ACCAACUAUCCAAGUGUUAUA | 3325 | 1783-1803 | A-315583 | UAUAACACUUGGAUAGUUGGUUG | 3511 | 1781-1803 |
| AD-159864 | A-315600 | CCAAGUGUUAUACCAACUAAA | 3326 | 1792-1812 | A-315601 | UUUAGUUGGUAUAACACUUGGAU | 3512 | 1790-1812 |
| AD-159488 | A-314848 | AGUAAUAUUUUAAGAUGGACU | 3327 | 1370-1390 | A-314849 | AGUCCAUCUUAAAAUAUUACUGC | 3513 | 1368-1390 |
| AD-159553 | A-314978 | AAAAUCCACAGCUAUAUCCUA | 3328 | 1435-1455 | A-314979 | UAGGAUAUAGCUGUGGAUUUUAC | 3514 | 1433-1455 |
| AD-159703 | A-315278 | UCCUUCACUGAACAUGCCUAA | 3329 | 1605-1625 | A-315279 | UUAGGCAUGUUCAGUGAAGGAGC | 3515 | 1603-1625 |
| AD-159708 | A-315288 | CACUGAACAUGCCUAGUCCAA | 3330 | 1610-1630 | A-315289 | UUGGACUAGGCAUGUUCAGUGAA | 3516 | 1608-1630 |
| AD-159866 | A-315604 | AAGUGUUAUACCAACUAAAAC | 3331 | 1794-1814 | A-315605 | GUUUUAGUUGGUAUAACACUUGG | 3517 | 1792-1814 |
| AD-159232 | A-314336 | UUCCACCAUGAUUAAGGGUCU | 3332 | 1091-1111 | A-314337 | AGACCCUUAAUCAUGGUGGAAAC | 3518 | 1089-1111 |
| AD-159712 | A-315296 | GAACAUGCCUAGUCCAACAUU | 3333 | 1614-1634 | A-315297 | AAUGUUGGACUAGGCAUGUUCAG | 3519 | 1612-1634 |
| AD-159808 | A-315488 | AUCAGUAGUGUACAUUACCAU | 3334 | 1729-1749 | A-315489 | AUGGUAAUGUACACUACUGAUAU | 3520 | 1727-1749 |
| AD-159862 | A-315596 | AUCCAAGUGUUAUACCAACUA | 3335 | 1790-1810 | A-315597 | UAGUUGGUAUAACACUUGGAUAG | 3521 | 1788-1810 |
| AD-158503 | A-312879 | CCAAGUCCAAUAUGGCAACUA | 3336 | 262-282 | A-312880 | UAGUUGCCAUAUUGGACUUGGAA | 3522 | 260-282 |
| AD-159412 | A-314696 | CAUUUCACUGUCUAGGCUACA | 3337 | 1290-1310 | A-314697 | UGUAGCCUAGACAGUGAAAUGAU | 3523 | 1288-1310 |
| AD-159558 | A-314988 | CCACAGCUAUAUCCUGAUGCU | 3338 | 1440-1460 | A-314989 | AGCAUCAGGAUAUAGCUGUGGAU | 3524 | 1438-1460 |
| AD-159705 | A-315282 | CUUCACUGAACAUGCCUAGUA | 3339 | 1607-1627 | A-315283 | UACUAGGCAUGUUCAGUGAAGGA | 3525 | 1605-1627 |
| AD-159113 | A-314098 | GUGGUUGAGAGUGCUUAUGAA | 3340 | 972-992 | A-314099 | UUCAUAAGCACUCUCAACCACCU | 3526 | 970-992 |
| AD-159806 | A-315484 | AUAUCAGUAGUGUACAUUACA | 3341 | 1727-1747 | A-315485 | UGUAAUGUACACUACUGAUAUAG | 3527 | 1725-1747 |
| AD-159853 | A-315578 | CAACCAACUAUCCAAGUGUUA | 3342 | 1781-1801 | A-315579 | UAACACUUGGAUAGUUGGUUGCA | 3528 | 1779-1801 |
| AD-159182 | A-314236 | GCAGAUUUGGCAGAGAGUAUA | 3343 | 1041-1061 | A-314237 | UAUACUCUCUGCCAAAUCUGCUA | 3529 | 1039-1061 |
| AD-159702 | A-315276 | CUCCUUCACUGAACAUGCCUA | 3344 | 1604-1624 | A-315277 | UAGGCAUGUUCAGUGAAGGAGCC | 3530 | 1602-1624 |
| AD-159715 | A-315302 | CAUGCCUAGUCCAACAUUUUU | 3345 | 1617-1637 | A-315303 | AAAAAUGUUGGACUAGGCAUGUU | 3531 | 1615-1637 |
| AD-158575 | A-313022 | UGCCAUCAGUAUCUUAAUGAA | 3346 | 377-397 | A-313023 | UUCAUUAAGAUACUGAUGGCACA | 3532 | 375-397 |
| AD-158576 | A-313024 | GCCAUCAGUAUCUUAAUGAAA | 3347 | 378-398 | A-313025 | UUUCAUUAAGAUACUGAUGGCAC | 3533 | 376-398 |
| AD-158684 | A-313240 | UUAGAACACCAAAGAUUGUCU | 3348 | 487-507 | A-313241 | AGACAAUCUUUGGUGUUCUAAGG | 3534 | 485-507 |
| AD-159410 | A-314692 | AUCAUUUCACUGUCUAGGCUA | 3349 | 1288-1308 | A-314693 | UAGCCUAGACAGUGAAAUGAUAU | 3535 | 1286-1308 |
| AD-159416 | A-314704 | UCACUGUCUAGGCUACAACAA | 3350 | 1294-1314 | A-314705 | UUGUUGUAGCCUAGACAGUGAAA | 3536 | 1292-1314 |
| AD-159857 | A-315586 | CAACUAUCCAAGUGUUAUACA | 3351 | 1785-1805 | A-315587 | UGUAUAACACUUGGAUAGUUGGU | 3537 | 1783-1805 |
| AD-158497 | A-312867 | UUGGUUCCAAGUCCAAUAUGA | 3352 | 256-276 | A-312868 | UCAUAUUGGACUUGGAACCAAAA | 3538 | 254-276 |
| AD-159124 | A-314120 | UGCUUAUGAGGUGAUCAAACU | 3353 | 983-1003 | A-314121 | AGUUUGAUCACCUCAUAAGCACU | 3539 | 981-1003 |
| AD-159312 | A-314496 | UCUCAGACCUUGUGAAGGUGA | 3354 | 1171-1191 | A-314497 | UCACCUUCACAAGGUCUGAGAUU | 3540 | 1169-1191 |
| AD-159552 | A-314976 | UAAAAUCCACAGCUAUAUCCU | 3355 | 1434-1454 | A-314977 | AGGAUAUAGCUGUGGAUUUUACA | 3541 | 1432-1454 |
| AD-159704 | A-315280 | CCUUCACUGAACAUGCCUAGU | 3356 | 1606-1626 | A-315281 | ACUAGGCAUGUUCAGUGAAGGAG | 3542 | 1604-1626 |
| AD-159737 | A-315346 | GGGAUCCAGUGUAUAAAUCCA | 3357 | 1657-1677 | A-315347 | UGGAUUUAUACACUGGAUCCCAG | 3543 | 1655-1677 |
| AD-159869 | A-315610 | CAAUAAACCUUGAACAGUGAA | 3358 | 1818-1838 | A-315611 | UUCACUGUUCAAGGUUUAUUGGG | 3544 | 1816-1838 |
| AD-158570 | A-313012 | GGCCUGUGCCAUCAGUAUCUU | 3359 | 371-391 | A-313013 | AAGAUACUGAUGGCACAGGCCAU | 3545 | 369-391 |
| AD-158618 | A-313108 | UUGUUGAUGUCAUCGAAGACA | 3360 | 421-441 | A-313109 | UGUCUUCGAUGACAUCAACAAGA | 3546 | 419-441 |
| AD-159184 | A-314240 | AGAUUUGGCAGAGAGUAUAAU | 3361 | 1043-1063 | A-314241 | AUUAUACUCUCUGCCAAAUCUGC | 3547 | 1041-1063 |
| AD-159231 | A-314334 | UUUCCACCAUGAUUAAGGGUA | 3362 | 1090-1110 | A-314335 | UACCCUUAAUCAUGGUGGAAACU | 3548 | 1088-1110 |
| AD-159423 | A-314718 | CUAGGCUACAACAGGAUUCUA | 3363 | 1301-1321 | A-314719 | UAGAAUCCUGUUGUAGCCUAGAC | 3549 | 1299-1321 |
| AD-159446 | A-314764 | UGGAGGUUGUGCAUGUUGUCA | 3364 | 1324-1344 | A-314765 | UGACAACAUGCACAACCUCCACC | 3550 | 1322-1344 |
| AD-159701 | A-315274 | GCUCCUUCACUGAACAUGCCU | 3365 | 1603-1623 | A-315275 | AGGCAUGUUCAGUGAAGGAGCCA | 3551 | 1601-1623 |
| AD-158494 | A-312861 | CUUUUGGUUCCAAGUCCAAUA | 3366 | 253-273 | A-312862 | UAUUGGACUUGGAACCAAAAGGA | 3552 | 251-273 |
| AD-158571 | A-313014 | GCCUGUGCCAUCAGUAUCUUA | 3367 | 372-392 | A-313015 | UAAGAUACUGAUGGCACAGGCCA | 3553 | 370-392 |
| AD-159125 | A-314122 | GCUUAUGAGGUGAUCAAACUA | 3368 | 984-1004 | A-314123 | UAGUUUGAUCACCUCAUAAGCAC | 3554 | 982-1004 |
| AD-159126 | A-314124 | CUUAUGAGGUGAUCAAACUCA | 3369 | 985-1005 | A-314125 | UGAGUUUGAUCACCUCAUAAGCA | 3555 | 983-1005 |
| AD-159287 | A-314446 | CCUUGCAUUUUGGGACAGAAU | 3370 | 1146-1166 | A-314447 | AUUCUGUCCCAAAAUGCAAGGAA | 3556 | 1144-1166 |
| AD-158499 | A-312871 | GGUUCCAAGUCCAAUAUGGCA | 3371 | 258-278 | A-312872 | UGCCAUAUUGGACUUGGAACCAA | 3557 | 256-278 |
| AD-159417 | A-314706 | CACUGUCUAGGCUACAACAGA | 3372 | 1295-1315 | A-314707 | UCUGUUGUAGCCUAGACAGUGAA | 3558 | 1293-1315 |
| AD-159418 | A-314708 | ACUGUCUAGGCUACAACAGGA | 3373 | 1296-1316 | A-314709 | UCCUGUUGUAGCCUAGACAGUGA | 3559 | 1294-1316 |
| AD-158550 | A-312972 | AAUAAGAUUACAGUUGUUGGA | 3374 | 333-353 | A-312973 | UCCAACAACUGUAAUCUUAUUCU | 3560 | 331-353 |
| AD-159116 | A-314104 | GUUGAGAGUGCUUAUGAGGUA | 3375 | 975-995 | A-314105 | UACCUCAUAAGCACUCUCAACCA | 3561 | 973-995 |
| AD-159421 | A-314714 | GUCUAGGCUACAACAGGAUUA | 3376 | 1299-1319 | A-314715 | UAAUCCUGUUGUAGCCUAGACAG | 3562 | 1297-1319 |
| AD-159422 | A-314716 | UCUAGGCUACAACAGGAUUCU | 3377 | 1300-1320 | A-314717 | AGAAUCCUGUUGUAGCCUAGACA | 3563 | 1298-1320 |
| AD-159445 | A-314762 | GUGGAGGUUGUGCAUGUUGUA | 3378 | 1323-1343 | A-314763 | UACAACAUGCACAACCUCCACCU | 3564 | 1321-1343 |
| AD-159130 | A-314132 | UGAGGUGAUCAAACUCAAAGA | 3379 | 989-1009 | A-314133 | UCUUUGAGUUUGAUCACCUCAUA | 3565 | 987-1009 |
| AD-159134 | A-314140 | GUGAUCAAACUCAAAGGCUAA | 3380 | 993-1013 | A-314141 | UUAGCCUUUGAGUUUGAUCACCU | 3566 | 991-1013 |
| AD-159343 | A-314558 | UGAGGAAGAGGCCCGUUUGAA | 3381 | 1202-1222 | A-314559 | UUCAAACGGGCCUCUUCCUCAGA | 3567 | 1200-1222 |
| AD-159105 | A-314082 | ACAAGCAGGUGGUUGAGAGUA | 3382 | 964-984 | A-314083 | UACUCUCAACCACCUGCUUGUGA | 3568 | 962-984 |
| AD-159183 | A-314238 | CAGAUUUGGCAGAGAGUAUAA | 3383 | 1042-1062 | A-314239 | UUAUACUCUCUGCCAAAUCUGCU | 3569 | 1040-1062 |
| AD-159123 | A-314118 | GUGCUUAUGAGGUGAUCAAAC | 3384 | 982-1002 | A-314119 | GUUUGAUCACCUCAUAAGCACUC | 3570 | 980-1002 |
| AD-159181 | A-314234 | AGCAGAUUUGGCAGAGAGUAU | 3385 | 1040-1060 | A-314235 | AUACUCUCUGCCAAAUCUGCUAC | 3571 | 1038-1060 |
| AD-159186 | A-314244 | AUUUGGCAGAGAGUAUAAUGA | 3386 | 1045-1065 | A-314245 | UCAUUAUACUCUCUGCCAAAUCU | 3572 | 1043-1065 |
| AD-159187 | A-314246 | UUUGGCAGAGAGUAUAAUGAA | 3387 | 1046-1066 | A-314247 | UUCAUUAUACUCUCUGCCAAAUC | 3573 | 1044-1066 |
| AD-159288 | A-314448 | CUUGCAUUUUGGGACAGAAUA | 3388 | 1147-1167 | A-314449 | UAUUCUGUCCCAAAAUGCAAGGA | 3574 | 1145-1167 |
| AD-159306 | A-314484 | AUGGAAUCUCAGACCUUGUGA | 3389 | 1165-1185 | A-314485 | UCACAAGGUCUGAGAUUCCAUUC | 3575 | 1163-1185 |
| AD-159559 | A-314990 | CACAGCUAUAUCCUGAUGCUA | 3390 | 1441-1461 | A-314991 | UAGCAUCAGGAUAUAGCUGUGGA | 3576 | 1439-1461 |
| AD-159344 | A-314560 | GAGGAAGAGGCCCGUUUGAAA | 3391 | 1203-1223 | A-314561 | UUUCAAACGGGCCUCUUCCUCAG | 3577 | 1201-1223 |
| AD-159341 | A-314554 | UCUGAGGAAGAGGCCCGUUUA | 3392 | 1200-1220 | A-314555 | UAAACGGGCCUCUUCCUCAGAAG | 3578 | 1198-1220 |
| AD-159729 | A-315330 | CACAUCCUGGGAUCCAGUGUA | 3393 | 1649-1669 | A-315331 | UACACUGGAUCCCAGGAUGUGAC | 3579 | 1647-1669 |
| AD-158674 | A-313220 | AGCCUUUUCCUUAGAACACCA | 3394 | 477-497 | A-313221 | UGGUGUUCUAAGGAAAAGGCUGC | 3580 | 475-497 |
| AD-159604 | A-315080 | UCAACUGGUUAGUGUGAAAUA | 3395 | 1486-1506 | A-315081 | UAUUUCACACUAACCAGUUGAAG | 3581 | 1484-1506 |

TABLE 5
MODIFIED HUMAN/CYNOMOLGUS CROSS-REACTIVE LDHA iRNA SEQUENCES

| | | SEQ | |
|---|---|---|---|
| Duplex | | ID | |
| Name | Sense Sequence 5′ to 3′ | NO | Antisense Sequence 5′ to 3′ |
| AD-159469 | ususuaucUfgAfUfCfugugauuaaaL96 | 3582 | usUfsuaaUfcAfCfagauCfaGfauaaasasa |
| AD-159607 | ascsugguUfaGfUfGfugaaauaguuL96 | 3583 | asAfscuaUfuUfCfacacUfaAfccagususg |
| AD-159713 | asascaugCfcUfAfGfuccaacauuuL96 | 3584 | asAfsaugUfuGfGfacuaGfgCfauguuscsa |
| AD-158504 | csasagucCfaAfUfAfuggcaacucuL96 | 3585 | asGfsaguUfgCfCfauauUfgGfacuugsgsa |
| AD-159233 | uscscaccAfuGfAfUfuaagggucuuL96 | 3586 | asAfsgacCfcUfUfaaucAfuGfguggasasa |
| AD-159411 | uscsauuuCfaCfUfGfucuaggcuaaL96 | 3587 | usUfsagcCfuAfGfacagUfgAfaaugasusa |
| AD-159462 | usgsuccuUfuUfUfAfucugaucuguL96 | 3588 | asCfsagaUfcAfGfauaaAfaAfggacasasc |
| AD-159742 | cscsagugUfaUfAfAfauccaauauaL96 | 3589 | usAfsuauUfgGfAfuuuaUfaCfacuggsasu |
| AD-159863 | uscscaagUfgUfUfAfuaccaacuaaL96 | 3590 | usUfsaguUfgGfUfauaaCfaCfuuggasusa |
| AD-158626 | gsuscaucGfaAfGfAfcaaauugaaaL96 | 3591 | usUfsucaAfuUfUfgucuUfcGfaugacsasu |
| AD-158687 | gsasacacCfaAfAfGfauugucucuaL96 | 3592 | usAfsgagAfcAfAfucuuUfgGfuguucsusa |
| AD-158688 | asascaccAfaAfGfAfuugucucugaL96 | 3593 | usCfsagaGfaCfAfaucuUfuGfguguuscsu |
| AD-159458 | asusguugUfcCfUfUfuuuaucugauL96 | 3594 | asUfscagAfuAfAfaaagGfaCfaacausgsc |
| AD-159519 | uscsaacuCfcUfGfAfaguuagaaauL96 | 3595 | asUfsuucUfaAfCfuucaGfgAfguugasusg |
| AD-159858 | asascuauCfcAfAfGfuguuauaccaL96 | 3596 | usGfsguaUfaAfCfacuuGfgAfuaguusgsg |
| AD-158681 | uscscuuaGfaAfCfAfccaaagauuaL96 | 3597 | usAfsaucUfuUfGfguguUfcUfaaggasasa |
| AD-159583 | gsgsuauuAfaUfCfUfuguguagucuL96 | 3598 | asGfsacuAfcAfCfaagaUfuAfauaccsasu |
| AD-159700 | gsgscuccUfuCfAfCfugaacaugcaL96 | 3599 | usGfscauGfuUfCfagugAfaGfgagccsasg |
| AD-159807 | usasucagUfaGfUfGfuacauuaccaL96 | 3600 | usGfsguaAfuGfUfacacUfaCfugauasusa |
| AD-158673 | csasgccuUfuUfCfCfuuagaacacaL96 | 3601 | usGfsuguUfcUfAfaggaAfaAfggcugscsc |
| AD-159608 | csusgguuAfgUfGfUfgaaauaguuaL96 | 3602 | usAfsacuAfuUfUfcacaCfuAfaccagsusu |
| AD-159803 | ascsuauaUfcAfGfUfaguguacauuL96 | 3603 | asAfsuguAfcAfCfuacuGfaUfauagususc |
| AD-159805 | usasuaucAfgUfAfGfuguacauuaaL96 | 3604 | usUfsaauGfuAfCfacuaCfuGfauauasgsu |
| AD-159489 | gsusaauaUfuUfUfAfagauggacuaL96 | 3605 | usAfsgucCfaUfCfuuaaAfaUfauuacsusg |
| AD-159495 | ususuuaaGfaUfGfGfacugggaaaaL96 | 3606 | usUfsuucCfcAfGfuccaUfcUfuaaaasusa |
| AD-159609 | usgsguuaGfuGfUfGfaaauaguucuL96 | 3607 | asGfsaacUfaUfUfucacAfcUfaaccasgsu |
| AD-159706 | ususcacuGfaAfCfAfugccuagucaL96 | 3608 | usGfsacuAfgGfCfauguUfcAfgugaasgsg |
| AD-159855 | ascscaacUfaUfCfCfaaguguuauaL96 | 3609 | usAfsuaaCfaCfUfuggaUfaGfuuggususg |
| AD-159864 | cscsaaguGfuUfAfUfaccaacuaaaL96 | 3610 | usUfsuagUfuGfGfuauaAfcAfcuuggsasu |
| AD-158491 | ususccuuUfuGfGfUfuccaaguccaL96 | 3611 | usGfsgacUfuGfGfaaccAfaAfaggaasusc |
| AD-158672 | gscsagccUfuUfUfCfcuuagaacaaL96 | 3612 | usUfsguuCfuAfAfggaaAfaGfgcugcscsa |
| AD-159488 | asgsuaauAfuUfUfUfaagauggacuL96 | 3613 | asGfsuccAfuCfUfuaaaAfuAfuuacusgsc |
| AD-159553 | asasaaucCfaCfAfGfcuauauccuaL96 | 3614 | usAfsggaUfaUfAfgcugUfgGfauuuusasc |
| AD-159703 | uscscuucAfcUfGfAfacaugccuaaL96 | 3615 | usUfsaggCfaUfGfuucaGfuGfaaggasgsc |
| AD-159708 | csascugaAfcAfUfGfccuaguccaaL96 | 3616 | usUfsggaCfuAfGfgcauGfuUfcagugsasa |
| AD-159866 | asasguguUfaUfAfCfcaacuaaaacL96 | 3617 | gsUfsuuuAfgUfUfgguaUfaAfcacuusgsg |
| AD-159232 | ususccacCfaUfGfAfuuaagggucuL96 | 3618 | asGfsaccCfuUfAfaucaUfgGfuggaasasc |
| AD-159712 | gsasacauGfcCfUfAfguccaacauuL96 | 3619 | asAfsuguUfgGfAfcuagGfcAfuguucsasg |
| AD-159808 | asuscaguAfgUfGfUfacauuaccauL96 | 3620 | asUfsgguAfaUfGfuacaCfuAfcugausasu |
| AD-159862 | asusccaaGfuGfUfUfauaccaacuaL96 | 3621 | usAfsguuGfgUfAfuaacAfcUfuggausasg |
| AD-158503 | cscsaaguCfcAfAfUfauggcaacuaL96 | 3622 | usAfsguuGfcCfAfuauuGfgAfcuuggsasa |
| AD-159311 | asuscucaGfaCfCfUfugugaagguaL96 | 3623 | usAfsccuUfcAfCfaaggUfcUfgagaususc |
| AD-159412 | csasuuucAfcUfGfUfcuaggcuacaL96 | 3624 | usGfsuagCfcUfAfgacaGfuGfaaaugsasu |
| AD-159558 | cscsacagCfuAfUfAfuccugaugcuL96 | 3625 | asGfscauCfaGfGfauauAfgCfuguggsasu |
| AD-159705 | csusucacUfgAfAfCfaugccuaguaL96 | 3626 | usAfscuaGfgCfAfuguuCfaGfugaagsgsa |
| AD-159113 | gsusgguuGfaGfAfGfugcuuaugaaL96 | 3627 | usUfscauAfaGfCfacucUfcAfaccacscsu |
| AD-159139 | csasaacuCfaAfAfGfgcuacacauaL96 | 3628 | usAfsuguGfuAfGfccuuUfgAfguuugsasu |
| AD-159806 | asusaucaGfuAfGfUfguacauuacaL96 | 3629 | usGfsuaaUfgUfAfcacuAfcUfgauausasg |
| AD-159853 | csasaccaAfcUfAfUfccaaguguuaL96 | 3630 | usAfsacaCfuUfGfgauaGfuUfgguugscsa |
| AD-158627 | uscsaucgAfaGfAfCfaaauugaagaL96 | 3631 | usCfsuucAfaUfUfugucUfuCfgaugascsa |
| AD-159182 | gscsagauUfuGfGfCfagagaguauaL96 | 3632 | usAfsuacUfcUfCfugccAfaAfucugcsusa |
| AD-159702 | csusccuuCfaCfUfGfaacaugccuaL96 | 3633 | usAfsggcAfuGfUfucagUfgAfaggagscsc |
| AD-159715 | csasugccUfaGfUfCfcaacauuuuuL96 | 3634 | asAfsaaaUfgUfUfggacUfaGfgcaugsusu |
| AD-158575 | usgsccauCfaGfUfAfucuuaaugaaL96 | 3635 | usUfscauUfaAfGfauacUfgAfuggcascsa |
| AD-158576 | gscscaucAfgUfAfUfcuuaaugaaaL96 | 3636 | usUfsucaUfuAfAfgauaCfuGfauggcsasc |
| AD-158684 | ususagaaCfaCfCfAfaagauugucuL96 | 3637 | asGfsacaAfuCfUfuuggUfgUfucuaasgsg |
| AD-159410 | asuscauuUfcAfCfUfgucuaggcuaL96 | 3638 | usAfsgccUfaGfAfcaguGfaAfaugausasu |
| AD-159416 | uscsacugUfcUfAfGfgcuacaacaaL96 | 3639 | usUfsguuGfuAfGfccuaGfaCfagugasasa |
| AD-159738 | gsgsauccAfgUfGfUfauaaauccaaL96 | 3640 | usUfsggaUfuUfAfuacaCfuGfgauccscsa |
| AD-159857 | csasacuaUfcCfAfAfguguuauacaL96 | 3641 | usGfsuauAfaCfAfcuugGfaUfaguugsgsu |
| AD-158497 | ususgguuCfcAfAfGfuccaauaugaL96 | 3642 | usCfsauaUfuGfGfacuuGfgAfaccaasasa |
| AD-159124 | usgscuuaUfgAfGfGfugaucaaacuL96 | 3643 | asGfsuuuGfaUfCfaccuCfaUfaagcascsu |
| AD-159140 | asasacucAfaAfGfGfcuacacaucaL96 | 3644 | usGfsaugUfgUfAfgccuUfuGfaguuusgsa |
| AD-159312 | uscsucagAfcCfUfUfgugaaggugaL96 | 3645 | usCfsaccUfuCfAfcaagGfuCfugagasusu |
| AD-159552 | usasaaauCfcAfCfAfgcuauauccuL96 | 3646 | asGfsgauAfuAfGfcuguGfgAfuuuuascsa |
| AD-159704 | cscsuucaCfuGfAfAfcaugccuaguL96 | 3647 | asCfsuagGfcAfUfguucAfgUfgaaggsasg |
| AD-159737 | gsgsgaucCfaGfUfGfuauaaauccaL96 | 3648 | usGfsgauUfuAfUfacacUfgGfaucccsasg |
| AD-159869 | csasauaaAfcCfUfUfgaacagugaaL96 | 3649 | usUfscacUfgUfUfcaagGfuUfuauugsgsg |
| AD-158570 | gsgsccugUfgCfCfAfucaguaucuuL96 | 3650 | asAfsgauAfcUfGfauggCfaCfaggccsasu |
| AD-158618 | ususguugAfuGfUfCfaucgaagacaL96 | 3651 | usGfsucuUfcGfAfugacAfuCfaacaasgsa |
| AD-159788 | gsgsaucuUfaUfUfUfugugaacuauL96 | 3652 | asUfsaguUfcAfCfaaaaUfaAfgauccsusu |
| AD-159786 | asasggauCfuUfAfUfuuugugaacuL96 | 3653 | asGfsuucAfcAfAfaauaAfgAfuccuususg |
| AD-159760 | asuscaugUfcUfUfGfugcauaauuaL96 | 3654 | usAfsauuAfuGfCfacaaGfaCfaugausasu |
| AD-159404 | usgsucauAfuCfAfUfuucacugucuL96 | 3655 | asGfsacaGfuGfAfaaugAfuAfugacasusc |
| AD-159406 | uscsauauCfaUfUfUfcacugucuaaL96 | 3656 | usUfsagaCfaGfUfgaaaUfgAfuaugascsa |
| AD-158536 | asusuuauAfaUfCfUfucuaaaggaaL96 | 3657 | usUfsccuUfuAfGfaagaUfuAfuaaauscsa |
| AD-159545 | usgsguuuGfuAfAfAfauccacagcuL96 | 3658 | asGfscugUfgGfAfuuuuAfcAfaaccasusu |
| AD-159574 | asusgcugGfaUfGfGfuauuaaucuuL96 | 3659 | asAfsgauUfaAfUfaccaUfcCfagcauscsa |
| AD-159802 | asascuauAfuCfAfGfuaguguacauL96 | 3660 | asUfsguaCfaCfUfacugAfuAfuaguuscsa |
| AD-159518 | asuscaacUfcCfUfGfaaguuagaaaL96 | 3661 | usUfsucuAfaCfUfucagGfaGfuugausgsu |
| AD-159577 | csusggauGfgUfAfUfuaaucuuguaL96 | 3662 | usAfscaaGfaUfUfaauaCfcAfuccagscsa |
| AD-159409 | usasucauUfuCfAfCfugucuaggcuL96 | 3663 | asGfsccuAfgAfCfagugAfaAfugauasusg |
| AD-159551 | gsusaaaaUfcCfAfCfagcuauaucaL96 | 3664 | usGfsauaUfaGfCfugugGfaUfuuuacsasa |
| AD-159276 | uscscuuaGfuGfUfUfccuugcauuuL96 | 3665 | asAfsaugCfaAfGfgaacAfcUfaaggasasg |
| AD-159407 | csasuaucAfuUfUfCfacugucuagaL96 | 3666 | usCfsuagAfcAfGfugaaAfuGfauaugsasc |
| AD-159515 | asascaucAfaCfUfCfcugaaguuaaL96 | 3667 | usUfsaacUfuCfAfggagUfuGfauguususu |
| AD-159570 | cscsugauGfcUfGfGfaugguauuaaL96 | 3668 | usUfsaauAfcCfAfuccaGfcAfucaggsasu |
| AD-159849 | asasugcaAfcCfAfAfcuauccaaguL96 | 3669 | asCfsuugGfaUfAfguugGfuUfgcauusgsu |
| AD-159252 | ususuacgGfaAfUfAfaaggaugauaL96 | 3670 | usAfsucaUfcCfUfuuauUfcCfguaaasgsa |
| AD-159275 | ususccuuAfgUfGfUfuccuugcauuL96 | 3671 | asAfsugcAfaGfGfaacaCfuAfaggaasgsa |
| AD-159848 | csasaugcAfaCfCfAfacuauccaaaL96 | 3672 | usUfsuggAfuAfGfuuggUfuGfcauugsusu |
| AD-159184 | asgsauuuGfgCfAfGfagaguauaauL96 | 3673 | asUfsuauAfcUfCfucugCfcAfaaucusgsc |
| AD-159231 | ususuccaCfcAfUfGfauuaaggguaL96 | 3674 | usAfscccUfuAfAfucauGfgUfggaaascsu |
| AD-159607 | ascsugguUfaGfUfGfugaaauaguuL96 | 3675 | asAfscuaUfuUfCfacacUfaAfccagususg |
| AD-158504 | csasagucCfaAfUfAfuggcaacucuL96 | 3676 | asGfsaguUfgCfCfauauUfgGfacuugsgsa |
| AD-159233 | uscscaccAfuGfAfUfuaagggucuuL96 | 3677 | asAfsgacCfcUfUfaaucAfuGfguggasasa |
| AD-159411 | uscsauuuCfaCfUfGfucuaggcuaaL96 | 3678 | usUfsagcCfuAfGfacagUfgAfaaugasusa |
| AD-159462 | usgsuccuUfuUfUfAfucugaucuguL96 | 3679 | asCfsagaUfcAfGfauaaAfaAfggacasasc |
| AD-159742 | cscsagugUfaUfAfAfauccaauauaL96 | 3680 | usAfsuauUfgGfAfuuuaUfaCfacuggsasu |
| AD-159863 | uscscaagUfgUfUfAfuaccaacuaaL96 | 3681 | usUfsaguUfgGfUfauaaCfaCfuuggasusa |
| AD-158687 | gsasacacCfaAfAfGfauugucucuaL96 | 3682 | usAfsgagAfcAfAfucuuUfgGfuguucsusa |
| AD-158688 | asascaccAfaAfGfAfuugucucugaL96 | 3683 | usCfsagaGfaCfAfaucuUfuGfguguuscsu |
| AD-159458 | asusguugUfcCfUfUfuuuaucugauL96 | 3684 | asUfscagAfuAfAfaaagGfaCfaacausgsc |
| AD-159519 | uscsaacuCfcUfGfAfaguuagaaauL96 | 3685 | asUfsuucUfaAfCfuucaGfgAfguugasusg |
| AD-159858 | asascuauCfcAfAfGfuguuauaccaL96 | 3686 | usGfsguaUfaAfCfacuuGfgAfuaguusgsg |
| AD-159583 | gsgsuauuAfaUfCfUfuguguagucuL96 | 3687 | asGfsacuAfcAfCfaagaUfuAfauaccsasu |
| AD-159700 | gsgscuccUfuCfAfCfugaacaugcaL96 | 3688 | usGfscauGfuUfCfagugAfaGfgagccsasg |
| AD-159807 | usasucagUfaGfUfGfuacauuaccaL96 | 3689 | usGfsguaAfuGfUfacacUfaCfugauasusa |
| AD-158673 | csasgccuUfuUfCfCfuuagaacacaL96 | 3690 | usGfsuguUfcUfAfaggaAfaAfggcugscsc |
| AD-159608 | csusgguuAfgUfGfUfgaaauaguuaL96 | 3691 | usAfsacuAfuUfUfcacaCfuAfaccagsusu |
| AD-159803 | ascsuauaUfcAfGfUfaguguacauuL96 | 3692 | asAfsuguAfcAfCfuacuGfaUfauagususc |
| AD-159805 | usasuaucAfgUfAfGfuguacauuaaL96 | 3693 | usUfsaauGfuAfCfacuaCfuGfauauasgsu |
| AD-159489 | gsusaauaUfuUfUfAfagauggacuaL96 | 3694 | usAfsgucCfaUfCfuuaaAfaUfauuacsusg |
| AD-159495 | ususuuaaGfaUfGfGfacugggaaaaL96 | 3695 | usUfsuucCfcAfGfuccaUfcUfuaaaasusa |
| AD-159706 | ususcacuGfaAfCfAfugccuagucaL96 | 3696 | usGfsacuAfgGfCfauguUfcAfgugaasgsg |
| AD-159855 | ascscaacUfaUfCfCfaaguguuauaL96 | 3697 | usAfsuaaCfaCfUfuggaUfaGfuuggususg |
| AD-159864 | cscsaaguGfuUfAfUfaccaacuaaaL96 | 3698 | usUfsuagUfuGfGfuauaAfcAfcuuggsasu |
| AD-159488 | asgsuaauAfuUfUfUfaagauggacuL96 | 3699 | asGfsuccAfuCfUfuaaaAfuAfuuacusgsc |
| AD-159553 | asasaaucCfaCfAfGfcuauauccuaL96 | 3700 | usAfsggaUfaUfAfgcugUfgGfauuuusasc |
| AD-159703 | uscscuucAfcUfGfAfacaugccuaaL96 | 3701 | usUfsaggCfaUfGfuucaGfuGfaaggasgsc |
| AD-159708 | csascugaAfcAfUfGfccuaguccaaL96 | 3702 | usUfsggaCfuAfGfgcauGfuUfcagugsasa |
| AD-159866 | asasguguUfaUfAfCfcaacuaaaacL96 | 3703 | gsUfsuuuAfgUfUfgguaUfaAfcacuusgsg |
| AD-159232 | ususccacCfaUfGfAfuuaagggucuL96 | 3704 | asGfsaccCfuUfAfaucaUfgGfuggaasasc |
| AD-159712 | gsasacauGfcCfUfAfguccaacauuL96 | 3705 | asAfsuguUfgGfAfcuagGfcAfuguucsasg |
| AD-159808 | asuscaguAfgUfGfUfacauuaccauL96 | 3706 | asUfsgguAfaUfGfuacaCfuAfcugausasu |
| AD-159862 | asusccaaGfuGfUfUfauaccaacuaL96 | 3707 | usAfsguuGfgUfAfuaacAfcUfuggausasg |
| AD-158503 | cscsaaguCfcAfAfUfauggcaacuaL96 | 3708 | usAfsguuGfcCfAfuauuGfgAfcuuggsasa |
| AD-159412 | csasuuucAfcUfGfUfcuaggcuacaL96 | 3709 | usGfsuagCfcUfAfgacaGfuGfaaaugsasu |
| AD-159558 | cscsacagCfuAfUfAfuccugaugcuL96 | 3710 | asGfscauCfaGfGfauauAfgCfuguggsasu |
| AD-159705 | csusucacUfgAfAfCfaugccuaguaL96 | 3711 | usAfscuaGfgCfAfuguuCfaGfugaagsgsa |
| AD-159113 | gsusgguuGfaGfAfGfugcuuaugaaL96 | 3712 | usUfscauAfaGfCfacucUfcAfaccacscsu |
| AD-159806 | asusaucaGfuAfGfUfguacauuacaL96 | 3713 | usGfsuaaUfgUfAfcacuAfcUfgauausasg |
| AD-159853 | csasaccaAfcUfAfUfccaaguguuaL96 | 3714 | usAfsacaCfuUfGfgauaGfuUfgguugscsa |
| AD-159182 | gscsagauUfuGfGfCfagagaguauaL96 | 3715 | usAfsuacUfcUfCfugccAfaAfucugcsusa |
| AD-159702 | csusccuuCfaCfUfGfaacaugccuaL96 | 3716 | usAfsggcAfuGfUfucagUfgAfaggagscsc |
| AD-159715 | csasugccUfaGfUfCfcaacauuuuuL96 | 3717 | asAfsaaaUfgUfUfggacUfaGfgcaugsusu |
| AD-158575 | usgsccauCfaGfUfAfucuuaaugaaL96 | 3718 | usUfscauUfaAfGfauacUfgAfuggcascsa |
| AD-158576 | gscscaucAfgUfAfUfcuuaaugaaaL96 | 3719 | usUfsucaUfuAfAfgauaCfuGfauggcsasc |
| AD-158684 | ususagaaCfaCfCfAfaagauugucuL96 | 3720 | asGfsacaAfuCfUfuuggUfgUfucuaasgsg |
| AD-159410 | asuscauuUfcAfCfUfgucuaggcuaL96 | 3721 | usAfsgccUfaGfAfcaguGfaAfaugausasu |
| AD-159416 | uscsacugUfcUfAfGfgcuacaacaaL96 | 3722 | usUfsguuGfuAfGfccuaGfaCfagugasasa |
| AD-159857 | csasacuaUfcCfAfAfguguuauacaL96 | 3723 | usGfsuauAfaCfAfcuugGfaUfaguugsgsu |
| AD-158497 | ususgguuCfcAfAfGfuccaauaugaL96 | 3724 | usCfsauaUfuGfGfacuuGfgAfaccaasasa |
| AD-159124 | usgscuuaUfgAfGfGfugaucaaacuL96 | 3725 | asGfsuuuGfaUfCfaccuCfaUfaagcascsu |
| AD-159312 | uscsucagAfcCfUfUfgugaaggugaL96 | 3726 | usCfsaccUfuCfAfcaagGfuCfugagasusu |
| AD-159552 | usasaaauCfcAfCfAfgcuauauccuL96 | 3727 | asGfsgauAfuAfGfcuguGfgAfuuuuascsa |
| AD-159704 | cscsuucaCfuGfAfAfcaugccuaguL96 | 3728 | asCfsuagGfcAfUfguucAfgUfgaaggsasg |
| AD-159737 | gsgsgaucCfaGfUfGfuauaaauccaL96 | 3729 | usGfsgauUfuAfUfacacUfgGfaucccsasg |
| AD-159869 | csasauaaAfcCfUfUfgaacagugaaL96 | 3730 | usUfscacUfgUfUfcaagGfuUfuauugsgsg |
| AD-158570 | gsgsccugUfgCfCfAfucaguaucuuL96 | 3731 | asAfsgauAfcUfGfauggCfaCfaggccsasu |
| AD-158618 | ususguugAfuGfUfCfaucgaagacaL96 | 3732 | usGfsucuUfcGfAfugacAfuCfaacaasgsa |
| AD-159184 | asgsauuuGfgCfAfGfagaguauaauL96 | 3733 | asUfsuauAfcUfCfucugCfcAfaaucusgsc |
| AD-159231 | ususuccaCfcAfUfGfauuaaggguaL96 | 3734 | usAfscccUfuAfAfucauGfgUfggaaascsu |
| AD-159423 | csusaggcUfaCfAfAfcaggauucuaL96 | 3735 | usAfsgaaUfcCfUfguugUfaGfccuagsasc |
| AD-159446 | usgsgaggUfuGfUfGfcauguugucaL96 | 3736 | usGfsacaAfcAfUfgcacAfaCfcuccascsc |
| AD-159701 | gscsuccuUfcAfCfUfgaacaugccuL96 | 3737 | asGfsgcaUfgUfUfcaguGfaAfggagcscsa |
| AD-158494 | csusuuugGfuUfCfCfaaguccaauaL96 | 3738 | usAfsuugGfaCfUfuggaAfcCfaaaagsgsa |
| AD-158571 | gscscuguGfcCfAfUfcaguaucuuaL96 | 3739 | usAfsagaUfaCfUfgaugGfcAfcaggcscsa |
| AD-159125 | gscsuuauGfaGfGfUfgaucaaacuaL96 | 3740 | usAfsguuUfgAfUfcaccUfcAfuaagcsasc |
| AD-159126 | csusuaugAfgGfUfGfaucaaacucaL96 | 3741 | usGfsaguUfuGfAfucacCfuCfauaagscsa |
| AD-159287 | cscsuugcAfuUfUfUfgggacagaauL96 | 3742 | asUfsucuGfuCfCfcaaaAfuGfcaaggsasa |
| AD-158499 | gsgsuuccAfaGfUfCfcaauauggcaL96 | 3743 | usGfsccaUfaUfUfggacUfuGfgaaccsasa |
| AD-159417 | csascuguCfuAfGfGfcuacaacagaL96 | 3744 | usCfsuguUfgUfAfgccuAfgAfcagugsasa |
| AD-159418 | ascsugucUfaGfGfCfuacaacaggaL96 | 3745 | usCfscugUfuGfUfagccUfaGfacagusgsa |
| AD-158550 | asasuaagAfuUfAfCfaguuguuggaL96 | 3746 | usCfscaaCfaAfCfuguaAfuCfuuauuscsu |
| AD-159116 | gsusugagAfgUfGfCfuuaugagguaL96 | 3747 | usAfsccuCfaUfAfagcaCfuCfucaacscsa |
| AD-159421 | gsuscuagGfcUfAfCfaacaggauuaL96 | 3748 | usAfsaucCfuGfUfuguaGfcCfuagacsasg |
| AD-159422 | uscsuaggCfuAfCfAfacaggauucuL96 | 3749 | asGfsaauCfcUfGfuuguAfgCfcuagascsa |
| AD-159445 | gsusggagGfuUfGfUfgcauguuguaL96 | 3750 | usAfscaaCfaUfGfcacaAfcCfuccacscsu |
| AD-159130 | usgsagguGfaUfCfAfaacucaaagaL96 | 3751 | usCfsuuuGfaGfUfuugaUfcAfccucasusa |
| AD-159134 | gsusgaucAfaAfCfUfcaaaggcuaaL96 | 3752 | usUfsagcCfuUfUfgaguUfuGfaucacscsu |
| AD-159343 | usgsaggaAfgAfGfGfcccguuugaaL96 | 3753 | usUfscaaAfcGfGfgccuCfuUfccucasgsa |
| AD-159105 | ascsaagcAfgGfUfGfguugagaguaL96 | 3754 | usAfscucUfcAfAfccacCfuGfcuugusgsa |
| AD-159183 | csasgauuUfgGfCfAfgagaguauaaL96 | 3755 | usUfsauaCfuCfUfcugcCfaAfaucugscsu |
| AD-159123 | gsusgcuuAfuGfAfGfgugaucaaacL96 | 3756 | gsUfsuugAfuCfAfccucAfuAfagcacsusc |
| AD-159181 | asgscagaUfuUfGfGfcagagaguauL96 | 3757 | asUfsacuCfuCfUfgccaAfaUfcugcusasc |
| AD-159186 | asusuuggCfaGfAfGfaguauaaugaL96 | 3758 | usCfsauuAfuAfCfucucUfgCfcaaauscsu |
| AD-159187 | ususuggcAfgAfGfAfguauaaugaaL96 | 3759 | usUfscauUfaUfAfcucuCfuGfccaaasusc |
| AD-159288 | csusugcaUfuUfUfGfggacagaauaL96 | 3760 | usAfsuucUfgUfCfccaaAfaUfgcaagsgsa |
| AD-159306 | asusggaaUfcUfCfAfgaccuugugaL96 | 3761 | usCfsacaAfgGfUfcugaGfaUfuccaususc |
| AD-159559 | csascagcUfaUfAfUfccugaugcuaL96 | 3762 | usAfsgcaUfcAfGfgauaUfaGfcugugsgsa |
| AD-159344 | gsasggaaGfaGfGfCfccguuugaaaL96 | 3763 | usUfsucaAfaCfGfggccUfcUfuccucsasg |
| AD-159341 | uscsugagGfaAfGfAfggcccguuuaL96 | 3764 | usAfsaacGfgGfCfcucuUfcCfucagasasg |
| AD-159729 | csascaucCfuGfGfGfauccaguguaL96 | 3765 | usAfscacUfgGfAfucccAfgGfaugugsasc |
| AD-158674 | asgsccuuUfuCfCfUfuagaacaccaL96 | 3766 | usGfsgugUfuCfUfaaggAfaAfaggcusgsc |
| AD-159604 | uscsaacuGfgUfUfAfgugugaaauaL96 | 3767 | usAfsuuuCfaCfAfcuaaCfcAfguugasasg |

| Duplex | | | |
|---|---|---|---|
| Name | SEQ ID NO | mRNA target sequence | SEQ ID NO |
| AD-159469 | 3768 | UUUUUAUCUGAUCUGUGAUUAAA | 3954 |
| AD-159607 | 3769 | CAACUGGUUAGUGUGAAAUAGUU | 3955 |
| AD-159713 | 3770 | UGAACAUGCCUAGUCCAACAUUU | 3956 |
| AD-158504 | 3771 | UCCAAGUCCAAUAUGGCAACUCU | 3957 |
| AD-159233 | 3772 | UUUCCACCAUGAUUAAGGGUCUU | 3958 |
| AD-159411 | 3773 | UAUCAUUUCACUGUCUAGGCUAC | 3959 |
| AD-159462 | 3774 | GUUGUCCUUUUUAUCUGAUCUGU | 3960 |
| AD-159742 | 3775 | AUCCAGUGUAUAAAUCCAAUAUC | 3961 |
| AD-159863 | 3776 | UAUCCAAGUGUUAUACCAACUAA | 3962 |
| AD-158626 | 3777 | AUGUCAUCGAAGACAAAUUGAAG | 3963 |
| AD-158687 | 3778 | UAGAACACCAAAGAUUGUCUCUG | 3964 |
| AD-158688 | 3779 | AGAACACCAAAGAUUGUCUCUGG | 3965 |
| AD-159458 | 3780 | GCAUGUUGUCCUUUUUAUCUGAU | 3966 |
| AD-159519 | 3781 | CAUCAACUCCUGAAGUUAGAAAU | 3967 |
| AD-159858 | 3782 | CCAACUAUCCAAGUGUUAUACCA | 3968 |
| AD-158681 | 3783 | UUUCCUUAGAACACCAAAGAUUG | 3969 |
| AD-159583 | 3784 | AUGGUAUUAAUCUUGUGUAGUCU | 3970 |
| AD-159700 | 3785 | CUGGCUCCUUCACUGAACAUGCC | 3971 |
| AD-159807 | 3786 | UAUAUCAGUAGUGUACAUUACCA | 3972 |
| AD-158673 | 3787 | GGCAGCCUUUUCCUUAGAACACC | 3973 |
| AD-159608 | 3788 | AACUGGUUAGUGUGAAAUAGUUC | 3974 |
| AD-159803 | 3789 | GAACUAUAUCAGUAGUGUACAUU | 3975 |
| AD-159805 | 3790 | ACUAUAUCAGUAGUGUACAUUAC | 3976 |
| AD-159489 | 3791 | CAGUAAUAUUUUAAGAUGGACUG | 3977 |
| AD-159495 | 3792 | UAUUUUAAGAUGGACUGGGAAAA | 3978 |
| AD-159609 | 3793 | ACUGGUUAGUGUGAAAUAGUUCU | 3979 |
| AD-159706 | 3794 | CCUUCACUGAACAUGCCUAGUCC | 3980 |
| AD-159855 | 3795 | CAACCAACUAUCCAAGUGUUAUA | 3981 |
| AD-159864 | 3796 | AUCCAAGUGUUAUACCAACUAAA | 3982 |
| AD-158491 | 3797 | GAUUCCUUUUGGUUCCAAGUCCA | 3983 |
| AD-158672 | 3798 | UGGCAGCCUUUUCCUUAGAACAC | 3984 |
| AD-159488 | 3799 | GCAGUAAUAUUUUAAGAUGGACU | 3985 |
| AD-159553 | 3800 | GUAAAAUCCACAGCUAUAUCCUG | 3986 |
| AD-159703 | 3801 | GCUCCUUCACUGAACAUGCCUAG | 3987 |
| AD-159708 | 3802 | UUCACUGAACAUGCCUAGUCCAA | 3988 |
| AD-159866 | 3803 | CCAAGUGUUAUACCAACUAAAAC | 3989 |
| AD-159232 | 3804 | GUUUCCACCAUGAUUAAGGGUCU | 3990 |
| AD-159712 | 3805 | CUGAACAUGCCUAGUCCAACAUU | 3991 |
| AD-159808 | 3806 | AUAUCAGUAGUGUACAUUACCAU | 3992 |
| AD-159862 | 3807 | CUAUCCAAGUGUUAUACCAACUA | 3993 |
| AD-158503 | 3808 | UUCCAAGUCCAAUAUGGCAACUC | 3994 |
| AD-159311 | 3809 | GAAUCUCAGACCUUGUGAAGGUG | 3995 |
| AD-159412 | 3810 | AUCAUUUCACUGUCUAGGCUACA | 3996 |
| AD-159558 | 3811 | AUCCACAGCUAUAUCCUGAUGCU | 3997 |
| AD-159705 | 3812 | UCCUUCACUGAACAUGCCUAGUC | 3998 |
| AD-159113 | 3813 | AGGUGGUUGAGAGUGCUUAUGAG | 3999 |
| AD-159139 | 3814 | AUCAAACUCAAAGGCUACACAUC | 4000 |
| AD-159806 | 3815 | CUAUAUCAGUAGUGUACAUUACC | 4001 |
| AD-159853 | 3816 | UGCAACCAACUAUCCAAGUGUUA | 4002 |
| AD-158627 | 3817 | UGUCAUCGAAGACAAAUUGAAGG | 4003 |
| AD-159182 | 3818 | UAGCAGAUUUGGCAGAGAGUAUA | 4004 |
| AD-159702 | 3819 | GGCUCCUUCACUGAACAUGCCUA | 4005 |
| AD-159715 | 3820 | AACAUGCCUAGUCCAACAUUUUU | 4006 |
| AD-158575 | 3821 | UGUGCCAUCAGUAUCUUAAUGAA | 4007 |
| AD-158576 | 3822 | GUGCCAUCAGUAUCUUAAUGAAG | 4008 |
| AD-158684 | 3823 | CCUUAGAACACCAAAGAUUGUCU | 4009 |
| AD-159410 | 3824 | AUAUCAUUUCACUGUCUAGGCUA | 4010 |
| AD-159416 | 3825 | UUUCACUGUCUAGGCUACAACAG | 4011 |
| AD-159738 | 3826 | UGGGAUCCAGUGUAUAAAUCCAA | 4012 |
| AD-159857 | 3827 | ACCAACUAUCCAAGUGUUAUACC | 4013 |
| AD-158497 | 3828 | UUUUGGUUCCAAGUCCAAUAUGG | 4014 |
| AD-159124 | 3829 | AGUGCUUAUGAGGUGAUCAAACU | 4015 |
| AD-159140 | 3830 | UCAAACUCAAAGGCUACACAUCC | 4016 |
| AD-159312 | 3831 | AAUCUCAGACCUUGUGAAGGUGA | 4017 |
| AD-159552 | 3832 | UGUAAAAUCCACAGCUAUAUCCU | 4018 |
| AD-159704 | 3833 | CUCCUUCACUGAACAUGCCUAGU | 4019 |
| AD-159737 | 3834 | CUGGGAUCCAGUGUAUAAAUCCA | 4020 |
| AD-159869 | 3835 | CCCAAUAAACCUUGAACAGUGAC | 4021 |
| AD-158570 | 3836 | AUGGCCUGUGCCAUCAGUAUCUU | 4022 |
| AD-158618 | 3837 | UCUUGUUGAUGUCAUCGAAGACA | 4023 |
| AD-159788 | 3838 | AAGGAUCUUAUUUUGUGAACUAU | 4024 |
| AD-159786 | 3839 | CAAAGGAUCUUAUUUUGUGAACU | 4025 |
| AD-159760 | 3840 | AUAUCAUGUCUUGUGCAUAAUUC | 4026 |
| AD-159404 | 3841 | GAUGUCAUAUCAUUUCACUGUCU | 4027 |
| AD-159406 | 3842 | UGUCAUAUCAUUUCACUGUCUAG | 4028 |
| AD-158536 | 3843 | UGAUUUAUAAUCUUCUAAAGGAA | 4029 |
| AD-159545 | 3844 | AAUGGUUUGUAAAAUCCACAGCU | 4030 |
| AD-159574 | 3845 | UGAUGCUGGAUGGUAUUAAUCUU | 4031 |
| AD-159802 | 3846 | UGAACUAUAUCAGUAGUGUACAU | 4032 |
| AD-159518 | 3847 | ACAUCAACUCCUGAAGUUAGAAA | 4033 |
| AD-159577 | 3848 | UGCUGGAUGGUAUUAAUCUUGUG | 4034 |
| AD-159409 | 3849 | CAUAUCAUUUCACUGUCUAGGCU | 4035 |
| AD-159551 | 3850 | UUGUAAAAUCCACAGCUAUAUCC | 4036 |
| AD-159276 | 3851 | CUUCCUUAGUGUUCCUUGCAUUU | 4037 |
| AD-159407 | 3852 | GUCAUAUCAUUUCACUGUCUAGG | 4038 |
| AD-159515 | 3853 | AAAACAUCAACUCCUGAAGUUAG | 4039 |
| AD-159570 | 3854 | AUCCUGAUGCUGGAUGGUAUUAA | 4040 |
| AD-159849 | 3855 | ACAAUGCAACCAACUAUCCAAGU | 4041 |
| AD-159252 | 3856 | UCUUUACGGAAUAAAGGAUGAUG | 4042 |
| AD-159275 | 3857 | UCUUCCUUAGUGUUCCUUGCAUU | 4043 |
| AD-159848 | 3858 | AACAAUGCAACCAACUAUCCAAG | 4044 |
| AD-159184 | 3859 | GCAGAUUUGGCAGAGAGUAUAAU | 4045 |
| AD-159231 | 3860 | AGUUUCCACCAUGAUUAAGGGUC | 4046 |
| AD-159607 | 3861 | CAACUGGUUAGUGUGAAAUAGUU | 4047 |
| AD-158504 | 3862 | UCCAAGUCCAAUAUGGCAACUCU | 4048 |
| AD-159233 | 3863 | UUUCCACCAUGAUUAAGGGUCUU | 4049 |
| AD-159411 | 3864 | UAUCAUUUCACUGUCUAGGCUAC | 4050 |
| AD-159462 | 3865 | GUUGUCCUUUUUAUCUGAUCUGU | 4051 |
| AD-159742 | 3866 | AUCCAGUGUAUAAAUCCAAUAUC | 4052 |
| AD-159863 | 3867 | UAUCCAAGUGUUAUACCAACUAA | 4053 |
| AD-158687 | 3868 | UAGAACACCAAAGAUUGUCUCUG | 4054 |
| AD-158688 | 3869 | AGAACACCAAAGAUUGUCUCUGG | 4055 |
| AD-159458 | 3870 | GCAUGUUGUCCUUUUUAUCUGAU | 4056 |
| AD-159519 | 3871 | CAUCAACUCCUGAAGUUAGAAAU | 4057 |
| AD-159858 | 3872 | CCAACUAUCCAAGUGUUAUACCA | 4058 |
| AD-159583 | 3873 | AUGGUAUUAAUCUUGUGUAGUCU | 4059 |
| AD-159700 | 3874 | CUGGCUCCUUCACUGAACAUGCC | 4060 |
| AD-159807 | 3875 | UAUAUCAGUAGUGUACAUUACCA | 4061 |
| AD-158673 | 3876 | GGCAGCCUUUUCCUUAGAACACC | 4062 |
| AD-159608 | 3877 | AACUGGUUAGUGUGAAAUAGUUC | 4063 |
| AD-159803 | 3878 | GAACUAUAUCAGUAGUGUACAUU | 4064 |
| AD-159805 | 3879 | ACUAUAUCAGUAGUGUACAUUAC | 4065 |
| AD-159489 | 3880 | CAGUAAUAUUUUAAGAUGGACUG | 4066 |
| AD-159495 | 3881 | UAUUUUAAGAUGGACUGGGAAAA | 4067 |
| AD-159706 | 3882 | CCUUCACUGAACAUGCCUAGUCC | 4068 |
| AD-159855 | 3883 | CAACCAACUAUCCAAGUGUUAUA | 4069 |
| AD-159864 | 3884 | AUCCAAGUGUUAUACCAACUAAA | 4070 |
| AD-159488 | 3885 | GCAGUAAUAUUUUAAGAUGGACU | 4071 |
| AD-159553 | 3886 | GUAAAAUCCACAGCUAUAUCCUG | 4072 |
| AD-159703 | 3887 | GCUCCUUCACUGAACAUGCCUAG | 4073 |
| AD-159708 | 3888 | UUCACUGAACAUGCCUAGUCCAA | 4074 |
| AD-159866 | 3889 | CCAAGUGUUAUACCAACUAAAAC | 4075 |
| AD-159232 | 3890 | GUUUCCACCAUGAUUAAGGGUCU | 4076 |
| AD-159712 | 3891 | CUGAACAUGCCUAGUCCAACAUU | 4077 |
| AD-159808 | 3892 | AUAUCAGUAGUGUACAUUACCAU | 4078 |
| AD-159862 | 3893 | CUAUCCAAGUGUUAUACCAACUA | 4079 |
| AD-158503 | 3894 | UUCCAAGUCCAAUAUGGCAACUC | 4080 |
| AD-159412 | 3895 | AUCAUUUCACUGUCUAGGCUACA | 4081 |
| AD-159558 | 3896 | AUCCACAGCUAUAUCCUGAUGCU | 4082 |
| AD-159705 | 3897 | UCCUUCACUGAACAUGCCUAGUC | 4083 |
| AD-159113 | 3898 | AGGUGGUUGAGAGUGCUUAUGAG | 4084 |
| AD-159806 | 3899 | CUAUAUCAGUAGUGUACAUUACC | 4085 |
| AD-159853 | 3900 | UGCAACCAACUAUCCAAGUGUUA | 4086 |
| AD-159182 | 3901 | UAGCAGAUUUGGCAGAGAGUAUA | 4087 |
| AD-159702 | 3902 | GGCUCCUUCACUGAACAUGCCUA | 4088 |
| AD-159715 | 3903 | AACAUGCCUAGUCCAACAUUUUU | 4089 |
| AD-158575 | 3904 | UGUGCCAUCAGUAUCUUAAUGAA | 4090 |
| AD-158576 | 3905 | GUGCCAUCAGUAUCUUAAUGAAG | 4091 |
| AD-158684 | 3906 | CCUUAGAACACCAAAGAUUGUCU | 4092 |
| AD-159410 | 3907 | AUAUCAUUUCACUGUCUAGGCUA | 4093 |
| AD-159416 | 3908 | UUUCACUGUCUAGGCUACAACAG | 4094 |
| AD-159857 | 3909 | ACCAACUAUCCAAGUGUUAUACC | 4095 |
| AD-158497 | 3910 | UUUUGGUUCCAAGUCCAAUAUGG | 4096 |
| AD-159124 | 3911 | AGUGCUUAUGAGGUGAUCAAACU | 4097 |
| AD-159312 | 3912 | AAUCUCAGACCUUGUGAAGGUGA | 4098 |
| AD-159552 | 3913 | UGUAAAAUCCACAGCUAUAUCCU | 4099 |
| AD-159704 | 3914 | CUCCUUCACUGAACAUGCCUAGU | 4100 |
| AD-159737 | 3915 | CUGGGAUCCAGUGUAUAAAUCCA | 4101 |
| AD-159869 | 3916 | CCCAAUAAACCUUGAACAGUGAC | 4102 |
| AD-158570 | 3917 | AUGGCCUGUGCCAUCAGUAUCUU | 4103 |
| AD-158618 | 3918 | UCUUGUUGAUGUCAUCGAAGACA | 4104 |
| AD-159184 | 3919 | GCAGAUUUGGCAGAGAGUAUAAU | 4105 |
| AD-159231 | 3920 | AGUUUCCACCAUGAUUAAGGGUC | 4106 |
| AD-159423 | 3921 | GUCUAGGCUACAACAGGAUUCUA | 4107 |
| AD-159446 | 3922 | GGUGGAGGUUGUGCAUGUUGUCC | 4108 |
| AD-159701 | 3923 | UGGCUCCUUCACUGAACAUGCCU | 4109 |
| AD-158494 | 3924 | UCCUUUUGGUUCCAAGUCCAAUA | 4110 |
| AD-158571 | 3925 | UGGCCUGUGCCAUCAGUAUCUUA | 4111 |
| AD-159125 | 3926 | GUGCUUAUGAGGUGAUCAAACUC | 4112 |
| AD-159126 | 3927 | UGCUUAUGAGGUGAUCAAACUCA | 4113 |
| AD-159287 | 3928 | UUCCUUGCAUUUUGGGACAGAAU | 4114 |
| AD-158499 | 3929 | UUGGUUCCAAGUCCAAUAUGGCA | 4115 |
| AD-159417 | 3930 | UUCACUGUCUAGGCUACAACAGG | 4116 |
| AD-159418 | 3931 | UCACUGUCUAGGCUACAACAGGA | 4117 |
| AD-158550 | 3932 | AGAAUAAGAUUACAGUUGUUGGG | 4118 |
| AD-159116 | 3933 | UGGUUGAGAGUGCUUAUGAGGUG | 4119 |
| AD-159421 | 3934 | CUGUCUAGGCUACAACAGGAUUC | 4120 |
| AD-159422 | 3935 | UGUCUAGGCUACAACAGGAUUCU | 4121 |
| AD-159445 | 3936 | AGGUGGAGGUUGUGCAUGUUGUC | 4122 |
| AD-159130 | 3937 | UAUGAGGUGAUCAAACUCAAAGG | 4123 |
| AD-159134 | 3938 | AGGUGAUCAAACUCAAAGGCUAC | 4124 |
| AD-159343 | 3939 | UCUGAGGAAGAGGCCCGUUUGAA | 4125 |
| AD-159105 | 3940 | UCACAAGCAGGUGGUUGAGAGUG | 4126 |
| AD-159183 | 3941 | AGCAGAUUUGGCAGAGAGUAUAA | 4127 |
| AD-159123 | 3942 | GAGUGCUUAUGAGGUGAUCAAAC | 4128 |
| AD-159181 | 3943 | GUAGCAGAUUUGGCAGAGAGUAU | 4129 |
| AD-159186 | 3944 | AGAUUUGGCAGAGAGUAUAAUGA | 4130 |
| AD-159187 | 3945 | GAUUUGGCAGAGAGUAUAAUGAA | 4131 |
| AD-159288 | 3946 | UCCUUGCAUUUUGGGACAGAAUG | 4132 |
| AD-159306 | 3947 | GAAUGGAAUCUCAGACCUUGUGA | 4133 |
| AD-159559 | 3948 | UCCACAGCUAUAUCCUGAUGCUG | 4134 |
| AD-159344 | 3949 | CUGAGGAAGAGGCCCGUUUGAAG | 4135 |
| AD-159341 | 3950 | CUUCUGAGGAAGAGGCCCGUUUG | 4136 |
| AD-159729 | 3951 | GUCACAUCCUGGGAUCCAGUGUA | 4137 |
| AD-158674 | 3952 | GCAGCCUUUUCCUUAGAACACCA | 4138 |
| AD-159604 | 3953 | CUUCAACUGGUUAGUGUGAAAUA | 4139 |

TABLE 6A
Single dose screen in Primary Mouse Hepatocytes

| Duplex Name | 10 nM | STDEV | 0.1 nM | STDEV |
|---|---|---|---|---|
| AD-84747 | 8.1 | 1.8 | 38.6 | 4.1 |
| AD-84748 | 58.2 | 11.9 | 77.0 | 14.5 |
| AD-84749 | 12.0 | 1.6 | 33.7 | 8.3 |
| AD-84750 | 9.9 | 1.4 | 38.1 | 9.7 |
| AD-84751 | 22.7 | 8.0 | 67.2 | 11.8 |
| AD-84752 | 23.6 | 3.5 | 54.5 | 21.7 |
| AD-84753 | 8.2 | 1.4 | 26.2 | 11.4 |
| AD-84754 | 29.7 | 7.3 | 41.7 | 2.9 |
| AD-84755 | 24.5 | 9.3 | 61.7 | 9.9 |
| AD-84756 | 5.2 | 0.8 | 32.8 | 15.6 |
| AD-84757 | 10.4 | 0.5 | 60.5 | 9.0 |
| AD-84758 | 18.7 | 5.8 | 49.9 | 20.9 |
| AD-84759 | 14.9 | 2.7 | 68.2 | 23.8 |
| AD-84760 | 39.2 | 4.8 | 53.3 | 19.5 |
| AD-84761 | 5.3 | 1.3 | 23.5 | 8.0 |
| AD-84762 | 5.4 | 1.0 | 24.4 | 2.5 |
| AD-84763 | 9.4 | 1.9 | 48.3 | 18.5 |
| AD-84764 | 9.3 | 1.5 | 46.8 | 19.3 |
| AD-84765 | 15.8 | 3.3 | 81.1 | 24.6 |
| AD-84766 | 35.6 | 5.9 | 77.6 | 36.9 |
| AD-84767 | 46.1 | 9.5 | 112.5 | 21.9 |
| AD-84768 | 14.4 | 3.2 | 73.2 | 33.0 |
| AD-84769 | 8.3 | 3.6 | 29.9 | 2.7 |
| AD-84770 | 8.1 | 3.1 | 35.0 | 4.8 |
| AD-84771 | 22.3 | 9.5 | 90.9 | 28.2 |
| AD-84772 | 11.4 | 5.4 | 56.4 | 11.3 |
| AD-84773 | 35.6 | 16.7 | 104.8 | 20.3 |
| AD-84774 | 40.5 | 16.0 | 98.4 | 35.0 |
| AD-84775 | 16.0 | 6.2 | 66.6 | 17.2 |
| AD-84776 | 26.6 | 13.9 | 82.9 | 26.3 |
| AD-84777 | 18.1 | 1.7 | 54.2 | 14.7 |
| AD-84778 | 21.9 | 7.2 | 92.5 | 30.9 |
| AD-84779 | 31.9 | 8.6 | 99.5 | 39.5 |
| AD-84780 | 15.4 | 2.7 | 53.8 | 35.9 |
| AD-84781 | 13.2 | 2.4 | 61.8 | 2.7 |
| AD-84782 | 14.4 | 4.1 | 67.9 | 33.3 |
| AD-84783 | 20.8 | 5.5 | 89.0 | 31.1 |
| AD-84784 | 15.6 | 3.0 | 50.3 | 19.4 |
| AD-84785 | 12.3 | 12.3 | 23.5 | 23.5 |
| AD-84786 | 4.7 | 4.7 | 35.3 | 35.3 |
| AD-84787 | 12.4 | 12.4 | 45.5 | 45.5 |
| AD-84788 | 2.3 | 2.3 | 7.8 | 7.8 |
| AD-84789 | 9.4 | 9.4 | 45.7 | 45.7 |
| AD-84790 | 2.5 | 2.5 | 12.8 | 12.8 |

TABLE 6B
Single dose screen in Hep3b

| | % of Human | |
|---|---|---|
| Duplex | Message | |
| Name | Remaining | STDEV |
| AD-159469 | 16.97 | 6.86 |
| AD-159607 | 25.01 | 8.34 |
| AD-159713 | 25.91 | 11.30 |
| AD-158504 | 21.90 | 8.34 |
| AD-159233 | 25.16 | 10.01 |
| AD-159411 | 22.65 | 8.86 |
| AD-159462 | 31.26 | 10.89 |
| AD-159742 | 26.31 | 4.08 |
| AD-159863 | 22.44 | 5.86 |
| AD-158626 | 11.06 | 9.33 |
| AD-158687 | 17.11 | 9.55 |
| AD-158688 | 16.22 | 11.59 |
| AD-159458 | 16.59 | 9.47 |
| AD-159519 | 16.60 | 2.85 |
| AD-159858 | 31.03 | 12.43 |
| AD-158681 | 12.52 | 5.04 |
| AD-159583 | 30.63 | 8.04 |
| AD-159700 | 60.23 | 11.10 |
| AD-159807 | 12.17 | 4.73 |
| AD-158673 | 7.41 | 0.92 |
| AD-159608 | 19.93 | 9.83 |
| AD-159803 | 29.79 | 8.75 |
| AD-159805 | 31.27 | 12.09 |
| AD-159489 | 50.07 | 7.60 |
| AD-159495 | 22.72 | 2.15 |
| AD-159609 | 17.39 | 9.56 |
| AD-159706 | 25.44 | 3.75 |
| AD-159855 | 16.67 | 12.67 |
| AD-159864 | 8.09 | 1.09 |
| AD-158491 | 29.16 | 14.26 |
| AD-158672 | 29.36 | 10.12 |
| AD-159488 | 31.40 | 6.20 |
| AD-159553 | 24.36 | 7.63 |
| AD-159703 | 16.04 | 4.80 |
| AD-159708 | 100.96 | 26.91 |
| AD-159866 | 26.91 | 5.95 |
| AD-159232 | 21.82 | 8.62 |
| AD-159712 | 30.31 | 3.10 |
| AD-159808 | 47.72 | 11.27 |
| AD-159862 | 18.26 | 6.31 |
| AD-158503 | 32.70 | 7.50 |
| AD-159311 | 18.45 | 3.39 |
| AD-159412 | 24.28 | 10.07 |
| AD-159558 | 34.02 | 4.51 |
| AD-159705 | 28.29 | 4.65 |
| AD-159113 | 17.03 | 7.27 |
| AD-159139 | 33.24 | 8.38 |
| AD-159806 | 25.80 | 17.42 |
| AD-159853 | 28.52 | 3.85 |
| AD-158627 | 35.28 | 9.47 |
| AD-159182 | 29.66 | 7.88 |
| AD-159702 | 37.01 | 11.07 |
| AD-159715 | 22.32 | 6.78 |
| AD-158575 | 18.91 | 11.44 |
| AD-158576 | 37.74 | 18.73 |
| AD-158684 | 15.69 | 9.50 |
| AD-159410 | 30.98 | 3.65 |
| AD-159416 | 42.29 | 20.80 |
| AD-159738 | 20.66 | 2.83 |
| AD-159857 | 28.70 | 8.69 |
| AD-158497 | 22.79 | 4.43 |
| AD-159124 | 16.84 | 7.19 |
| AD-159140 | 30.90 | 7.50 |
| AD-159312 | 70.66 | 21.57 |
| AD-159552 | 29.86 | 7.83 |
| AD-159704 | 44.45 | 7.57 |
| AD-159737 | 29.05 | 8.48 |
| AD-159869 | 28.46 | 9.39 |
| AD-158570 | 31.18 | 7.43 |
| AD-158618 | 27.03 | 8.54 |
| AD-159788 | 19.87 | 9.21 |
| AD-159786 | 31.83 | 27.17 |
| AD-159760 | 32.68 | 18.79 |
| AD-159404 | 47.91 | 22.88 |
| AD-159406 | 23.84 | 10.41 |
| AD-158536 | 30.88 | 20.74 |
| AD-159545 | 84.72 | 26.81 |
| AD-159574 | 29.96 | 20.03 |
| AD-159802 | 24.57 | 9.29 |
| AD-159518 | 29.06 | 16.06 |
| AD-159577 | 34.39 | 12.83 |
| AD-159409 | 50.02 | 25.26 |
| AD-159551 | 33.79 | 11.99 |
| AD-159276 | 40.09 | 13.96 |
| AD-159407 | 37.47 | 9.59 |
| AD-159515 | 41.82 | 19.54 |
| AD-159570 | 12.41 | 3.87 |
| AD-159849 | 25.67 | 14.76 |
| AD-159252 | 14.25 | 4.14 |
| AD-159275 | 22.30 | 13.03 |
| AD-159848 | 34.58 | 13.52 |
| AD-159184 | 30.50 | 8.60 |
| AD-159231 | 103.27 | 9.11 |
| AD-159607 | 16.73 | 1.97 |
| AD-158504 | 11.46 | 1.78 |
| AD-159233 | 15.90 | 3.55 |
| AD-159411 | 9.04 | 1.84 |
| AD-159462 | 16.08 | 7.18 |
| AD-159742 | 10.92 | 3.23 |
| AD-159863 | 8.82 | 2.51 |
| AD-158687 | 14.93 | 6.23 |
| AD-158688 | 15.77 | 5.03 |
| AD-159458 | 14.85 | 9.10 |
| AD-159519 | 20.25 | 9.24 |
| AD-159858 | 22.20 | 14.11 |
| AD-159583 | 20.01 | 1.53 |
| AD-159700 | 56.12 | 12.02 |
| AD-159807 | 16.73 | 7.03 |
| AD-158673 | 6.01 | 2.09 |
| AD-159608 | 13.52 | 6.68 |
| AD-159803 | 30.47 | 10.26 |
| AD-159805 | 10.28 | 1.16 |
| AD-159489 | 24.20 | 2.91 |
| AD-159495 | 22.32 | 13.94 |
| AD-159706 | 30.61 | 17.66 |
| AD-159855 | 9.32 | 1.46 |
| AD-159864 | 10.64 | 2.41 |
| AD-159488 | 19.16 | 6.42 |
| AD-159553 | 21.69 | 13.77 |
| AD-159703 | 12.05 | 1.69 |
| AD-159708 | 68.53 | 3.86 |
| AD-159866 | 32.03 | 21.42 |
| AD-159232 | 11.99 | 1.77 |
| AD-159712 | 37.95 | 11.97 |
| AD-159808 | 15.66 | 5.30 |
| AD-159862 | 14.03 | 6.78 |
| AD-158503 | 38.82 | 12.61 |
| AD-159412 | 34.58 | 22.60 |
| AD-159558 | 44.20 | 9.58 |
| AD-159705 | 29.96 | 11.90 |
| AD-159113 | 9.61 | 0.94 |
| AD-159806 | 11.45 | 1.10 |
| AD-159853 | 18.04 | 5.87 |
| AD-159182 | 11.32 | 2.80 |
| AD-159702 | 16.90 | 2.27 |
| AD-159715 | 18.48 | 10.27 |
| AD-158575 | 12.02 | 1.74 |
| AD-158576 | 20.78 | 6.11 |
| AD-158684 | 11.37 | 7.57 |
| AD-159410 | 29.86 | 7.02 |
| AD-159416 | 46.73 | 11.03 |
| AD-159857 | 24.36 | 5.16 |
| AD-158497 | 30.17 | 3.74 |
| AD-159124 | 25.97 | 4.90 |
| AD-159312 | 70.74 | 5.44 |
| AD-159552 | 41.03 | 6.19 |
| AD-159704 | 35.64 | 15.41 |
| AD-159737 | 20.64 | 4.47 |
| AD-159869 | 32.80 | 5.77 |
| AD-158570 | 30.61 | 6.04 |
| AD-158618 | 23.25 | 8.74 |
| AD-159184 | 25.44 | 9.61 |
| AD-159231 | 84.40 | 6.16 |
| AD-159423 | 14.14 | 2.24 |
| AD-159446 | 24.93 | 8.57 |
| AD-159701 | 50.20 | 3.80 |
| AD-158494 | 11.88 | 2.84 |
| AD-158571 | 46.81 | 7.47 |
| AD-159125 | 15.81 | 2.66 |
| AD-159126 | 29.28 | 8.63 |
| AD-159287 | 25.25 | 2.91 |
| AD-158499 | 29.76 | 5.51 |
| AD-159417 | 32.69 | 6.45 |
| AD-159418 | 24.84 | 7.31 |
| AD-158550 | 28.87 | 4.53 |
| AD-159116 | 26.12 | 2.58 |
| AD-159421 | 22.32 | 3.28 |
| AD-159422 | 24.24 | 7.34 |
| AD-159445 | 33.50 | 10.14 |
| AD-159130 | 24.80 | 4.33 |
| AD-159134 | 10.46 | 1.12 |
| AD-159343 | 34.97 | 8.91 |
| AD-159105 | 92.74 | 4.56 |
| AD-159183 | 41.08 | 12.03 |
| AD-159123 | 33.69 | 9.55 |
| AD-159181 | 32.21 | 14.92 |
| AD-159186 | 24.30 | 1.21 |
| AD-159187 | 46.71 | 2.58 |
| AD-159288 | 21.07 | 2.58 |
| AD-159306 | 30.47 | 5.46 |
| AD-159559 | 34.55 | 6.09 |
| AD-159344 | 14.12 | 7.20 |
| AD-159341 | 19.39 | 9.18 |
| AD-159729 | 49.48 | 4.73 |
| AD-158674 | 15.18 | 2.82 |
| AD-159604 | 23.15 | 13.21 |

TABLE 7
Modified Human/Mouse/Cyno/Rat, Mouse, Mouse/Rat, and Human/Cyno Cross-Reactive HAO1 iRNA Sequences

| | | | | SEQ | |
|---|---|---|---|---|---|
| Duplex | | SEQ ID | | ID | |
| Name | Sense Strand Sequence 5′ to 3′ | NO: | Antisense Strand Sequence 5′ to 3′ | NO: | Species |
| AD-62933 | GfsasAfuGfuGfaAfAfGfuCfaUfcGfaCfaAfL96 | 4140 | usUfsgUfcGfaUfgAfcuuUfcAfcAfuUfcsusg | 89 | Hs/Mm |
| AD-62939 | UfsusUfuCfaAfuGfGfGfuGfuCfcUfaGfgAfL96 | 4141 | usCfscUfaGfgAfcAfcccAfuUfgAfaAfasgsu | 90 | Hs/Mm |
| AD-62944 | GfsasAfaGfuCfaUfCfGfaCfaAfgAfcAfuUfL96 | 4142 | asAfsuGfuCfuUfgUfcgaUfgAfcUfuUfcsasc | 91 | Hs/Mm |
| AD-62949 | UfscsAfuCfgAfcAfAfGfaCfaUfuGfgUfgAfL96 | 4143 | usCfsaCfcAfaUfgUfcuuGfuCfgAfuGfascsu | 92 | Hs/Mm |
| AD-62954 | UfsusUfcAfaUfgGfGfUfgUfcCfuAfgGfaAfL96 | 4144 | usUfscCfuAfgGfaCfaccCfaUfuGfaAfasasg | 93 | Hs/Mm |
| AD-62959 | AfsasUfgGfgUfgUfCfCfuAfgGfaAfcCfuUfL96 | 4145 | asAfsgGfuUfcCfuAfggaCfaCfcCfaUfusgsa | 94 | Hs/Mm |
| AD-62964 | GfsasCfaGfuGfcAfCfAfaUfaUfuUfuCfcAfL96 | 4146 | usGfsgAfaAfaUfaUfuguGfcAfcUfgUfcsasg | 95 | Hs/Mm |
| AD-62969 | AfscsUfuUfuCfaAfUfGfgGfuGfuCfcUfaAfL96 | 4147 | usUfsaGfgAfcAfcCfcauUfgAfaAfaGfuscsa | 96 | Hs/Mm |
| AD-62934 | AfsasGfuCfaUfcGfAfCfaAfgAfcAfuUfgAfL96 | 4148 | usCfsaAfuGfuCfuUfgucGfaUfgAfcUfususc | 97 | Hs/Mm |
| AD-62940 | AfsusCfgAfcAfaGfAfCfaUfuGfgUfgAfgAfL96 | 4149 | usCfsuCfaCfcAfaUfgucUfuGfuCfgAfusgsa | 98 | Hs/Mm |
| AD-62945 | GfsgsGfaGfaAfaGfGfUfgUfuCfaAfgAfuAfL96 | 4150 | usAfsuCfuUfgAfaCfaccUfuUfcUfcCfcscsc | 99 | Hs/Mm |
| AD-62950 | CfsusUfuUfcAfaUfGfGfgUfgUfcCfuAfgAfL96 | 29 | usCfsuAfgGfaCfaCfccaUfuGfaAfaAfgsusc | 100 | Hs/Mm |
| AD-62955 | UfscsAfaUfgGfgUfGfUfcCfuAfgGfaAfcAfL96 | 30 | usGfsuUfcCfuAfgGfacaCfcCfaUfuGfasasa | 101 | Hs/Mm |
| AD-62960 | UfsusGfaCfuUfuUfCfAfaUfgGfgUfgUfcAfL96 | 31 | usGfsaCfaCfcCfaUfugaAfaAfgUfcAfasasa | 102 | Hs/Mm |
| AD-62965 | AfsasAfgUfcAfuCfGfAfcAfaGfaCfaUfuAfL96 | 32 | usAfsaUfgUfcUfuGfucgAfuGfaCfuUfuscsa | 103 | Hs/Mm |
| AD-62970 | CfsasGfgGfgGfaGfAfAfaGfgUfgUfuCfaAfL96 | 33 | usUfsgAfaCfaCfcUfuucUfcCfcCfcUfgsgsa | 104 | Hs/Mm |
| AD-62935 | CfsasUfuGfgUfgAfGfGfaAfaAfaUfcCfuUfL96 | 34 | asAfsgGfaUfuUfuUfccuCfaCfcAfaUfgsusc | 105 | Hs/Mm |
| AD-62941 | AfscsAfuUfgGfuGfAfGfgAfaAfaAfuCfcUfL96 | 35 | asGfsgAfuUfuUfuCfcucAfcCfaAfuGfuscsu | 106 | Hs/Mm |
| AD-62946 | AfsgsGfgGfgAfgAfAfAfgGfuGfuUfcAfaAfL96 | 36 | usUfsuGfaAfcAfcCfuuuCfuCfcCfcCfusgsg | 107 | Hs/Mm |
| AD-62951 | AfsusGfgUfgGfuAfAfUfuUfgUfgAfuUfuUfL96 | 37 | asAfsaAfuCfaCfaAfauuAfcCfaCfcAfuscsc | 108 | Hs |
| AD-62956 | GfsasCfuUfgCfaUfCfCfuGfgAfaAfuAfuAfL96 | 38 | usAfsuAfuUfuCfcAfggaUfgCfaAfgUfcscsa | 109 | Hs |
| AD-62961 | GfsgsAfaGfgGfaAfGfGfuAfgAfaGfuCfuUfL96 | 39 | asAfsgAfcUfuCfuAfccuUfcCfcUfuCfcsasc | 110 | Hs |
| AD-62966 | UfsgsUfcUfuCfuGfUfUfuAfgAfuUfuCfcUfL96 | 40 | asGfsgAfaAfuCfuAfaacAfgAfaGfaCfasgsg | 111 | Hs |
| AD-62971 | CfsusUfuGfgCfuGfUfUfuCfcAfaGfaUfcUfL96 | 41 | asGfsaUfcUfuGfgAfaacAfgCfcAfaAfgsgsa | 112 | Hs |
| AD-62936 | AfsasUfgUfgUfuUfGfGfgCfaAfcGfuCfaUfL96 | 42 | asUfsgAfcGfuUfgCfccaAfaCfaCfaUfususu | 113 | Hs |
| AD-62942 | UfsgsUfgAfcUfgUfGfGfaCfaCfcCfcUfuAfL96 | 43 | usAfsaGfgGfgUfgUfccaCfaGfuCfaCfasasa | 114 | Hs |
| AD-62947 | GfsasUfgGfgGfuGfCfCfaGfcUfaCfuAfuUfL96 | 44 | asAfsuAfgUfaGfcUfggcAfcCfcCfaUfcscsa | 115 | Hs |
| AD-62952 | GfsasAfaAfuGfuGfUfUfuGfgGfcAfaCfgUfL96 | 45 | asCfsgUfuGfcCfcAfaacAfcAfuUfuUfcsasa | 116 | Hs |
| AD-62957 | GfsgsCfuGfuUfuCfCfAfaGfaUfcUfgAfcAfL96 | 46 | usGfsuCfaGfaUfcUfuggAfaAfcAfgCfcsasa | 117 | Hs |
| AD-62962 | UfscsCfaAfcAfaAfAfUfaGfcCfaCfcCfcUfL96 | 47 | asGfsgGfgUfgGfcUfauuUfuGfuUfgGfasasa | 118 | Hs |
| AD-62967 | GfsusCfuUfcUfgUfUfUfaGfaUfuUfcCfuUfL96 | 48 | asAfsgGfaAfaUfcUfaaaCfaGfaAfgAfcsasg | 119 | Hs |
| AD-62972 | UfsgsGfaAfgGfgAfAfGfgUfaGfaAfgUfcUfL96 | 49 | asGfsaCfuUfcUfaCfcuuCfcCfuUfcCfascsa | 120 | Hs |
| AD-62937 | UfscsCfuUfuGfgCfUfGfuUfuCfcAfaGfaUfL96 | 50 | asUfscUfuGfgAfaAfcagCfcAfaAfgGfasusu | 121 | Hs |
| AD-62943 | CfsasUfcUfcUfcAfGfCfuGfgGfaUfgAfuAfL96 | 51 | usAfsuCfaUfcCfcAfgcuGfaGfaGfaUfgsgsg | 122 | Hs |
| AD-62948 | GfsgsGfgUfgCfcAfGfCfuAfcUfaUfuGfaUfL96 | 52 | asUfscAfaUfaGfuAfgcuGfgCfaCfcCfcsasu | 123 | Hs |
| AD-62953 | AfsusGfuGfuUfuGfGfGfcAfaCfgUfcAfuAfL96 | 53 | usAfsuGfaCfgUfuGfcccAfaAfcAfcAfususu | 124 | Hs |
| AD-62958 | CfsusGfuUfuAfgAfUfUfuCfcUfuAfaGfaAfL96 | 54 | usUfscUfuAfaGfgAfaauCfuAfaAfcAfgsasa | 125 | Hs |
| AD-62963 | AfsgsAfaAfgAfaAfUfGfgAfcUfuGfcAfuAfL96 | 55 | usAfsuGfcAfaGfuCfcauUfuCfuUfuCfusasg | 126 | Hs |
| AD-62968 | GfscsAfuCfcUfgGfAfAfaUfaUfaUfuAfaAfL96 | 56 | usUfsuAfaUfaUfaUfuucCfaGfgAfuGfcsasa | 127 | Hs |
| AD-62973 | CfscsUfgUfcAfgAfCfCfaUfgGfgAfaCfuAfL96 | 57 | usAfsgUfuCfcCfaUfgguCfuGfaCfaGfgscsu | 128 | Hs |
| AD-62938 | AfsasAfcAfuGfgUfGfUfgGfaUfgGfgAfuAfL96 | 58 | usAfsuCfcCfaUfcCfacaCfcAfuGfuUfusasa | 129 | Hs |
| AD-62974 | CfsusCfaGfgAfuGfAfAfaAfaUfuUfuGfaAfL96 | 59 | usUfscAfaAfaUfuUfuucAfuCfcUfgAfgsusu | 130 | Hs |
| AD-62978 | CfsasGfcAfuGfuAfUfUfaCfuUfgAfcAfaAfL96 | 60 | usUfsuGfuCfaAfgUfaauAfcAfuGfcUfgsasa | 131 | Hs |
| AD-62982 | UfsasUfgAfaCfaAfCfAfuGfcUfaAfaUfcAfL96 | 61 | usGfsaUfuUfaGfcAfuguUfgUfuCfaUfasasu | 132 | Hs |
| AD-62986 | AfsusAfuAfuCfcAfAfAfuGfuUfuUfaGfgAfL96 | 62 | usCfscUfaAfaAfcAfuuuGfgAfuAfuAfususc | 133 | Hs |
| AD-62990 | CfscsAfgAfuGfgAfAfGfcUfgUfaUfcCfaAfL96 | 63 | usUfsgGfaUfaCfaGfcuuCfcAfuCfuGfgsasa | 134 | Hs |
| AD-62994 | GfsasCfuUfuCfaUfCfCfuGfgAfaAfuAfuAfL96 | 64 | usAfsuAfuUfuCfcAfggaUfgAfaAfgUfcscsa | 135 | Hs |
| AD-62998 | CfscsCfcGfgCfuAfAfUfuUfgUfaUfcAfaUfL96 | 65 | asUfsuGfaUfaCfaAfauuAfgCfcGfgGfgsgsa | 136 | Hs |
| AD-63002 | UfsusAfaAfcAfuGfGfCfuUfgAfaUfgGfgAfL96 | 66 | usCfscCfaUfuCfaAfgccAfuGfuUfuAfascsa | 137 | Hs |
| AD-62975 | AfsasUfgUfgUfuUfAfGfaCfaAfcGfuCfaUfL96 | 67 | asUfsgAfcGfuUfgUfcuaAfaCfaCfaUfususu | 138 | Mm |
| AD-62979 | AfscsUfaAfaGfgAfAfGfaAfuUfcCfgGfuUfL96 | 68 | asAfscCfgGfaAfuUfcuuCfcUfuUfaGfusasu | 139 | Mm |
| AD-62983 | UfsasUfaUfcCfaAfAfUfgUfuUfuAfgGfaUfL96 | 69 | asUfscCfuAfaAfaCfauuUfgGfaUfaUfasusu | 140 | Mm |
| AD-62987 | GfsusGfcGfgAfaAfGfGfcAfcUfgAfuGfuUfL96 | 70 | asAfscAfuCfaGfuGfccuUfuCfcGfcAfcsasc | 141 | Mm |
| AD-62991 | UfsasAfaAfcAfgUfGfGfuUfcUfuAfaAfuUfL96 | 71 | asAfsuUfuAfaGfaAfccaCfuGfuUfuUfasasa | 142 | Mm |
| AD-62995 | AfsusGfaAfaAfaUfUfUfuGfaAfaCfcAfgUfL96 | 72 | asCfsuGfgUfuUfcAfaaaUfuUfuUfcAfuscsc | 143 | Mm |
| AD-62999 | AfsasCfaAfaAfuAfGfCfaAfuCfcCfuUfuUfL96 | 73 | asAfsaAfgGfgAfuUfgcuAfuUfuUfgUfusgsg | 144 | Mm |
| AD-63003 | CfsusGfaAfaCfaGfAfUfcUfgUfcGfaCfuUfL96 | 74 | asAfsgUfcGfaCfaGfaucUfgUfuUfcAfgscsa | 145 | Mm |
| AD-62976 | UfsusGfuUfgCfaAfAfGfgGfcAfuUfuUfgAfL96 | 75 | usCfsaAfaAfuGfcCfcuuUfgCfaAfcAfasusu | 146 | Mm |
| AD-62980 | CfsusCfaUfuGfuUfUfAfuUfaAfcCfuGfuAfL96 | 76 | usAfscAfgGfuUfaAfuaaAfcAfaUfgAfgsasu | 147 | Mm |
| AD-62984 | CfsasAfcAfaAfaUfAfGfcAfaUfcCfcUfuUfL96 | 77 | asAfsaGfgGfaUfuGfcuaUfuUfuGfuUfgsgsa | 148 | Mm |
| AD-62992 | CfsasUfuGfuUfuAfUfUfaAfcCfuGfuAfuUfL96 | 78 | asAfsuAfcAfgGfuUfaauAfaAfcAfaUfgsasg | 149 | Mm |
| AD-62996 | UfsasUfcAfgCfuGfGfGfaAfgAfuAfuCfaAfL96 | 79 | usUfsgAfuAfuCfuUfcccAfgCfuGfaUfasgsa | 150 | Mm |
| AD-63000 | UfsgsUfcCfuAfgGfAfAfcCfuUfuUfaGfaAfL96 | 80 | usUfscUfaAfaAfgGfuucCfuAfgGfaCfascsc | 151 | Mm |
| AD-63004 | UfscsCfaAfcAfaAfAfUfaGfcAfaUfcCfcUfL96 | 81 | asGfsgGfaUfuGfcUfauuUfuGfuUfgGfasasa | 152 | Mm |
| AD-62977 | GfsgsUfgUfgCfgGfAfAfaGfgCfaCfuGfaUfL96 | 82 | asUfscAfgUfgCfcUfuucCfgCfaCfaCfcscsc | 153 | Mm |
| AD-62981 | UfsusGfaAfaCfcAfGfUfaCfuUfuAfuCfaUfL96 | 83 | asUfsgAfuAfaAfgUfacuGfgUfuUfcAfasasa | 154 | Mm |
| AD-62985 | UfsasCfuUfcCfaAfAfGfuCfuAfuAfuAfuAfL96 | 84 | usAfsuAfuAfuAfgAfcuuUfgGfaAfgUfascsu | 155 | Mm |
| AD-62989 | UfscsCfuAfgGfaAfCfCfuUfuUfaGfaAfaUfL96 | 85 | asUfsuUfcUfaAfaAfgguUfcCfuAfgGfascsa | 156 | Mm |
| AD-62993 | CfsusCfcUfgAfgGfAfAfaAfuUfuUfgGfaAfL96 | 86 | usUfscCfaAfaAfuUfuucCfuCfaGfgAfgsasa | 157 | Mm |
| AD-62997 | GfscsUfcCfgGfaAfUfGfuUfgCfuGfaAfaUfL96 | 87 | asUfsuUfcAfgCfaAfcauUfcCfgGfaGfcsasu | 158 | Mm |
| AD-63001 | GfsusGfuUfuGfuGfGfGfgAfgAfcCfaAfuAfL96 | 88 | usAfsuUfgGfuCfuCfcccAfcAfaAfcAfcsasg | 159 | Mm |

TABLE 8
Additional Modified Human/Mouse/Cyno/Rat, Human/Mouse/
Rat, Human/Mouse/Cyno, Mouse, Mouse/Rat, and Human/
Cyno Cross-Reactive HAO1 iRNA Sequences

| Duplex | | |
|---|---|---|
| Name | Sense Strand Sequence 5′ to 3′ | SEQ ID NO: |
| AD-62933.2 | GfsasAfuGfuGfaAfAfGfuCfaUfcGfaCfaAfL96 | 4140 |
| AD-62939.2 | UfsusUfuCfaAfuGfGfGfuGfuCfcUfaGfgAfL96 | 4141 |
| AD-62944.2 | GfsasAfaGfuCfaUfCfGfaCfaAfgAfcAfuUfL96 | 4142 |
| AD-62949.2 | UfscsAfuCfgAfcAfAfGfaCfaUfuGfgUfgAfL96 | 4143 |
| AD-62954.2 | UfsusUfcAfaUfgGfGfUfgUfcCfuAfgGfaAfL96 | 4144 |
| AD-62959.2 | AfsasUfgGfgUfgUfCfCfuAfgGfaAfcCfuUfL96 | 4145 |
| AD-62964.2 | GfsasCfaGfuGfcAfCfAfaUfaUfuUfuCfcAfL96 | 4146 |
| AD-62969.2 | AfscsUfuUfuCfaAfUfGfgGfuGfuCfcUfaAfL96 | 4147 |
| AD-62934.2 | AfsasGfuCfaUfcGfAfCfaAfgAfcAfuUfgAfL96 | 4148 |
| AD-62940.2 | AfsusCfgAfcAfaGfAfCfaUfuGfgUfgAfgAfL96 | 4149 |
| AD-62945.2 | GfsgsGfaGfaAfaGfGfUfgUfuCfaAfgAfuAfL96 | 4150 |
| AD-62950.2 | CfsusUfuUfcAfaUfGfGfgUfgUfcCfuAfgAfL96 | 29 |
| AD-62955.2 | UfscsAfaUfgGfgUfGfUfcCfuAfgGfaAfcAfL96 | 30 |
| AD-62960.2 | UfsusGfaCfuUfuUfCfAfaUfgGfgUfgUfcAfL96 | 31 |
| AD-62965.2 | AfsasAfgUfcAfuCfGfAfcAfaGfaCfaUfuAfL96 | 32 |
| AD-62970.2 | CfsasGfgGfgGfaGfAfAfaGfgUfgUfuCfaAfL96 | 33 |
| AD-62935.2 | CfsasUfuGfgUfgAfGfGfaAfaAfaUfcCfuUfL96 | 34 |
| AD-62941.2 | AfscsAfuUfgGfuGfAfGfgAfaAfaAfuCfcUfL96 | 35 |
| AD-62946.2 | AfsgsGfgGfgAfgAfAfAfgGfuGfuUfcAfaAfL96 | 36 |
| AD-62951.2 | AfsusGfgUfgGfuAfAfUfuUfgUfgAfuUfuUfL96 | 37 |
| AD-62956.2 | GfsasCfuUfgCfaUfCfCfuGfgAfaAfuAfuAfL96 | 38 |
| AD-62961.2 | GfsgsAfaGfgGfaAfGfGfuAfgAfaGfuCfuUfL96 | 39 |
| AD-62966.2 | UfsgsUfcUfuCfuGfUfUfuAfgAfuUfuCfcUfL96 | 40 |
| AD-62971.2 | CfsusUfuGfgCfuGfUfUfuCfcAfaGfaUfcUfL96 | 41 |
| AD-62936.2 | AfsasUfgUfgUfuUfGfGfgCfaAfcGfuCfaUfL96 | 42 |
| AD-62942.2 | UfsgsUfgAfcUfgUfGfGfaCfaCfcCfcUfuAfL96 | 43 |
| AD-62947.2 | GfsasUfgGfgGfuGfCfCfaGfcUfaCfuAfuUfL96 | 44 |
| AD-62952.2 | GfsasAfaAfuGfuGfUfUfuGfgGfcAfaCfgUfL96 | 45 |
| AD-62957.2 | GfsgsCfuGfuUfuCfCfAfaGfaUfcUfgAfcAfL96 | 46 |
| AD-62962.2 | UfscsCfaAfcAfaAfAfUfaGfcCfaCfcCfcUfL96 | 47 |
| AD-62967.2 | GfsusCfuUfcUfgUfUfUfaGfaUfuUfcCfuUfL96 | 48 |
| AD-62972.2 | UfsgsGfaAfgGfgAfAfGfgUfaGfaAfgUfcUfL96 | 49 |
| AD-62937.2 | UfscsCfuUfuGfgCfUfGfuUfuCfcAfaGfaUfL96 | 50 |
| AD-62943.2 | CfsasUfcUfcUfcAfGfCfuGfgGfaUfgAfuAfL96 | 51 |
| AD-62948.2 | GfsgsGfgUfgCfcAfGfCfuAfcUfaUfuGfaUfL96 | 52 |
| AD-62953.2 | AfsusGfuGfuUfuGfGfGfcAfaCfgUfcAfuAfL96 | 53 |
| AD-62958.2 | CfsusGfuUfuAfgAfUfUfuCfcUfuAfaGfaAfL96 | 54 |
| AD-62963.2 | AfsgsAfaAfgAfaAfUfGfgAfcUfuGfcAfuAfL96 | 55 |
| AD-62968.2 | GfscsAfuCfcUfgGfAfAfaUfaUfaUfuAfaAfL96 | 56 |
| AD-62973.2 | CfscsUfgUfcAfgAfCfCfaUfgGfgAfaCfuAfL96 | 57 |
| AD-62938.2 | AfsasAfcAfuGfgUfGfUfgGfaUfgGfgAfuAfL96 | 58 |
| AD-62974.2 | CfsusCfaGfgAfuGfAfAfaAfaUfuUfuGfaAfL96 | 59 |
| AD-62978.2 | CfsasGfcAfuGfuAfUfUfaCfuUfgAfcAfaAfL96 | 60 |
| AD-62982.2 | UfsasUfgAfaCfaAfCfAfuGfcUfaAfaUfcAfL96 | 61 |
| AD-62986.2 | AfsusAfuAfuCfcAfAfAfuGfuUfuUfaGfgAfL96 | 62 |
| AD-62990.2 | CfscsAfgAfuGfgAfAfGfcUfgUfaUfcCfaAfL96 | 63 |
| AD-62994.2 | GfsasCfuUfuCfaUfCfCfuGfgAfaAfuAfuAfL96 | 64 |
| AD-62998.2 | CfscsCfcGfgCfuAfAfUfuUfgUfaUfcAfaUfL96 | 65 |
| AD-63002.2 | UfsusAfaAfcAfuGfGfCfuUfgAfaUfgGfgAfL96 | 66 |
| AD-62975.2 | AfsasUfgUfgUfuUfAfGfaCfaAfcGfuCfaUfL96 | 67 |
| AD-62979.2 | AfscsUfaAfaGfgAfAfGfaAfuUfcCfgGfuUfL96 | 68 |
| AD-62983.2 | UfsasUfaUfcCfaAfAfUfgUfuUfuAfgGfaUfL96 | 69 |
| AD-62987.2 | GfsusGfcGfgAfaAfGfGfcAfcUfgAfuGfuUfL96 | 70 |
| AD-62991.2 | UfsasAfaAfcAfgUfGfGfuUfcUfuAfaAfuUfL96 | 71 |
| AD-62995.2 | AfsusGfaAfaAfaUfUfUfuGfaAfaCfcAfgUfL96 | 72 |
| AD-62999.2 | AfsasCfaAfaAfuAfGfCfaAfuCfcCfuUfuUfL96 | 73 |
| AD-63003.2 | CfsusGfaAfaCfaGfAfUfcUfgUfcGfaCfuUfL96 | 74 |
| AD-62976.2 | UfsusGfuUfgCfaAfAfGfgGfcAfuUfuUfgAfL96 | 75 |
| AD-62980.2 | CfsusCfaUfuGfuUfUfAfuUfaAfcCfuGfuAfL96 | 76 |
| AD-62984.2 | CfsasAfcAfaAfaUfAfGfcAfaUfcCfcUfuUfL96 | 77 |
| AD-62992.2 | CfsasUfuGfuUfuAfUfUfaAfcCfuGfuAfuUfL96 | 78 |
| AD-62996.2 | UfsasUfcAfgCfuGfGfGfaAfgAfuAfuCfaAfL96 | 79 |
| AD-63000.2 | UfsgsUfcCfuAfgGfAfAfcCfuUfuUfaGfaAfL96 | 80 |
| AD-63004.2 | UfscsCfaAfcAfaAfAfUfaGfcAfaUfcCfcUfL96 | 81 |
| AD-62977.2 | GfsgsUfgUfgCfgGfAfAfaGfgCfaCfuGfaUfL96 | 82 |
| AD-62981.2 | UfsusGfaAfaCfcAfGfUfaCfuUfuAfuCfaUfL96 | 83 |
| AD-62985.2 | UfsasCfuUfcCfaAfAfGfuCfuAfuAfuAfuAfL96 | 84 |
| AD-62989.2 | UfscsCfuAfgGfaAfCfCfuUfuUfaGfaAfaUfL96 | 85 |
| AD-62993.2 | CfsusCfcUfgAfgGfAfAfaAfuUfuUfgGfaAfL96 | 86 |
| AD-62997.2 | GfscsUfcCfgGfaAfUfGfuUfgCfuGfaAfaUfL96 | 87 |
| AD-63001.2 | GfsusGfuUfuGfuGfGfGfgAfgAfcCfaAfuAfL96 | 88 |
| AD-62933.1 | GfsasAfuGfuGfaAfAfGfuCfaUfcGfaCfaAfL96 | 160 |
| AD-65630.1 | Y44gsasAfuGfuGfaAfAfGfuCfaUfcGfaCfaAfL96 | 161 |
| AD-65636.1 | gsasauguGfaAfAfGfucauCfgacaaL96 | 162 |
| AD-65642.1 | gsasauguGfaAfAfGfucaucgacaaL96 | 163 |
| AD-65647.1 | gsasauguGfaaAfGfucaucgacaaL96 | 164 |
| AD-65652.1 | gsasauguGfaaaGfucaucGfacaaL96 | 165 |
| AD-65657.1 | gsasaugugaaaGfucaucGfacaaL96 | 166 |
| AD-65662.1 | gsasauguGfaaaGfucaucgacaaL96 | 167 |
| AD-65625.1 | AfsusGfuGfaAfAfGfuCfaUfcGfaCfaAfL96 | 168 |
| AD-65631.1 | asusguGfaAfAfGfucaucgacaaL96 | 169 |
| AD-65637.1 | GfsasAfuGfuGfaAfAfGfuCfaUfcGfaCfaAfL96 | 170 |
| AD-65643.1 | GfsasAfuGfuGfaAfAfGfuCfaUfcGfaCfaAfL96 | 171 |
| AD-65648.1 | GfsasAfuGfuGfaAfAfGfuCfaUfcGfaCfaAfL96 | 172 |
| AD-65653.1 | GfsasAfuGfuGfaAfAfGfuCfaUfcGfaCfaAfL96 | 173 |
| AD-65658.1 | GfsasAfuGfuGfaAfAfGfuCfaUfcGfaCfaAfL96 | 174 |
| AD-65663.1 | gsasauguGfaAfAfGfucaucgacaaL96 | 175 |
| AD-65626.1 | gsasauguGfaAfAfGfucaucgacaaL96 | 176 |
| AD-65638.1 | gsasauguGfaaAfGfucaucgacaaL96 | 177 |
| AD-65644.1 | gsasauguGfaaAfGfucaucgacaaL96 | 178 |
| AD-65649.1 | gsasauguGfaaAfGfucaucgacaaL96 | 179 |
| AD-65654.1 | gsasaugugaaagucau(Cgn)gacaaL96 | 180 |
| AD-65659.1 | gsasaugdTgaaagucau(Cgn)gacaaL96 | 181 |
| AD-65627.1 | gsasaudGugaaadGucau(Cgn)gacaaL96 | 182 |
| AD-65633.1 | gsasaugdTgaaadGucau(Cgn)gacaaL96 | 183 |
| AD-65639.1 | gsasaugudGaaadGucau(Cgn)gacaaL96 | 184 |
| AD-65645.1 | gsasaugugaaadGucaucdGacaaL96 | 185 |
| AD-65650.1 | gsasaugugaaadGucaucdTacaaL96 | 186 |
| AD-65655.1 | gsasaugugaaadGucaucY34acaaL96 | 187 |
| AD-65660.1 | gsasaugugaaadGucadTcdTacaaL96 | 188 |
| AD-65665.1 | gsasaugugaaadGucaucdGadCaaL96 | 189 |
| AD-65628.1 | gsasaugugaaadGucaucdTadCaaL96 | 190 |
| AD-65634.1 | gsasaugugaaadGucaucY34adCaaL96 | 191 |
| AD-65646.1 | GfsasAfuGfuGfaAfAfGfuCfaUfcGfaCfaAfL96 | 192 |
| AD-65656.1 | GfsasAfuGfuGfaAfAfGfuCfaUfcGfaCfaAfL96 | 193 |
| AD-65661.1 | GfsasAfuGfuGfaAfAfGfuCfaUfcGfaCfaAfL96 | 194 |
| AD-65666.1 | GfsasAfuGfuGfaAfAfGfuCfaUfcGfaCfaAfL96 | 195 |
| AD-65629.1 | GfsasAfuGfuGfaAfAfGfuCfaUfcGfaCfaAfL96 | 196 |
| AD-65635.1 | GfsasAfuGfuGfaAfAfGfuCfaUfcGfaCfaAfL96 | 197 |
| AD-65641.1 | gsasaugugaaadGucau(Cgn)gacaaL96 | 198 |
| AD-62994.1 | GfsasCfuUfuCfaUfCfCfuGfgAfaAfuAfuAfL96 | 199 |
| AD-65595.1 | gsascuuuCfaUfCfCfuggaAfauauaL96 | 200 |
| AD-65600.1 | gsascuuuCfaUfCfCfuggaaauauaL96 | 201 |
| AD-65610.1 | gsascuuuCfaucCfuggaaAfuauaL96 | 202 |
| AD-65615.1 | gsascuuucaucCfuggaaAfuauaL96 | 203 |
| AD-65620.1 | gsascuuuCfaucCfuggaaauauaL96 | 204 |
| AD-65584.1 | CfsusUfuCfaUfCfCfuGfgAfaAfuAfuAfL96 | 205 |
| AD-65590.1 | csusuuCfaUfCfCfuggaaauauaL96 | 206 |
| AD-65596.1 | GfsasCfuUfuCfaUfCfCfuGfgAfaAfuAfuAfL96 | 207 |
| AD-65601.1 | GfsasCfuUfuCfaUfCfCfuGfgAfaAfuAfuAfL96 | 208 |
| AD-65606.1 | GfsasCfuUfuCfaUfCfCfuGfgAfaAfuAfuAfL96 | 209 |
| AD-65611.1 | GfsasCfuUfuCfaUfCfCfuGfgAfaAfuAfuAfL96 | 210 |
| AD-65616.1 | GfsasCfuUfuCfaUfCfCfuGfgAfaAfuAfuAfL96 | 211 |
| AD-65621.1 | gsascuuuCfaUfCfCfuggaaauauaL96 | 212 |
| AD-65585.1 | gsascuuuCfaUfCfCfuggaaauauaL96 | 213 |
| AD-65591.1 | gsascuuuCfaUfCfCfuggaaauauaL96 | 214 |
| AD-65597.1 | gsascuuuCfauCfCfuggaaauauaL96 | 215 |
| AD-65602.1 | gsascuuuCfauCfCfuggaaauauaL96 | 216 |
| AD-65607.1 | gsascuuuCfauCfCfuggaaauauaL96 | 217 |
| AD-65612.1 | gsascuuucauccuggaa(Agn)uauaL96 | 218 |
| AD-65622.1 | gsascuuucaucdCuggaa(Agn)uauaL96 | 219 |
| AD-65586.1 | gsascudTucaucdCuggaa(Agn)uauaL96 | 220 |
| AD-65592.1 | gsascuudTcaucdCuggaa(Agn)uauaL96 | 221 |
| AD-65598.1 | gsascuuudCaucdCuggaa(Agn)uauaL96 | 222 |
| AD-65603.1 | gsascuuucaucdCuggaadAuauaL96 | 223 |
| AD-65608.1 | gsascuuucaucdCuggaadTuauaL96 | 224 |
| AD-65613.1 | gsascuuucaucdCuggaaY34uauaL96 | 225 |
| AD-65618.1 | gsascuuucaucdCuggdAadTuauaL96 | 226 |
| AD-65623.1 | gsascuuucaucdCuggaadTudAuaL96 | 227 |
| AD-65587.1 | gsascuuucaucdCuggaa(Agn)udAuaL96 | 228 |
| AD-65593.1 | gsascuudTcaucdCuggaadAudAuaL96 | 229 |
| AD-65599.1 | GfsasCfuUfuCfaUfCfCfuGfgAfaAfuAfuAfL96 | 230 |
| AD-65604.1 | GfsasCfuUfuCfaUfCfCfuGfgAfaAfuAfuAfL96 | 231 |
| AD-65609.1 | GfsasCfuUfuCfaUfCfCfuGfgAfaAfuAfuAfL96 | 232 |
| AD-65614.1 | GfsasCfuUfuCfaUfCfCfuGfgAfaAfuAfuAfL96 | 233 |
| AD-65619.1 | GfsasCfuUfuCfaUfCfCfuGfgAfaAfuAfuAfL96 | 234 |
| AD-65624.1 | GfsasCfuUfuCfaUfCfCfuGfgAfaAfuAfuAfL96 | 235 |
| AD-65588.1 | GfsasCfuUfuCfaUfCfCfuGfgAfaAfuAfuAfL96 | 236 |
| AD-65594.1 | gsascuuucaucdCuggaa(Agn)uauaL96 | 237 |
| AD-68309.1 | asgsaaagGfuGfUfUfcaagaugucaL96 | 238 |
| AD-68303.1 | csasuccuGfgAfAfAfuauauuaacuL96 | 239 |
| AD-65626.5 | gsasauguGfaAfAfGfucaucgacaaL96 | 240 |
| AD-68295.1 | asgsugcaCfaAfUfAfuuuucccauaL96 | 241 |
| AD-68273.1 | gsasaaguCfaUfCfGfacaagacauuL96 | 242 |
| AD-68297.1 | asasugugAfaAfGfUfcaucgacaaaL96 | 243 |
| AD-68287.1 | csusggaaAfuAfUfAfuuaacuguuaL96 | 244 |
| AD-68300.1 | asusuuucCfcAfUfCfuguauuauuuL96 | 245 |
| AD-68306.1 | usgsucguUfcUfUfUfuccaacaaaaL96 | 246 |
| AD-68292.1 | asusccugGfaAfAfUfauauuaacuaL96 | 247 |
| AD-68298.1 | gscsauuuUfgAfGfAfggugaugauaL96 | 248 |
| AD-68277.1 | csasggggGfaGfAfAfagguguucaaL96 | 249 |
| AD-68289.1 | gsgsaaauAfuAfUfUfaacuguuaaaL96 | 250 |
| AD-68272.1 | csasuuggUfgAfGfGfaaaaauccuuL96 | 251 |
| AD-68282.1 | gsgsgagaAfaGfGfUfguucaagauaL96 | 252 |
| AD-68285.1 | gsgscauuUfuGfAfGfaggugaugauL96 | 253 |
| AD-68290.1 | usascaaaGfgGfUfGfucguucuuuuL96 | 254 |
| AD-68296.1 | usgsggauCfuUfGfGfugucgaaucaL96 | 255 |
| AD-68288.1 | csusgacaGfuGfCfAfcaauauuuuaL96 | 256 |
| AD-68299.1 | csasgugcAfcAfAfUfauuuucccauL96 | 257 |
| AD-68275.1 | ascsuuuuCfaAfUfGfgguguccuaaL96 | 258 |
| AD-68274.1 | ascsauugGfuGfAfGfgaaaaauccuL96 | 259 |
| AD-68294.1 | ususgcuuUfuGfAfCfuuuucaaugaL96 | 260 |
| AD-68302.1 | csasuuuuGfaGfAfGfgugaugaugaL96 | 261 |
| AD-68279.1 | ususgacuUfuUfCfAfaugggugucaL96 | 262 |
| AD-68304.1 | csgsacuuCfuGfUfUfuuaggacagaL96 | 263 |
| AD-68286.1 | csuscugaGfuGfGfGfugccagaauaL96 | 264 |
| AD-68291.1 | gsgsgugcCfaGfAfAfugugaaaguaL96 | 265 |
| AD-68283.1 | uscsaaugGfgUfGfUfccuaggaacaL96 | 266 |
| AD-68280.1 | asasagucAfuCfGfAfcaagacauuaL96 | 267 |
| AD-68293.1 | asusuuugAfgAfGfGfugaugaugcaL96 | 268 |
| AD-68276.1 | asuscgacAfaGfAfCfauuggugagaL96 | 269 |
| AD-68308.1 | gsgsugccAfgAfAfUfgugaaagucaL96 | 270 |
| AD-68278.1 | gsascaguGfcAfCfAfauauuuuccaL96 | 271 |
| AD-68307.1 | ascsaaagAfgAfCfAfcugugcagaaL96 | 272 |
| AD-68284.1 | ususuucaAfuGfGfGfuguccuaggaL96 | 273 |
| AD-68301.1 | cscsguuuCfcAfAfGfaucugacaguL96 | 274 |
| AD-68281.1 | asgsggggAfgAfAfAfgguguucaaaL96 | 275 |
| AD-68305.1 | asgsucauCfgAfCfAfagacauugguL96 | 276 |

| Duplex | | SEQ | |
|---|---|---|---|
| Name | Antisense Strand Sequence 5′ to 3′ | ID NO: | Species |
| AD-62933.2 | usUfsgUfcGfaUfgAfcuuUfcAfcAfuUfcsusg | 89 | Hs/Mm |
| AD-62939.2 | usCfscUfaGfgAfcAfcccAfuUfgAfaAfasgsu | 90 | Hs/Mm |
| AD-62944.2 | asAfsuGfuCfuUfgUfcgaUfgAfcUfuUfcsasc | 91 | Hs/Mm |
| AD-62949.2 | usCfsaCfcAfaUfgUfcuuGfuCfgAfuGfascsu | 92 | Hs/Mm |
| AD-62954.2 | usUfscCfuAfgGfaCfaccCfaUfuGfaAfasasg | 93 | Hs/Mm |
| AD-62959.2 | asAfsgGfuUfcCfuAfggaCfaCfcCfaUfusgsa | 94 | Hs/Mm |
| AD-62964.2 | usGfsgAfaAfaUfaUfuguGfcAfcUfgUfcsasg | 95 | Hs/Mm |
| AD-62969.2 | usUfsaGfgAfcAfcCfcauUfgAfaAfaGfuscsa | 96 | Hs/Mm |
| AD-62934.2 | usCfsaAfuGfuCfuUfgucGfaUfgAfcUfususc | 97 | Hs/Mm |
| AD-62940.2 | usCfsuCfaCfcAfaUfgucUfuGfuCfgAfusgsa | 98 | Hs/Mm |
| AD-62945.2 | usAfsuCfuUfgAfaCfaccUfuUfcUfcCfcscsc | 99 | Hs/Mm |
| AD-62950.2 | usCfsuAfgGfaCfaCfccaUfuGfaAfaAfgsusc | 100 | Hs/Mm |
| AD-62955.2 | usGfsuUfcCfuAfgGfacaCfcCfaUfuGfasasa | 101 | Hs/Mm |
| AD-62960.2 | usGfsaCfaCfcCfaUfugaAfaAfgUfcAfasasa | 102 | Hs/Mm |
| AD-62965.2 | usAfsaUfgUfcUfuGfucgAfuGfaCfuUfuscsa | 103 | Hs/Mm |
| AD-62970.2 | usUfsgAfaCfaCfcUfuucUfcCfcCfcUfgsgsa | 104 | Hs/Mm |
| AD-62935.2 | asAfsgGfaUfuUfuUfccuCfaCfcAfaUfgsusc | 105 | Hs/Mm |
| AD-62941.2 | asGfsgAfuUfuUfuCfcucAfcCfaAfuGfuscsu | 106 | Hs/Mm |
| AD-62946.2 | usUfsuGfaAfcAfcCfuuuCfuCfcCfcCfusgsg | 107 | Hs/Mm |
| AD-62951.2 | asAfsaAfuCfaCfaAfauuAfcCfaCfcAfuscsc | 108 | Hs |
| AD-62956.2 | usAfsuAfuUfuCfcAfggaUfgCfaAfgUfcscsa | 109 | Hs |
| AD-62961.2 | asAfsgAfcUfuCfuAfccuUfcCfcUfuCfcsasc | 110 | Hs |
| AD-62966.2 | asGfsgAfaAfuCfuAfaacAfgAfaGfaCfasgsg | 111 | Hs |
| AD-62971.2 | asGfsaUfcUfuGfgAfaacAfgCfcAfaAfgsgsa | 112 | Hs |
| AD-62936.2 | asUfsgAfcGfuUfgCfccaAfaCfaCfaUfususu | 113 | Hs |
| AD-62942.2 | usAfsaGfgGfgUfgUfccaCfaGfuCfaCfasasa | 114 | Hs |
| AD-62947.2 | asAfsuAfgUfaGfcUfggcAfcCfcCfaUfcscsa | 115 | Hs |
| AD-62952.2 | asCfsgUfuGfcCfcAfaacAfcAfuUfuUfcsasa | 116 | Hs |
| AD-62957.2 | usGfsuCfaGfaUfcUfuggAfaAfcAfgCfcsasa | 117 | Hs |
| AD-62962.2 | asGfsgGfgUfgGfcUfauuUfuGfuUfgGfasasa | 118 | Hs |
| AD-62967.2 | asAfsgGfaAfaUfcUfaaaCfaGfaAfgAfcsasg | 119 | Hs |
| AD-62972.2 | asGfsaCfuUfcUfaCfcuuCfcCfuUfcCfascsa | 120 | Hs |
| AD-62937.2 | asUfscUfuGfgAfaAfcagCfcAfaAfgGfasusu | 121 | Hs |
| AD-62943.2 | usAfsuCfaUfcCfcAfgcuGfaGfaGfaUfgsgsg | 122 | Hs |
| AD-62948.2 | asUfscAfaUfaGfuAfgcuGfgCfaCfcCfcsasu | 123 | Hs |
| AD-62953.2 | usAfsuGfaCfgUfuGfcccAfaAfcAfcAfususu | 124 | Hs |
| AD-62958.2 | usUfscUfuAfaGfgAfaauCfuAfaAfcAfgsasa | 125 | Hs |
| AD-62963.2 | usAfsuGfcAfaGfuCfcauUfuCfuUfuCfusasg | 126 | Hs |
| AD-62968.2 | usUfsuAfaUfaUfaUfuucCfaGfgAfuGfcsasa | 127 | Hs |
| AD-62973.2 | usAfsgUfuCfcCfaUfgguCfuGfaCfaGfgscsu | 128 | Hs |
| AD-62938.2 | usAfsuCfcCfaUfcCfacaCfcAfuGfuUfusasa | 129 | Hs |
| AD-62974.2 | usUfscAfaAfaUfuUfuucAfuCfcUfgAfgsusu | 130 | Hs |
| AD-62978.2 | usUfsuGfuCfaAfgUfaauAfcAfuGfcUfgsasa | 131 | Hs |
| AD-62982.2 | usGfsaUfuUfaGfcAfuguUfgUfuCfaUfasasu | 132 | Hs |
| AD-62986.2 | usCfscUfaAfaAfcAfuuuGfgAfuAfuAfususc | 133 | Hs |
| AD-62990.2 | usUfsgGfaUfaCfaGfcuuCfcAfuCfuGfgsasa | 134 | Hs |
| AD-62994.2 | usAfsuAfuUfuCfcAfggaUfgAfaAfgUfcscsa | 135 | Hs |
| AD-62998.2 | asUfsuGfaUfaCfaAfauuAfgCfcGfgGfgsgsa | 136 | Hs |
| AD-63002.2 | usCfscCfaUfuCfaAfgccAfuGfuUfuAfascsa | 137 | Hs |
| AD-62975.2 | asUfsgAfcGfuUfgUfcuaAfaCfaCfaUfususu | 138 | Mm |
| AD-62979.2 | asAfscCfgGfaAfuUfcuuCfcUfuUfaGfusasu | 139 | Mm |
| AD-62983.2 | asUfscCfuAfaAfaCfauuUfgGfaUfaUfasusu | 140 | Mm |
| AD-62987.2 | asAfscAfuCfaGfuGfccuUfuCfcGfcAfcsasc | 141 | Mm |
| AD-62991.2 | asAfsuUfuAfaGfaAfccaCfuGfuUfuUfasasa | 142 | Mm |
| AD-62995.2 | asCfsuGfgUfuUfcAfaaaUfuUfuUfcAfuscsc | 143 | Mm |
| AD-62999.2 | asAfsaAfgGfgAfuUfgcuAfuUfuUfgUfusgsg | 144 | Mm |
| AD-63003.2 | asAfsgUfcGfaCfaGfaucUfgUfuUfcAfgscsa | 145 | Mm |
| AD-62976.2 | usCfsaAfaAfuGfcCfcuuUfgCfaAfcAfasusu | 146 | Mm |
| AD-62980.2 | usAfscAfgGfuUfaAfuaaAfcAfaUfgAfgsasu | 147 | Mm |
| AD-62984.2 | asAfsaGfgGfaUfuGfcuaUfuUfuGfuUfgsgsa | 148 | Mm |
| AD-62992.2 | asAfsuAfcAfgGfuUfaauAfaAfcAfaUfgsasg | 149 | Mm |
| AD-62996.2 | usUfsgAfuAfuCfuUfcccAfgCfuGfaUfasgsa | 150 | Mm |
| AD-63000.2 | usUfscUfaAfaAfgGfuucCfuAfgGfaCfascsc | 151 | Mm |
| AD-63004.2 | asGfsgGfaUfuGfcUfauuUfuGfuUfgGfasasa | 152 | Mm |
| AD-62977.2 | asUfscAfgUfgCfcUfuucCfgCfaCfaCfcscsc | 153 | Mm |
| AD-62981.2 | asUfsgAfuAfaAfgUfacuGfgUfuUfcAfasasa | 154 | Mm |
| AD-62985.2 | usAfsuAfuAfuAfgAfcuuUfgGfaAfgUfascsu | 155 | Mm |
| AD-62989.2 | asUfsuUfcUfaAfaAfgguUfcCfuAfgGfascsa | 156 | Mm |
| AD-62993.2 | usUfscCfaAfaAfuUfuucCfuCfaGfgAfgsasa | 157 | Mm |
| AD-62997.2 | asUfsuUfcAfgCfaAfcauUfcCfgGfaGfcsasu | 158 | Mm |
| AD-63001.2 | usAfsuUfgGfuCfuCfcccAfcAfaAfcAfcsasg | 159 | Mm |
| AD-62933.1 | usUfsgUfcGfaUfgAfcuuUfcAfcAfuUfcsusg | 277 | |
| AD-65630.1 | PusUfsgUfcGfaUfgAfcuuUfcAfcAfuUfcsusg | 278 | |
| AD-65636.1 | usUfsgUfcGfaUfgAfcuuUfcAfcAfuUfcsusg | 279 | |
| AD-65642.1 | usUfsgUfcGfaUfgAfcuuUfcAfcAfuUfcsusg | 280 | |
| AD-65647.1 | usUfsgUfcGfaUfgAfcuuUfcAfcAfuUfcsusg | 281 | |
| AD-65652.1 | usUfsgUfcGfaUfgAfcuuUfcAfcAfuUfcsusg | 282 | |
| AD-65657.1 | usUfsgUfcGfaUfgAfcuuUfcAfcAfuUfcsusg | 283 | |
| AD-65662.1 | usUfsgUfcGfaUfgAfcuuUfcAfcAfuUfcsusg | 284 | |
| AD-65625.1 | usUfsgUfcGfaUfgAfcuuUfcAfcAfususc | 285 | |
| AD-65631.1 | usUfsgucGfaugacuuUfcAfcaususc | 286 | |
| AD-65637.1 | usUfsgucGfaUfgAfcuuUfcAfcauucsusg | 287 | |
| AD-65643.1 | usUfsgucGfaUfGfacuuUfcAfcauucsusg | 288 | |
| AD-65648.1 | usUfsgucGfaugacuuUfcAfcauucsusg | 289 | |
| AD-65653.1 | usUfsgucGfaugacuuUfcacauucsusg | 290 | |
| AD-65658.1 | usUfsgucgaugacuuUfcacauucsusg | 291 | |
| AD-65663.1 | usUfsgucGfaUfgAfcuuUfcAfcauucsusg | 292 | |
| AD-65626.1 | usUfsgucGfaUfGfacuuUfcAfcauucsusg | 293 | |
| AD-65638.1 | usUfsgucGfaUfgAfcuuUfcAfcauucsusg | 294 | |
| AD-65644.1 | usUfsgucGfaUfGfacuuUfcAfcauucsusg | 295 | |
| AD-65649.1 | usUfsgucGfaugacuuUfcAfcauucsusg | 296 | |
| AD-65654.1 | usUfsgUfcGfaUfgAfcuuUfcAfcAfuUfcsusg | 297 | |
| AD-65659.1 | usUfsgUfcGfaUfgAfcuuUfcAfcAfuUfcsusg | 298 | |
| AD-65627.1 | usUfsgUfcGfaUfgAfcuuUfcAfcAfuUfcsusg | 299 | |
| AD-65633.1 | usUfsgUfcGfaUfgAfcuuUfcAfcAfuUfcsusg | 300 | |
| AD-65639.1 | usUfsgUfcGfaUfgAfcuuUfcAfcAfuUfcsusg | 301 | |
| AD-65645.1 | usUfsgUfcGfaUfgAfcuuUfcAfcAfuUfcsusg | 302 | |
| AD-65650.1 | usUfsgUfcGfaUfgAfcuuUfcAfcAfuUfcsusg | 303 | |
| AD-65655.1 | usUfsgUfcGfaUfgAfcuuUfcAfcAfuUfcsusg | 304 | |
| AD-65660.1 | usUfsgUfcGfaUfgAfcuuUfcAfcAfuUfcsusg | 305 | |
| AD-65665.1 | usUfsgUfcGfaUfgAfcuuUfcAfcAfuUfcsusg | 306 | |
| AD-65628.1 | usUfsgUfcGfaUfgAfcuuUfcAfcAfuUfcsusg | 307 | |
| AD-65634.1 | usUfsgUfcGfaUfgAfcuuUfcAfcAfuUfcsusg | 308 | |
| AD-65646.1 | usdTsgucgaugdAcuudTcacauucsusg | 309 | |
| AD-65656.1 | usUsgucgaugacuudTcacauucsusg | 310 | |
| AD-65661.1 | usdTsgucdGaugacuudTcacauucsusg | 311 | |
| AD-65666.1 | usUsgucdGaugacuudTcacauucsusg | 312 | |
| AD-65629.1 | usdTsgucgaugacuudTcdAcauucsusg | 313 | |
| AD-65635.1 | usdTsgucdGaugacuudTcdAcauucsusg | 314 | |
| AD-65641.1 | usdTsgucgaugdAcuudTcacauucsusg | 315 | |
| AD-62994.1 | usAfsuAfuUfuCfcAfggaUfgAfaAfgUfcscsa | 316 | |
| AD-65595.1 | usAfsuAfuUfuCfcAfggaUfgAfaAfgUfcscsa | 317 | |
| AD-65600.1 | usAfsuAfuUfuCfcAfggaUfgAfaAfgUfcscsa | 318 | |
| AD-65610.1 | usAfsuAfuUfuCfcAfggaUfgAfaAfgUfcscsa | 319 | |
| AD-65615.1 | usAfsuAfuUfuCfcAfggaUfgAfaAfgUfcscsa | 320 | |
| AD-65620.1 | usAfsuAfuUfuCfcAfggaUfgAfaAfgUfcscsa | 321 | |
| AD-65584.1 | usAfsuAfuUfuCfcAfggaUfgAfaAfgsusc | 322 | |
| AD-65590.1 | usAfsuauUfuccaggaUfgAfaagsusc | 323 | |
| AD-65596.1 | usAfsuauUfuCfcAfggaUfgAfaagucscsa | 324 | |
| AD-65601.1 | usAfsuauUfuCfCfaggaUfgAfaagucscsa | 325 | |
| AD-65606.1 | usAfsuauUfuccaggaUfgAfaagucscsa | 326 | |
| AD-65611.1 | usAfsuauUfuccaggaUfgaaagucscsa | 327 | |
| AD-65616.1 | usAfsuauuuccaggaUfgaaagucscsa | 328 | |
| AD-65621.1 | usAfsuauUfuCfcAfggaUfgAfaagucscsa | 329 | |
| AD-65585.1 | usAfsuauUfuCfCfaggaUfgAfaagucscsa | 330 | |
| AD-65591.1 | usAfsuauUfuccaggaUfgAfaagucscsa | 331 | |
| AD-65597.1 | usAfsuauUfuCfcAfggaUfgAfaagucscsa | 332 | |
| AD-65602.1 | usAfsuauUfuCfCfaggaUfgAfaagucscsa | 333 | |
| AD-65607.1 | usAfsuauUfuccaggaUfgAfaagucscsa | 334 | |
| AD-65612.1 | usAfsuAfuUfuCfcAfggaUfgAfaAfgUfcscsa | 335 | |
| AD-65622.1 | usAfsuAfuUfuCfcAfggaUfgAfaAfgUfcscsa | 336 | |
| AD-65586.1 | usAfsuAfuUfuCfcAfggaUfgAfaAfgUfcscsa | 337 | |
| AD-65592.1 | usAfsuAfuUfuCfcAfggaUfgAfaAfgUfcscsa | 338 | |
| AD-65598.1 | usAfsuAfuUfuCfcAfggaUfgAfaAfgUfcscsa | 339 | |
| AD-65603.1 | usAfsuAfuUfuCfcAfggaUfgAfaAfgUfcscsa | 340 | |
| AD-65608.1 | usAfsuAfuUfuCfcAfggaUfgAfaAfgUfcscsa | 341 | |
| AD-65613.1 | usAfsuAfuUfuCfcAfggaUfgAfaAfgUfcscsa | 342 | |
| AD-65618.1 | usAfsuAfuUfuCfcAfggaUfgAfaAfgUfcscsa | 343 | |
| AD-65623.1 | usAfsuAfuUfuCfcAfggaUfgAfaAfgUfcscsa | 344 | |
| AD-65587.1 | usAfsuAfuUfuCfcAfggaUfgAfaAfgUfcscsa | 345 | |
| AD-65593.1 | usAfsuAfuUfuCfcAfggaUfgAfaAfgUfcscsa | 346 | |
| AD-65599.1 | usdAsuauuuccdAggadTgaaagucscsa | 347 | |
| AD-65604.1 | usdAsuauuuccaggadTgaaagucscsa | 348 | |
| AD-65609.1 | usAsuauuuccaggadTgaaagucscsa | 349 | |
| AD-65614.1 | usdAsuaudTuccaggadTgaaagucscsa | 350 | |
| AD-65619.1 | usAsuaudTuccaggadTgaaagucscsa | 351 | |
| AD-65624.1 | usdAsuauuuccaggadTgdAaagucscsa | 352 | |
| AD-65588.1 | usdAsuaudTuccaggadTgdAaagucscsa | 353 | |
| AD-65594.1 | usdAsuauuuccdAggadTgaaagucscsa | 354 | |
| AD-68309.1 | usGfsacaUfcUfUfgaacAfcCfuuucuscsc | 355 | |
| AD-68303.1 | asGfsuuaAfuAfUfauuuCfcAfggaugsasa | 356 | |
| AD-65626.5 | usUfsgucGfaUfGfacuuUfcAfcauucsusg | 357 | |
| AD-68295.1 | usAfsuggGfaAfAfauauUfgUfgcacusgsu | 358 | |
| AD-68273.1 | asAfsuguCfuUfGfucgaUfgAfcuuucsasc | 359 | |
| AD-68297.1 | usUfsuguCfgAfUfgacuUfuCfacauuscsu | 360 | |
| AD-68287.1 | usAfsacaGfuUfAfauauAfuUfuccagsgsa | 361 | |
| AD-68300.1 | asAfsauaAfuAfCfagauGfgGfaaaausasu | 362 | |
| AD-68306.1 | usUfsuugUfuGfGfaaaaGfaAfcgacascsc | 363 | |
| AD-68292.1 | usAfsguuAfaUfAfuauuUfcCfaggausgsa | 364 | |
| AD-68298.1 | usAfsucaUfcAfCfcucuCfaAfaaugcscsc | 365 | |
| AD-68277.1 | usUfsgaaCfaCfCfuuucUfcCfcccugsgsa | 366 | |
| AD-68289.1 | usUfsuaaCfaGfUfuaauAfuAfuuuccsasg | 367 | |
| AD-68272.1 | asAfsggaUfuUfUfuccuCfaCfcaaugsusc | 368 | |
| AD-68282.1 | usAfsucuUfgAfAfcaccUfuUfcucccscsc | 369 | |
| AD-68285.1 | asUfscauCfaCfCfucucAfaAfaugccscsu | 370 | |
| AD-68290.1 | asAfsaagAfaCfGfacacCfcUfuuguasusu | 371 | |
| AD-68296.1 | usGfsauuCfgAfCfaccaAfgAfucccasusu | 372 | |
| AD-68288.1 | usAfsaaaUfaUfUfgugcAfcUfgucagsasu | 373 | |
| AD-68299.1 | asUfsgggAfaAfAfuauuGfuGfcacugsusc | 374 | |
| AD-68275.1 | usUfsaggAfcAfCfccauUfgAfaaaguscsa | 375 | |
| AD-68274.1 | asGfsgauUfuUfUfccucAfcCfaauguscsu | 376 | |
| AD-68294.1 | usCfsauuGfaAfAfagucAfaAfagcaasusg | 377 | |
| AD-68302.1 | usCfsaucAfuCfAfccucUfcAfaaaugscsc | 378 | |
| AD-68279.1 | usGfsacaCfcCfAfuugaAfaAfgucaasasa | 379 | |
| AD-68304.1 | usCfsuguCfcUfAfaaacAfgAfagucgsasc | 380 | |
| AD-68286.1 | usAfsuucUfgGfCfacccAfcUfcagagscsc | 381 | |
| AD-68291.1 | usAfscuuUfcAfCfauucUfgGfcacccsasc | 382 | |
| AD-68283.1 | usGfsuucCfuAfGfgacaCfcCfauugasasa | 383 | |
| AD-68280.1 | usAfsaugUfcUfUfgucgAfuGfacuuuscsa | 384 | |
| AD-68293.1 | usGfscauCfaUfCfaccuCfuCfaaaausgsc | 385 | |
| AD-68276.1 | usCfsucaCfcAfAfugucUfuGfucgausgsa | 386 | |
| AD-68308.1 | usGfsacuUfuCfAfcauuCfuGfgcaccscsa | 387 | |
| AD-68278.1 | usGfsgaaAfaUfAfuuguGfcAfcugucsasg | 388 | |
| AD-68307.1 | usUfscugCfaCfAfguguCfuCfuuuguscsa | 389 | |
| AD-68284.1 | usCfscuaGfgAfCfacccAfuUfgaaaasgsu | 390 | |
| AD-68301.1 | asCfsuguCfaGfAfucuuGfgAfaacggscsc | 391 | |
| AD-68281.1 | usUfsugaAfcAfCfcuuuCfuCfccccusgsg | 392 | |
| AD-68305.1 | asCfscaaUfgUfCfuuguCfgAfugacususu | 393 | |

TABLE 9
Unmodified Human/Mouse/Cyno/Rat, Human/Mouse/Cyno, and Human/Cyno Cross-Reactive
HAO1 iRNA Sequences

| | SEQ | | SEQ | | |
|---|---|---|---|---|---|
| Duplex | ID | | ID | | Position in |
| Name | NO: | Sense Strand Sequence 5′ to 3′ | NO: | Antisense Strand Sequence 5′ to 3′ | NM_017545.2 |
| AD-62933 | 394 | GAAUGUGAAAGUCAUCGACAA | 443 | UUGUCGAUGACUUUCACAUUCUG | 1072-1094 |
| AD-62939 | 395 | UUUUCAAUGGGUGUCCUAGGA | 444 | UCCUAGGACACCCAUUGAAAAGU | 1302-1324 |
| AD-62944 | 396 | GAAAGUCAUCGACAAGACAUU | 445 | AAUGUCUUGUCGAUGACUUUCAC | 1078-1100 |
| AD-62949 | 397 | UCAUCGACAAGACAUUGGUGA | 446 | UCACCAAUGUCUUGUCGAUGACU | 1083-1105 |
| AD-62954 | 398 | UUUCAAUGGGUGUCCUAGGAA | 447 | UUCCUAGGACACCCAUUGAAAAG | 1303-1325 |
| AD-62959 | 399 | AAUGGGUGUCCUAGGAACCUU | 448 | AAGGUUCCUAGGACACCCAUUGA | 1307-1329 |
| AD-62964 | 400 | GACAGUGCACAAUAUUUUCCA | 449 | UGGAAAAUAUUGUGCACUGUCAG | 1134-1156_C21A |
| AD-62969 | 401 | ACUUUUCAAUGGGUGUCCUAA | 450 | UUAGGACACCCAUUGAAAAGUCA | 1300-1322_G21A |
| AD-62934 | 402 | AAGUCAUCGACAAGACAUUGA | 451 | UCAAUGUCUUGUCGAUGACUUUC | 1080-1102_G21A |
| AD-62940 | 403 | AUCGACAAGACAUUGGUGAGA | 452 | UCUCACCAAUGUCUUGUCGAUGA | 1085-1107_G21A |
| AD-62945 | 404 | GGGAGAAAGGUGUUCAAGAUA | 453 | UAUCUUGAACACCUUUCUCCCCC | 996-1018_G21A |
| AD-62950 | 405 | CUUUUCAAUGGGUGUCCUAGA | 454 | UCUAGGACACCCAUUGAAAAGUC | 1301-1323_G21A |
| AD-62955 | 406 | UCAAUGGGUGUCCUAGGAACA | 455 | UGUUCCUAGGACACCCAUUGAAA | 1305-1327_C21A |
| AD-62960 | 407 | UUGACUUUUCAAUGGGUGUCA | 456 | UGACACCCAUUGAAAAGUCAAAA | 1297-1319_C21A |
| AD-62965 | 408 | AAAGUCAUCGACAAGACAUUA | 457 | UAAUGUCUUGUCGAUGACUUUCA | 1079-1101_G21A |
| AD-62970 | 409 | CAGGGGGAGAAAGGUGUUCAA | 458 | UUGAACACCUUUCUCCCCCUGGA | 992-1014 |
| AD-62935 | 410 | CAUUGGUGAGGAAAAAUCCUU | 459 | AAGGAUUUUUCCUCACCAAUGUC | 1095-1117 |
| AD-62941 | 411 | ACAUUGGUGAGGAAAAAUCCU | 460 | AGGAUUUUUCCUCACCAAUGUCU | 1094-1116 |
| AD-62946 | 412 | AGGGGGAGAAAGGUGUUCAAA | 461 | UUUGAACACCUUUCUCCCCCUGG | 993-1015_G21A |
| AD-62974 | 413 | CUCAGGAUGAAAAAUUUUGAA | 462 | UUCAAAAUUUUUCAUCCUGAGUU | 563-585 |
| AD-62978 | 414 | CAGCAUGUAUUACUUGACAAA | 463 | UUUGUCAAGUAAUACAUGCUGAA | 1173-1195 |
| AD-62982 | 415 | UAUGAACAACAUGCUAAAUCA | 464 | UGAUUUAGCAUGUUGUUCAUAAU | 53-75 |
| AD-62986 | 416 | AUAUAUCCAAAUGUUUUAGGA | 465 | UCCUAAAACAUUUGGAUAUAUUC | 1679-1701 |
| AD-62990 | 417 | CCAGAUGGAAGCUGUAUCCAA | 466 | UUGGAUACAGCUUCCAUCUGGAA | 156-178 |
| AD-62994 | 418 | GACUUUCAUCCUGGAAAUAUA | 467 | UAUAUUUCCAGGAUGAAAGUCCA | 1341-1363 |
| AD-62998 | 419 | CCCCGGCUAAUUUGUAUCAAU | 468 | AUUGAUACAAAUUAGCCGGGGGA | 29-51 |
| AD-63002 | 420 | UUAAACAUGGCUUGAAUGGGA | 469 | UCCCAUUCAAGCCAUGUUUAACA | 765-787 |
| AD-62975 | 421 | AAUGUGUUUAGACAACGUCAU | 470 | AUGACGUUGUCUAAACACAUUUU | 1388-1410 |
| AD-62979 | 422 | ACUAAAGGAAGAAUUCCGGUU | 471 | AACCGGAAUUCUUCCUUUAGUAU | 1027-1049 |
| AD-62983 | 423 | UAUAUCCAAAUGUUUUAGGAU | 472 | AUCCUAAAACAUUUGGAUAUAUU | 1680-1702 |
| AD-62987 | 424 | GUGCGGAAAGGCACUGAUGUU | 473 | AACAUCAGUGCCUUUCCGCACAC | 902-924 |
| AD-62991 | 425 | UAAAACAGUGGUUCUUAAAUU | 474 | AAUUUAAGAACCACUGUUUUAAA | 1521-1543 |
| AD-62995 | 426 | AUGAAAAAUUUUGAAACCAGU | 475 | ACUGGUUUCAAAAUUUUUCAUCC | 569-591 |
| AD-62999 | 427 | AACAAAAUAGCAAUCCCUUUU | 476 | AAAAGGGAUUGCUAUUUUGUUGG | 1264-1286 |
| AD-63003 | 428 | CUGAAACAGAUCUGUCGACUU | 477 | AAGUCGACAGAUCUGUUUCAGCA | 195-217 |
| AD-62976 | 429 | UUGUUGCAAAGGGCAUUUUGA | 478 | UCAAAAUGCCCUUUGCAACAAUU | 720-742 |
| AD-62980 | 430 | CUCAUUGUUUAUUAACCUGUA | 479 | UACAGGUUAAUAAACAAUGAGAU | 1483-1505 |
| AD-62984 | 431 | CAACAAAAUAGCAAUCCCUUU | 480 | AAAGGGAUUGCUAUUUUGUUGGA | 1263-1285 |
| AD-62992 | 432 | CAUUGUUUAUUAACCUGUAUU | 481 | AAUACAGGUUAAUAAACAAUGAG | 1485-1507 |
| AD-62996 | 433 | UAUCAGCUGGGAAGAUAUCAA | 482 | UUGAUAUCUUCCCAGCUGAUAGA | 670-692 |
| AD-63000 | 434 | UGUCCUAGGAACCUUUUAGAA | 483 | UUCUAAAAGGUUCCUAGGACACC | 1313-1335 |
| AD-63004 | 435 | UCCAACAAAAUAGCAAUCCCU | 484 | AGGGAUUGCUAUUUUGUUGGAAA | 1261-1283 |
| AD-62977 | 436 | GGUGUGCGGAAAGGCACUGAU | 485 | AUCAGUGCCUUUCCGCACACCCC | 899-921 |
| AD-62981 | 437 | UUGAAACCAGUACUUUAUCAU | 486 | AUGAUAAAGUACUGGUUUCAAAA | 579-601 |
| AD-62985 | 438 | UACUUCCAAAGUCUAUAUAUA | 487 | UAUAUAUAGACUUUGGAAGUACU | 75-97_G21A |
| AD-62989 | 439 | UCCUAGGAACCUUUUAGAAAU | 488 | AUUUCUAAAAGGUUCCUAGGACA | 1315-1337_G21U |
| AD-62993 | 440 | CUCCUGAGGAAAAUUUUGGAA | 489 | UUCCAAAAUUUUCCUCAGGAGAA | 603-625_G21A |
| AD-62997 | 441 | GCUCCGGAAUGUUGCUGAAAU | 490 | AUUUCAGCAACAUUCCGGAGCAU | 181-203_C21U |
| AD-63001 | 442 | GUGUUUGUGGGGAGACCAAUA | 491 | UAUUGGUCUCCCCACAAACACAG | 953-975_C21A |

TABLE 10
Unmodified Mouse and Mouse/Rat HAO1 iRNA Sequences

| | SEQ | | SEQ | | |
|---|---|---|---|---|---|
| Duplex | ID | | ID | | Position in |
| Name | NO: | Sense strand sequence 5′ to 3′ | NO: | Antisense strand sequence 5′ to 3′ | NM_010403.2 |
| AD-62951 | 492 | AUGGUGGUAAUUUGUGAUUUU | 514 | AAAAUCACAAAUUACCACCAUCC | 1642-1664 |
| AD-62956 | 493 | GACUUGCAUCCUGGAAAUAUA | 515 | UAUAUUUCCAGGAUGCAAGUCCA | 1338-1360 |
| AD-62961 | 494 | GGAAGGGAAGGUAGAAGUCUU | 516 | AAGACUUCUACCUUCCCUUCCAC | 864-886 |
| AD-62966 | 495 | UGUCUUCUGUUUAGAUUUCCU | 517 | AGGAAAUCUAAACAGAAGACAGG | 1506-1528 |
| AD-62971 | 496 | CUUUGGCUGUUUCCAAGAUCU | 518 | AGAUCUUGGAAACAGCCAAAGGA | 1109-1131 |
| AD-62936 | 497 | AAUGUGUUUGGGCAACGUCAU | 519 | AUGACGUUGCCCAAACACAUUUU | 1385-1407 |
| AD-62942 | 498 | UGUGACUGUGGACACCCCUUA | 520 | UAAGGGGUGUCCACAGUCACAAA | 486-508 |
| AD-62947 | 499 | GAUGGGGUGCCAGCUACUAUU | 521 | AAUAGUAGCUGGCACCCCAUCCA | 814-836 |
| AD-62952 | 500 | GAAAAUGUGUUUGGGCAACGU | 522 | ACGUUGCCCAAACACAUUUUCAA | 1382-1404 |
| AD-62957 | 501 | GGCUGUUUCCAAGAUCUGACA | 523 | UGUCAGAUCUUGGAAACAGCCAA | 1113-1135 |
| AD-62962 | 502 | UCCAACAAAAUAGCCACCCCU | 524 | AGGGGUGGCUAUUUUGUUGGAAA | 1258-1280 |
| AD-62967 | 503 | GUCUUCUGUUUAGAUUUCCUU | 525 | AAGGAAAUCUAAACAGAAGACAG | 1507-1529 |
| AD-62972 | 504 | UGGAAGGGAAGGUAGAAGUCU | 526 | AGACUUCUACCUUCCCUUCCACA | 863-885 |
| AD-62937 | 505 | UCCUUUGGCUGUUUCCAAGAU | 527 | AUCUUGGAAACAGCCAAAGGAUU | 1107-1129 |
| AD-62943 | 506 | CAUCUCUCAGCUGGGAUGAUA | 528 | UAUCAUCCCAGCUGAGAGAUGGG | 662-684 |
| AD-62948 | 507 | GGGGUGCCAGCUACUAUUGAU | 529 | AUCAAUAGUAGCUGGCACCCCAU | 817-839 |
| AD-62953 | 508 | AUGUGUUUGGGCAACGUCAUA | 530 | UAUGACGUUGCCCAAACACAUUU | 1386-1408_C21A |
| AD-62958 | 509 | CUGUUUAGAUUUCCUUAAGAA | 531 | UUCUUAAGGAAAUCUAAACAGAA | 1512-1534_C21A |
| AD-62963 | 510 | AGAAAGAAAUGGACUUGCAUA | 532 | UAUGCAAGUCCAUUUCUUUCUAG | 1327-1349_C21A |
| AD-62968 | 511 | GCAUCCUGGAAAUAUAUUAAA | 533 | UUUAAUAUAUUUCCAGGAUGCAA | 1343-1365_C21A |
| AD-62973 | 512 | CCUGUCAGACCAUGGGAACUA | 534 | UAGUUCCCAUGGUCUGACAGGCU | 308-330_G21A |
| AD-62938 | 513 | AAACAUGGUGUGGAUGGGAUA | 535 | UAUCCCAUCCACACCAUGUUUAA | 763-785_C21A |

TABLE 11
Additional Unmodified Human/Cyno/Mouse/Rat, Human/Mouse/Cyno, Human/Cyno, and Mouse/Rat

| | SEQ | | | | |
|---|---|---|---|---|---|
| | ID | | SEQ ID | | Position in |
| Duplex Name | NO: | Sense strand sequence 5′ to 3′ | NO: | Antisense strand sequence 5′ to 3′ | NM_017545.2 |
| AD-62933.2 | 394 | GAAUGUGAAAGUCAUCGACAA | 443 | UUGUCGAUGACUUUCACAUUCUG | 1072-1094 |
| AD-62939.2 | 395 | UUUUCAAUGGGUGUCCUAGGA | 444 | UCCUAGGACACCCAUUGAAAAGU | 1302-1324 |
| AD-62944.2 | 396 | GAAAGUCAUCGACAAGACAUU | 445 | AAUGUCUUGUCGAUGACUUUCAC | 1078-1100 |
| AD-62949.2 | 397 | UCAUCGACAAGACAUUGGUGA | 446 | UCACCAAUGUCUUGUCGAUGACU | 1083-1105 |
| AD-62954.2 | 398 | UUUCAAUGGGUGUCCUAGGAA | 447 | UUCCUAGGACACCCAUUGAAAAG | 1303-1325 |
| AD-62959.2 | 399 | AAUGGGUGUCCUAGGAACCUU | 448 | AAGGUUCCUAGGACACCCAUUGA | 1307-1329 |
| AD-62964.2 | 400 | GACAGUGCACAAUAUUUUCCA | 449 | UGGAAAAUAUUGUGCACUGUCAG | 1134-1156_C21A |
| AD-62969.2 | 401 | ACUUUUCAAUGGGUGUCCUAA | 450 | UUAGGACACCCAUUGAAAAGUCA | 1300-1322_G21A |
| AD-62934.2 | 402 | AAGUCAUCGACAAGACAUUGA | 451 | UCAAUGUCUUGUCGAUGACUUUC | 1080-1102_G21A |
| AD-62940.2 | 403 | AUCGACAAGACAUUGGUGAGA | 452 | UCUCACCAAUGUCUUGUCGAUGA | 1085-1107_G21A |
| AD-62945.2 | 404 | GGGAGAAAGGUGUUCAAGAUA | 453 | UAUCUUGAACACCUUUCUCCCCC | 996-1018_G21A |
| AD-62950.2 | 405 | CUUUUCAAUGGGUGUCCUAGA | 454 | UCUAGGACACCCAUUGAAAAGUC | 1301-1323_G21A |
| AD-62955.2 | 406 | UCAAUGGGUGUCCUAGGAACA | 455 | UGUUCCUAGGACACCCAUUGAAA | 1305-1327_C21A |
| AD-62960.2 | 407 | UUGACUUUUCAAUGGGUGUCA | 456 | UGACACCCAUUGAAAAGUCAAAA | 1297-1319_C21A |
| AD-62965.2 | 408 | AAAGUCAUCGACAAGACAUUA | 457 | UAAUGUCUUGUCGAUGACUUUCA | 1079-1101_G21A |
| AD-62970.2 | 409 | CAGGGGGAGAAAGGUGUUCAA | 458 | UUGAACACCUUUCUCCCCCUGGA | 992-1014 |
| AD-62935.2 | 410 | CAUUGGUGAGGAAAAAUCCUU | 459 | AAGGAUUUUUCCUCACCAAUGUC | 1095-1117 |
| AD-62941.2 | 411 | ACAUUGGUGAGGAAAAAUCCU | 460 | AGGAUUUUUCCUCACCAAUGUCU | 1094-1116 |
| AD-62946.2 | 412 | AGGGGGAGAAAGGUGUUCAAA | 461 | UUUGAACACCUUUCUCCCCCUGG | 993-1015_G21A |
| AD-62974.2 | 413 | CUCAGGAUGAAAAAUUUUGAA | 462 | UUCAAAAUUUUUCAUCCUGAGUU | 563-585 |
| AD-62978.2 | 414 | CAGCAUGUAUUACUUGACAAA | 463 | UUUGUCAAGUAAUACAUGCUGAA | 1173-1195 |
| AD-62982.2 | 415 | UAUGAACAACAUGCUAAAUCA | 464 | UGAUUUAGCAUGUUGUUCAUAAU | 53-75 |
| AD-62986.2 | 416 | AUAUAUCCAAAUGUUUUAGGA | 465 | UCCUAAAACAUUUGGAUAUAUUC | 1679-1701 |
| AD-62990.2 | 417 | CCAGAUGGAAGCUGUAUCCAA | 466 | UUGGAUACAGCUUCCAUCUGGAA | 156-178 |
| AD-62994.2 | 418 | GACUUUCAUCCUGGAAAUAUA | 467 | UAUAUUUCCAGGAUGAAAGUCCA | 1341-1363 |
| AD-62998.2 | 419 | CCCCGGCUAAUUUGUAUCAAU | 468 | AUUGAUACAAAUUAGCCGGGGGA | 29-51 |
| AD-63002.2 | 420 | UUAAACAUGGCUUGAAUGGGA | 469 | UCCCAUUCAAGCCAUGUUUAACA | 765-787 |
| AD-62975.2 | 421 | AAUGUGUUUAGACAACGUCAU | 470 | AUGACGUUGUCUAAACACAUUUU | 1388-1410 |
| AD-62979.2 | 422 | ACUAAAGGAAGAAUUCCGGUU | 471 | AACCGGAAUUCUUCCUUUAGUAU | 1027-1049 |
| AD-62983.2 | 423 | UAUAUCCAAAUGUUUUAGGAU | 472 | AUCCUAAAACAUUUGGAUAUAUU | 1680-1702 |
| AD-62987.2 | 424 | GUGCGGAAAGGCACUGAUGUU | 473 | AACAUCAGUGCCUUUCCGCACAC | 902-924 |
| AD-62991.2 | 425 | UAAAACAGUGGUUCUUAAAUU | 474 | AAUUUAAGAACCACUGUUUUAAA | 1521-1543 |
| AD-62995.2 | 426 | AUGAAAAAUUUUGAAACCAGU | 475 | ACUGGUUUCAAAAUUUUUCAUCC | 569-591 |
| AD-62999.2 | 427 | AACAAAAUAGCAAUCCCUUUU | 476 | AAAAGGGAUUGCUAUUUUGUUGG | 1264-1286 |
| AD-63003.2 | 428 | CUGAAACAGAUCUGUCGACUU | 477 | AAGUCGACAGAUCUGUUUCAGCA | 195-217 |
| AD-62976.2 | 429 | UUGUUGCAAAGGGCAUUUUGA | 478 | UCAAAAUGCCCUUUGCAACAAUU | 720-742 |
| AD-62980.2 | 430 | CUCAUUGUUUAUUAACCUGUA | 479 | UACAGGUUAAUAAACAAUGAGAU | 1483-1505 |
| AD-62984.2 | 431 | CAACAAAAUAGCAAUCCCUUU | 480 | AAAGGGAUUGCUAUUUUGUUGGA | 1263-1285 |
| AD-62992.2 | 432 | CAUUGUUUAUUAACCUGUAUU | 481 | AAUACAGGUUAAUAAACAAUGAG | 1485-1507 |
| AD-62996.2 | 433 | UAUCAGCUGGGAAGAUAUCAA | 482 | UUGAUAUCUUCCCAGCUGAUAGA | 670-692 |
| AD-63000.2 | 434 | UGUCCUAGGAACCUUUUAGAA | 483 | UUCUAAAAGGUUCCUAGGACACC | 1313-1335 |
| AD-63004.2 | 435 | UCCAACAAAAUAGCAAUCCCU | 484 | AGGGAUUGCUAUUUUGUUGGAAA | 1261-1283 |
| AD-62977.2 | 436 | GGUGUGCGGAAAGGCACUGAU | 485 | AUCAGUGCCUUUCCGCACACCCC | 899-921 |
| AD-62981.2 | 437 | UUGAAACCAGUACUUUAUCAU | 486 | AUGAUAAAGUACUGGUUUCAAAA | 579-601 |
| AD-62985.2 | 438 | UACUUCCAAAGUCUAUAUAUA | 487 | UAUAUAUAGACUUUGGAAGUACU | 75-97_G21A |
| AD-62989.2 | 439 | UCCUAGGAACCUUUUAGAAAU | 488 | AUUUCUAAAAGGUUCCUAGGACA | 1315-1337_G21U |
| AD-62993.2 | 440 | CUCCUGAGGAAAAUUUUGGAA | 489 | UUCCAAAAUUUUCCUCAGGAGAA | 603-625_G21A |
| AD-62997.2 | 441 | GCUCCGGAAUGUUGCUGAAAU | 490 | AUUUCAGCAACAUUCCGGAGCAU | 181-203_C21U |
| AD-63001.2 | 442 | GUGUUUGUGGGGAGACCAAUA | 491 | UAUUGGUCUCCCCACAAACACAG | 953-975_C21A |
| AD-62951.2 | 492 | AUGGUGGUAAUUUGUGAUUUU | 514 | AAAAUCACAAAUUACCACCAUCC | 1642-1664 |
| AD-62956.2 | 493 | GACUUGCAUCCUGGAAAUAUA | 515 | UAUAUUUCCAGGAUGCAAGUCCA | 1338-1360 |
| AD-62961.2 | 494 | GGAAGGGAAGGUAGAAGUCUU | 516 | AAGACUUCUACCUUCCCUUCCAC | 864-886 |
| AD-62966.2 | 495 | UGUCUUCUGUUUAGAUUUCCU | 517 | AGGAAAUCUAAACAGAAGACAGG | 1506-1528 |
| AD-62971.2 | 496 | CUUUGGCUGUUUCCAAGAUCU | 518 | AGAUCUUGGAAACAGCCAAAGGA | 1109-1131 |
| AD-62936.2 | 497 | AAUGUGUUUGGGCAACGUCAU | 519 | AUGACGUUGCCCAAACACAUUUU | 1385-1407 |
| AD-62942.2 | 498 | UGUGACUGUGGACACCCCUUA | 520 | UAAGGGGUGUCCACAGUCACAAA | 486-508 |
| AD-62947.2 | 499 | GAUGGGGUGCCAGCUACUAUU | 521 | AAUAGUAGCUGGCACCCCAUCCA | 814-836 |
| AD-62952.2 | 500 | GAAAAUGUGUUUGGGCAACGU | 522 | ACGUUGCCCAAACACAUUUUCAA | 1382-1404 |
| AD-62957.2 | 501 | GGCUGUUUCCAAGAUCUGACA | 523 | UGUCAGAUCUUGGAAACAGCCAA | 1113-1135 |
| AD-62962.2 | 502 | UCCAACAAAAUAGCCACCCCU | 524 | AGGGGUGGCUAUUUUGUUGGAAA | 1258-1280 |
| AD-62967.2 | 503 | GUCUUCUGUUUAGAUUUCCUU | 525 | AAGGAAAUCUAAACAGAAGACAG | 1507-1529 |
| AD-62972.2 | 504 | UGGAAGGGAAGGUAGAAGUCU | 526 | AGACUUCUACCUUCCCUUCCACA | 863-885 |
| AD-62937.2 | 505 | UCCUUUGGCUGUUUCCAAGAU | 527 | AUCUUGGAAACAGCCAAAGGAUU | 1107-1129 |
| AD-62943.2 | 506 | CAUCUCUCAGCUGGGAUGAUA | 528 | UAUCAUCCCAGCUGAGAGAUGGG | 662-684 |
| AD-62948.2 | 507 | GGGGUGCCAGCUACUAUUGAU | 529 | AUCAAUAGUAGCUGGCACCCCAU | 817-839 |
| AD-62953.2 | 508 | AUGUGUUUGGGCAACGUCAUA | 530 | UAUGACGUUGCCCAAACACAUUU | 1386-1408_C21A |
| AD-62958.2 | 509 | CUGUUUAGAUUUCCUUAAGAA | 531 | UUCUUAAGGAAAUCUAAACAGAA | 1512-1534_C21A |
| AD-62963.2 | 510 | AGAAAGAAAUGGACUUGCAUA | 532 | UAUGCAAGUCCAUUUCUUUCUAG | 1327-1349_C21A |
| AD-62968.2 | 511 | GCAUCCUGGAAAUAUAUUAAA | 533 | UUUAAUAUAUUUCCAGGAUGCAA | 1343-1365_C21A |
| AD-62973.2 | 512 | CCUGUCAGACCAUGGGAACUA | 534 | UAGUUCCCAUGGUCUGACAGGCU | 308-330_G21A |
| AD-62938.2 | 513 | AAACAUGGUGUGGAUGGGAUA | 535 | UAUCCCAUCCACACCAUGUUUAA | 763-785_C21A |
| AD-62933.1 | 536 | GAAUGUGAAAGUCAUCGACAA | 653 | UUGUCGAUGACUUUCACAUUCUG | 1072-1094 |
| AD-65630.1 | 537 | GAAUGUGAAAGUCAUCGACAA | 654 | UUGUCGAUGACUUUCACAUUCUG | 1072-1094 |
| AD-65636.1 | 538 | GAAUGUGAAAGUCAUCGACAA | 655 | UUGUCGAUGACUUUCACAUUCUG | 1072-1094 |
| AD-65642.1 | 539 | GAAUGUGAAAGUCAUCGACAA | 656 | UUGUCGAUGACUUUCACAUUCUG | 1072-1094 |
| AD-65647.1 | 540 | GAAUGUGAAAGUCAUCGACAA | 657 | UUGUCGAUGACUUUCACAUUCUG | 1072-1094 |
| AD-65652.1 | 541 | GAAUGUGAAAGUCAUCGACAA | 658 | UUGUCGAUGACUUUCACAUUCUG | 1072-1094 |
| AD-65657.1 | 542 | GAAUGUGAAAGUCAUCGACAA | 659 | UUGUCGAUGACUUUCACAUUCUG | 1072-1094 |
| AD-65662.1 | 543 | GAAUGUGAAAGUCAUCGACAA | 660 | UUGUCGAUGACUUUCACAUUCUG | 1072-1094 |
| AD-65625.1 | 544 | AUGUGAAAGUCAUCGACAA | 661 | UUGUCGAUGACUUUCACAUUC | 1072-1094 |
| AD-65631.1 | 545 | AUGUGAAAGUCAUCGACAA | 662 | UUGUCGAUGACUUUCACAUUC | 1072-1094 |
| AD-65637.1 | 546 | GAAUGUGAAAGUCAUCGACAA | 663 | UUGUCGAUGACUUUCACAUUCUG | 1072-1094 |
| AD-65643.1 | 547 | GAAUGUGAAAGUCAUCGACAA | 664 | UUGUCGAUGACUUUCACAUUCUG | 1072-1094 |
| AD-65648.1 | 548 | GAAUGUGAAAGUCAUCGACAA | 665 | UUGUCGAUGACUUUCACAUUCUG | 1072-1094 |
| AD-65653.1 | 549 | GAAUGUGAAAGUCAUCGACAA | 666 | UUGUCGAUGACUUUCACAUUCUG | 1072-1094 |
| AD-65658.1 | 550 | GAAUGUGAAAGUCAUCGACAA | 667 | UUGUCGAUGACUUUCACAUUCUG | 1072-1094 |
| AD-65663.1 | 551 | GAAUGUGAAAGUCAUCGACAA | 668 | UUGUCGAUGACUUUCACAUUCUG | 1072-1094 |
| AD-65626.1 | 552 | GAAUGUGAAAGUCAUCGACAA | 669 | UUGUCGAUGACUUUCACAUUCUG | 1072-1094 |
| AD-65638.1 | 553 | GAAUGUGAAAGUCAUCGACAA | 670 | UUGUCGAUGACUUUCACAUUCUG | 1072-1094 |
| AD-65644.1 | 554 | GAAUGUGAAAGUCAUCGACAA | 671 | UUGUCGAUGACUUUCACAUUCUG | 1072-1094 |
| AD-65649.1 | 555 | GAAUGUGAAAGUCAUCGACAA | 672 | UUGUCGAUGACUUUCACAUUCUG | 1072-1094 |
| AD-65654.1 | 556 | GAAUGUGAAAGUCAUCGACAA | 673 | UUGUCGAUGACUUUCACAUUCUG | 1072-1094 |
| AD-65659.1 | 557 | GAAUGTGAAAGUCAUCGACAA | 674 | UUGUCGAUGACUUUCACAUUCUG | 1072-1094 |
| AD-65627.1 | 558 | GAAUGUGAAAGUCAUCGACAA | 675 | UUGUCGAUGACUUUCACAUUCUG | 1072-1094 |
| AD-65633.1 | 559 | GAAUGTGAAAGUCAUCGACAA | 676 | UUGUCGAUGACUUUCACAUUCUG | 1072-1094 |
| AD-65639.1 | 560 | GAAUGUGAAAGUCAUCGACAA | 677 | UUGUCGAUGACUUUCACAUUCUG | 1072-1094 |
| AD-65645.1 | 561 | GAAUGUGAAAGUCAUCGACAA | 678 | UUGUCGAUGACUUUCACAUUCUG | 1072-1094 |
| AD-65650.1 | 562 | GAAUGUGAAAGUCAUCTACAA | 679 | UUGUCGAUGACUUUCACAUUCUG | 1072-1094 |
| AD-65655.1 | 563 | GAAUGUGAAAGUCAUCACAA | 680 | UUGUCGAUGACUUUCACAUUCUG | 1072-1094 |
| AD-65660.1 | 564 | GAAUGUGAAAGUCATCTACAA | 681 | UUGUCGAUGACUUUCACAUUCUG | 1072-1094 |
| AD-65665.1 | 565 | GAAUGUGAAAGUCAUCGACAA | 682 | UUGUCGAUGACUUUCACAUUCUG | 1072-1094 |
| AD-65628.1 | 566 | GAAUGUGAAAGUCAUCTACAA | 683 | UUGUCGAUGACUUUCACAUUCUG | 1072-1094 |
| AD-65634.1 | 567 | GAAUGUGAAAGUCAUCACAA | 684 | UUGUCGAUGACUUUCACAUUCUG | 1072-1094 |
| AD-65646.1 | 568 | GAAUGUGAAAGUCAUCGACAA | 685 | UTGUCGAUGACUUTCACAUUCUG | 1072-1094 |
| AD-65656.1 | 569 | GAAUGUGAAAGUCAUCGACAA | 686 | UUGUCGAUGACUUTCACAUUCUG | 1072-1094 |
| AD-65661.1 | 570 | GAAUGUGAAAGUCAUCGACAA | 687 | UTGUCGAUGACUUTCACAUUCUG | 1072-1094 |
| AD-65666.1 | 571 | GAAUGUGAAAGUCAUCGACAA | 688 | UUGUCGAUGACUUTCACAUUCUG | 1072-1094 |
| AD-65629.1 | 572 | GAAUGUGAAAGUCAUCGACAA | 689 | UTGUCGAUGACUUTCACAUUCUG | 1072-1094 |
| AD-65635.1 | 573 | GAAUGUGAAAGUCAUCGACAA | 690 | UTGUCGAUGACUUTCACAUUCUG | 1072-1094 |
| AD-65641.1 | 574 | GAAUGUGAAAGUCAUCGACAA | 691 | UTGUCGAUGACUUTCACAUUCUG | 1072-1094 |
| AD-62994.1 | 575 | GACUUUCAUCCUGGAAAUAUA | 692 | UAUAUUUCCAGGAUGAAAGUCCA | 1341-1363 |
| AD-65595.1 | 576 | GACUUUCAUCCUGGAAAUAUA | 693 | UAUAUUUCCAGGAUGAAAGUCCA | 1341-1363 |
| AD-65600.1 | 577 | GACUUUCAUCCUGGAAAUAUA | 694 | UAUAUUUCCAGGAUGAAAGUCCA | 1341-1363 |
| AD-65610.1 | 578 | GACUUUCAUCCUGGAAAUAUA | 695 | UAUAUUUCCAGGAUGAAAGUCCA | 1341-1363 |
| AD-65615.1 | 579 | GACUUUCAUCCUGGAAAUAUA | 696 | UAUAUUUCCAGGAUGAAAGUCCA | 1341-1363 |
| AD-65620.1 | 580 | GACUUUCAUCCUGGAAAUAUA | 697 | UAUAUUUCCAGGAUGAAAGUCCA | 1341-1363 |
| AD-65584.1 | 581 | CUUUCAUCCUGGAAAUAUA | 698 | UAUAUUUCCAGGAUGAAAGUC | 1341-1361 |
| AD-65590.1 | 582 | CUUUCAUCCUGGAAAUAUA | 699 | UAUAUUUCCAGGAUGAAAGUC | 1341-1361 |
| AD-65596.1 | 583 | GACUUUCAUCCUGGAAAUAUA | 700 | UAUAUUUCCAGGAUGAAAGUCCA | 1341-1363 |
| AD-65601.1 | 584 | GACUUUCAUCCUGGAAAUAUA | 701 | UAUAUUUCCAGGAUGAAAGUCCA | 1341-1363 |
| AD-65606.1 | 585 | GACUUUCAUCCUGGAAAUAUA | 702 | UAUAUUUCCAGGAUGAAAGUCCA | 1341-1363 |
| AD-65611.1 | 586 | GACUUUCAUCCUGGAAAUAUA | 703 | UAUAUUUCCAGGAUGAAAGUCCA | 1341-1363 |
| AD-65616.1 | 587 | GACUUUCAUCCUGGAAAUAUA | 704 | UAUAUUUCCAGGAUGAAAGUCCA | 1341-1363 |
| AD-65621.1 | 588 | GACUUUCAUCCUGGAAAUAUA | 705 | UAUAUUUCCAGGAUGAAAGUCCA | 1341-1363 |
| AD-65585.1 | 589 | GACUUUCAUCCUGGAAAUAUA | 706 | UAUAUUUCCAGGAUGAAAGUCCA | 1341-1363 |
| AD-65591.1 | 590 | GACUUUCAUCCUGGAAAUAUA | 707 | UAUAUUUCCAGGAUGAAAGUCCA | 1341-1363 |
| AD-65597.1 | 591 | GACUUUCAUCCUGGAAAUAUA | 708 | UAUAUUUCCAGGAUGAAAGUCCA | 1341-1363 |
| AD-65602.1 | 592 | GACUUUCAUCCUGGAAAUAUA | 709 | UAUAUUUCCAGGAUGAAAGUCCA | 1341-1363 |
| AD-65607.1 | 593 | GACUUUCAUCCUGGAAAUAUA | 710 | UAUAUUUCCAGGAUGAAAGUCCA | 1341-1363 |
| AD-65612.1 | 594 | GACUUUCAUCCUGGAAAUAUA | 711 | UAUAUUUCCAGGAUGAAAGUCCA | 1341-1363 |
| AD-65622.1 | 595 | GACUUUCAUCCUGGAAAUAUA | 712 | UAUAUUUCCAGGAUGAAAGUCCA | 1341-1363 |
| AD-65586.1 | 596 | GACUTUCAUCCUGGAAAUAUA | 713 | UAUAUUUCCAGGAUGAAAGUCCA | 1341-1363 |
| AD-65592.1 | 597 | GACUUTCAUCCUGGAAAUAUA | 714 | UAUAUUUCCAGGAUGAAAGUCCA | 1341-1363 |
| AD-65598.1 | 598 | GACUUUCAUCCUGGAAAUAUA | 715 | UAUAUUUCCAGGAUGAAAGUCCA | 1341-1363 |
| AD-65603.1 | 599 | GACUUUCAUCCUGGAAAUAUA | 716 | UAUAUUUCCAGGAUGAAAGUCCA | 1341-1363 |
| AD-65608.1 | 600 | GACUUUCAUCCUGGAATUAUA | 717 | UAUAUUUCCAGGAUGAAAGUCCA | 1341-1363 |
| AD-65613.1 | 601 | GACUUUCAUCCUGGAAUAUA | 718 | UAUAUUUCCAGGAUGAAAGUCCA | 1341-1363 |
| AD-65618.1 | 602 | GACUUUCAUCCUGGAATUAUA | 719 | UAUAUUUCCAGGAUGAAAGUCCA | 1341-1363 |
| AD-65623.1 | 603 | GACUUUCAUCCUGGAATUAUA | 720 | UAUAUUUCCAGGAUGAAAGUCCA | 1341-1363 |
| AD-65587.1 | 604 | GACUUUCAUCCUGGAAAUAUA | 721 | UAUAUUUCCAGGAUGAAAGUCCA | 1341-1363 |
| AD-65593.1 | 605 | GACUUTCAUCCUGGAAAUAUA | 722 | UAUAUUUCCAGGAUGAAAGUCCA | 1341-1363 |
| AD-65599.1 | 606 | GACUUUCAUCCUGGAAAUAUA | 723 | UAUAUUUCCAGGATGAAAGUCCA | 1341-1363 |
| AD-65604.1 | 607 | GACUUUCAUCCUGGAAAUAUA | 724 | UAUAUUUCCAGGATGAAAGUCCA | 1341-1363 |
| AD-65609.1 | 608 | GACUUUCAUCCUGGAAAUAUA | 725 | UAUAUUUCCAGGATGAAAGUCCA | 1341-1363 |
| AD-65614.1 | 609 | GACUUUCAUCCUGGAAAUAUA | 726 | UAUAUTUCCAGGATGAAAGUCCA | 1341-1363 |
| AD-65619.1 | 610 | GACUUUCAUCCUGGAAAUAUA | 727 | UAUAUTUCCAGGATGAAAGUCCA | 1341-1363 |
| AD-65624.1 | 611 | GACUUUCAUCCUGGAAAUAUA | 728 | UAUAUUUCCAGGATGAAAGUCCA | 1341-1363 |
| AD-65588.1 | 612 | GACUUUCAUCCUGGAAAUAUA | 729 | UAUAUTUCCAGGATGAAAGUCCA | 1341-1363 |
| AD-65594.1 | 613 | GACUUUCAUCCUGGAAAUAUA | 730 | UAUAUUUCCAGGATGAAAGUCCA | 1341-1363 |
| AD-68309.1 | 614 | AGAAAGGUGUUCAAGAUGUCA | 731 | UGACAUCUUGAACACCUUUCUCC | 1001-1022_C21A |
| AD-68303.1 | 615 | CAUCCUGGAAAUAUAUUAACU | 732 | AGUUAAUAUAUUUCCAGGAUGAA | 1349-1370 |
| AD-65626.5 | 616 | GAAUGUGAAAGUCAUCGACAA | 733 | UUGUCGAUGACUUUCACAUUCUG | 1072-1094 |
| AD-68295.1 | 617 | AGUGCACAAUAUUUUCCCAUA | 734 | UAUGGGAAAAUAUUGUGCACUGU | 1139-1160_C21A |
| AD-68273.1 | 618 | GAAAGUCAUCGACAAGACAUU | 735 | AAUGUCUUGUCGAUGACUUUCAC | 1080-1100 |
| AD-68297.1 | 619 | AAUGUGAAAGUCAUCGACAAA | 736 | UUUGUCGAUGACUUUCACAUUCU | 1075-1096_G21A |
| AD-68287.1 | 620 | CUGGAAAUAUAUUAACUGUUA | 737 | UAACAGUUAAUAUAUUUCCAGGA | 1353-1374 |
| AD-68300.1 | 621 | AUUUUCCCAUCUGUAUUAUUU | 738 | AAAUAAUACAGAUGGGAAAAUAU | 1149-1170 |
| AD-68306.1 | 622 | UGUCGUUCUUUUCCAACAAAA | 739 | UUUUGUUGGAAAAGAACGACACC | 1252-1273 |
| AD-68292.1 | 623 | AUCCUGGAAAUAUAUUAACUA | 740 | UAGUUAAUAUAUUUCCAGGAUGA | 1350-1371_G21A |
| AD-68298.1 | 624 | GCAUUUUGAGAGGUGAUGAUA | 741 | UAUCAUCACCUCUCAAAAUGCCC | 734-755_G21A |
| AD-68277.1 | 625 | CAGGGGGAGAAAGGUGUUCAA | 742 | UUGAACACCUUUCUCCCCCUGGA | 994-1014 |
| AD-68289.1 | 626 | GGAAAUAUAUUAACUGUUAAA | 743 | UUUAACAGUUAAUAUAUUUCCAG | 1355-1376 |
| AD-68272.1 | 627 | CAUUGGUGAGGAAAAAUCCUU | 744 | AAGGAUUUUUCCUCACCAAUGUC | 1097-1117 |
| AD-68282.1 | 628 | GGGAGAAAGGUGUUCAAGAUA | 745 | UAUCUUGAACACCUUUCUCCCCC | 998-1018_G21A |
| AD-68285.1 | 629 | GGCAUUUUGAGAGGUGAUGAU | 746 | AUCAUCACCUCUCAAAAUGCCCU | 733-754 |
| AD-68290.1 | 630 | UACAAAGGGUGUCGUUCUUUU | 747 | AAAAGAACGACACCCUUUGUAUU | 1243-1264 |
| AD-68296.1 | 631 | UGGGAUCUUGGUGUCGAAUCA | 748 | UGAUUCGACACCAAGAUCCCAUU | 783-804 |
| AD-68288.1 | 632 | CUGACAGUGCACAAUAUUUUA | 749 | UAAAAUAUUGUGCACUGUCAGAU | 1134-1155_C21A |
| AD-68299.1 | 633 | CAGUGCACAAUAUUUUCCCAU | 750 | AUGGGAAAAUAUUGUGCACUGUC | 1138-1159 |
| AD-68275.1 | 634 | ACUUUUCAAUGGGUGUCCUAA | 751 | UUAGGACACCCAUUGAAAAGUCA | 1302-1322_G21A |
| AD-68274.1 | 635 | ACAUUGGUGAGGAAAAAUCCU | 752 | AGGAUUUUUCCUCACCAAUGUCU | 1096-1116 |
| AD-68294.1 | 636 | UUGCUUUUGACUUUUCAAUGA | 753 | UCAUUGAAAAGUCAAAAGCAAUG | 1293-1314_G21A |
| AD-68302.1 | 637 | CAUUUUGAGAGGUGAUGAUGA | 754 | UCAUCAUCACCUCUCAAAAUGCC | 735-756_C21A |
| AD-68279.1 | 638 | UUGACUUUUCAAUGGGUGUCA | 755 | UGACACCCAUUGAAAAGUCAAAA | 1299-1319_C21A |
| AD-68304.1 | 639 | CGACUUCUGUUUUAGGACAGA | 756 | UCUGUCCUAAAACAGAAGUCGAC | 212-233 |
| AD-68286.1 | 640 | CUCUGAGUGGGUGCCAGAAUA | 757 | UAUUCUGGCACCCACUCAGAGCC | 1058-1079_G21A |
| AD-68291.1 | 641 | GGGUGCCAGAAUGUGAAAGUA | 758 | UACUUUCACAUUCUGGCACCCAC | 1066-1087_C21A |
| AD-68283.1 | 642 | UCAAUGGGUGUCCUAGGAACA | 759 | UGUUCCUAGGACACCCAUUGAAA | 1307-1327_C21A |
| AD-68280.1 | 643 | AAAGUCAUCGACAAGACAUUA | 760 | UAAUGUCUUGUCGAUGACUUUCA | 1081-1101_G21A |
| AD-68293.1 | 644 | AUUUUGAGAGGUGAUGAUGCA | 761 | UGCAUCAUCACCUCUCAAAAUGC | 736-757_C21A |
| AD-68276.1 | 645 | AUCGACAAGACAUUGGUGAGA | 762 | UCUCACCAAUGUCUUGUCGAUGA | 1087-1107_G21A |
| AD-68308.1 | 646 | GGUGCCAGAAUGUGAAAGUCA | 763 | UGACUUUCACAUUCUGGCACCCA | 1067-1088 |
| AD-68278.1 | 647 | GACAGUGCACAAUAUUUUCCA | 764 | UGGAAAAUAUUGUGCACUGUCAG | 1136-1156_C21A |
| AD-68307.1 | 648 | ACAAAGAGACACUGUGCAGAA | 765 | UUCUGCACAGUGUCUCUUUGUCA | 1191-1212_G21A |
| AD-68284.1 | 649 | UUUUCAAUGGGUGUCCUAGGA | 766 | UCCUAGGACACCCAUUGAAAAGU | 1304-1324 |
| AD-68301.1 | 650 | CCGUUUCCAAGAUCUGACAGU | 767 | ACUGUCAGAUCUUGGAAACGGCC | 1121-1142 |
| AD-68281.1 | 651 | AGGGGGAGAAAGGUGUUCAAA | 768 | UUUGAACACCUUUCUCCCCCUGG | 995-1015_G21A |
| AD-68305.1 | 652 | AGUCAUCGACAAGACAUUGGU | 769 | ACCAAUGUCUUGUCGAUGACUUU | 1083-1104 |

TABLE 12
Additional Human/Mouse/Cyno HAO1 Modified and Unmodified Sense Strand iRNA Sequences

| | | Unmodified sense strand sequence | |
|---|---|---|---|
| Duplex Name | Modified sense strand sequence 5′ to 3′ | 5′ to 3′ | SEQ ID NO: |
| AD-40257.1 | uucAAuGGGuGuccuAGGAdTsdT | UUCAAUGGGUGUCCUAGGA | 770 & 771 |
| AD-40257.2 | uucAAuGGGuGuccuAGGAdTsdT | UUCAAUGGGUGUCCUAGGA | 770 & 771 |
| AD-63102.1 | AcAAcuGGAGGGAcAucGudTsdT | ACAACUGGAGGGACAUCGU | 772 & 773 |
| AD-63102.2 | AcAAcuGGAGGGAcAucGudTsdT | ACAACUGGAGGGACAUCGU | 772 & 773 |
| AD-63102.3 | AcAAcuGGAGGGAcAucGudTsdT | ACAACUGGAGGGACAUCGU | 772 & 773 |

TABLE 13
Additional Human/Mouse/
Cyno HAO1 Modified and Unmodified Antisense Strand iRNA Sequences

| | Modified antisense strand sequence 5′ | Unmodified antisense strand | |
|---|---|---|---|
| Duplex Name | to 3′ | sequence 5′ to 3′ | SEQ ID NO: |
| AD-40257.1 | UCCuAGGAcACCcAUUGAAdTsdT | UCCUAGGACACCCAUUGAA | 774 & 775 |
| AD-40257.2 | UCCuAGGAcACCcAUUGAAdTsdT | UCCUAGGACACCCAUUGAA | 774 & 775 |
| AD-63102.1 | ACGAUGUCCCUCcAGUUGUdTsdT | ACGAUGUCCCUCCAGUUGU | 776 & 777 |
| AD-63102.2 | ACGAUGUCCCUCcAGUUGUdTsdT | ACGAUGUCCCUCCAGUUGU | 776 & 777 |
| AD-63102.3 | ACGAUGUCCCUCcAGUUGUdTsdT | ACGAUGUCCCUCCAGUUGU | 776 & 777 |

TABLE 14
Additional Human/Cyno/Mouse/Rat and Human/Cyno/
Rat HAO1 Modified Sense Strand iRNA Sequences

| Duplex Name | Modified sense strand sequence | SEQ ID NO: |
|---|---|---|
| AD-62989.2 | UfscsCfuAfgGfaAfCfCfuUfuUfaGfaAfaUfL96 | 778 |
| AD-62994.2 | GfsasCfuUfuCfaUfCfCfuGfgAfaAfuAfuAfL96 | 779 |
| AD-62933.2 | GfsasAfuGfuGfaAfAfGfuCfaUfcGfaCfaAfL96 | 780 |
| AD-62935.2 | CfsasUfuGfgUfgAfGfGfaAfaAfaUfcCfuUfL96 | 781 |
| AD-62940.2 | AfsusCfgAfcAfaGfAfCfaUfuGfgUfgAfgAfL96 | 782 |
| AD-62941.2 | AfscsAfuUfgGfuGfAfGfgAfaAfaAfuCfcUfL96 | 783 |
| AD-62944.2 | GfsasAfaGfuCfaUfCfGfaCfaAfgAfcAfuUfL96 | 784 |
| AD-62965.2 | AfsasAfgUfcAfuCfGfAfcAfaGfaCfaUfuAfL96 | 785 |

TABLE 15
Additional Human/Cyno/Mouse/Rat and Human/Cyno/
Rat HAO1 Modified Antisense Strand iRNA Sequences

| | | SEQ |
|---|---|---|
| | | ID |
| Duplex Name | Modified antisense strand | NO: |
| AD-62989.2 | asUfsuUfcUfaAfaAfgguUfcCfuAfgGfascsa | 786 |
| AD-62994.2 | usAfsuAfuUfuCfcAfggaUfgAfaAfgUfcscsa | 787 |
| AD-62933.2 | usUfsgUfcGfaUfgAfcuuUfcAfcAfuUfcsusg | 788 |
| AD-62935.2 | asAfsgGfaUfuUfuUfccuCfaCfcAfaUfgsusc | 789 |
| AD-62940.2 | usCfsuCfaCfcAfaUfgucUfuGfuCfgAfusgsa | 790 |
| AD-62941.2 | asGfsgAfuUfuUfuCfcucAfcCfaAfuGfuscsu | 791 |
| AD-62944.2 | asAfsuGfuCfuUfgUfcgaUfgAfcUfuUfcsasc | 792 |
| AD-62965.2 | usAfsaUfgUfcUfuGfucgAfuGfaCfuUfuscsa | 793 |

TABLE 16
Additional Human Unmodified and Modifieded Sense and Antisense Strand
HAO1 iRNA Sequences Targeting NM_017545.2

| | SEQ ID | | SEQ ID | | |
|---|---|---|---|---|---|
| Unmodified sequence 5′ to 3′ | NO: | Modified sequence 5′ to 3′ | NO: | Strand | Length |
| AUGUAUGUUACUUCUUAGAGA | 794 | asusguauGfuUfAfCfuucuuagagaL96 | 1890 | sense | 21 |
| UCUCUAAGAAGUAACAUACAUCC | 795 | usCfsucuAfaGfAfaguaAfcAfuacauscsc | 1891 | antisense | 23 |
| UGUAUGUUACUUCUUAGAGAG | 796 | usgsuaugUfuAfCfUfucuuagagagL96 | 1892 | sense | 21 |
| CUCUCUAAGAAGUAACAUACAUC | 797 | csUfscucUfaAfGfaaguAfaCfauacasusc | 1893 | antisense | 23 |
| UAGGAUGUAUGUUACUUCUUA | 798 | usasggauGfuAfUfGfuuacuucuuaL96 | 1894 | sense | 21 |
| UAAGAAGUAACAUACAUCCUAAA | 799 | usAfsagaAfgUfAfacauAfcAfuccuasasa | 1895 | antisense | 23 |
| UUAGGAUGUAUGUUACUUCUU | 800 | ususaggaUfgUfAfUfguuacuucuuL96 | 1896 | sense | 21 |
| AAGAAGUAACAUACAUCCUAAAA | 801 | asAfsgaaGfuAfAfcauaCfaUfccuaasasa | 1897 | antisense | 23 |
| AGAAAGGUGUUCAAGAUGUCC | 802 | asgsaaagGfuGfUfUfcaagauguccL96 | 1898 | sense | 21 |
| GGACAUCUUGAACACCUUUCUCC | 803 | gsGfsacaUfcUfUfgaacAfcCfuuucuscsc | 1899 | antisense | 23 |
| GAAAGGUGUUCAAGAUGUCCU | 804 | gsasaaggUfgUfUfCfaagauguccuL96 | 1900 | sense | 21 |
| AGGACAUCUUGAACACCUUUCUC | 805 | asGfsgacAfuCfUfugaaCfaCfcuuucsusc | 1901 | antisense | 23 |
| GGGGAGAAAGGUGUUCAAGAU | 806 | gsgsggagAfaAfGfGfuguucaagauL96 | 1902 | sense | 21 |
| AUCUUGAACACCUUUCUCCCCCU | 807 | asUfscuuGfaAfCfaccuUfuCfuccccscsu | 1903 | antisense | 23 |
| GGGGGAGAAAGGUGUUCAAGA | 808 | gsgsgggaGfaAfAfGfguguucaagaL96 | 1904 | sense | 21 |
| UCUUGAACACCUUUCUCCCCCUG | 809 | usCfsuugAfaCfAfccuuUfcUfcccccsusg | 1905 | antisense | 23 |
| AGAAACUUUGGCUGAUAAUAU | 810 | asgsaaacUfuUfGfGfcugauaauauL96 | 1906 | sense | 21 |
| AUAUUAUCAGCCAAAGUUUCUUC | 811 | asUfsauuAfuCfAfgccaAfaGfuuucususc | 1907 | antisense | 23 |
| GAAACUUUGGCUGAUAAUAUU | 812 | gsasaacuUfuGfGfCfugauaauauuL96 | 1908 | sense | 21 |
| AAUAUUAUCAGCCAAAGUUUCUU | 813 | asAfsuauUfaUfCfagccAfaAfguuucsusu | 1909 | antisense | 23 |
| AUGAAGAAACUUUGGCUGAUA | 814 | asusgaagAfaAfCfUfuuggcugauaL96 | 1910 | sense | 21 |
| UAUCAGCCAAAGUUUCUUCAUCA | 815 | usAfsucaGfcCfAfaaguUfuCfuucauscsa | 1911 | antisense | 23 |
| GAUGAAGAAACUUUGGCUGAU | 816 | gsasugaaGfaAfAfCfuuuggcugauL96 | 1912 | sense | 21 |
| AUCAGCCAAAGUUUCUUCAUCAU | 817 | asUfscagCfcAfAfaguuUfcUfucaucsasu | 1913 | antisense | 23 |
| AAGGCACUGAUGUUCUGAAAG | 818 | asasggcaCfuGfAfUfguucugaaagL96 | 1914 | sense | 21 |
| CUUUCAGAACAUCAGUGCCUUUC | 819 | csUfsuucAfgAfAfcaucAfgUfgccuususc | 1915 | antisense | 23 |
| AGGCACUGAUGUUCUGAAAGC | 820 | asgsgcacUfgAfUfGfuucugaaagcL96 | 1916 | sense | 21 |
| GCUUUCAGAACAUCAGUGCCUUU | 821 | gsCfsuuuCfaGfAfacauCfaGfugccususu | 1917 | antisense | 23 |
| CGGAAAGGCACUGAUGUUCUG | 822 | csgsgaaaGfgCfAfCfugauguucugL96 | 1918 | sense | 21 |
| CAGAACAUCAGUGCCUUUCCGCA | 823 | csAfsgaaCfaUfCfagugCfcUfuuccgscsa | 1919 | antisense | 23 |
| GCGGAAAGGCACUGAUGUUCU | 824 | gscsggaaAfgGfCfAfcugauguucuL96 | 1920 | sense | 21 |
| AGAACAUCAGUGCCUUUCCGCAC | 825 | asGfsaacAfuCfAfgugcCfuUfuccgcsasc | 1921 | antisense | 23 |
| AGAAGACUGACAUCAUUGCCA | 826 | asgsaagaCfuGfAfCfaucauugccaL96 | 1922 | sense | 21 |
| UGGCAAUGAUGUCAGUCUUCUCA | 827 | usGfsgcaAfuGfAfugucAfgUfcuucuscsa | 1923 | antisense | 23 |
| GAAGACUGACAUCAUUGCCAA | 828 | gsasagacUfgAfCfAfucauugccaaL96 | 1924 | sense | 21 |
| UUGGCAAUGAUGUCAGUCUUCUC | 829 | usUfsggcAfaUfGfauguCfaGfucuucsusc | 1925 | antisense | 23 |
| GCUGAGAAGACUGACAUCAUU | 830 | gscsugagAfaGfAfCfugacaucauuL96 | 1926 | sense | 21 |
| AAUGAUGUCAGUCUUCUCAGCCA | 831 | asAfsugaUfgUfCfagucUfuCfucagcscsa | 1927 | antisense | 23 |
| GGCUGAGAAGACUGACAUCAU | 832 | gsgscugaGfaAfGfAfcugacaucauL96 | 1928 | sense | 21 |
| AUGAUGUCAGUCUUCUCAGCCAU | 833 | asUfsgauGfuCfAfgucuUfcUfcagccsasu | 1929 | antisense | 23 |
| UAAUGCCUGAUUCACAACUUU | 834 | usasaugcCfuGfAfUfucacaacuuuL96 | 1930 | sense | 21 |
| AAAGUUGUGAAUCAGGCAUUACC | 835 | asAfsaguUfgUfGfaaucAfgGfcauuascsc | 1931 | antisense | 23 |
| AAUGCCUGAUUCACAACUUUG | 836 | asasugccUfgAfUfUfcacaacuuugL96 | 1932 | sense | 21 |
| CAAAGUUGUGAAUCAGGCAUUAC | 837 | csAfsaagUfuGfUfgaauCfaGfgcauusasc | 1933 | antisense | 23 |
| UUGGUAAUGCCUGAUUCACAA | 838 | ususgguaAfuGfCfCfugauucacaaL96 | 1934 | sense | 21 |
| UUGUGAAUCAGGCAUUACCAACA | 839 | usUfsgugAfaUfCfaggcAfuUfaccaascsa | 1935 | antisense | 23 |
| GUUGGUAAUGCCUGAUUCACA | 840 | gsusugguAfaUfGfCfcugauucacaL96 | 1936 | sense | 21 |
| UGUGAAUCAGGCAUUACCAACAC | 841 | usGfsugaAfuCfAfggcaUfuAfccaacsasc | 1937 | antisense | 23 |
| UAUCAAAUGGCUGAGAAGACU | 842 | usasucaaAfuGfGfCfugagaagacuL96 | 1938 | sense | 21 |
| AGUCUUCUCAGCCAUUUGAUAUC | 843 | asGfsucuUfcUfCfagccAfuUfugauasusc | 1939 | antisense | 23 |
| AUCAAAUGGCUGAGAAGACUG | 844 | asuscaaaUfgGfCfUfgagaagacugL96 | 1940 | sense | 21 |
| CAGUCUUCUCAGCCAUUUGAUAU | 845 | csAfsgucUfuCfUfcagcCfaUfuugausasu | 1941 | antisense | 23 |
| AAGAUAUCAAAUGGCUGAGAA | 846 | asasgauaUfcAfAfAfuggcugagaaL96 | 1942 | sense | 21 |
| UUCUCAGCCAUUUGAUAUCUUCC | 847 | usUfscucAfgCfCfauuuGfaUfaucuuscsc | 1943 | antisense | 23 |
| GAAGAUAUCAAAUGGCUGAGA | 848 | gsasagauAfuCfAfAfauggcugagaL96 | 1944 | sense | 21 |
| UCUCAGCCAUUUGAUAUCUUCCC | 849 | usCfsucaGfcCfAfuuugAfuAfucuucscsc | 1945 | antisense | 23 |
| UCUGACAGUGCACAAUAUUUU | 850 | uscsugacAfgUfGfCfacaauauuuuL96 | 1946 | sense | 21 |
| AAAAUAUUGUGCACUGUCAGAUC | 851 | asAfsaauAfuUfGfugcaCfuGfucagasusc | 1947 | antisense | 23 |
| CUGACAGUGCACAAUAUUUUC | 852 | csusgacaGfuGfCfAfcaauauuuucL96 | 1948 | sense | 21 |
| GAAAAUAUUGUGCACUGUCAGAU | 853 | gsAfsaaaUfaUfUfgugcAfcUfgucagsasu | 1949 | antisense | 23 |
| AAGAUCUGACAGUGCACAAUA | 854 | asasgaucUfgAfCfAfgugcacaauaL96 | 1950 | sense | 21 |
| UAUUGUGCACUGUCAGAUCUUGG | 855 | usAfsuugUfgCfAfcuguCfaGfaucuusgsg | 1951 | antisense | 23 |
| CAAGAUCUGACAGUGCACAAU | 856 | csasagauCfuGfAfCfagugcacaauL96 | 1952 | sense | 21 |
| AUUGUGCACUGUCAGAUCUUGGA | 857 | asUfsuguGfcAfCfugucAfgAfucuugsgsa | 1953 | antisense | 23 |
| ACUGAUGUUCUGAAAGCUCUG | 858 | ascsugauGfuUfCfUfgaaagcucugL96 | 1954 | sense | 21 |
| CAGAGCUUUCAGAACAUCAGUGC | 859 | csAfsgagCfuUfUfcagaAfcAfucagusgsc | 1955 | antisense | 23 |
| CUGAUGUUCUGAAAGCUCUGG | 860 | csusgaugUfuCfUfGfaaagcucuggL96 | 1956 | sense | 21 |
| CCAGAGCUUUCAGAACAUCAGUG | 861 | csCfsagaGfcUfUfucagAfaCfaucagsusg | 1957 | antisense | 23 |
| AGGCACUGAUGUUCUGAAAGC | 862 | asgsgcacUfgAfUfGfuucugaaagcL96 | 1958 | sense | 21 |
| GCUUUCAGAACAUCAGUGCCUUU | 863 | gsCfsuuuCfaGfAfacauCfaGfugccususu | 1959 | antisense | 23 |
| AAGGCACUGAUGUUCUGAAAG | 864 | asasggcaCfuGfAfUfguucugaaagL96 | 1960 | sense | 21 |
| CUUUCAGAACAUCAGUGCCUUUC | 865 | csUfsuucAfgAfAfcaucAfgUfgccuususc | 1961 | antisense | 23 |
| AACAACAUGCUAAAUCAGUAC | 866 | asascaacAfuGfCfUfaaaucaguacL96 | 1962 | sense | 21 |
| GUACUGAUUUAGCAUGUUGUUCA | 867 | gsUfsacuGfaUfUfuagcAfuGfuuguuscsa | 1963 | antisense | 23 |
| ACAACAUGCUAAAUCAGUACU | 868 | ascsaacaUfgCfUfAfaaucaguacuL96 | 1964 | sense | 21 |
| AGUACUGAUUUAGCAUGUUGUUC | 869 | asGfsuacUfgAfUfuuagCfaUfguugususc | 1965 | antisense | 23 |
| UAUGAACAACAUGCUAAAUCA | 870 | usasugaaCfaAfCfAfugcuaaaucaL96 | 1966 | sense | 21 |
| UGAUUUAGCAUGUUGUUCAUAAU | 871 | usGfsauuUfaGfCfauguUfgUfucauasasu | 1967 | antisense | 23 |
| UUAUGAACAACAUGCUAAAUC | 872 | ususaugaAfcAfAfCfaugcuaaaucL96 | 1968 | sense | 21 |
| GAUUUAGCAUGUUGUUCAUAAUC | 873 | gsAfsuuuAfgCfAfuguuGfuUfcauaasusc | 1969 | antisense | 23 |
| UCUUUAGUGUCUGAAUAUAUC | 874 | uscsuuuaGfuGfUfCfugaauauaucL96 | 1970 | sense | 21 |
| GAUAUAUUCAGACACUAAAGAUG | 875 | gsAfsuauAfuUfCfagacAfcUfaaagasusg | 1971 | antisense | 23 |
| CUUUAGUGUCUGAAUAUAUCC | 876 | csusuuagUfgUfCfUfgaauauauccL96 | 1972 | sense | 21 |
| GGAUAUAUUCAGACACUAAAGAU | 877 | gsGfsauaUfaUfUfcagaCfaCfuaaagsasu | 1973 | antisense | 23 |
| CACAUCUUUAGUGUCUGAAUA | 878 | csascaucUfuUfAfGfugucugaauaL96 | 1974 | sense | 21 |
| UAUUCAGACACUAAAGAUGUGAU | 879 | usAfsuucAfgAfCfacuaAfaGfaugugsasu | 1975 | antisense | 23 |
| UCACAUCUUUAGUGUCUGAAU | 880 | uscsacauCfuUfUfAfgugucugaauL96 | 1976 | sense | 21 |
| AUUCAGACACUAAAGAUGUGAUU | 881 | asUfsucaGfaCfAfcuaaAfgAfugugasusu | 1977 | antisense | 23 |
| UGAUACUUCUUUGAAUGUAGA | 882 | usgsauacUfuCfUfUfugaauguagaL96 | 1978 | sense | 21 |
| UCUACAUUCAAAGAAGUAUCACC | 883 | usCfsuacAfuUfCfaaagAfaGfuaucascsc | 1979 | antisense | 23 |
| GAUACUUCUUUGAAUGUAGAU | 884 | gsasuacuUfcUfUfUfgaauguagauL96 | 1980 | sense | 21 |
| AUCUACAUUCAAAGAAGUAUCAC | 885 | asUfscuaCfaUfUfcaaaGfaAfguaucsasc | 1981 | antisense | 23 |
| UUGGUGAUACUUCUUUGAAUG | 886 | ususggugAfuAfCfUfucuuugaaugL96 | 1982 | sense | 21 |
| CAUUCAAAGAAGUAUCACCAAUU | 887 | csAfsuucAfaAfGfaaguAfuCfaccaasusu | 1983 | antisense | 23 |
| AUUGGUGAUACUUCUUUGAAU | 888 | asusugguGfaUfAfCfuucuuugaauL96 | 1984 | sense | 21 |
| AUUCAAAGAAGUAUCACCAAUUA | 889 | asUfsucaAfaGfAfaguaUfcAfccaaususa | 1985 | antisense | 23 |
| AAUAACCUGUGAAAAUGCUCC | 890 | asasuaacCfuGfUfGfaaaaugcuccL96 | 1986 | sense | 21 |
| GGAGCAUUUUCACAGGUUAUUGC | 891 | gsGfsagcAfuUfUfucacAfgGfuuauusgsc | 1987 | antisense | 23 |
| AUAACCUGUGAAAAUGCUCCC | 892 | asusaaccUfgUfGfAfaaaugcucccL96 | 1988 | sense | 21 |
| GGGAGCAUUUUCACAGGUUAUUG | 893 | gsGfsgagCfaUfUfuucaCfaGfguuaususg | 1989 | antisense | 23 |
| UAGCAAUAACCUGUGAAAAUG | 894 | usasgcaaUfaAfCfCfugugaaaaugL96 | 1990 | sense | 21 |
| CAUUUUCACAGGUUAUUGCUAUC | 895 | csAfsuuuUfcAfCfagguUfaUfugcuasusc | 1991 | antisense | 23 |
| AUAGCAAUAACCUGUGAAAAU | 896 | asusagcaAfuAfAfCfcugugaaaauL96 | 1992 | sense | 21 |
| AUUUUCACAGGUUAUUGCUAUCC | 897 | asUfsuuuCfaCfAfgguuAfuUfgcuauscsc | 1993 | antisense | 23 |
| AAUCACAUCUUUAGUGUCUGA | 898 | asasucacAfuCfUfUfuagugucugaL96 | 1994 | sense | 21 |
| UCAGACACUAAAGAUGUGAUUGG | 899 | usCfsagaCfaCfUfaaagAfuGfugauusgsg | 1995 | antisense | 23 |
| AUCACAUCUUUAGUGUCUGAA | 900 | asuscacaUfcUfUfUfagugucugaaL96 | 1996 | sense | 21 |
| UUCAGACACUAAAGAUGUGAUUG | 901 | usUfscagAfcAfCfuaaaGfaUfgugaususg | 1997 | antisense | 23 |
| UUCCAAUCACAUCUUUAGUGU | 902 | ususccaaUfcAfCfAfucuuuaguguL96 | 1998 | sense | 21 |
| ACACUAAAGAUGUGAUUGGAAAU | 903 | asCfsacuAfaAfGfauguGfaUfuggaasasu | 1999 | antisense | 23 |
| UUUCCAAUCACAUCUUUAGUG | 904 | ususuccaAfuCfAfCfaucuuuagugL96 | 2000 | sense | 21 |
| CACUAAAGAUGUGAUUGGAAAUC | 905 | csAfscuaAfaGfAfugugAfuUfggaaasusc | 2001 | antisense | 23 |
| ACGGGCAUGAUGUUGAGUUCC | 906 | ascsgggcAfuGfAfUfguugaguuccL96 | 2002 | sense | 21 |
| GGAACUCAACAUCAUGCCCGUUC | 907 | gsGfsaacUfcAfAfcaucAfuGfcccgususc | 2003 | antisense | 23 |
| CGGGCAUGAUGUUGAGUUCCU | 908 | csgsggcaUfgAfUfGfuugaguuccuL96 | 2004 | sense | 21 |
| AGGAACUCAACAUCAUGCCCGUU | 909 | asGfsgaaCfuCfAfacauCfaUfgcccgsusu | 2005 | antisense | 23 |
| GGGAACGGGCAUGAUGUUGAG | 910 | gsgsgaacGfgGfCfAfugauguugagL96 | 2006 | sense | 21 |
| CUCAACAUCAUGCCCGUUCCCAG | 911 | csUfscaaCfaUfCfaugcCfcGfuucccsasg | 2007 | antisense | 23 |
| UGGGAACGGGCAUGAUGUUGA | 912 | usgsggaaCfgGfGfCfaugauguugaL96 | 2008 | sense | 21 |
| UCAACAUCAUGCCCGUUCCCAGG | 913 | usCfsaacAfuCfAfugccCfgUfucccasgsg | 2009 | antisense | 23 |
| ACUAAGGUGAAAAGAUAAUGA | 914 | ascsuaagGfuGfAfAfaagauaaugaL96 | 2010 | sense | 21 |
| UCAUUAUCUUUUCACCUUAGUGU | 915 | usCfsauuAfuCfUfuuucAfcCfuuagusgsu | 2011 | antisense | 23 |
| CUAAGGUGAAAAGAUAAUGAU | 916 | csusaaggUfgAfAfAfagauaaugauL96 | 2012 | sense | 21 |
| AUCAUUAUCUUUUCACCUUAGUG | 917 | asUfscauUfaUfCfuuuuCfaCfcuuagsusg | 2013 | antisense | 23 |
| AAACACUAAGGUGAAAAGAUA | 918 | asasacacUfaAfGfGfugaaaagauaL96 | 2014 | sense | 21 |
| UAUCUUUUCACCUUAGUGUUUGC | 919 | usAfsucuUfuUfCfaccuUfaGfuguuusgsc | 2015 | antisense | 23 |
| CAAACACUAAGGUGAAAAGAU | 920 | csasaacaCfuAfAfGfgugaaaagauL96 | 2016 | sense | 21 |
| AUCUUUUCACCUUAGUGUUUGCU | 921 | asUfscuuUfuCfAfccuuAfgUfguuugscsu | 2017 | antisense | 23 |
| AGGUAGCACUGGAGAGAAUUG | 922 | asgsguagCfaCfUfGfgagagaauugL96 | 2018 | sense | 21 |
| CAAUUCUCUCCAGUGCUACCUUC | 923 | csAfsauuCfuCfUfccagUfgCfuaccususc | 2019 | antisense | 23 |
| GGUAGCACUGGAGAGAAUUGG | 924 | gsgsuagcAfcUfGfGfagagaauuggL96 | 2020 | sense | 21 |
| CCAAUUCUCUCCAGUGCUACCUU | 925 | csCfsaauUfcUfCfuccaGfuGfcuaccsusu | 2021 | antisense | 23 |
| GAGAAGGUAGCACUGGAGAGA | 926 | gsasgaagGfuAfGfCfacuggagagaL96 | 2022 | sense | 21 |
| UCUCUCCAGUGCUACCUUCUCAA | 927 | usCfsucuCfcAfGfugcuAfcCfuucucsasa | 2023 | antisense | 23 |
| UGAGAAGGUAGCACUGGAGAG | 928 | usgsagaaGfgUfAfGfcacuggagagL96 | 2024 | sense | 21 |
| CUCUCCAGUGCUACCUUCUCAAA | 929 | csUfscucCfaGfUfgcuaCfcUfucucasasa | 2025 | antisense | 23 |
| AGUGGACUUGCUGCAUAUGUG | 930 | asgsuggaCfuUfGfCfugcauaugugL96 | 2026 | sense | 21 |
| CACAUAUGCAGCAAGUCCACUGU | 931 | csAfscauAfuGfCfagcaAfgUfccacusgsu | 2027 | antisense | 23 |
| GUGGACUUGCUGCAUAUGUGG | 932 | gsusggacUfuGfCfUfgcauauguggL96 | 2028 | sense | 21 |
| CCACAUAUGCAGCAAGUCCACUG | 933 | csCfsacaUfaUfGfcagcAfaGfuccacsusg | 2029 | antisense | 23 |
| CGACAGUGGACUUGCUGCAUA | 934 | csgsacagUfgGfAfCfuugcugcauaL96 | 2030 | sense | 21 |
| UAUGCAGCAAGUCCACUGUCGUC | 935 | usAfsugcAfgCfAfagucCfaCfugucgsusc | 2031 | antisense | 23 |
| ACGACAGUGGACUUGCUGCAU | 936 | ascsgacaGfuGfGfAfcuugcugcauL96 | 2032 | sense | 21 |
| AUGCAGCAAGUCCACUGUCGUCU | 937 | asUfsgcaGfcAfAfguccAfcUfgucguscsu | 2033 | antisense | 23 |
| AAGGUGUUCAAGAUGUCCUCG | 938 | asasggugUfuCfAfAfgauguccucgL96 | 2034 | sense | 21 |
| CGAGGACAUCUUGAACACCUUUC | 939 | csGfsaggAfcAfUfcuugAfaCfaccuususc | 2035 | antisense | 23 |
| AGGUGUUCAAGAUGUCCUCGA | 940 | asgsguguUfcAfAfGfauguccucgaL96 | 2036 | sense | 21 |
| UCGAGGACAUCUUGAACACCUUU | 941 | usCfsgagGfaCfAfucuuGfaAfcaccususu | 2037 | antisense | 23 |
| GAGAAAGGUGUUCAAGAUGUC | 942 | gsasgaaaGfgUfGfUfucaagaugucL96 | 2038 | sense | 21 |
| GACAUCUUGAACACCUUUCUCCC | 943 | gsAfscauCfuUfGfaacaCfcUfuucucscsc | 2039 | antisense | 23 |
| GGAGAAAGGUGUUCAAGAUGU | 944 | gsgsagaaAfgGfUfGfuucaagauguL96 | 2040 | sense | 21 |
| ACAUCUUGAACACCUUUCUCCCC | 945 | asCfsaucUfuGfAfacacCfuUfucuccscsc | 2041 | antisense | 23 |
| AACCGUCUGGAUGAUGUGCGU | 946 | asasccguCfuGfGfAfugaugugcguL96 | 2042 | sense | 21 |
| ACGCACAUCAUCCAGACGGUUGC | 947 | asCfsgcaCfaUfCfauccAfgAfcgguusgsc | 2043 | antisense | 23 |
| ACCGUCUGGAUGAUGUGCGUA | 948 | ascscgucUfgGfAfUfgaugugcguaL96 | 2044 | sense | 21 |
| UACGCACAUCAUCCAGACGGUUG | 949 | usAfscgcAfcAfUfcaucCfaGfacggususg | 2045 | antisense | 23 |
| GGGCAACCGUCUGGAUGAUGU | 950 | gsgsgcaaCfcGfUfCfuggaugauguL96 | 2046 | sense | 21 |
| ACAUCAUCCAGACGGUUGCCCAG | 951 | asCfsaucAfuCfCfagacGfgUfugcccsasg | 2047 | antisense | 23 |
| UGGGCAACCGUCUGGAUGAUG | 952 | usgsggcaAfcCfGfUfcuggaugaugL96 | 2048 | sense | 21 |
| CAUCAUCCAGACGGUUGCCCAGG | 953 | csAfsucaUfcCfAfgacgGfuUfgcccasgsg | 2049 | antisense | 23 |
| GAAACUUUGGCUGAUAAUAUU | 954 | gsasaacuUfuGfGfCfugauaauauuL96 | 2050 | sense | 21 |
| AAUAUUAUCAGCCAAAGUUUCUU | 955 | asAfsuauUfaUfCfagccAfaAfguuucsusu | 2051 | antisense | 23 |
| AAACUUUGGCUGAUAAUAUUG | 956 | asasacuuUfgGfCfUfgauaauauugL96 | 2052 | sense | 21 |
| CAAUAUUAUCAGCCAAAGUUUCU | 957 | csAfsauaUfuAfUfcagcCfaAfaguuuscsu | 2053 | antisense | 23 |
| UGAAGAAACUUUGGCUGAUAA | 958 | usgsaagaAfaCfUfUfuggcugauaaL96 | 2054 | sense | 21 |
| UUAUCAGCCAAAGUUUCUUCAUC | 959 | usUfsaucAfgCfCfaaagUfuUfcuucasusc | 2055 | antisense | 23 |
| AUGAAGAAACUUUGGCUGAUA | 960 | asusgaagAfaAfCfUfuuggcugauaL96 | 2056 | sense | 21 |
| UAUCAGCCAAAGUUUCUUCAUCA | 961 | usAfsucaGfcCfAfaaguUfuCfuucauscsa | 2057 | antisense | 23 |
| AAAGGUGUUCAAGAUGUCCUC | 962 | asasagguGfuUfCfAfagauguccucL96 | 2058 | sense | 21 |
| GAGGACAUCUUGAACACCUUUCU | 963 | gsAfsggaCfaUfCfuugaAfcAfccuuuscsu | 2059 | antisense | 23 |
| AAGGUGUUCAAGAUGUCCUCG | 964 | asasggugUfuCfAfAfgauguccucgL96 | 2060 | sense | 21 |
| CGAGGACAUCUUGAACACCUUUC | 965 | csGfsaggAfcAfUfcuugAfaCfaccuususc | 2061 | antisense | 23 |
| GGAGAAAGGUGUUCAAGAUGU | 966 | gsgsagaaAfgGfUfGfuucaagauguL96 | 2062 | sense | 21 |
| ACAUCUUGAACACCUUUCUCCCC | 967 | asCfsaucUfuGfAfacacCfuUfucuccscsc | 2063 | antisense | 23 |
| GGGAGAAAGGUGUUCAAGAUG | 968 | gsgsgagaAfaGfGfUfguucaagaugL96 | 2064 | sense | 21 |
| CAUCUUGAACACCUUUCUCCCCC | 969 | csAfsucuUfgAfAfcaccUfuUfcucccscsc | 2065 | antisense | 23 |
| AAAUCAGUACUUCCAAAGUCU | 970 | asasaucaGfuAfCfUfuccaaagucuL96 | 2066 | sense | 21 |
| AGACUUUGGAAGUACUGAUUUAG | 971 | asGfsacuUfuGfGfaaguAfcUfgauuusasg | 2067 | antisense | 23 |
| AAUCAGUACUUCCAAAGUCUA | 972 | asasucagUfaCfUfUfccaaagucuaL96 | 2068 | sense | 21 |
| UAGACUUUGGAAGUACUGAUUUA | 973 | usAfsgacUfuUfGfgaagUfaCfugauususa | 2069 | antisense | 23 |
| UGCUAAAUCAGUACUUCCAAA | 974 | usgscuaaAfuCfAfGfuacuuccaaaL96 | 2070 | sense | 21 |
| UUUGGAAGUACUGAUUUAGCAUG | 975 | usUfsuggAfaGfUfacugAfuUfuagcasusg | 2071 | antisense | 23 |
| AUGCUAAAUCAGUACUUCCAA | 976 | asusgcuaAfaUfCfAfguacuuccaaL96 | 2072 | sense | 21 |
| UUGGAAGUACUGAUUUAGCAUGU | 977 | usUfsggaAfgUfAfcugaUfuUfagcausgsu | 2073 | antisense | 23 |
| ACAUCUUUAGUGUCUGAAUAU | 978 | ascsaucuUfuAfGfUfgucugaauauL96 | 2074 | sense | 21 |
| AUAUUCAGACACUAAAGAUGUGA | 979 | asUfsauuCfaGfAfcacuAfaAfgaugusgsa | 2075 | antisense | 23 |
| CAUCUUUAGUGUCUGAAUAUA | 980 | csasucuuUfaGfUfGfucugaauauaL96 | 2076 | sense | 21 |
| UAUAUUCAGACACUAAAGAUGUG | 981 | usAfsuauUfcAfGfacacUfaAfagaugsusg | 2077 | antisense | 23 |
| AAUCACAUCUUUAGUGUCUGA | 982 | asasucacAfuCfUfUfuagugucugaL96 | 2078 | sense | 21 |
| UCAGACACUAAAGAUGUGAUUGG | 983 | usCfsagaCfaCfUfaaagAfuGfugauusgsg | 2079 | antisense | 23 |
| CAAUCACAUCUUUAGUGUCUG | 984 | csasaucaCfaUfCfUfuuagugucugL96 | 2080 | sense | 21 |
| CAGACACUAAAGAUGUGAUUGGA | 985 | csAfsgacAfcUfAfaagaUfgUfgauugsgsa | 2081 | antisense | 23 |
| GCAUGUAUUACUUGACAAAGA | 986 | gscsauguAfuUfAfCfuugacaaagaL96 | 2082 | sense | 21 |
| UCUUUGUCAAGUAAUACAUGCUG | 987 | usCfsuuuGfuCfAfaguaAfuAfcaugcsusg | 2083 | antisense | 23 |
| CAUGUAUUACUUGACAAAGAG | 988 | csasuguaUfuAfCfUfugacaaagagL96 | 2084 | sense | 21 |
| CUCUUUGUCAAGUAAUACAUGCU | 989 | csUfscuuUfgUfCfaaguAfaUfacaugscsu | 2085 | antisense | 23 |
| UUCAGCAUGUAUUACUUGACA | 990 | ususcagcAfuGfUfAfuuacuugacaL96 | 2086 | sense | 21 |
| UGUCAAGUAAUACAUGCUGAAAA | 991 | usGfsucaAfgUfAfauacAfuGfcugaasasa | 2087 | antisense | 23 |
| UUUCAGCAUGUAUUACUUGAC | 992 | ususucagCfaUfGfUfauuacuugacL96 | 2088 | sense | 21 |
| GUCAAGUAAUACAUGCUGAAAAA | 993 | gsUfscaaGfuAfAfuacaUfgCfugaaasasa | 2089 | antisense | 23 |
| AUGUUACUUCUUAGAGAGAAA | 994 | asusguuaCfuUfCfUfuagagagaaaL96 | 2090 | sense | 21 |
| UUUCUCUCUAAGAAGUAACAUAC | 995 | usUfsucuCfuCfUfaagaAfgUfaacausasc | 2091 | antisense | 23 |
| UGUUACUUCUUAGAGAGAAAU | 996 | usgsuuacUfuCfUfUfagagagaaauL96 | 2092 | sense | 21 |
| AUUUCUCUCUAAGAAGUAACAUA | 997 | asUfsuucUfcUfCfuaagAfaGfuaacasusa | 2093 | antisense | 23 |
| AUGUAUGUUACUUCUUAGAGA | 998 | asusguauGfuUfAfCfuucuuagagaL96 | 2094 | sense | 21 |
| UCUCUAAGAAGUAACAUACAUCC | 999 | usCfsucuAfaGfAfaguaAfcAfuacauscsc | 2095 | antisense | 23 |
| GAUGUAUGUUACUUCUUAGAG | 1000 | gsasuguaUfgUfUfAfcuucuuagagL96 | 2096 | sense | 21 |
| CUCUAAGAAGUAACAUACAUCCU | 1001 | csUfscuaAfgAfAfguaaCfaUfacaucscsu | 2097 | antisense | 23 |
| ACAACUUUGAGAAGGUAGCAC | 1002 | ascsaacuUfuGfAfGfaagguagcacL96 | 2098 | sense | 21 |
| GUGCUACCUUCUCAAAGUUGUGA | 1003 | gsUfsgcuAfcCfUfucucAfaAfguugusgsa | 2099 | antisense | 23 |
| CAACUUUGAGAAGGUAGCACU | 1004 | csasacuuUfgAfGfAfagguagcacuL96 | 2100 | sense | 21 |
| AGUGCUACCUUCUCAAAGUUGUG | 1005 | asGfsugcUfaCfCfuucuCfaAfaguugsusg | 2101 | antisense | 23 |
| AUUCACAACUUUGAGAAGGUA | 1006 | asusucacAfaCfUfUfugagaagguaL96 | 2102 | sense | 21 |
| UACCUUCUCAAAGUUGUGAAUCA | 1007 | usAfsccuUfcUfCfaaagUfuGfugaauscsa | 2103 | antisense | 23 |
| GAUUCACAACUUUGAGAAGGU | 1008 | gsasuucaCfaAfCfUfuugagaagguL96 | 2104 | sense | 21 |
| ACCUUCUCAAAGUUGUGAAUCAG | 1009 | asCfscuuCfuCfAfaaguUfgUfgaaucsasg | 2105 | antisense | 23 |
| AACAUGCUAAAUCAGUACUUC | 1010 | asascaugCfuAfAfAfucaguacuucL96 | 2106 | sense | 21 |
| GAAGUACUGAUUUAGCAUGUUGU | 1011 | gsAfsaguAfcUfGfauuuAfgCfauguusgsu | 2107 | antisense | 23 |
| ACAUGCUAAAUCAGUACUUCC | 1012 | ascsaugcUfaAfAfUfcaguacuuccL96 | 2108 | sense | 21 |
| GGAAGUACUGAUUUAGCAUGUUG | 1013 | gsGfsaagUfaCfUfgauuUfaGfcaugususg | 2109 | antisense | 23 |
| GAACAACAUGCUAAAUCAGUA | 1014 | gsasacaaCfaUfGfCfuaaaucaguaL96 | 2110 | sense | 21 |
| UACUGAUUUAGCAUGUUGUUCAU | 1015 | usAfscugAfuUfUfagcaUfgUfuguucsasu | 2111 | antisense | 23 |
| UGAACAACAUGCUAAAUCAGU | 1016 | usgsaacaAfcAfUfGfcuaaaucaguL96 | 2112 | sense | 21 |
| ACUGAUUUAGCAUGUUGUUCAUA | 1017 | asCfsugaUfuUfAfgcauGfuUfguucasusa | 2113 | antisense | 23 |
| AAACCAGUACUUUAUCAUUUU | 1018 | asasaccaGfuAfCfUfuuaucauuuuL96 | 2114 | sense | 21 |
| AAAAUGAUAAAGUACUGGUUUCA | 1019 | asAfsaauGfaUfAfaaguAfcUfgguuuscsa | 2115 | antisense | 23 |
| AACCAGUACUUUAUCAUUUUC | 1020 | asasccagUfaCfUfUfuaucauuuucL96 | 2116 | sense | 21 |
| GAAAAUGAUAAAGUACUGGUUUC | 1021 | gsAfsaaaUfgAfUfaaagUfaCfugguususc | 2117 | antisense | 23 |
| UUUGAAACCAGUACUUUAUCA | 1022 | ususugaaAfcCfAfGfuacuuuaucaL96 | 2118 | sense | 21 |
| UGAUAAAGUACUGGUUUCAAAAU | 1023 | usGfsauaAfaGfUfacugGfuUfucaaasasu | 2119 | antisense | 23 |
| UUUUGAAACCAGUACUUUAUC | 1024 | ususuugaAfaCfCfAfguacuuuaucL96 | 2120 | sense | 21 |
| GAUAAAGUACUGGUUUCAAAAUU | 1025 | gsAfsuaaAfgUfAfcuggUfuUfcaaaasusu | 2121 | antisense | 23 |
| GAGAAGAUGGGCUACAAGGCC | 1026 | gsasgaagAfuGfGfGfcuacaaggccL96 | 2122 | sense | 21 |
| GGCCUUGUAGCCCAUCUUCUCUG | 1027 | gsGfsccuUfgUfAfgcccAfuCfuucucsusg | 2123 | antisense | 23 |
| AGAAGAUGGGCUACAAGGCCA | 1028 | asgsaagaUfgGfGfCfuacaaggccaL96 | 2124 | sense | 21 |
| UGGCCUUGUAGCCCAUCUUCUCU | 1029 | usGfsgccUfuGfUfagccCfaUfcuucuscsu | 2125 | antisense | 23 |
| GGCAGAGAAGAUGGGCUACAA | 1030 | gsgscagaGfaAfGfAfugggcuacaaL96 | 2126 | sense | 21 |
| UUGUAGCCCAUCUUCUCUGCCUG | 1031 | usUfsguaGfcCfCfaucuUfcUfcugccsusg | 2127 | antisense | 23 |
| AGGCAGAGAAGAUGGGCUACA | 1032 | asgsgcagAfgAfAfGfaugggcuacaL96 | 2128 | sense | 21 |
| UGUAGCCCAUCUUCUCUGCCUGC | 1033 | usGfsuagCfcCfAfucuuCfuCfugccusgsc | 2129 | antisense | 23 |
| AACGGGCAUGAUGUUGAGUUC | 1034 | asascgggCfaUfGfAfuguugaguucL96 | 2130 | sense | 21 |
| GAACUCAACAUCAUGCCCGUUCC | 1035 | gsAfsacuCfaAfCfaucaUfgCfccguuscsc | 2131 | antisense | 23 |
| ACGGGCAUGAUGUUGAGUUCC | 1036 | ascsgggcAfuGfAfUfguugaguuccL96 | 2132 | sense | 21 |
| GGAACUCAACAUCAUGCCCGUUC | 1037 | gsGfsaacUfcAfAfcaucAfuGfcccgususc | 2133 | antisense | 23 |
| UGGGAACGGGCAUGAUGUUGA | 1038 | usgsggaaCfgGfGfCfaugauguugaL96 | 2134 | sense | 21 |
| UCAACAUCAUGCCCGUUCCCAGG | 1039 | usCfsaacAfuCfAfugccCfgUfucccasgsg | 2135 | antisense | 23 |
| CUGGGAACGGGCAUGAUGUUG | 1040 | csusgggaAfcGfGfGfcaugauguugL96 | 2136 | sense | 21 |
| CAACAUCAUGCCCGUUCCCAGGG | 1041 | csAfsacaUfcAfUfgcccGfuUfcccagsgsg | 2137 | antisense | 23 |
| AUGUGGCUAAAGCAAUAGACC | 1042 | asusguggCfuAfAfAfgcaauagaccL96 | 2138 | sense | 21 |
| GGUCUAUUGCUUUAGCCACAUAU | 1043 | gsGfsucuAfuUfGfcuuuAfgCfcacausasu | 2139 | antisense | 23 |
| UGUGGCUAAAGCAAUAGACCC | 1044 | usgsuggcUfaAfAfGfcaauagacccL96 | 2140 | sense | 21 |
| GGGUCUAUUGCUUUAGCCACAUA | 1045 | gsGfsgucUfaUfUfgcuuUfaGfccacasusa | 2141 | antisense | 23 |
| GCAUAUGUGGCUAAAGCAAUA | 1046 | gscsauauGfuGfGfCfuaaagcaauaL96 | 2142 | sense | 21 |
| UAUUGCUUUAGCCACAUAUGCAG | 1047 | usAfsuugCfuUfUfagccAfcAfuaugcsasg | 2143 | antisense | 23 |
| UGCAUAUGUGGCUAAAGCAAU | 1048 | usgscauaUfgUfGfGfcuaaagcaauL96 | 2144 | sense | 21 |
| AUUGCUUUAGCCACAUAUGCAGC | 1049 | asUfsugcUfuUfAfgccaCfaUfaugcasgsc | 2145 | antisense | 23 |
| AGGAUGCUCCGGAAUGUUGCU | 1050 | asgsgaugCfuCfCfGfgaauguugcuL96 | 2146 | sense | 21 |
| AGCAACAUUCCGGAGCAUCCUUG | 1051 | asGfscaaCfaUfUfccggAfgCfauccususg | 2147 | antisense | 23 |
| GGAUGCUCCGGAAUGUUGCUG | 1052 | gsgsaugcUfcCfGfGfaauguugcugL96 | 2148 | sense | 21 |
| CAGCAACAUUCCGGAGCAUCCUU | 1053 | csAfsgcaAfcAfUfuccgGfaGfcauccsusu | 2149 | antisense | 23 |
| UCCAAGGAUGCUCCGGAAUGU | 1054 | uscscaagGfaUfGfCfuccggaauguL96 | 2150 | sense | 21 |
| ACAUUCCGGAGCAUCCUUGGAUA | 1055 | asCfsauuCfcGfGfagcaUfcCfuuggasusa | 2151 | antisense | 23 |
| AUCCAAGGAUGCUCCGGAAUG | 1056 | asusccaaGfgAfUfGfcuccggaaugL96 | 2152 | sense | 21 |
| CAUUCCGGAGCAUCCUUGGAUAC | 1057 | csAfsuucCfgGfAfgcauCfcUfuggausasc | 2153 | antisense | 23 |
| UCACAUCUUUAGUGUCUGAAU | 1058 | uscsacauCfuUfUfAfgugucugaauL96 | 2154 | sense | 21 |
| AUUCAGACACUAAAGAUGUGAUU | 1059 | asUfsucaGfaCfAfcuaaAfgAfugugasusu | 2155 | antisense | 23 |
| CACAUCUUUAGUGUCUGAAUA | 1060 | csascaucUfuUfAfGfugucugaauaL96 | 2156 | sense | 21 |
| UAUUCAGACACUAAAGAUGUGAU | 1061 | usAfsuucAfgAfCfacuaAfaGfaugugsasu | 2157 | antisense | 23 |
| CCAAUCACAUCUUUAGUGUCU | 1062 | cscsaaucAfcAfUfCfuuuagugucuL96 | 2158 | sense | 21 |
| AGACACUAAAGAUGUGAUUGGAA | 1063 | asGfsacaCfuAfAfagauGfuGfauuggsasa | 2159 | antisense | 23 |
| UCCAAUCACAUCUUUAGUGUC | 1064 | uscscaauCfaCfAfUfcuuuagugucL96 | 2160 | sense | 21 |
| GACACUAAAGAUGUGAUUGGAAA | 1065 | gsAfscacUfaAfAfgaugUfgAfuuggasasa | 2161 | antisense | 23 |
| AAAUGUGUUUAGACAACGUCA | 1066 | asasauguGfuUfUfAfgacaacgucaL96 | 2162 | sense | 21 |
| UGACGUUGUCUAAACACAUUUUC | 1067 | usGfsacgUfuGfUfcuaaAfcAfcauuususc | 2163 | antisense | 23 |
| AAUGUGUUUAGACAACGUCAU | 1068 | asasugugUfuUfAfGfacaacgucauL96 | 2164 | sense | 21 |
| AUGACGUUGUCUAAACACAUUUU | 1069 | asUfsgacGfuUfGfucuaAfaCfacauususu | 2165 | antisense | 23 |
| UUGAAAAUGUGUUUAGACAAC | 1070 | ususgaaaAfuGfUfGfuuuagacaacL96 | 2166 | sense | 21 |
| GUUGUCUAAACACAUUUUCAAUG | 1071 | gsUfsuguCfuAfAfacacAfuUfuucaasusg | 2167 | antisense | 23 |
| AUUGAAAAUGUGUUUAGACAA | 1072 | asusugaaAfaUfGfUfguuuagacaaL96 | 2168 | sense | 21 |
| UUGUCUAAACACAUUUUCAAUGU | 1073 | usUfsgucUfaAfAfcacaUfuUfucaausgsu | 2169 | antisense | 23 |
| UACUAAAGGAAGAAUUCCGGU | 1074 | usascuaaAfgGfAfAfgaauuccgguL96 | 2170 | sense | 21 |
| ACCGGAAUUCUUCCUUUAGUAUC | 1075 | asCfscggAfaUfUfcuucCfuUfuaguasusc | 2171 | antisense | 23 |
| ACUAAAGGAAGAAUUCCGGUU | 1076 | ascsuaaaGfgAfAfGfaauuccgguuL96 | 2172 | sense | 21 |
| AACCGGAAUUCUUCCUUUAGUAU | 1077 | asAfsccgGfaAfUfucuuCfcUfuuagusasu | 2173 | antisense | 23 |
| GAGAUACUAAAGGAAGAAUUC | 1078 | gsasgauaCfuAfAfAfggaagaauucL96 | 2174 | sense | 21 |
| GAAUUCUUCCUUUAGUAUCUCGA | 1079 | gsAfsauuCfuUfCfcuuuAfgUfaucucsgsa | 2175 | antisense | 23 |
| CGAGAUACUAAAGGAAGAAUU | 1080 | csgsagauAfcUfAfAfaggaagaauuL96 | 2176 | sense | 21 |
| AAUUCUUCCUUUAGUAUCUCGAG | 1081 | asAfsuucUfuCfCfuuuaGfuAfucucgsasg | 2177 | antisense | 23 |
| AACUUUGGCUGAUAAUAUUGC | 1082 | asascuuuGfgCfUfGfauaauauugcL96 | 2178 | sense | 21 |
| GCAAUAUUAUCAGCCAAAGUUUC | 1083 | gsCfsaauAfuUfAfucagCfcAfaaguususc | 2179 | antisense | 23 |
| ACUUUGGCUGAUAAUAUUGCA | 1084 | ascsuuugGfcUfGfAfuaauauugcaL96 | 2180 | sense | 21 |
| UGCAAUAUUAUCAGCCAAAGUUU | 1085 | usGfscaaUfaUfUfaucaGfcCfaaagususu | 2181 | antisense | 23 |
| AAGAAACUUUGGCUGAUAAUA | 1086 | asasgaaaCfuUfUfGfgcugauaauaL96 | 2182 | sense | 21 |
| UAUUAUCAGCCAAAGUUUCUUCA | 1087 | usAfsuuaUfcAfGfccaaAfgUfuucuuscsa | 2183 | antisense | 23 |
| GAAGAAACUUUGGCUGAUAAU | 1088 | gsasagaaAfcUfUfUfggcugauaauL96 | 2184 | sense | 21 |
| AUUAUCAGCCAAAGUUUCUUCAU | 1089 | asUfsuauCfaGfCfcaaaGfuUfucuucsasu | 2185 | antisense | 23 |
| AAAUGGCUGAGAAGACUGACA | 1090 | asasauggCfuGfAfGfaagacugacaL96 | 2186 | sense | 21 |
| UGUCAGUCUUCUCAGCCAUUUGA | 1091 | usGfsucaGfuCfUfucucAfgCfcauuusgsa | 2187 | antisense | 23 |
| AAUGGCUGAGAAGACUGACAU | 1092 | asasuggcUfgAfGfAfagacugacauL96 | 2188 | sense | 21 |
| AUGUCAGUCUUCUCAGCCAUUUG | 1093 | asUfsgucAfgUfCfuucuCfaGfccauususg | 2189 | antisense | 23 |
| UAUCAAAUGGCUGAGAAGACU | 1094 | usasucaaAfuGfGfCfugagaagacuL96 | 2190 | sense | 21 |
| AGUCUUCUCAGCCAUUUGAUAUC | 1095 | asGfsucuUfcUfCfagccAfuUfugauasusc | 2191 | antisense | 23 |
| AUAUCAAAUGGCUGAGAAGAC | 1096 | asusaucaAfaUfGfGfcugagaagacL96 | 2192 | sense | 21 |
| GUCUUCUCAGCCAUUUGAUAUCU | 1097 | gsUfscuuCfuCfAfgccaUfuUfgauauscsu | 2193 | antisense | 23 |
| GUGGUUCUUAAAUUGUAAGCU | 1098 | gsusgguuCfuUfAfAfauuguaagcuL96 | 2194 | sense | 21 |
| AGCUUACAAUUUAAGAACCACUG | 1099 | asGfscuuAfcAfAfuuuaAfgAfaccacsusg | 2195 | antisense | 23 |
| UGGUUCUUAAAUUGUAAGCUC | 1100 | usgsguucUfuAfAfAfuuguaagcucL96 | 2196 | sense | 21 |
| GAGCUUACAAUUUAAGAACCACU | 1101 | gsAfsgcuUfaCfAfauuuAfaGfaaccascsu | 2197 | antisense | 23 |
| AACAGUGGUUCUUAAAUUGUA | 1102 | asascaguGfgUfUfCfuuaaauuguaL96 | 2198 | sense | 21 |
| UACAAUUUAAGAACCACUGUUUU | 1103 | usAfscaaUfuUfAfagaaCfcAfcuguususu | 2199 | antisense | 23 |
| AAACAGUGGUUCUUAAAUUGU | 1104 | asasacagUfgGfUfUfcuuaaauuguL96 | 2200 | sense | 21 |
| ACAAUUUAAGAACCACUGUUUUA | 1105 | asCfsaauUfuAfAfgaacCfaCfuguuususa | 2201 | antisense | 23 |
| AAGUCAUCGACAAGACAUUGG | 1106 | asasgucaUfcGfAfCfaagacauuggL96 | 2202 | sense | 21 |
| CCAAUGUCUUGUCGAUGACUUUC | 1107 | csCfsaauGfuCfUfugucGfaUfgacuususc | 2203 | antisense | 23 |
| AGUCAUCGACAAGACAUUGGU | 1108 | asgsucauCfgAfCfAfagacauugguL96 | 2204 | sense | 21 |
| ACCAAUGUCUUGUCGAUGACUUU | 1109 | asCfscaaUfgUfCfuuguCfgAfugacususu | 2205 | antisense | 23 |
| GUGAAAGUCAUCGACAAGACA | 1110 | gsusgaaaGfuCfAfUfcgacaagacaL96 | 2206 | sense | 21 |
| UGUCUUGUCGAUGACUUUCACAU | 1111 | usGfsucuUfgUfCfgaugAfcUfuucacsasu | 2207 | antisense | 23 |
| UGUGAAAGUCAUCGACAAGAC | 1112 | usgsugaaAfgUfCfAfucgacaagacL96 | 2208 | sense | 21 |
| GUCUUGUCGAUGACUUUCACAUU | 1113 | gsUfscuuGfuCfGfaugaCfuUfucacasusu | 2209 | antisense | 23 |
| GAUAAUAUUGCAGCAUUUUCC | 1114 | gsasuaauAfuUfGfCfagcauuuuccL96 | 2210 | sense | 21 |
| GGAAAAUGCUGCAAUAUUAUCAG | 1115 | gsGfsaaaAfuGfCfugcaAfuAfuuaucsasg | 2211 | antisense | 23 |
| AUAAUAUUGCAGCAUUUUCCA | 1116 | asusaauaUfuGfCfAfgcauuuuccaL96 | 2212 | sense | 21 |
| UGGAAAAUGCUGCAAUAUUAUCA | 1117 | usGfsgaaAfaUfGfcugcAfaUfauuauscsa | 2213 | antisense | 23 |
| GGCUGAUAAUAUUGCAGCAUU | 1118 | gsgscugaUfaAfUfAfuugcagcauuL96 | 2214 | sense | 21 |
| AAUGCUGCAAUAUUAUCAGCCAA | 1119 | asAfsugcUfgCfAfauauUfaUfcagccsasa | 2215 | antisense | 23 |
| UGGCUGAUAAUAUUGCAGCAU | 1120 | usgsgcugAfuAfAfUfauugcagcauL96 | 2216 | sense | 21 |
| AUGCUGCAAUAUUAUCAGCCAAA | 1121 | asUfsgcuGfcAfAfuauuAfuCfagccasasa | 2217 | antisense | 23 |
| GCUAAUUUGUAUCAAUGAUUA | 1122 | gscsuaauUfuGfUfAfucaaugauuaL96 | 2218 | sense | 21 |
| UAAUCAUUGAUACAAAUUAGCCG | 1123 | usAfsaucAfuUfGfauacAfaAfuuagcscsg | 2219 | antisense | 23 |
| CUAAUUUGUAUCAAUGAUUAU | 1124 | csusaauuUfgUfAfUfcaaugauuauL96 | 2220 | sense | 21 |
| AUAAUCAUUGAUACAAAUUAGCC | 1125 | asUfsaauCfaUfUfgauaCfaAfauuagscsc | 2221 | antisense | 23 |
| CCCGGCUAAUUUGUAUCAAUG | 1126 | cscscggcUfaAfUfUfuguaucaaugL96 | 2222 | sense | 21 |
| CAUUGAUACAAAUUAGCCGGGGG | 1127 | csAfsuugAfuAfCfaaauUfaGfccgggsgsg | 2223 | antisense | 23 |
| CCCCGGCUAAUUUGUAUCAAU | 1128 | cscsccggCfuAfAfUfuuguaucaauL96 | 2224 | sense | 21 |
| AUUGAUACAAAUUAGCCGGGGGA | 1129 | asUfsugaUfaCfAfaauuAfgCfcggggsgsa | 2225 | antisense | 23 |
| UAAUUGGUGAUACUUCUUUGA | 1130 | usasauugGfuGfAfUfacuucuuugaL96 | 2226 | sense | 21 |
| UCAAAGAAGUAUCACCAAUUACC | 1131 | usCfsaaaGfaAfGfuaucAfcCfaauuascsc | 2227 | antisense | 23 |
| AAUUGGUGAUACUUCUUUGAA | 1132 | asasuuggUfgAfUfAfcuucuuugaaL96 | 2228 | sense | 21 |
| UUCAAAGAAGUAUCACCAAUUAC | 1133 | usUfscaaAfgAfAfguauCfaCfcaauusasc | 2229 | antisense | 23 |
| GCGGUAAUUGGUGAUACUUCU | 1134 | gscsgguaAfuUfGfGfugauacuucuL96 | 2230 | sense | 21 |
| AGAAGUAUCACCAAUUACCGCCA | 1135 | asGfsaagUfaUfCfaccaAfuUfaccgcscsa | 2231 | antisense | 23 |
| GGCGGUAAUUGGUGAUACUUC | 1136 | gsgscgguAfaUfUfGfgugauacuucL96 | 2232 | sense | 21 |
| GAAGUAUCACCAAUUACCGCCAC | 1137 | gsAfsaguAfuCfAfccaaUfuAfccgccsasc | 2233 | antisense | 23 |
| CAGUGGUUCUUAAAUUGUAAG | 1138 | csasguggUfuCfUfUfaaauuguaagL96 | 2234 | sense | 21 |
| CUUACAAUUUAAGAACCACUGUU | 1139 | csUfsuacAfaUfUfuaagAfaCfcacugsusu | 2235 | antisense | 23 |
| AGUGGUUCUUAAAUUGUAAGC | 1140 | asgsugguUfcUfUfAfaauuguaagcL96 | 2236 | sense | 21 |
| GCUUACAAUUUAAGAACCACUGU | 1141 | gsCfsuuaCfaAfUfuuaaGfaAfccacusgsu | 2237 | antisense | 23 |
| AAAACAGUGGUUCUUAAAUUG | 1142 | asasaacaGfuGfGfUfucuuaaauugL96 | 2238 | sense | 21 |
| CAAUUUAAGAACCACUGUUUUAA | 1143 | csAfsauuUfaAfGfaaccAfcUfguuuusasa | 2239 | antisense | 23 |
| UAAAACAGUGGUUCUUAAAUU | 1144 | usasaaacAfgUfGfGfuucuuaaauuL96 | 2240 | sense | 21 |
| AAUUUAAGAACCACUGUUUUAAA | 1145 | asAfsuuuAfaGfAfaccaCfuGfuuuuasasa | 2241 | antisense | 23 |
| ACCUGUAUUCUGUUUACAUGU | 1146 | ascscuguAfuUfCfUfguuuacauguL96 | 2242 | sense | 21 |
| ACAUGUAAACAGAAUACAGGUUA | 1147 | asCfsaugUfaAfAfcagaAfuAfcaggususa | 2243 | antisense | 23 |
| CCUGUAUUCUGUUUACAUGUC | 1148 | cscsuguaUfuCfUfGfuuuacaugucL96 | 2244 | sense | 21 |
| GACAUGUAAACAGAAUACAGGUU | 1149 | gsAfscauGfuAfAfacagAfaUfacaggsusu | 2245 | antisense | 23 |
| AUUAACCUGUAUUCUGUUUAC | 1150 | asusuaacCfuGfUfAfuucuguuuacL96 | 2246 | sense | 21 |
| GUAAACAGAAUACAGGUUAAUAA | 1151 | gsUfsaaaCfaGfAfauacAfgGfuuaausasa | 2247 | antisense | 23 |
| UAUUAACCUGUAUUCUGUUUA | 1152 | usasuuaaCfcUfGfUfauucuguuuaL96 | 2248 | sense | 21 |
| UAAACAGAAUACAGGUUAAUAAA | 1153 | usAfsaacAfgAfAfuacaGfgUfuaauasasa | 2249 | antisense | 23 |
| AAGAAACUUUGGCUGAUAAUA | 1154 | asasgaaaCfuUfUfGfgcugauaauaL96 | 2250 | sense | 21 |
| UAUUAUCAGCCAAAGUUUCUUCA | 1155 | usAfsuuaUfcAfGfccaaAfgUfuucuuscsa | 2251 | antisense | 23 |
| AGAAACUUUGGCUGAUAAUAU | 1156 | asgsaaacUfuUfGfGfcugauaauauL96 | 2252 | sense | 21 |
| AUAUUAUCAGCCAAAGUUUCUUC | 1157 | asUfsauuAfuCfAfgccaAfaGfuuucususc | 2253 | antisense | 23 |
| GAUGAAGAAACUUUGGCUGAU | 1158 | gsasugaaGfaAfAfCfuuuggcugauL96 | 2254 | sense | 21 |
| AUCAGCCAAAGUUUCUUCAUCAU | 1159 | asUfscagCfcAfAfaguuUfcUfucaucsasu | 2255 | antisense | 23 |
| UGAUGAAGAAACUUUGGCUGA | 1160 | usgsaugaAfgAfAfAfcuuuggcugaL96 | 2256 | sense | 21 |
| UCAGCCAAAGUUUCUUCAUCAUU | 1161 | usCfsagcCfaAfAfguuuCfuUfcaucasusu | 2257 | antisense | 23 |
| GAAAGGUGUUCAAGAUGUCCU | 1162 | gsasaaggUfgUfUfCfaagauguccuL96 | 2258 | sense | 21 |
| AGGACAUCUUGAACACCUUUCUC | 1163 | asGfsgacAfuCfUfugaaCfaCfcuuucsusc | 2259 | antisense | 23 |
| AAAGGUGUUCAAGAUGUCCUC | 1164 | asasagguGfuUfCfAfagauguccucL96 | 2260 | sense | 21 |
| GAGGACAUCUUGAACACCUUUCU | 1165 | gsAfsggaCfaUfCfuugaAfcAfccuuuscsu | 2261 | antisense | 23 |
| GGGAGAAAGGUGUUCAAGAUG | 1166 | gsgsgagaAfaGfGfUfguucaagaugL96 | 2262 | sense | 21 |
| CAUCUUGAACACCUUUCUCCCCC | 1167 | csAfsucuUfgAfAfcaccUfuUfcucccscsc | 2263 | antisense | 23 |
| GGGGAGAAAGGUGUUCAAGAU | 1168 | gsgsggagAfaAfGfGfuguucaagauL96 | 2264 | sense | 21 |
| AUCUUGAACACCUUUCUCCCCCU | 1169 | asUfscuuGfaAfCfaccuUfuCfuccccscsu | 2265 | antisense | 23 |
| AUCUUGGUGUCGAAUCAUGGG | 1170 | asuscuugGfuGfUfCfgaaucaugggL96 | 2266 | sense | 21 |
| CCCAUGAUUCGACACCAAGAUCC | 1171 | csCfscauGfaUfUfcgacAfcCfaagauscsc | 2267 | antisense | 23 |
| UCUUGGUGUCGAAUCAUGGGG | 1172 | uscsuuggUfgUfCfGfaaucauggggL96 | 2268 | sense | 21 |
| CCCCAUGAUUCGACACCAAGAUC | 1173 | csCfsccaUfgAfUfucgaCfaCfcaagasusc | 2269 | antisense | 23 |
| UGGGAUCUUGGUGUCGAAUCA | 1174 | usgsggauCfuUfGfGfugucgaaucaL96 | 2270 | sense | 21 |
| UGAUUCGACACCAAGAUCCCAUU | 1175 | usGfsauuCfgAfCfaccaAfgAfucccasusu | 2271 | antisense | 23 |
| AUGGGAUCUUGGUGUCGAAUC | 1176 | asusgggaUfcUfUfGfgugucgaaucL96 | 2272 | sense | 21 |
| GAUUCGACACCAAGAUCCCAUUC | 1177 | gsAfsuucGfaCfAfccaaGfaUfcccaususc | 2273 | antisense | 23 |
| GCUACAAGGCCAUAUUUGUGA | 1178 | gscsuacaAfgGfCfCfauauuugugaL96 | 2274 | sense | 21 |
| UCACAAAUAUGGCCUUGUAGCCC | 1179 | usCfsacaAfaUfAfuggcCfuUfguagcscsc | 2275 | antisense | 23 |
| CUACAAGGCCAUAUUUGUGAC | 1180 | csusacaaGfgCfCfAfuauuugugacL96 | 2276 | sense | 21 |
| GUCACAAAUAUGGCCUUGUAGCC | 1181 | gsUfscacAfaAfUfauggCfcUfuguagscsc | 2277 | antisense | 23 |
| AUGGGCUACAAGGCCAUAUUU | 1182 | asusgggcUfaCfAfAfggccauauuuL96 | 2278 | sense | 21 |
| AAAUAUGGCCUUGUAGCCCAUCU | 1183 | asAfsauaUfgGfCfcuugUfaGfcccauscsu | 2279 | antisense | 23 |
| GAUGGGCUACAAGGCCAUAUU | 1184 | gsasugggCfuAfCfAfaggccauauuL96 | 2280 | sense | 21 |
| AAUAUGGCCUUGUAGCCCAUCUU | 1185 | asAfsuauGfgCfCfuuguAfgCfccaucsusu | 2281 | antisense | 23 |
| ACUGGAGAGAAUUGGAAUGGG | 1186 | ascsuggaGfaGfAfAfuuggaaugggL96 | 2282 | sense | 21 |
| CCCAUUCCAAUUCUCUCCAGUGC | 1187 | csCfscauUfcCfAfauucUfcUfccagusgsc | 2283 | antisense | 23 |
| CUGGAGAGAAUUGGAAUGGGU | 1188 | csusggagAfgAfAfUfuggaauggguL96 | 2284 | sense | 21 |
| ACCCAUUCCAAUUCUCUCCAGUG | 1189 | asCfsccaUfuCfCfaauuCfuCfuccagsusg | 2285 | antisense | 23 |
| UAGCACUGGAGAGAAUUGGAA | 1190 | usasgcacUfgGfAfGfagaauuggaaL96 | 2286 | sense | 21 |
| UUCCAAUUCUCUCCAGUGCUACC | 1191 | usUfsccaAfuUfCfucucCfaGfugcuascsc | 2287 | antisense | 23 |
| GUAGCACUGGAGAGAAUUGGA | 1192 | gsusagcaCfuGfGfAfgagaauuggaL96 | 2288 | sense | 21 |
| UCCAAUUCUCUCCAGUGCUACCU | 1193 | usCfscaaUfuCfUfcuccAfgUfgcuacscsu | 2289 | antisense | 23 |
| ACAGUGGACACACCUUACCUG | 1194 | ascsagugGfaCfAfCfaccuuaccugL96 | 2290 | sense | 21 |
| CAGGUAAGGUGUGUCCACUGUCA | 1195 | csAfsgguAfaGfGfugugUfcCfacuguscsa | 2291 | antisense | 23 |
| CAGUGGACACACCUUACCUGG | 1196 | csasguggAfcAfCfAfccuuaccuggL96 | 2292 | sense | 21 |
| CCAGGUAAGGUGUGUCCACUGUC | 1197 | csCfsaggUfaAfGfguguGfuCfcacugsusc | 2293 | antisense | 23 |
| UGUGACAGUGGACACACCUUA | 1198 | usgsugacAfgUfGfGfacacaccuuaL96 | 2294 | sense | 21 |
| UAAGGUGUGUCCACUGUCACAAA | 1199 | usAfsaggUfgUfGfuccaCfuGfucacasasa | 2295 | antisense | 23 |
| UUGUGACAGUGGACACACCUU | 1200 | ususgugaCfaGfUfGfgacacaccuuL96 | 2296 | sense | 21 |
| AAGGUGUGUCCACUGUCACAAAU | 1201 | asAfsgguGfuGfUfccacUfgUfcacaasasu | 2297 | antisense | 23 |
| GAAGACUGACAUCAUUGCCAA | 1202 | gsasagacUfgAfCfAfucauugccaaL96 | 2298 | sense | 21 |
| UUGGCAAUGAUGUCAGUCUUCUC | 1203 | usUfsggcAfaUfGfauguCfaGfucuucsusc | 2299 | antisense | 23 |
| AAGACUGACAUCAUUGCCAAU | 1204 | asasgacuGfaCfAfUfcauugccaauL96 | 2300 | sense | 21 |
| AUUGGCAAUGAUGUCAGUCUUCU | 1205 | asUfsuggCfaAfUfgaugUfcAfgucuuscsu | 2301 | antisense | 23 |
| CUGAGAAGACUGACAUCAUUG | 1206 | csusgagaAfgAfCfUfgacaucauugL96 | 2302 | sense | 21 |
| CAAUGAUGUCAGUCUUCUCAGCC | 1207 | csAfsaugAfuGfUfcaguCfuUfcucagscsc | 2303 | antisense | 23 |
| GCUGAGAAGACUGACAUCAUU | 1208 | gscsugagAfaGfAfCfugacaucauuL96 | 2304 | sense | 21 |
| AAUGAUGUCAGUCUUCUCAGCCA | 1209 | asAfsugaUfgUfCfagucUfuCfucagcscsa | 2305 | antisense | 23 |
| GCUCAGGUUCAAAGUGUUGGU | 1210 | gscsucagGfuUfCfAfaaguguugguL96 | 2306 | sense | 21 |
| ACCAACACUUUGAACCUGAGCUU | 1211 | asCfscaaCfaCfUfuugaAfcCfugagcsusu | 2307 | antisense | 23 |
| CUCAGGUUCAAAGUGUUGGUA | 1212 | csuscaggUfuCfAfAfaguguugguaL96 | 2308 | sense | 21 |
| UACCAACACUUUGAACCUGAGCU | 1213 | usAfsccaAfcAfCfuuugAfaCfcugagscsu | 2309 | antisense | 23 |
| GUAAGCUCAGGUUCAAAGUGU | 1214 | gsusaagcUfcAfGfGfuucaaaguguL96 | 2310 | sense | 21 |
| ACACUUUGAACCUGAGCUUACAA | 1215 | asCfsacuUfuGfAfaccuGfaGfcuuacsasa | 2311 | antisense | 23 |
| UGUAAGCUCAGGUUCAAAGUG | 1216 | usgsuaagCfuCfAfGfguucaaagugL96 | 2312 | sense | 21 |
| CACUUUGAACCUGAGCUUACAAU | 1217 | csAfscuuUfgAfAfccugAfgCfuuacasasu | 2313 | antisense | 23 |
| AUGUAUUACUUGACAAAGAGA | 1218 | asusguauUfaCfUfUfgacaaagagaL96 | 2314 | sense | 21 |
| UCUCUUUGUCAAGUAAUACAUGC | 1219 | usCfsucuUfuGfUfcaagUfaAfuacausgsc | 2315 | antisense | 23 |
| UGUAUUACUUGACAAAGAGAC | 1220 | usgsuauuAfcUfUfGfacaaagagacL96 | 2316 | sense | 21 |
| GUCUCUUUGUCAAGUAAUACAUG | 1221 | gsUfscucUfuUfGfucaaGfuAfauacasusg | 2317 | antisense | 23 |
| CAGCAUGUAUUACUUGACAAA | 1222 | csasgcauGfuAfUfUfacuugacaaaL96 | 2318 | sense | 21 |
| UUUGUCAAGUAAUACAUGCUGAA | 1223 | usUfsuguCfaAfGfuaauAfcAfugcugsasa | 2319 | antisense | 23 |
| UCAGCAUGUAUUACUUGACAA | 1224 | uscsagcaUfgUfAfUfuacuugacaaL96 | 2320 | sense | 21 |
| UUGUCAAGUAAUACAUGCUGAAA | 1225 | usUfsgucAfaGfUfaauaCfaUfgcugasasa | 2321 | antisense | 23 |
| CUGCAACUGUAUAUCUACAAG | 1226 | csusgcaaCfuGfUfAfuaucuacaagL96 | 2322 | sense | 21 |
| CUUGUAGAUAUACAGUUGCAGCC | 1227 | csUfsuguAfgAfUfauacAfgUfugcagscsc | 2323 | antisense | 23 |
| UGCAACUGUAUAUCUACAAGG | 1228 | usgscaacUfgUfAfUfaucuacaaggL96 | 2324 | sense | 21 |
| CCUUGUAGAUAUACAGUUGCAGC | 1229 | csCfsuugUfaGfAfuauaCfaGfuugcasgsc | 2325 | antisense | 23 |
| UUGGCUGCAACUGUAUAUCUA | 1230 | ususggcuGfcAfAfCfuguauaucuaL96 | 2326 | sense | 21 |
| UAGAUAUACAGUUGCAGCCAACG | 1231 | usAfsgauAfuAfCfaguuGfcAfgccaascsg | 2327 | antisense | 23 |
| GUUGGCUGCAACUGUAUAUCU | 1232 | gsusuggcUfgCfAfAfcuguauaucuL96 | 2328 | sense | 21 |
| AGAUAUACAGUUGCAGCCAACGA | 1233 | asGfsauaUfaCfAfguugCfaGfccaacsgsa | 2329 | antisense | 23 |
| CAAAUGAUGAAGAAACUUUGG | 1234 | csasaaugAfuGfAfAfgaaacuuuggL96 | 2330 | sense | 21 |
| CCAAAGUUUCUUCAUCAUUUGCC | 1235 | csCfsaaaGfuUfUfcuucAfuCfauuugscsc | 2331 | antisense | 23 |
| AAAUGAUGAAGAAACUUUGGC | 1236 | asasaugaUfgAfAfGfaaacuuuggcL96 | 2332 | sense | 21 |
| GCCAAAGUUUCUUCAUCAUUUGC | 1237 | gsCfscaaAfgUfUfucuuCfaUfcauuusgsc | 2333 | antisense | 23 |
| GGGGCAAAUGAUGAAGAAACU | 1238 | gsgsggcaAfaUfGfAfugaagaaacuL96 | 2334 | sense | 21 |
| AGUUUCUUCAUCAUUUGCCCCAG | 1239 | asGfsuuuCfuUfCfaucaUfuUfgccccsasg | 2335 | antisense | 23 |
| UGGGGCAAAUGAUGAAGAAAC | 1240 | usgsgggcAfaAfUfGfaugaagaaacL96 | 2336 | sense | 21 |
| GUUUCUUCAUCAUUUGCCCCAGA | 1241 | gsUfsuucUfuCfAfucauUfuGfccccasgsa | 2337 | antisense | 23 |
| CAAAGGGUGUCGUUCUUUUCC | 1242 | csasaaggGfuGfUfCfguucuuuuccL96 | 2338 | sense | 21 |
| GGAAAAGAACGACACCCUUUGUA | 1243 | gsGfsaaaAfgAfAfcgacAfcCfcuuugsusa | 2339 | antisense | 23 |
| AAAGGGUGUCGUUCUUUUCCA | 1244 | asasagggUfgUfCfGfuucuuuuccaL96 | 2340 | sense | 21 |
| UGGAAAAGAACGACACCCUUUGU | 1245 | usGfsgaaAfaGfAfacgaCfaCfccuuusgsu | 2341 | antisense | 23 |
| AAUACAAAGGGUGUCGUUCUU | 1246 | asasuacaAfaGfGfGfugucguucuuL96 | 2342 | sense | 21 |
| AAGAACGACACCCUUUGUAUUGA | 1247 | asAfsgaaCfgAfCfacccUfuUfguauusgsa | 2343 | antisense | 23 |
| CAAUACAAAGGGUGUCGUUCU | 1248 | csasauacAfaAfGfGfgugucguucuL96 | 2344 | sense | 21 |
| AGAACGACACCCUUUGUAUUGAA | 1249 | asGfsaacGfaCfAfcccuUfuGfuauugsasa | 2345 | antisense | 23 |
| AAAGGCACUGAUGUUCUGAAA | 1250 | asasaggcAfcUfGfAfuguucugaaaL96 | 2346 | sense | 21 |
| UUUCAGAACAUCAGUGCCUUUCC | 1251 | usUfsucaGfaAfCfaucaGfuGfccuuuscsc | 2347 | antisense | 23 |
| AAGGCACUGAUGUUCUGAAAG | 1252 | asasggcaCfuGfAfUfguucugaaagL96 | 2348 | sense | 21 |
| CUUUCAGAACAUCAGUGCCUUUC | 1253 | csUfsuucAfgAfAfcaucAfgUfgccuususc | 2349 | antisense | 23 |
| GCGGAAAGGCACUGAUGUUCU | 1254 | gscsggaaAfgGfCfAfcugauguucuL96 | 2350 | sense | 21 |
| AGAACAUCAGUGCCUUUCCGCAC | 1255 | asGfsaacAfuCfAfgugcCfuUfuccgcsasc | 2351 | antisense | 23 |
| UGCGGAAAGGCACUGAUGUUC | 1256 | usgscggaAfaGfGfCfacugauguucL96 | 2352 | sense | 21 |
| GAACAUCAGUGCCUUUCCGCACA | 1257 | gsAfsacaUfcAfGfugccUfuUfccgcascsa | 2353 | antisense | 23 |
| AAGGAUGCUCCGGAAUGUUGC | 1258 | asasggauGfcUfCfCfggaauguugcL96 | 2354 | sense | 21 |
| GCAACAUUCCGGAGCAUCCUUGG | 1259 | gsCfsaacAfuUfCfcggaGfcAfuccuusgsg | 2355 | antisense | 23 |
| AGGAUGCUCCGGAAUGUUGCU | 1260 | asgsgaugCfuCfCfGfgaauguugcuL96 | 2356 | sense | 21 |
| AGCAACAUUCCGGAGCAUCCUUG | 1261 | asGfscaaCfaUfUfccggAfgCfauccususg | 2357 | antisense | 23 |
| AUCCAAGGAUGCUCCGGAAUG | 1262 | asusccaaGfgAfUfGfcuccggaaugL96 | 2358 | sense | 21 |
| CAUUCCGGAGCAUCCUUGGAUAC | 1263 | csAfsuucCfgGfAfgcauCfcUfuggausasc | 2359 | antisense | 23 |
| UAUCCAAGGAUGCUCCGGAAU | 1264 | usasuccaAfgGfAfUfgcuccggaauL96 | 2360 | sense | 21 |
| AUUCCGGAGCAUCCUUGGAUACA | 1265 | asUfsuccGfgAfGfcaucCfuUfggauascsa | 2361 | antisense | 23 |
| AAUGGGUGGCGGUAAUUGGUG | 1266 | asasugggUfgGfCfGfguaauuggugL96 | 2362 | sense | 21 |
| CACCAAUUACCGCCACCCAUUCC | 1267 | csAfsccaAfuUfAfccgcCfaCfccauuscsc | 2363 | antisense | 23 |
| AUGGGUGGCGGUAAUUGGUGA | 1268 | asusggguGfgCfGfGfuaauuggugaL96 | 2364 | sense | 21 |
| UCACCAAUUACCGCCACCCAUUC | 1269 | usCfsaccAfaUfUfaccgCfcAfcccaususc | 2365 | antisense | 23 |
| UUGGAAUGGGUGGCGGUAAUU | 1270 | ususggaaUfgGfGfUfggcgguaauuL96 | 2366 | sense | 21 |
| AAUUACCGCCACCCAUUCCAAUU | 1271 | asAfsuuaCfcGfCfcaccCfaUfuccaasusu | 2367 | antisense | 23 |
| AUUGGAAUGGGUGGCGGUAAU | 1272 | asusuggaAfuGfGfGfuggcgguaauL96 | 2368 | sense | 21 |
| AUUACCGCCACCCAUUCCAAUUC | 1273 | asUfsuacCfgCfCfacccAfuUfccaaususc | 2369 | antisense | 23 |
| GGAAAGGCACUGAUGUUCUGA | 1274 | gsgsaaagGfcAfCfUfgauguucugaL96 | 2370 | sense | 21 |
| UCAGAACAUCAGUGCCUUUCCGC | 1275 | usCfsagaAfcAfUfcaguGfcCfuuuccsgsc | 2371 | antisense | 23 |
| GAAAGGCACUGAUGUUCUGAA | 1276 | gsasaaggCfaCfUfGfauguucugaaL96 | 2372 | sense | 21 |
| UUCAGAACAUCAGUGCCUUUCCG | 1277 | usUfscagAfaCfAfucagUfgCfcuuucscsg | 2373 | antisense | 23 |
| GUGCGGAAAGGCACUGAUGUU | 1278 | gsusgcggAfaAfGfGfcacugauguuL96 | 2374 | sense | 21 |
| AACAUCAGUGCCUUUCCGCACAC | 1279 | asAfscauCfaGfUfgccuUfuCfcgcacsasc | 2375 | antisense | 23 |
| UGUGCGGAAAGGCACUGAUGU | 1280 | usgsugcgGfaAfAfGfgcacugauguL96 | 2376 | sense | 21 |
| ACAUCAGUGCCUUUCCGCACACC | 1281 | asCfsaucAfgUfGfccuuUfcCfgcacascsc | 2377 | antisense | 23 |
| AAUUGUAAGCUCAGGUUCAAA | 1282 | asasuuguAfaGfCfUfcagguucaaaL96 | 2378 | sense | 21 |
| UUUGAACCUGAGCUUACAAUUUA | 1283 | usUfsugaAfcCfUfgagcUfuAfcaauususa | 2379 | antisense | 23 |
| AUUGUAAGCUCAGGUUCAAAG | 1284 | asusuguaAfgCfUfCfagguucaaagL96 | 2380 | sense | 21 |
| CUUUGAACCUGAGCUUACAAUUU | 1285 | csUfsuugAfaCfCfugagCfuUfacaaususu | 2381 | antisense | 23 |
| CUUAAAUUGUAAGCUCAGGUU | 1286 | csusuaaaUfuGfUfAfagcucagguuL96 | 2382 | sense | 21 |
| AACCUGAGCUUACAAUUUAAGAA | 1287 | asAfsccuGfaGfCfuuacAfaUfuuaagsasa | 2383 | antisense | 23 |
| UCUUAAAUUGUAAGCUCAGGU | 1288 | uscsuuaaAfuUfGfUfaagcucagguL96 | 2384 | sense | 21 |
| ACCUGAGCUUACAAUUUAAGAAC | 1289 | asCfscugAfgCfUfuacaAfuUfuaagasasc | 2385 | antisense | 23 |
| GCAAACACUAAGGUGAAAAGA | 1290 | gscsaaacAfcUfAfAfggugaaaagaL96 | 2386 | sense | 21 |
| UCUUUUCACCUUAGUGUUUGCUA | 1291 | usCfsuuuUfcAfCfcuuaGfuGfuuugcsusa | 2387 | antisense | 23 |
| CAAACACUAAGGUGAAAAGAU | 1292 | csasaacaCfuAfAfGfgugaaaagauL96 | 2388 | sense | 21 |
| AUCUUUUCACCUUAGUGUUUGCU | 1293 | asUfscuuUfuCfAfccuuAfgUfguuugscsu | 2389 | antisense | 23 |
| GGUAGCAAACACUAAGGUGAA | 1294 | gsgsuagcAfaAfCfAfcuaaggugaaL96 | 2390 | sense | 21 |
| UUCACCUUAGUGUUUGCUACCUC | 1295 | usUfscacCfuUfAfguguUfuGfcuaccsusc | 2391 | antisense | 23 |
| AGGUAGCAAACACUAAGGUGA | 1296 | asgsguagCfaAfAfCfacuaaggugaL96 | 2392 | sense | 21 |
| UCACCUUAGUGUUUGCUACCUCC | 1297 | usCfsaccUfuAfGfuguuUfgCfuaccuscsc | 2393 | antisense | 23 |
| AGGUAGCAAACACUAAGGUGA | 1298 | asgsguagCfaAfAfCfacuaaggugaL96 | 2394 | sense | 21 |
| UCACCUUAGUGUUUGCUACCUCC | 1299 | usCfsaccUfuAfGfuguuUfgCfuaccuscsc | 2395 | antisense | 23 |
| GGUAGCAAACACUAAGGUGAA | 1300 | gsgsuagcAfaAfCfAfcuaaggugaaL96 | 2396 | sense | 21 |
| UUCACCUUAGUGUUUGCUACCUC | 1301 | usUfscacCfuUfAfguguUfuGfcuaccsusc | 2397 | antisense | 23 |
| UUGGAGGUAGCAAACACUAAG | 1302 | ususggagGfuAfGfCfaaacacuaagL96 | 2398 | sense | 21 |
| CUUAGUGUUUGCUACCUCCAAUU | 1303 | csUfsuagUfgUfUfugcuAfcCfuccaasusu | 2399 | antisense | 23 |
| AUUGGAGGUAGCAAACACUAA | 1304 | asusuggaGfgUfAfGfcaaacacuaaL96 | 2400 | sense | 21 |
| UUAGUGUUUGCUACCUCCAAUUU | 1305 | usUfsaguGfuUfUfgcuaCfcUfccaaususu | 2401 | antisense | 23 |
| UAAAGUGCUGUAUCCUUUAGU | 1306 | usasaaguGfcUfGfUfauccuuuaguL96 | 2402 | sense | 21 |
| ACUAAAGGAUACAGCACUUUAGC | 1307 | asCfsuaaAfgGfAfuacaGfcAfcuuuasgsc | 2403 | antisense | 23 |
| AAAGUGCUGUAUCCUUUAGUA | 1308 | asasagugCfuGfUfAfuccuuuaguaL96 | 2404 | sense | 21 |
| UACUAAAGGAUACAGCACUUUAG | 1309 | usAfscuaAfaGfGfauacAfgCfacuuusasg | 2405 | antisense | 23 |
| AGGCUAAAGUGCUGUAUCCUU | 1310 | asgsgcuaAfaGfUfGfcuguauccuuL96 | 2406 | sense | 21 |
| AAGGAUACAGCACUUUAGCCUGC | 1311 | asAfsggaUfaCfAfgcacUfuUfagccusgsc | 2407 | antisense | 23 |
| CAGGCUAAAGUGCUGUAUCCU | 1312 | csasggcuAfaAfGfUfgcuguauccuL96 | 2408 | sense | 21 |
| AGGAUACAGCACUUUAGCCUGCC | 1313 | asGfsgauAfcAfGfcacuUfuAfgccugscsc | 2409 | antisense | 23 |
| AAGACAUUGGUGAGGAAAAAU | 1314 | asasgacaUfuGfGfUfgaggaaaaauL96 | 2410 | sense | 21 |
| AUUUUUCCUCACCAAUGUCUUGU | 1315 | asUfsuuuUfcCfUfcaccAfaUfgucuusgsu | 2411 | antisense | 23 |
| AGACAUUGGUGAGGAAAAAUC | 1316 | asgsacauUfgGfUfGfaggaaaaaucL96 | 2412 | sense | 21 |
| GAUUUUUCCUCACCAAUGUCUUG | 1317 | gsAfsuuuUfuCfCfucacCfaAfugucususg | 2413 | antisense | 23 |
| CGACAAGACAUUGGUGAGGAA | 1318 | csgsacaaGfaCfAfUfuggugaggaaL96 | 2414 | sense | 21 |
| UUCCUCACCAAUGUCUUGUCGAU | 1319 | usUfsccuCfaCfCfaaugUfcUfugucgsasu | 2415 | antisense | 23 |
| UCGACAAGACAUUGGUGAGGA | 1320 | uscsgacaAfgAfCfAfuuggugaggaL96 | 2416 | sense | 21 |
| UCCUCACCAAUGUCUUGUCGAUG | 1321 | usCfscucAfcCfAfauguCfuUfgucgasusg | 2417 | antisense | 23 |
| AAGAUGUCCUCGAGAUACUAA | 1322 | asasgaugUfcCfUfCfgagauacuaaL96 | 2418 | sense | 21 |
| UUAGUAUCUCGAGGACAUCUUGA | 1323 | usUfsaguAfuCfUfcgagGfaCfaucuusgsa | 2419 | antisense | 23 |
| AGAUGUCCUCGAGAUACUAAA | 1324 | asgsauguCfcUfCfGfagauacuaaaL96 | 2420 | sense | 21 |
| UUUAGUAUCUCGAGGACAUCUUG | 1325 | usUfsuagUfaUfCfucgaGfgAfcaucususg | 2421 | antisense | 23 |
| GUUCAAGAUGUCCUCGAGAUA | 1326 | gsusucaaGfaUfGfUfccucgagauaL96 | 2422 | sense | 21 |
| UAUCUCGAGGACAUCUUGAACAC | 1327 | usAfsucuCfgAfGfgacaUfcUfugaacsasc | 2423 | antisense | 23 |
| UGUUCAAGAUGUCCUCGAGAU | 1328 | usgsuucaAfgAfUfGfuccucgagauL96 | 2424 | sense | 21 |
| AUCUCGAGGACAUCUUGAACACC | 1329 | asUfscucGfaGfGfacauCfuUfgaacascsc | 2425 | antisense | 23 |
| GAGAAAGGUGUUCAAGAUGUC | 1330 | gsasgaaaGfgUfGfUfucaagaugucL96 | 2426 | sense | 21 |
| GACAUCUUGAACACCUUUCUCCC | 1331 | gsAfscauCfuUfGfaacaCfcUfuucucscsc | 2427 | antisense | 23 |
| AGAAAGGUGUUCAAGAUGUCC | 1332 | asgsaaagGfuGfUfUfcaagauguccL96 | 2428 | sense | 21 |
| GGACAUCUUGAACACCUUUCUCC | 1333 | gsGfsacaUfcUfUfgaacAfcCfuuucuscsc | 2429 | antisense | 23 |
| GGGGGAGAAAGGUGUUCAAGA | 1334 | gsgsgggaGfaAfAfGfguguucaagaL96 | 2430 | sense | 21 |
| UCUUGAACACCUUUCUCCCCCUG | 1335 | usCfsuugAfaCfAfccuuUfcUfcccccsusg | 2431 | antisense | 23 |
| AGGGGGAGAAAGGUGUUCAAG | 1336 | asgsggggAfgAfAfAfgguguucaagL96 | 2432 | sense | 21 |
| CUUGAACACCUUUCUCCCCCUGG | 1337 | csUfsugaAfcAfCfcuuuCfuCfccccusgsg | 2433 | antisense | 23 |
| GCUGGGAAGAUAUCAAAUGGC | 1338 | gscsugggAfaGfAfUfaucaaauggcL96 | 2434 | sense | 21 |
| GCCAUUUGAUAUCUUCCCAGCUG | 1339 | gsCfscauUfuGfAfuaucUfuCfccagcsusg | 2435 | antisense | 23 |
| CUGGGAAGAUAUCAAAUGGCU | 1340 | csusgggaAfgAfUfAfucaaauggcuL96 | 2436 | sense | 21 |
| AGCCAUUUGAUAUCUUCCCAGCU | 1341 | asGfsccaUfuUfGfauauCfuUfcccagscsu | 2437 | antisense | 23 |
| AUCAGCUGGGAAGAUAUCAAA | 1342 | asuscagcUfgGfGfAfagauaucaaaL96 | 2438 | sense | 21 |
| UUUGAUAUCUUCCCAGCUGAUAG | 1343 | usUfsugaUfaUfCfuuccCfaGfcugausasg | 2439 | antisense | 23 |
| UAUCAGCUGGGAAGAUAUCAA | 1344 | usasucagCfuGfGfGfaagauaucaaL96 | 2440 | sense | 21 |
| UUGAUAUCUUCCCAGCUGAUAGA | 1345 | usUfsgauAfuCfUfucccAfgCfugauasgsa | 2441 | antisense | 23 |
| UCUGUCGACUUCUGUUUUAGG | 1346 | uscsugucGfaCfUfUfcuguuuuaggL96 | 2442 | sense | 21 |
| CCUAAAACAGAAGUCGACAGAUC | 1347 | csCfsuaaAfaCfAfgaagUfcGfacagasusc | 2443 | antisense | 23 |
| CUGUCGACUUCUGUUUUAGGA | 1348 | csusgucgAfcUfUfCfuguuuuaggaL96 | 2444 | sense | 21 |
| UCCUAAAACAGAAGUCGACAGAU | 1349 | usCfscuaAfaAfCfagaaGfuCfgacagsasu | 2445 | antisense | 23 |
| CAGAUCUGUCGACUUCUGUUU | 1350 | csasgaucUfgUfCfGfacuucuguuuL96 | 2446 | sense | 21 |
| AAACAGAAGUCGACAGAUCUGUU | 1351 | asAfsacaGfaAfGfucgaCfaGfaucugsusu | 2447 | antisense | 23 |
| ACAGAUCUGUCGACUUCUGUU | 1352 | ascsagauCfuGfUfCfgacuucuguuL96 | 2448 | sense | 21 |
| AACAGAAGUCGACAGAUCUGUUU | 1353 | asAfscagAfaGfUfcgacAfgAfucugususu | 2449 | antisense | 23 |
| UACUUCUUUGAAUGUAGAUUU | 1354 | usascuucUfuUfGfAfauguagauuuL96 | 2450 | sense | 21 |
| AAAUCUACAUUCAAAGAAGUAUC | 1355 | asAfsaucUfaCfAfuucaAfaGfaaguasusc | 2451 | antisense | 23 |
| ACUUCUUUGAAUGUAGAUUUC | 1356 | ascsuucuUfuGfAfAfuguagauuucL96 | 2452 | sense | 21 |
| GAAAUCUACAUUCAAAGAAGUAU | 1357 | gsAfsaauCfuAfCfauucAfaAfgaagusasu | 2453 | antisense | 23 |
| GUGAUACUUCUUUGAAUGUAG | 1358 | gsusgauaCfuUfCfUfuugaauguagL96 | 2454 | sense | 21 |
| CUACAUUCAAAGAAGUAUCACCA | 1359 | csUfsacaUfuCfAfaagaAfgUfaucacscsa | 2455 | antisense | 23 |
| GGUGAUACUUCUUUGAAUGUA | 1360 | gsgsugauAfcUfUfCfuuugaauguaL96 | 2456 | sense | 21 |
| UACAUUCAAAGAAGUAUCACCAA | 1361 | usAfscauUfcAfAfagaaGfuAfucaccsasa | 2457 | antisense | 23 |
| UGGGAAGAUAUCAAAUGGCUG | 1362 | usgsggaaGfaUfAfUfcaaauggcugL96 | 2458 | sense | 21 |
| CAGCCAUUUGAUAUCUUCCCAGC | 1363 | csAfsgccAfuUfUfgauaUfcUfucccasgsc | 2459 | antisense | 23 |
| GGGAAGAUAUCAAAUGGCUGA | 1364 | gsgsgaagAfuAfUfCfaaauggcugaL96 | 2460 | sense | 21 |
| UCAGCCAUUUGAUAUCUUCCCAG | 1365 | usCfsagcCfaUfUfugauAfuCfuucccsasg | 2461 | antisense | 23 |
| CAGCUGGGAAGAUAUCAAAUG | 1366 | csasgcugGfgAfAfGfauaucaaaugL96 | 2462 | sense | 21 |
| CAUUUGAUAUCUUCCCAGCUGAU | 1367 | csAfsuuuGfaUfAfucuuCfcCfagcugsasu | 2463 | antisense | 23 |
| UCAGCUGGGAAGAUAUCAAAU | 1368 | uscsagcuGfgGfAfAfgauaucaaauL96 | 2464 | sense | 21 |
| AUUUGAUAUCUUCCCAGCUGAUA | 1369 | asUfsuugAfuAfUfcuucCfcAfgcugasusa | 2465 | antisense | 23 |
| UCCAAAGUCUAUAUAUGACUA | 1370 | uscscaaaGfuCfUfAfuauaugacuaL96 | 2466 | sense | 21 |
| UAGUCAUAUAUAGACUUUGGAAG | 1371 | usAfsgucAfuAfUfauagAfcUfuuggasasg | 2467 | antisense | 23 |
| CCAAAGUCUAUAUAUGACUAU | 1372 | cscsaaagUfcUfAfUfauaugacuauL96 | 2468 | sense | 21 |
| AUAGUCAUAUAUAGACUUUGGAA | 1373 | asUfsaguCfaUfAfuauaGfaCfuuuggsasa | 2469 | antisense | 23 |
| UACUUCCAAAGUCUAUAUAUG | 1374 | usascuucCfaAfAfGfucuauauaugL96 | 2470 | sense | 21 |
| CAUAUAUAGACUUUGGAAGUACU | 1375 | csAfsuauAfuAfGfacuuUfgGfaaguascsu | 2471 | antisense | 23 |
| GUACUUCCAAAGUCUAUAUAU | 1376 | gsusacuuCfcAfAfAfgucuauauauL96 | 2472 | sense | 21 |
| AUAUAUAGACUUUGGAAGUACUG | 1377 | asUfsauaUfaGfAfcuuuGfgAfaguacsusg | 2473 | antisense | 23 |
| UUAUGAACAACAUGCUAAAUC | 1378 | ususaugaAfcAfAfCfaugcuaaaucL96 | 2474 | sense | 21 |
| GAUUUAGCAUGUUGUUCAUAAUC | 1379 | gsAfsuuuAfgCfAfuguuGfuUfcauaasusc | 2475 | antisense | 23 |
| UAUGAACAACAUGCUAAAUCA | 1380 | usasugaaCfaAfCfAfugcuaaaucaL96 | 2476 | sense | 21 |
| UGAUUUAGCAUGUUGUUCAUAAU | 1381 | usGfsauuUfaGfCfauguUfgUfucauasasu | 2477 | antisense | 23 |
| AUGAUUAUGAACAACAUGCUA | 1382 | asusgauuAfuGfAfAfcaacaugcuaL96 | 2478 | sense | 21 |
| UAGCAUGUUGUUCAUAAUCAUUG | 1383 | usAfsgcaUfgUfUfguucAfuAfaucaususg | 2479 | antisense | 23 |
| AAUGAUUAUGAACAACAUGCU | 1384 | asasugauUfaUfGfAfacaacaugcuL96 | 2480 | sense | 21 |
| AGCAUGUUGUUCAUAAUCAUUGA | 1385 | asGfscauGfuUfGfuucaUfaAfucauusgsa | 2481 | antisense | 23 |
| AAUUCCCCACUUCAAUACAAA | 1386 | asasuuccCfcAfCfUfucaauacaaaL96 | 2482 | sense | 21 |
| UUUGUAUUGAAGUGGGGAAUUAC | 1387 | usUfsuguAfuUfGfaaguGfgGfgaauusasc | 2483 | antisense | 23 |
| AUUCCCCACUUCAAUACAAAG | 1388 | asusucccCfaCfUfUfcaauacaaagL96 | 2484 | sense | 21 |
| CUUUGUAUUGAAGUGGGGAAUUA | 1389 | csUfsuugUfaUfUfgaagUfgGfggaaususa | 2485 | antisense | 23 |
| CUGUAAUUCCCCACUUCAAUA | 1390 | csusguaaUfuCfCfCfcacuucaauaL96 | 2486 | sense | 21 |
| UAUUGAAGUGGGGAAUUACAGAC | 1391 | usAfsuugAfaGfUfggggAfaUfuacagsasc | 2487 | antisense | 23 |
| UCUGUAAUUCCCCACUUCAAU | 1392 | uscsuguaAfuUfCfCfccacuucaauL96 | 2488 | sense | 21 |
| AUUGAAGUGGGGAAUUACAGACU | 1393 | asUfsugaAfgUfGfgggaAfuUfacagascsu | 2489 | antisense | 23 |
| UGAUGUGCGUAACAGAUUCAA | 1394 | usgsauguGfcGfUfAfacagauucaaL96 | 2490 | sense | 21 |
| UUGAAUCUGUUACGCACAUCAUC | 1395 | usUfsgaaUfcUfGfuuacGfcAfcaucasusc | 2491 | antisense | 23 |
| GAUGUGCGUAACAGAUUCAAA | 1396 | gsasugugCfgUfAfAfcagauucaaaL96 | 2492 | sense | 21 |
| UUUGAAUCUGUUACGCACAUCAU | 1397 | usUfsugaAfuCfUfguuaCfgCfacaucsasu | 2493 | antisense | 23 |
| UGGAUGAUGUGCGUAACAGAU | 1398 | usgsgaugAfuGfUfGfcguaacagauL96 | 2494 | sense | 21 |
| AUCUGUUACGCACAUCAUCCAGA | 1399 | asUfscugUfuAfCfgcacAfuCfauccasgsa | 2495 | antisense | 23 |
| CUGGAUGAUGUGCGUAACAGA | 1400 | csusggauGfaUfGfUfgcguaacagaL96 | 2496 | sense | 21 |
| UCUGUUACGCACAUCAUCCAGAC | 1401 | usCfsuguUfaCfGfcacaUfcAfuccagsasc | 2497 | antisense | 23 |
| GAAUGGGUGGCGGUAAUUGGU | 1402 | gsasauggGfuGfGfCfgguaauugguL96 | 2498 | sense | 21 |
| ACCAAUUACCGCCACCCAUUCCA | 1403 | asCfscaaUfuAfCfcgccAfcCfcauucscsa | 2499 | antisense | 23 |
| AAUGGGUGGCGGUAAUUGGUG | 1404 | asasugggUfgGfCfGfguaauuggugL96 | 2500 | sense | 21 |
| CACCAAUUACCGCCACCCAUUCC | 1405 | csAfsccaAfuUfAfccgcCfaCfccauuscsc | 2501 | antisense | 23 |
| AUUGGAAUGGGUGGCGGUAAU | 1406 | asusuggaAfuGfGfGfuggcgguaauL96 | 2502 | sense | 21 |
| AUUACCGCCACCCAUUCCAAUUC | 1407 | asUfsuacCfgCfCfacccAfuUfccaaususc | 2503 | antisense | 23 |
| AAUUGGAAUGGGUGGCGGUAA | 1408 | asasuuggAfaUfGfGfguggcgguaaL96 | 2504 | sense | 21 |
| UUACCGCCACCCAUUCCAAUUCU | 1409 | usUfsaccGfcCfAfcccaUfuCfcaauuscsu | 2505 | antisense | 23 |
| UCCGGAAUGUUGCUGAAACAG | 1410 | uscscggaAfuGfUfUfgcugaaacagL96 | 2506 | sense | 21 |
| CUGUUUCAGCAACAUUCCGGAGC | 1411 | csUfsguuUfcAfGfcaacAfuUfccggasgsc | 2507 | antisense | 23 |
| CCGGAAUGUUGCUGAAACAGA | 1412 | cscsggaaUfgUfUfGfcugaaacagaL96 | 2508 | sense | 21 |
| UCUGUUUCAGCAACAUUCCGGAG | 1413 | usCfsuguUfuCfAfgcaaCfaUfuccggsasg | 2509 | antisense | 23 |
| AUGCUCCGGAAUGUUGCUGAA | 1414 | asusgcucCfgGfAfAfuguugcugaaL96 | 2510 | sense | 21 |
| UUCAGCAACAUUCCGGAGCAUCC | 1415 | usUfscagCfaAfCfauucCfgGfagcauscsc | 2511 | antisense | 23 |
| GAUGCUCCGGAAUGUUGCUGA | 1416 | gsasugcuCfcGfGfAfauguugcugaL96 | 2512 | sense | 21 |
| UCAGCAACAUUCCGGAGCAUCCU | 1417 | usCfsagcAfaCfAfuuccGfgAfgcaucscsu | 2513 | antisense | 23 |
| UGUCCUCGAGAUACUAAAGGA | 1418 | usgsuccuCfgAfGfAfuacuaaaggaL96 | 2514 | sense | 21 |
| UCCUUUAGUAUCUCGAGGACAUC | 1419 | usCfscuuUfaGfUfaucuCfgAfggacasusc | 2515 | antisense | 23 |
| GUCCUCGAGAUACUAAAGGAA | 1420 | gsusccucGfaGfAfUfacuaaaggaaL96 | 2516 | sense | 21 |
| UUCCUUUAGUAUCUCGAGGACAU | 1421 | usUfsccuUfuAfGfuaucUfcGfaggacsasu | 2517 | antisense | 23 |
| AAGAUGUCCUCGAGAUACUAA | 1422 | asasgaugUfcCfUfCfgagauacuaaL96 | 2518 | sense | 21 |
| UUAGUAUCUCGAGGACAUCUUGA | 1423 | usUfsaguAfuCfUfcgagGfaCfaucuusgsa | 2519 | antisense | 23 |
| CAAGAUGUCCUCGAGAUACUA | 1424 | csasagauGfuCfCfUfcgagauacuaL96 | 2520 | sense | 21 |
| UAGUAUCUCGAGGACAUCUUGAA | 1425 | usAfsguaUfcUfCfgaggAfcAfucuugsasa | 2521 | antisense | 23 |
| ACAACAUGCUAAAUCAGUACU | 1426 | ascsaacaUfgCfUfAfaaucaguacuL96 | 2522 | sense | 21 |
| AGUACUGAUUUAGCAUGUUGUUC | 1427 | asGfsuacUfgAfUfuuagCfaUfguugususc | 2523 | antisense | 23 |
| CAACAUGCUAAAUCAGUACUU | 1428 | csasacauGfcUfAfAfaucaguacuuL96 | 2524 | sense | 21 |
| AAGUACUGAUUUAGCAUGUUGUU | 1429 | asAfsguaCfuGfAfuuuaGfcAfuguugsusu | 2525 | antisense | 23 |
| AUGAACAACAUGCUAAAUCAG | 1430 | asusgaacAfaCfAfUfgcuaaaucagL96 | 2526 | sense | 21 |
| CUGAUUUAGCAUGUUGUUCAUAA | 1431 | csUfsgauUfuAfGfcaugUfuGfuucausasa | 2527 | antisense | 23 |
| UAUGAACAACAUGCUAAAUCA | 1432 | usasugaaCfaAfCfAfugcuaaaucaL96 | 2528 | sense | 21 |
| UGAUUUAGCAUGUUGUUCAUAAU | 1433 | usGfsauuUfaGfCfauguUfgUfucauasasu | 2529 | antisense | 23 |
| GCCAAGGCUGUGUUUGUGGGG | 1434 | gscscaagGfcUfGfUfguuuguggggL96 | 2530 | sense | 21 |
| CCCCACAAACACAGCCUUGGCGC | 1435 | csCfsccaCfaAfAfcacaGfcCfuuggcsgsc | 2531 | antisense | 23 |
| CCAAGGCUGUGUUUGUGGGGA | 1436 | cscsaaggCfuGfUfGfuuuguggggaL96 | 2532 | sense | 21 |
| UCCCCACAAACACAGCCUUGGCG | 1437 | usCfscccAfcAfAfacacAfgCfcuuggscsg | 2533 | antisense | 23 |
| UGGCGCCAAGGCUGUGUUUGU | 1438 | usgsgcgcCfaAfGfGfcuguguuuguL96 | 2534 | sense | 21 |
| ACAAACACAGCCUUGGCGCCAAG | 1439 | asCfsaaaCfaCfAfgccuUfgGfcgccasasg | 2535 | antisense | 23 |
| UUGGCGCCAAGGCUGUGUUUG | 1440 | ususggcgCfcAfAfGfgcuguguuugL96 | 2536 | sense | 21 |
| CAAACACAGCCUUGGCGCCAAGA | 1441 | csAfsaacAfcAfGfccuuGfgCfgccaasgsa | 2537 | antisense | 23 |
| UGAAAGCUCUGGCUCUUGGCG | 1442 | usgsaaagCfuCfUfGfgcucuuggcgL96 | 2538 | sense | 21 |
| CGCCAAGAGCCAGAGCUUUCAGA | 1443 | csGfsccaAfgAfGfccagAfgCfuuucasgsa | 2539 | antisense | 23 |
| GAAAGCUCUGGCUCUUGGCGC | 1444 | gsasaagcUfcUfGfGfcucuuggcgcL96 | 2540 | sense | 21 |
| GCGCCAAGAGCCAGAGCUUUCAG | 1445 | gsCfsgccAfaGfAfgccaGfaGfcuuucsasg | 2541 | antisense | 23 |
| GUUCUGAAAGCUCUGGCUCUU | 1446 | gsusucugAfaAfGfCfucuggcucuuL96 | 2542 | sense | 21 |
| AAGAGCCAGAGCUUUCAGAACAU | 1447 | asAfsgagCfcAfGfagcuUfuCfagaacsasu | 2543 | antisense | 23 |
| UGUUCUGAAAGCUCUGGCUCU | 1448 | usgsuucuGfaAfAfGfcucuggcucuL96 | 2544 | sense | 21 |
| AGAGCCAGAGCUUUCAGAACAUC | 1449 | asGfsagcCfaGfAfgcuuUfcAfgaacasusc | 2545 | antisense | 23 |
| CAGCCACUAUUGAUGUUCUGC | 1450 | csasgccaCfuAfUfUfgauguucugcL96 | 2546 | sense | 21 |
| GCAGAACAUCAAUAGUGGCUGGC | 1451 | gsCfsagaAfcAfUfcaauAfgUfggcugsgsc | 2547 | antisense | 23 |
| AGCCACUAUUGAUGUUCUGCC | 1452 | asgsccacUfaUfUfGfauguucugccL96 | 2548 | sense | 21 |
| GGCAGAACAUCAAUAGUGGCUGG | 1453 | gsGfscagAfaCfAfucaaUfaGfuggcusgsg | 2549 | antisense | 23 |
| GUGCCAGCCACUAUUGAUGUU | 1454 | gsusgccaGfcCfAfCfuauugauguuL96 | 2550 | sense | 21 |
| AACAUCAAUAGUGGCUGGCACCC | 1455 | asAfscauCfaAfUfagugGfcUfggcacscsc | 2551 | antisense | 23 |
| GGUGCCAGCCACUAUUGAUGU | 1456 | gsgsugccAfgCfCfAfcuauugauguL96 | 2552 | sense | 21 |
| ACAUCAAUAGUGGCUGGCACCCC | 1457 | asCfsaucAfaUfAfguggCfuGfgcaccscsc | 2553 | antisense | 23 |
| ACAAGGACCGAGAAGUCACCA | 1458 | ascsaaggAfcCfGfAfgaagucaccaL96 | 2554 | sense | 21 |
| UGGUGACUUCUCGGUCCUUGUAG | 1459 | usGfsgugAfcUfUfcucgGfuCfcuugusasg | 2555 | antisense | 23 |
| CAAGGACCGAGAAGUCACCAA | 1460 | csasaggaCfcGfAfGfaagucaccaaL96 | 2556 | sense | 21 |
| UUGGUGACUUCUCGGUCCUUGUA | 1461 | usUfsgguGfaCfUfucucGfgUfccuugsusa | 2557 | antisense | 23 |
| AUCUACAAGGACCGAGAAGUC | 1462 | asuscuacAfaGfGfAfccgagaagucL96 | 2558 | sense | 21 |
| GACUUCUCGGUCCUUGUAGAUAU | 1463 | gsAfscuuCfuCfGfguccUfuGfuagausasu | 2559 | antisense | 23 |
| UAUCUACAAGGACCGAGAAGU | 1464 | usasucuaCfaAfGfGfaccgagaaguL96 | 2560 | sense | 21 |
| ACUUCUCGGUCCUUGUAGAUAUA | 1465 | asCfsuucUfcGfGfuccuUfgUfagauasusa | 2561 | antisense | 23 |
| CAGAAUGUGAAAGUCAUCGAC | 1466 | csasgaauGfuGfAfAfagucaucgacL96 | 2562 | sense | 21 |
| GUCGAUGACUUUCACAUUCUGGC | 1467 | gsUfscgaUfgAfCfuuucAfcAfuucugsgsc | 2563 | antisense | 23 |
| AGAAUGUGAAAGUCAUCGACA | 1468 | asgsaaugUfgAfAfAfgucaucgacaL96 | 2564 | sense | 21 |
| UGUCGAUGACUUUCACAUUCUGG | 1469 | usGfsucgAfuGfAfcuuuCfaCfauucusgsg | 2565 | antisense | 23 |
| GUGCCAGAAUGUGAAAGUCAU | 1470 | gsusgccaGfaAfUfGfugaaagucauL96 | 2566 | sense | 21 |
| AUGACUUUCACAUUCUGGCACCC | 1471 | asUfsgacUfuUfCfacauUfcUfggcacscsc | 2567 | antisense | 23 |
| GGUGCCAGAAUGUGAAAGUCA | 1472 | gsgsugccAfgAfAfUfgugaaagucaL96 | 2568 | sense | 21 |
| UGACUUUCACAUUCUGGCACCCA | 1473 | usGfsacuUfuCfAfcauuCfuGfgcaccscsa | 2569 | antisense | 23 |
| AGAUGUCCUCGAGAUACUAAA | 1474 | asgsauguCfcUfCfGfagauacuaaaL96 | 2570 | sense | 21 |
| UUUAGUAUCUCGAGGACAUCUUG | 1475 | usUfsuagUfaUfCfucgaGfgAfcaucususg | 2571 | antisense | 23 |
| GAUGUCCUCGAGAUACUAAAG | 1476 | gsasugucCfuCfGfAfgauacuaaagL96 | 2572 | sense | 21 |
| CUUUAGUAUCUCGAGGACAUCUU | 1477 | csUfsuuaGfuAfUfcucgAfgGfacaucsusu | 2573 | antisense | 23 |
| UUCAAGAUGUCCUCGAGAUAC | 1478 | ususcaagAfuGfUfCfcucgagauacL96 | 2574 | sense | 21 |
| GUAUCUCGAGGACAUCUUGAACA | 1479 | gsUfsaucUfcGfAfggacAfuCfuugaascsa | 2575 | antisense | 23 |
| GUUCAAGAUGUCCUCGAGAUA | 1480 | gsusucaaGfaUfGfUfccucgagauaL96 | 2576 | sense | 21 |
| UAUCUCGAGGACAUCUUGAACAC | 1481 | usAfsucuCfgAfGfgacaUfcUfugaacsasc | 2577 | antisense | 23 |
| GUGGACUUGCUGCAUAUGUGG | 1482 | gsusggacUfuGfCfUfgcauauguggL96 | 2578 | sense | 21 |
| CCACAUAUGCAGCAAGUCCACUG | 1483 | csCfsacaUfaUfGfcagcAfaGfuccacsusg | 2579 | antisense | 23 |
| UGGACUUGCUGCAUAUGUGGC | 1484 | usgsgacuUfgCfUfGfcauauguggcL96 | 2580 | sense | 21 |
| GCCACAUAUGCAGCAAGUCCACU | 1485 | gsCfscacAfuAfUfgcagCfaAfguccascsu | 2581 | antisense | 23 |
| GACAGUGGACUUGCUGCAUAU | 1486 | gsascaguGfgAfCfUfugcugcauauL96 | 2582 | sense | 21 |
| AUAUGCAGCAAGUCCACUGUCGU | 1487 | asUfsaugCfaGfCfaaguCfcAfcugucsgsu | 2583 | antisense | 23 |
| CGACAGUGGACUUGCUGCAUA | 1488 | csgsacagUfgGfAfCfuugcugcauaL96 | 2584 | sense | 21 |
| UAUGCAGCAAGUCCACUGUCGUC | 1489 | usAfsugcAfgCfAfagucCfaCfugucgsusc | 2585 | antisense | 23 |
| AACCAGUACUUUAUCAUUUUC | 1490 | asasccagUfaCfUfUfuaucauuuucL96 | 2586 | sense | 21 |
| GAAAAUGAUAAAGUACUGGUUUC | 1491 | gsAfsaaaUfgAfUfaaagUfaCfugguususc | 2587 | antisense | 23 |
| ACCAGUACUUUAUCAUUUUCU | 1492 | ascscaguAfcUfUfUfaucauuuucuL96 | 2588 | sense | 21 |
| AGAAAAUGAUAAAGUACUGGUUU | 1493 | asGfsaaaAfuGfAfuaaaGfuAfcuggususu | 2589 | antisense | 23 |
| UUGAAACCAGUACUUUAUCAU | 1494 | ususgaaaCfcAfGfUfacuuuaucauL96 | 2590 | sense | 21 |
| AUGAUAAAGUACUGGUUUCAAAA | 1495 | asUfsgauAfaAfGfuacuGfgUfuucaasasa | 2591 | antisense | 23 |
| UUUGAAACCAGUACUUUAUCA | 1496 | ususugaaAfcCfAfGfuacuuuaucaL96 | 2592 | sense | 21 |
| UGAUAAAGUACUGGUUUCAAAAU | 1497 | usGfsauaAfaGfUfacugGfuUfucaaasasu | 2593 | antisense | 23 |
| CGAGAAGUCACCAAGAAGCUA | 1498 | csgsagaaGfuCfAfCfcaagaagcuaL96 | 2594 | sense | 21 |
| UAGCUUCUUGGUGACUUCUCGGU | 1499 | usAfsgcuUfcUfUfggugAfcUfucucgsgsu | 2595 | antisense | 23 |
| GAGAAGUCACCAAGAAGCUAG | 1500 | gsasgaagUfcAfCfCfaagaagcuagL96 | 2596 | sense | 21 |
| CUAGCUUCUUGGUGACUUCUCGG | 1501 | csUfsagcUfuCfUfugguGfaCfuucucsgsg | 2597 | antisense | 23 |
| GGACCGAGAAGUCACCAAGAA | 1502 | gsgsaccgAfgAfAfGfucaccaagaaL96 | 2598 | sense | 21 |
| UUCUUGGUGACUUCUCGGUCCUU | 1503 | usUfscuuGfgUfGfacuuCfuCfgguccsusu | 2599 | antisense | 23 |
| AGGACCGAGAAGUCACCAAGA | 1504 | asgsgaccGfaGfAfAfgucaccaagaL96 | 2600 | sense | 21 |
| UCUUGGUGACUUCUCGGUCCUUG | 1505 | usCfsuugGfuGfAfcuucUfcGfguccususg | 2601 | antisense | 23 |
| UCAAAGUGUUGGUAAUGCCUG | 1506 | uscsaaagUfgUfUfGfguaaugccugL96 | 2602 | sense | 21 |
| CAGGCAUUACCAACACUUUGAAC | 1507 | csAfsggcAfuUfAfccaaCfaCfuuugasasc | 2603 | antisense | 23 |
| CAAAGUGUUGGUAAUGCCUGA | 1508 | csasaaguGfuUfGfGfuaaugccugaL96 | 2604 | sense | 21 |
| UCAGGCAUUACCAACACUUUGAA | 1509 | usCfsaggCfaUfUfaccaAfcAfcuuugsasa | 2605 | antisense | 23 |
| AGGUUCAAAGUGUUGGUAAUG | 1510 | asgsguucAfaAfGfUfguugguaaugL96 | 2606 | sense | 21 |
| CAUUACCAACACUUUGAACCUGA | 1511 | csAfsuuaCfcAfAfcacuUfuGfaaccusgsa | 2607 | antisense | 23 |
| CAGGUUCAAAGUGUUGGUAAU | 1512 | csasgguuCfaAfAfGfuguugguaauL96 | 2608 | sense | 21 |
| AUUACCAACACUUUGAACCUGAG | 1513 | asUfsuacCfaAfCfacuuUfgAfaccugsasg | 2609 | antisense | 23 |
| UAUUACUUGACAAAGAGACAC | 1514 | usasuuacUfuGfAfCfaaagagacacL96 | 2610 | sense | 21 |
| GUGUCUCUUUGUCAAGUAAUACA | 1515 | gsUfsgucUfcUfUfugucAfaGfuaauascsa | 2611 | antisense | 23 |
| AUUACUUGACAAAGAGACACU | 1516 | asusuacuUfgAfCfAfaagagacacuL96 | 2612 | sense | 21 |
| AGUGUCUCUUUGUCAAGUAAUAC | 1517 | asGfsuguCfuCfUfuuguCfaAfguaausasc | 2613 | antisense | 23 |
| CAUGUAUUACUUGACAAAGAG | 1518 | csasuguaUfuAfCfUfugacaaagagL96 | 2614 | sense | 21 |
| CUCUUUGUCAAGUAAUACAUGCU | 1519 | csUfscuuUfgUfCfaaguAfaUfacaugscsu | 2615 | antisense | 23 |
| GCAUGUAUUACUUGACAAAGA | 1520 | gscsauguAfuUfAfCfuugacaaagaL96 | 2616 | sense | 21 |
| UCUUUGUCAAGUAAUACAUGCUG | 1521 | usCfsuuuGfuCfAfaguaAfuAfcaugcsusg | 2617 | antisense | 23 |
| AAAGUCAUCGACAAGACAUUG | 1522 | asasagucAfuCfGfAfcaagacauugL96 | 2618 | sense | 21 |
| CAAUGUCUUGUCGAUGACUUUCA | 1523 | csAfsaugUfcUfUfgucgAfuGfacuuuscsa | 2619 | antisense | 23 |
| AAGUCAUCGACAAGACAUUGG | 1524 | asasgucaUfcGfAfCfaagacauuggL96 | 2620 | sense | 21 |
| CCAAUGUCUUGUCGAUGACUUUC | 1525 | csCfsaauGfuCfUfugucGfaUfgacuususc | 2621 | antisense | 23 |
| UGUGAAAGUCAUCGACAAGAC | 1526 | usgsugaaAfgUfCfAfucgacaagacL96 | 2622 | sense | 21 |
| GUCUUGUCGAUGACUUUCACAUU | 1527 | gsUfscuuGfuCfGfaugaCfuUfucacasusu | 2623 | antisense | 23 |
| AUGUGAAAGUCAUCGACAAGA | 1528 | asusgugaAfaGfUfCfaucgacaagaL96 | 2624 | sense | 21 |
| UCUUGUCGAUGACUUUCACAUUC | 1529 | usCfsuugUfcGfAfugacUfuUfcacaususc | 2625 | antisense | 23 |
| AUAUGUGGCUAAAGCAAUAGA | 1530 | asusauguGfgCfUfAfaagcaauagaL96 | 2626 | sense | 21 |
| UCUAUUGCUUUAGCCACAUAUGC | 1531 | usCfsuauUfgCfUfuuagCfcAfcauausgsc | 2627 | antisense | 23 |
| UAUGUGGCUAAAGCAAUAGAC | 1532 | usasugugGfcUfAfAfagcaauagacL96 | 2628 | sense | 21 |
| GUCUAUUGCUUUAGCCACAUAUG | 1533 | gsUfscuaUfuGfCfuuuaGfcCfacauasusg | 2629 | antisense | 23 |
| CUGCAUAUGUGGCUAAAGCAA | 1534 | csusgcauAfuGfUfGfgcuaaagcaaL96 | 2630 | sense | 21 |
| UUGCUUUAGCCACAUAUGCAGCA | 1535 | usUfsgcuUfuAfGfccacAfuAfugcagscsa | 2631 | antisense | 23 |
| GCUGCAUAUGUGGCUAAAGCA | 1536 | gscsugcaUfaUfGfUfggcuaaagcaL96 | 2632 | sense | 21 |
| UGCUUUAGCCACAUAUGCAGCAA | 1537 | usGfscuuUfaGfCfcacaUfaUfgcagcsasa | 2633 | antisense | 23 |
| AGACGACAGUGGACUUGCUGC | 1538 | asgsacgaCfaGfUfGfgacuugcugcL96 | 2634 | sense | 21 |
| GCAGCAAGUCCACUGUCGUCUCC | 1539 | gsCfsagcAfaGfUfccacUfgUfcgucuscsc | 2635 | antisense | 23 |
| GACGACAGUGGACUUGCUGCA | 1540 | gsascgacAfgUfGfGfacuugcugcaL96 | 2636 | sense | 21 |
| UGCAGCAAGUCCACUGUCGUCUC | 1541 | usGfscagCfaAfGfuccaCfuGfucgucsusc | 2637 | antisense | 23 |
| UUGGAGACGACAGUGGACUUG | 1542 | ususggagAfcGfAfCfaguggacuugL96 | 2638 | sense | 21 |
| CAAGUCCACUGUCGUCUCCAAAA | 1543 | csAfsaguCfcAfCfugucGfuCfuccaasasa | 2639 | antisense | 23 |
| UUUGGAGACGACAGUGGACUU | 1544 | ususuggaGfaCfGfAfcaguggacuuL96 | 2640 | sense | 21 |
| AAGUCCACUGUCGUCUCCAAAAU | 1545 | asAfsgucCfaCfUfgucgUfcUfccaaasasu | 2641 | antisense | 23 |
| GGCCACCUCCUCAAUUGAAGA | 1546 | gsgsccacCfuCfCfUfcaauugaagaL96 | 2642 | sense | 21 |
| UCUUCAAUUGAGGAGGUGGCCCA | 1547 | usCfsuucAfaUfUfgaggAfgGfuggccscsa | 2643 | antisense | 23 |
| GCCACCUCCUCAAUUGAAGAA | 1548 | gscscaccUfcCfUfCfaauugaagaaL96 | 2644 | sense | 21 |
| UUCUUCAAUUGAGGAGGUGGCCC | 1549 | usUfscuuCfaAfUfugagGfaGfguggcscsc | 2645 | antisense | 23 |
| CCUGGGCCACCUCCUCAAUUG | 1550 | cscsugggCfcAfCfCfuccucaauugL96 | 2646 | sense | 21 |
| CAAUUGAGGAGGUGGCCCAGGAA | 1551 | csAfsauuGfaGfGfagguGfgCfccaggsasa | 2647 | antisense | 23 |
| UCCUGGGCCACCUCCUCAAUU | 1552 | uscscuggGfcCfAfCfcuccucaauuL96 | 2648 | sense | 21 |
| AAUUGAGGAGGUGGCCCAGGAAC | 1553 | asAfsuugAfgGfAfggugGfcCfcaggasasc | 2649 | antisense | 23 |
| UGUAUGUUACUUCUUAGAGAG | 1554 | usgsuaugUfuAfCfUfucuuagagagL96 | 2650 | sense | 21 |
| CUCUCUAAGAAGUAACAUACAUC | 1555 | csUfscucUfaAfGfaaguAfaCfauacasusc | 2651 | antisense | 23 |
| GUAUGUUACUUCUUAGAGAGA | 1556 | gsusauguUfaCfUfUfcuuagagagaL96 | 2652 | sense | 21 |
| UCUCUCUAAGAAGUAACAUACAU | 1557 | usCfsucuCfuAfAfgaagUfaAfcauacsasu | 2653 | antisense | 23 |
| AGGAUGUAUGUUACUUCUUAG | 1558 | asgsgaugUfaUfGfUfuacuucuuagL96 | 2654 | sense | 21 |
| CUAAGAAGUAACAUACAUCCUAA | 1559 | csUfsaagAfaGfUfaacaUfaCfauccusasa | 2655 | antisense | 23 |
| UAGGAUGUAUGUUACUUCUUA | 1560 | usasggauGfuAfUfGfuuacuucuuaL96 | 2656 | sense | 21 |
| UAAGAAGUAACAUACAUCCUAAA | 1561 | usAfsagaAfgUfAfacauAfcAfuccuasasa | 2657 | antisense | 23 |
| AAAUGUUUUAGGAUGUAUGUU | 1562 | asasauguUfuUfAfGfgauguauguuL96 | 2658 | sense | 21 |
| AACAUACAUCCUAAAACAUUUGG | 1563 | asAfscauAfcAfUfccuaAfaAfcauuusgsg | 2659 | antisense | 23 |
| AAUGUUUUAGGAUGUAUGUUA | 1564 | asasuguuUfuAfGfGfauguauguuaL96 | 2660 | sense | 21 |
| UAACAUACAUCCUAAAACAUUUG | 1565 | usAfsacaUfaCfAfuccuAfaAfacauususg | 2661 | antisense | 23 |
| AUCCAAAUGUUUUAGGAUGUA | 1566 | asusccaaAfuGfUfUfuuaggauguaL96 | 2662 | sense | 21 |
| UACAUCCUAAAACAUUUGGAUAU | 1567 | usAfscauCfcUfAfaaacAfuUfuggausasu | 2663 | antisense | 23 |
| UAUCCAAAUGUUUUAGGAUGU | 1568 | usasuccaAfaUfGfUfuuuaggauguL96 | 2664 | sense | 21 |
| ACAUCCUAAAACAUUUGGAUAUA | 1569 | asCfsaucCfuAfAfaacaUfuUfggauasusa | 2665 | antisense | 23 |
| AUGGGUGGCGGUAAUUGGUGA | 1570 | asusggguGfgCfGfGfuaauuggugaL96 | 2666 | sense | 21 |
| UCACCAAUUACCGCCACCCAUUC | 1571 | usCfsaccAfaUfUfaccgCfcAfcccaususc | 2667 | antisense | 23 |
| UGGGUGGCGGUAAUUGGUGAU | 1572 | usgsggugGfcGfGfUfaauuggugauL96 | 2668 | sense | 21 |
| AUCACCAAUUACCGCCACCCAUU | 1573 | asUfscacCfaAfUfuaccGfcCfacccasusu | 2669 | antisense | 23 |
| UGGAAUGGGUGGCGGUAAUUG | 1574 | usgsgaauGfgGfUfGfgcgguaauugL96 | 2670 | sense | 21 |
| CAAUUACCGCCACCCAUUCCAAU | 1575 | csAfsauuAfcCfGfccacCfcAfuuccasasu | 2671 | antisense | 23 |
| UUGGAAUGGGUGGCGGUAAUU | 1576 | ususggaaUfgGfGfUfggcgguaauuL96 | 2672 | sense | 21 |
| AAUUACCGCCACCCAUUCCAAUU | 1577 | asAfsuuaCfcGfCfcaccCfaUfuccaasusu | 2673 | antisense | 23 |
| UUCAAAGUGUUGGUAAUGCCU | 1578 | ususcaaaGfuGfUfUfgguaaugccuL96 | 2674 | sense | 21 |
| AGGCAUUACCAACACUUUGAACC | 1579 | asGfsgcaUfuAfCfcaacAfcUfuugaascsc | 2675 | antisense | 23 |
| UCAAAGUGUUGGUAAUGCCUG | 1580 | uscsaaagUfgUfUfGfguaaugccugL96 | 2676 | sense | 21 |
| CAGGCAUUACCAACACUUUGAAC | 1581 | csAfsggcAfuUfAfccaaCfaCfuuugasasc | 2677 | antisense | 23 |
| CAGGUUCAAAGUGUUGGUAAU | 1582 | csasgguuCfaAfAfGfuguugguaauL96 | 2678 | sense | 21 |
| AUUACCAACACUUUGAACCUGAG | 1583 | asUfsuacCfaAfCfacuuUfgAfaccugsasg | 2679 | antisense | 23 |
| UCAGGUUCAAAGUGUUGGUAA | 1584 | uscsagguUfcAfAfAfguguugguaaL96 | 2680 | sense | 21 |
| UUACCAACACUUUGAACCUGAGC | 1585 | usUfsaccAfaCfAfcuuuGfaAfccugasgsc | 2681 | antisense | 23 |
| CCACCUCCUCAAUUGAAGAAG | 1586 | cscsaccuCfcUfCfAfauugaagaagL96 | 2682 | sense | 21 |
| CUUCUUCAAUUGAGGAGGUGGCC | 1587 | csUfsucuUfcAfAfuugaGfgAfgguggscsc | 2683 | antisense | 23 |
| CACCUCCUCAAUUGAAGAAGU | 1588 | csasccucCfuCfAfAfuugaagaaguL96 | 2684 | sense | 21 |
| ACUUCUUCAAUUGAGGAGGUGGC | 1589 | asCfsuucUfuCfAfauugAfgGfaggugsgsc | 2685 | antisense | 23 |
| UGGGCCACCUCCUCAAUUGAA | 1590 | usgsggccAfcCfUfCfcucaauugaaL96 | 2686 | sense | 21 |
| UUCAAUUGAGGAGGUGGCCCAGG | 1591 | usUfscaaUfuGfAfggagGfuGfgcccasgsg | 2687 | antisense | 23 |
| CUGGGCCACCUCCUCAAUUGA | 1592 | csusgggcCfaCfCfUfccucaauugaL96 | 2688 | sense | 21 |
| UCAAUUGAGGAGGUGGCCCAGGA | 1593 | usCfsaauUfgAfGfgaggUfgGfcccagsgsa | 2689 | antisense | 23 |
| GAGUGGGUGCCAGAAUGUGAA | 1594 | gsasguggGfuGfCfCfagaaugugaaL96 | 2690 | sense | 21 |
| UUCACAUUCUGGCACCCACUCAG | 1595 | usUfscacAfuUfCfuggcAfcCfcacucsasg | 2691 | antisense | 23 |
| AGUGGGUGCCAGAAUGUGAAA | 1596 | asgsugggUfgCfCfAfgaaugugaaaL96 | 2692 | sense | 21 |
| UUUCACAUUCUGGCACCCACUCA | 1597 | usUfsucaCfaUfUfcuggCfaCfccacuscsa | 2693 | antisense | 23 |
| CUCUGAGUGGGUGCCAGAAUG | 1598 | csuscugaGfuGfGfGfugccagaaugL96 | 2694 | sense | 21 |
| CAUUCUGGCACCCACUCAGAGCC | 1599 | csAfsuucUfgGfCfacccAfcUfcagagscsc | 2695 | antisense | 23 |
| GCUCUGAGUGGGUGCCAGAAU | 1600 | gscsucugAfgUfGfGfgugccagaauL96 | 2696 | sense | 21 |
| AUUCUGGCACCCACUCAGAGCCA | 1601 | asUfsucuGfgCfAfcccaCfuCfagagcscsa | 2697 | antisense | 23 |
| GCACUGAUGUUCUGAAAGCUC | 1602 | gscsacugAfuGfUfUfcugaaagcucL96 | 2698 | sense | 21 |
| GAGCUUUCAGAACAUCAGUGCCU | 1603 | gsAfsgcuUfuCfAfgaacAfuCfagugcscsu | 2699 | antisense | 23 |
| CACUGAUGUUCUGAAAGCUCU | 1604 | csascugaUfgUfUfCfugaaagcucuL96 | 2700 | sense | 21 |
| AGAGCUUUCAGAACAUCAGUGCC | 1605 | asGfsagcUfuUfCfagaaCfaUfcagugscsc | 2701 | antisense | 23 |
| AAAGGCACUGAUGUUCUGAAA | 1606 | asasaggcAfcUfGfAfuguucugaaaL96 | 2702 | sense | 21 |
| UUUCAGAACAUCAGUGCCUUUCC | 1607 | usUfsucaGfaAfCfaucaGfuGfccuuuscsc | 2703 | antisense | 23 |
| GAAAGGCACUGAUGUUCUGAA | 1608 | gsasaaggCfaCfUfGfauguucugaaL96 | 2704 | sense | 21 |
| UUCAGAACAUCAGUGCCUUUCCG | 1609 | usUfscagAfaCfAfucagUfgCfcuuucscsg | 2705 | antisense | 23 |
| GGGAAGGUGGAAGUCUUCCUG | 1610 | gsgsgaagGfuGfGfAfagucuuccugL96 | 2706 | sense | 21 |
| CAGGAAGACUUCCACCUUCCCUU | 1611 | csAfsggaAfgAfCfuuccAfcCfuucccsusu | 2707 | antisense | 23 |
| GGAAGGUGGAAGUCUUCCUGG | 1612 | gsgsaaggUfgGfAfAfgucuuccuggL96 | 2708 | sense | 21 |
| CCAGGAAGACUUCCACCUUCCCU | 1613 | csCfsaggAfaGfAfcuucCfaCfcuuccscsu | 2709 | antisense | 23 |
| GGAAGGGAAGGUGGAAGUCUU | 1614 | gsgsaaggGfaAfGfGfuggaagucuuL96 | 2710 | sense | 21 |
| AAGACUUCCACCUUCCCUUCCAC | 1615 | asAfsgacUfuCfCfaccuUfcCfcuuccsasc | 2711 | antisense | 23 |
| UGGAAGGGAAGGUGGAAGUCU | 1616 | usgsgaagGfgAfAfGfguggaagucuL96 | 2712 | sense | 21 |
| AGACUUCCACCUUCCCUUCCACA | 1617 | asGfsacuUfcCfAfccuuCfcCfuuccascsa | 2713 | antisense | 23 |
| UGCUAAAUCAGUACUUCCAAA | 1618 | usgscuaaAfuCfAfGfuacuuccaaaL96 | 2714 | sense | 21 |
| UUUGGAAGUACUGAUUUAGCAUG | 1619 | usUfsuggAfaGfUfacugAfuUfuagcasusg | 2715 | antisense | 23 |
| GCUAAAUCAGUACUUCCAAAG | 1620 | gscsuaaaUfcAfGfUfacuuccaaagL96 | 2716 | sense | 21 |
| CUUUGGAAGUACUGAUUUAGCAU | 1621 | csUfsuugGfaAfGfuacuGfaUfuuagcsasu | 2717 | antisense | 23 |
| AACAUGCUAAAUCAGUACUUC | 1622 | asascaugCfuAfAfAfucaguacuucL96 | 2718 | sense | 21 |
| GAAGUACUGAUUUAGCAUGUUGU | 1623 | gsAfsaguAfcUfGfauuuAfgCfauguusgsu | 2719 | antisense | 23 |
| CAACAUGCUAAAUCAGUACUU | 1624 | csasacauGfcUfAfAfaucaguacuuL96 | 2720 | sense | 21 |
| AAGUACUGAUUUAGCAUGUUGUU | 1625 | asAfsguaCfuGfAfuuuaGfcAfuguugsusu | 2721 | antisense | 23 |
| CCACAACUCAGGAUGAAAAAU | 1626 | cscsacaaCfuCfAfGfgaugaaaaauL96 | 2722 | sense | 21 |
| AUUUUUCAUCCUGAGUUGUGGCG | 1627 | asUfsuuuUfcAfUfccugAfgUfuguggscsg | 2723 | antisense | 23 |
| CACAACUCAGGAUGAAAAAUU | 1628 | csascaacUfcAfGfGfaugaaaaauuL96 | 2724 | sense | 21 |
| AAUUUUUCAUCCUGAGUUGUGGC | 1629 | asAfsuuuUfuCfAfuccuGfaGfuugugsgsc | 2725 | antisense | 23 |
| GCCGCCACAACUCAGGAUGAA | 1630 | gscscgccAfcAfAfCfucaggaugaaL96 | 2726 | sense | 21 |
| UUCAUCCUGAGUUGUGGCGGCAG | 1631 | usUfscauCfcUfGfaguuGfuGfgcggcsasg | 2727 | antisense | 23 |
| UGCCGCCACAACUCAGGAUGA | 1632 | usgsccgcCfaCfAfAfcucaggaugaL96 | 2728 | sense | 21 |
| UCAUCCUGAGUUGUGGCGGCAGU | 1633 | usCfsaucCfuGfAfguugUfgGfcggcasgsu | 2729 | antisense | 23 |
| GCAACCGUCUGGAUGAUGUGC | 1634 | gscsaaccGfuCfUfGfgaugaugugcL96 | 2730 | sense | 21 |
| GCACAUCAUCCAGACGGUUGCCC | 1635 | gsCfsacaUfcAfUfccagAfcGfguugcscsc | 2731 | antisense | 23 |
| CAACCGUCUGGAUGAUGUGCG | 1636 | csasaccgUfcUfGfGfaugaugugcgL96 | 2732 | sense | 21 |
| CGCACAUCAUCCAGACGGUUGCC | 1637 | csGfscacAfuCfAfuccaGfaCfgguugscsc | 2733 | antisense | 23 |
| CUGGGCAACCGUCUGGAUGAU | 1638 | csusgggcAfaCfCfGfucuggaugauL96 | 2734 | sense | 21 |
| AUCAUCCAGACGGUUGCCCAGGU | 1639 | asUfscauCfcAfGfacggUfuGfcccagsgsu | 2735 | antisense | 23 |
| CCUGGGCAACCGUCUGGAUGA | 1640 | cscsugggCfaAfCfCfgucuggaugaL96 | 2736 | sense | 21 |
| UCAUCCAGACGGUUGCCCAGGUA | 1641 | usCfsaucCfaGfAfcgguUfgCfccaggsusa | 2737 | antisense | 23 |
| GCAAAUGAUGAAGAAACUUUG | 1642 | gscsaaauGfaUfGfAfagaaacuuugL96 | 2738 | sense | 21 |
| CAAAGUUUCUUCAUCAUUUGCCC | 1643 | csAfsaagUfuUfCfuucaUfcAfuuugcscsc | 2739 | antisense | 23 |
| CAAAUGAUGAAGAAACUUUGG | 1644 | csasaaugAfuGfAfAfgaaacuuuggL96 | 2740 | sense | 21 |
| CCAAAGUUUCUUCAUCAUUUGCC | 1645 | csCfsaaaGfuUfUfcuucAfuCfauuugscsc | 2741 | antisense | 23 |
| UGGGGCAAAUGAUGAAGAAAC | 1646 | usgsgggcAfaAfUfGfaugaagaaacL96 | 2742 | sense | 21 |
| GUUUCUUCAUCAUUUGCCCCAGA | 1647 | gsUfsuucUfuCfAfucauUfuGfccccasgsa | 2743 | antisense | 23 |
| CUGGGGCAAAUGAUGAAGAAA | 1648 | csusggggCfaAfAfUfgaugaagaaaL96 | 2744 | sense | 21 |
| UUUCUUCAUCAUUUGCCCCAGAC | 1649 | usUfsucuUfcAfUfcauuUfgCfcccagsasc | 2745 | antisense | 23 |
| CCAAGGCUGUGUUUGUGGGGA | 1650 | cscsaaggCfuGfUfGfuuuguggggaL96 | 2746 | sense | 21 |
| UCCCCACAAACACAGCCUUGGCG | 1651 | usCfscccAfcAfAfacacAfgCfcuuggscsg | 2747 | antisense | 23 |
| CAAGGCUGUGUUUGUGGGGAG | 1652 | csasaggcUfgUfGfUfuuguggggagL96 | 2748 | sense | 21 |
| CUCCCCACAAACACAGCCUUGGC | 1653 | csUfscccCfaCfAfaacaCfaGfccuugsgsc | 2749 | antisense | 23 |
| GGCGCCAAGGCUGUGUUUGUG | 1654 | gsgscgccAfaGfGfCfuguguuugugL96 | 2750 | sense | 21 |
| CACAAACACAGCCUUGGCGCCAA | 1655 | csAfscaaAfcAfCfagccUfuGfgcgccsasa | 2751 | antisense | 23 |
| UGGCGCCAAGGCUGUGUUUGU | 1656 | usgsgcgcCfaAfGfGfcuguguuuguL96 | 2752 | sense | 21 |
| ACAAACACAGCCUUGGCGCCAAG | 1657 | asCfsaaaCfaCfAfgccuUfgGfcgccasasg | 2753 | antisense | 23 |
| ACUGCCGCCACAACUCAGGAU | 1658 | ascsugccGfcCfAfCfaacucaggauL96 | 2754 | sense | 21 |
| AUCCUGAGUUGUGGCGGCAGUUU | 1659 | asUfsccuGfaGfUfugugGfcGfgcagususu | 2755 | antisense | 23 |
| CUGCCGCCACAACUCAGGAUG | 1660 | csusgccgCfcAfCfAfacucaggaugL96 | 2756 | sense | 21 |
| CAUCCUGAGUUGUGGCGGCAGUU | 1661 | csAfsuccUfgAfGfuuguGfgCfggcagsusu | 2757 | antisense | 23 |
| UCAAACUGCCGCCACAACUCA | 1662 | uscsaaacUfgCfCfGfccacaacucaL96 | 2758 | sense | 21 |
| UGAGUUGUGGCGGCAGUUUGAAU | 1663 | usGfsaguUfgUfGfgcggCfaGfuuugasasu | 2759 | antisense | 23 |
| UUCAAACUGCCGCCACAACUC | 1664 | ususcaaaCfuGfCfCfgccacaacucL96 | 2760 | sense | 21 |
| GAGUUGUGGCGGCAGUUUGAAUC | 1665 | gsAfsguuGfuGfGfcggcAfgUfuugaasusc | 2761 | antisense | 23 |
| GGGAAGAUAUCAAAUGGCUGA | 1666 | gsgsgaagAfuAfUfCfaaauggcugaL96 | 2762 | sense | 21 |
| UCAGCCAUUUGAUAUCUUCCCAG | 1667 | usCfsagcCfaUfUfugauAfuCfuucccsasg | 2763 | antisense | 23 |
| GGAAGAUAUCAAAUGGCUGAG | 1668 | gsgsaagaUfaUfCfAfaauggcugagL96 | 2764 | sense | 21 |
| CUCAGCCAUUUGAUAUCUUCCCA | 1669 | csUfscagCfcAfUfuugaUfaUfcuuccscsa | 2765 | antisense | 23 |
| AGCUGGGAAGAUAUCAAAUGG | 1670 | asgscuggGfaAfGfAfuaucaaauggL96 | 2766 | sense | 21 |
| CCAUUUGAUAUCUUCCCAGCUGA | 1671 | csCfsauuUfgAfUfaucuUfcCfcagcusgsa | 2767 | antisense | 23 |
| CAGCUGGGAAGAUAUCAAAUG | 1672 | csasgcugGfgAfAfGfauaucaaaugL96 | 2768 | sense | 21 |
| CAUUUGAUAUCUUCCCAGCUGAU | 1673 | csAfsuuuGfaUfAfucuuCfcCfagcugsasu | 2769 | antisense | 23 |
| AAUCAGUACUUCCAAAGUCUA | 1674 | asasucagUfaCfUfUfccaaagucuaL96 | 2770 | sense | 21 |
| UAGACUUUGGAAGUACUGAUUUA | 1675 | usAfsgacUfuUfGfgaagUfaCfugauususa | 2771 | antisense | 23 |
| AUCAGUACUUCCAAAGUCUAU | 1676 | asuscaguAfcUfUfCfcaaagucuauL96 | 2772 | sense | 21 |
| AUAGACUUUGGAAGUACUGAUUU | 1677 | asUfsagaCfuUfUfggaaGfuAfcugaususu | 2773 | antisense | 23 |
| GCUAAAUCAGUACUUCCAAAG | 1678 | gscsuaaaUfcAfGfUfacuuccaaagL96 | 2774 | sense | 21 |
| CUUUGGAAGUACUGAUUUAGCAU | 1679 | csUfsuugGfaAfGfuacuGfaUfuuagcsasu | 2775 | antisense | 23 |
| UGCUAAAUCAGUACUUCCAAA | 1680 | usgscuaaAfuCfAfGfuacuuccaaaL96 | 2776 | sense | 21 |
| UUUGGAAGUACUGAUUUAGCAUG | 1681 | usUfsuggAfaGfUfacugAfuUfuagcasusg | 2777 | antisense | 23 |
| UCAGCAUGCCAAUAUGUGUGG | 1682 | uscsagcaUfgCfCfAfauauguguggL96 | 2778 | sense | 21 |
| CCACACAUAUUGGCAUGCUGACC | 1683 | csCfsacaCfaUfAfuuggCfaUfgcugascsc | 2779 | antisense | 23 |
| CAGCAUGCCAAUAUGUGUGGG | 1684 | csasgcauGfcCfAfAfuaugugugggL96 | 2780 | sense | 21 |
| CCCACACAUAUUGGCAUGCUGAC | 1685 | csCfscacAfcAfUfauugGfcAfugcugsasc | 2781 | antisense | 23 |
| AGGGUCAGCAUGCCAAUAUGU | 1686 | asgsggucAfgCfAfUfgccaauauguL96 | 2782 | sense | 21 |
| ACAUAUUGGCAUGCUGACCCUCU | 1687 | asCfsauaUfuGfGfcaugCfuGfacccuscsu | 2783 | antisense | 23 |
| GAGGGUCAGCAUGCCAAUAUG | 1688 | gsasggguCfaGfCfAfugccaauaugL96 | 2784 | sense | 21 |
| CAUAUUGGCAUGCUGACCCUCUG | 1689 | csAfsuauUfgGfCfaugcUfgAfcccucsusg | 2785 | antisense | 23 |
| GCAUAUGUGGCUAAAGCAAUA | 1690 | gscsauauGfuGfGfCfuaaagcaauaL96 | 2786 | sense | 21 |
| UAUUGCUUUAGCCACAUAUGCAG | 1691 | usAfsuugCfuUfUfagccAfcAfuaugcsasg | 2787 | antisense | 23 |
| CAUAUGUGGCUAAAGCAAUAG | 1692 | csasuaugUfgGfCfUfaaagcaauagL96 | 2788 | sense | 21 |
| CUAUUGCUUUAGCCACAUAUGCA | 1693 | csUfsauuGfcUfUfuagcCfaCfauaugscsa | 2789 | antisense | 23 |
| UGCUGCAUAUGUGGCUAAAGC | 1694 | usgscugcAfuAfUfGfuggcuaaagcL96 | 2790 | sense | 21 |
| GCUUUAGCCACAUAUGCAGCAAG | 1695 | gsCfsuuuAfgCfCfacauAfuGfcagcasasg | 2791 | antisense | 23 |
| UUGCUGCAUAUGUGGCUAAAG | 1696 | ususgcugCfaUfAfUfguggcuaaagL96 | 2792 | sense | 21 |
| CUUUAGCCACAUAUGCAGCAAGU | 1697 | csUfsuuaGfcCfAfcauaUfgCfagcaasgsu | 2793 | antisense | 23 |
| AAAUGAUGAAGAAACUUUGGC | 1698 | asasaugaUfgAfAfGfaaacuuuggcL96 | 2794 | sense | 21 |
| GCCAAAGUUUCUUCAUCAUUUGC | 1699 | gsCfscaaAfgUfUfucuuCfaUfcauuusgsc | 2795 | antisense | 23 |
| AAUGAUGAAGAAACUUUGGCU | 1700 | asasugauGfaAfGfAfaacuuuggcuL96 | 2796 | sense | 21 |
| AGCCAAAGUUUCUUCAUCAUUUG | 1701 | asGfsccaAfaGfUfuucuUfcAfucauususg | 2797 | antisense | 23 |
| GGGCAAAUGAUGAAGAAACUU | 1702 | gsgsgcaaAfuGfAfUfgaagaaacuuL96 | 2798 | sense | 21 |
| AAGUUUCUUCAUCAUUUGCCCCA | 1703 | asAfsguuUfcUfUfcaucAfuUfugcccscsa | 2799 | antisense | 23 |
| GGGGCAAAUGAUGAAGAAACU | 1704 | gsgsggcaAfaUfGfAfugaagaaacuL96 | 2800 | sense | 21 |
| AGUUUCUUCAUCAUUUGCCCCAG | 1705 | asGfsuuuCfuUfCfaucaUfuUfgccccsasg | 2801 | antisense | 23 |
| GAGAUACUAAAGGAAGAAUUC | 1706 | gsasgauaCfuAfAfAfggaagaauucL96 | 2802 | sense | 21 |
| GAAUUCUUCCUUUAGUAUCUCGA | 1707 | gsAfsauuCfuUfCfcuuuAfgUfaucucsgsa | 2803 | antisense | 23 |
| AGAUACUAAAGGAAGAAUUCC | 1708 | asgsauacUfaAfAfGfgaagaauuccL96 | 2804 | sense | 21 |
| GGAAUUCUUCCUUUAGUAUCUCG | 1709 | gsGfsaauUfcUfUfccuuUfaGfuaucuscsg | 2805 | antisense | 23 |
| CCUCGAGAUACUAAAGGAAGA | 1710 | cscsucgaGfaUfAfCfuaaaggaagaL96 | 2806 | sense | 21 |
| UCUUCCUUUAGUAUCUCGAGGAC | 1711 | usCfsuucCfuUfUfaguaUfcUfcgaggsasc | 2807 | antisense | 23 |
| UCCUCGAGAUACUAAAGGAAG | 1712 | uscscucgAfgAfUfAfcuaaaggaagL96 | 2808 | sense | 21 |
| CUUCCUUUAGUAUCUCGAGGACA | 1713 | csUfsuccUfuUfAfguauCfuCfgaggascsa | 2809 | antisense | 23 |
| ACAACUCAGGAUGAAAAAUUU | 1714 | ascsaacuCfaGfGfAfugaaaaauuuL96 | 2810 | sense | 21 |
| AAAUUUUUCAUCCUGAGUUGUGG | 1715 | asAfsauuUfuUfCfauccUfgAfguugusgsg | 2811 | antisense | 23 |
| CAACUCAGGAUGAAAAAUUUU | 1716 | csasacucAfgGfAfUfgaaaaauuuuL96 | 2812 | sense | 21 |
| AAAAUUUUUCAUCCUGAGUUGUG | 1717 | asAfsaauUfuUfUfcaucCfuGfaguugsusg | 2813 | antisense | 23 |
| CGCCACAACUCAGGAUGAAAA | 1718 | csgsccacAfaCfUfCfaggaugaaaaL96 | 2814 | sense | 21 |
| UUUUCAUCCUGAGUUGUGGCGGC | 1719 | usUfsuucAfuCfCfugagUfuGfuggcgsgsc | 2815 | antisense | 23 |
| CCGCCACAACUCAGGAUGAAA | 1720 | cscsgccaCfaAfCfUfcaggaugaaaL96 | 2816 | sense | 21 |
| UUUCAUCCUGAGUUGUGGCGGCA | 1721 | usUfsucaUfcCfUfgaguUfgUfggcggscsa | 2817 | antisense | 23 |
| AGGGAAGGUGGAAGUCUUCCU | 1722 | asgsggaaGfgUfGfGfaagucuuccuL96 | 2818 | sense | 21 |
| AGGAAGACUUCCACCUUCCCUUC | 1723 | asGfsgaaGfaCfUfuccaCfcUfucccususc | 2819 | antisense | 23 |
| GGGAAGGUGGAAGUCUUCCUG | 1724 | gsgsgaagGfuGfGfAfagucuuccugL96 | 2820 | sense | 21 |
| CAGGAAGACUUCCACCUUCCCUU | 1725 | csAfsggaAfgAfCfuuccAfcCfuucccsusu | 2821 | antisense | 23 |
| UGGAAGGGAAGGUGGAAGUCU | 1726 | usgsgaagGfgAfAfGfguggaagucuL96 | 2822 | sense | 21 |
| AGACUUCCACCUUCCCUUCCACA | 1727 | asGfsacuUfcCfAfccuuCfcCfuuccascsa | 2823 | antisense | 23 |
| GUGGAAGGGAAGGUGGAAGUC | 1728 | gsusggaaGfgGfAfAfgguggaagucL96 | 2824 | sense | 21 |
| GACUUCCACCUUCCCUUCCACAG | 1729 | gsAfscuuCfcAfCfcuucCfcUfuccacsasg | 2825 | antisense | 23 |
| GGCGAGCUUGCCACUGUGAGA | 1730 | gsgscgagCfuUfGfCfcacugugagaL96 | 2826 | sense | 21 |
| UCUCACAGUGGCAAGCUCGCCGU | 1731 | usCfsucaCfaGfUfggcaAfgCfucgccsgsu | 2827 | antisense | 23 |
| GCGAGCUUGCCACUGUGAGAG | 1732 | gscsgagcUfuGfCfCfacugugagagL96 | 2828 | sense | 21 |
| CUCUCACAGUGGCAAGCUCGCCG | 1733 | csUfscucAfcAfGfuggcAfaGfcucgcscsg | 2829 | antisense | 23 |
| GGACGGCGAGCUUGCCACUGU | 1734 | gsgsacggCfgAfGfCfuugccacuguL96 | 2830 | sense | 21 |
| ACAGUGGCAAGCUCGCCGUCCAC | 1735 | asCfsaguGfgCfAfagcuCfgCfcguccsasc | 2831 | antisense | 23 |
| UGGACGGCGAGCUUGCCACUG | 1736 | usgsgacgGfcGfAfGfcuugccacugL96 | 2832 | sense | 21 |
| CAGUGGCAAGCUCGCCGUCCACA | 1737 | csAfsgugGfcAfAfgcucGfcCfguccascsa | 2833 | antisense | 23 |
| AUGUGCGUAACAGAUUCAAAC | 1738 | asusgugcGfuAfAfCfagauucaaacL96 | 2834 | sense | 21 |
| GUUUGAAUCUGUUACGCACAUCA | 1739 | gsUfsuugAfaUfCfuguuAfcGfcacauscsa | 2835 | antisense | 23 |
| UGUGCGUAACAGAUUCAAACU | 1740 | usgsugcgUfaAfCfAfgauucaaacuL96 | 2836 | sense | 21 |
| AGUUUGAAUCUGUUACGCACAUC | 1741 | asGfsuuuGfaAfUfcuguUfaCfgcacasusc | 2837 | antisense | 23 |
| GAUGAUGUGCGUAACAGAUUC | 1742 | gsasugauGfuGfCfGfuaacagauucL96 | 2838 | sense | 21 |
| GAAUCUGUUACGCACAUCAUCCA | 1743 | gsAfsaucUfgUfUfacgcAfcAfucaucscsa | 2839 | antisense | 23 |
| GGAUGAUGUGCGUAACAGAUU | 1744 | gsgsaugaUfgUfGfCfguaacagauuL96 | 2840 | sense | 21 |
| AAUCUGUUACGCACAUCAUCCAG | 1745 | asAfsucuGfuUfAfcgcaCfaUfcauccsasg | 2841 | antisense | 23 |
| GGGUCAGCAUGCCAAUAUGUG | 1746 | gsgsgucaGfcAfUfGfccaauaugugL96 | 2842 | sense | 21 |
| CACAUAUUGGCAUGCUGACCCUC | 1747 | csAfscauAfuUfGfgcauGfcUfgacccsusc | 2843 | antisense | 23 |
| GGUCAGCAUGCCAAUAUGUGU | 1748 | gsgsucagCfaUfGfCfcaauauguguL96 | 2844 | sense | 21 |
| ACACAUAUUGGCAUGCUGACCCU | 1749 | asCfsacaUfaUfUfggcaUfgCfugaccscsu | 2845 | antisense | 23 |
| CAGAGGGUCAGCAUGCCAAUA | 1750 | csasgaggGfuCfAfGfcaugccaauaL96 | 2846 | sense | 21 |
| UAUUGGCAUGCUGACCCUCUGUC | 1751 | usAfsuugGfcAfUfgcugAfcCfcucugsusc | 2847 | antisense | 23 |
| ACAGAGGGUCAGCAUGCCAAU | 1752 | ascsagagGfgUfCfAfgcaugccaauL96 | 2848 | sense | 21 |
| AUUGGCAUGCUGACCCUCUGUCC | 1753 | asUfsuggCfaUfGfcugaCfcCfucuguscsc | 2849 | antisense | 23 |
| GCUUGAAUGGGAUCUUGGUGU | 1754 | gscsuugaAfuGfGfGfaucuugguguL96 | 2850 | sense | 21 |
| ACACCAAGAUCCCAUUCAAGCCA | 1755 | asCfsaccAfaGfAfucccAfuUfcaagcscsa | 2851 | antisense | 23 |
| CUUGAAUGGGAUCUUGGUGUC | 1756 | csusugaaUfgGfGfAfucuuggugucL96 | 2852 | sense | 21 |
| GACACCAAGAUCCCAUUCAAGCC | 1757 | gsAfscacCfaAfGfauccCfaUfucaagscsc | 2853 | antisense | 23 |
| CAUGGCUUGAAUGGGAUCUUG | 1758 | csasuggcUfuGfAfAfugggaucuugL96 | 2854 | sense | 21 |
| CAAGAUCCCAUUCAAGCCAUGUU | 1759 | csAfsagaUfcCfCfauucAfaGfccaugsusu | 2855 | antisense | 23 |
| ACAUGGCUUGAAUGGGAUCUU | 1760 | ascsauggCfuUfGfAfaugggaucuuL96 | 2856 | sense | 21 |
| AAGAUCCCAUUCAAGCCAUGUUU | 1761 | asAfsgauCfcCfAfuucaAfgCfcaugususu | 2857 | antisense | 23 |
| UCAAAUGGCUGAGAAGACUGA | 1762 | uscsaaauGfgCfUfGfagaagacugaL96 | 2858 | sense | 21 |
| UCAGUCUUCUCAGCCAUUUGAUA | 1763 | usCfsaguCfuUfCfucagCfcAfuuugasusa | 2859 | antisense | 23 |
| CAAAUGGCUGAGAAGACUGAC | 1764 | csasaaugGfcUfGfAfgaagacugacL96 | 2860 | sense | 21 |
| GUCAGUCUUCUCAGCCAUUUGAU | 1765 | gsUfscagUfcUfUfcucaGfcCfauuugsasu | 2861 | antisense | 23 |
| GAUAUCAAAUGGCUGAGAAGA | 1766 | gsasuaucAfaAfUfGfgcugagaagaL96 | 2862 | sense | 21 |
| UCUUCUCAGCCAUUUGAUAUCUU | 1767 | usCfsuucUfcAfGfccauUfuGfauaucsusu | 2863 | antisense | 23 |
| AGAUAUCAAAUGGCUGAGAAG | 1768 | asgsauauCfaAfAfUfggcugagaagL96 | 2864 | sense | 21 |
| CUUCUCAGCCAUUUGAUAUCUUC | 1769 | csUfsucuCfaGfCfcauuUfgAfuaucususc | 2865 | antisense | 23 |
| GAAAGUCAUCGACAAGACAUU | 1770 | gsasaaguCfaUfCfGfacaagacauuL96 | 2866 | sense | 21 |
| AAUGUCUUGUCGAUGACUUUCAC | 1771 | asAfsuguCfuUfGfucgaUfgAfcuuucsasc | 2867 | antisense | 23 |
| AAAGUCAUCGACAAGACAUUG | 1772 | asasagucAfuCfGfAfcaagacauugL96 | 2868 | sense | 21 |
| CAAUGUCUUGUCGAUGACUUUCA | 1773 | csAfsaugUfcUfUfgucgAfuGfacuuuscsa | 2869 | antisense | 23 |
| AUGUGAAAGUCAUCGACAAGA | 1774 | asusgugaAfaGfUfCfaucgacaagaL96 | 2870 | sense | 21 |
| UCUUGUCGAUGACUUUCACAUUC | 1775 | usCfsuugUfcGfAfugacUfuUfcacaususc | 2871 | antisense | 23 |
| AAUGUGAAAGUCAUCGACAAG | 1776 | asasugugAfaAfGfUfcaucgacaagL96 | 2872 | sense | 21 |
| CUUGUCGAUGACUUUCACAUUCU | 1777 | csUfsuguCfgAfUfgacuUfuCfacauuscsu | 2873 | antisense | 23 |
| GGCUAAUUUGUAUCAAUGAUU | 1778 | gsgscuaaUfuUfGfUfaucaaugauuL96 | 2874 | sense | 21 |
| AAUCAUUGAUACAAAUUAGCCGG | 1779 | asAfsucaUfuGfAfuacaAfaUfuagccsgsg | 2875 | antisense | 23 |
| GCUAAUUUGUAUCAAUGAUUA | 1780 | gscsuaauUfuGfUfAfucaaugauuaL96 | 2876 | sense | 21 |
| UAAUCAUUGAUACAAAUUAGCCG | 1781 | usAfsaucAfuUfGfauacAfaAfuuagcscsg | 2877 | antisense | 23 |
| CCCCGGCUAAUUUGUAUCAAU | 1782 | cscsccggCfuAfAfUfuuguaucaauL96 | 2878 | sense | 21 |
| AUUGAUACAAAUUAGCCGGGGGA | 1783 | asUfsugaUfaCfAfaauuAfgCfcggggsgsa | 2879 | antisense | 23 |
| CCCCCGGCUAAUUUGUAUCAA | 1784 | cscscccgGfcUfAfAfuuuguaucaaL96 | 2880 | sense | 21 |
| UUGAUACAAAUUAGCCGGGGGAG | 1785 | usUfsgauAfcAfAfauuaGfcCfgggggsasg | 2881 | antisense | 23 |
| UGUCGACUUCUGUUUUAGGAC | 1786 | usgsucgaCfuUfCfUfguuuuaggacL96 | 2882 | sense | 21 |
| GUCCUAAAACAGAAGUCGACAGA | 1787 | gsUfsccuAfaAfAfcagaAfgUfcgacasgsa | 2883 | antisense | 23 |
| GUCGACUUCUGUUUUAGGACA | 1788 | gsuscgacUfuCfUfGfuuuuaggacaL96 | 2884 | sense | 21 |
| UGUCCUAAAACAGAAGUCGACAG | 1789 | usGfsuccUfaAfAfacagAfaGfucgacsasg | 2885 | antisense | 23 |
| GAUCUGUCGACUUCUGUUUUA | 1790 | gsasucugUfcGfAfCfuucuguuuuaL96 | 2886 | sense | 21 |
| UAAAACAGAAGUCGACAGAUCUG | 1791 | usAfsaaaCfaGfAfagucGfaCfagaucsusg | 2887 | antisense | 23 |
| AGAUCUGUCGACUUCUGUUUU | 1792 | asgsaucuGfuCfGfAfcuucuguuuuL96 | 2888 | sense | 21 |
| AAAACAGAAGUCGACAGAUCUGU | 1793 | asAfsaacAfgAfAfgucgAfcAfgaucusgsu | 2889 | antisense | 23 |
| CCGAGAAGUCACCAAGAAGCU | 1794 | cscsgagaAfgUfCfAfccaagaagcuL96 | 2890 | sense | 21 |
| AGCUUCUUGGUGACUUCUCGGUC | 1795 | asGfscuuCfuUfGfgugaCfuUfcucggsusc | 2891 | antisense | 23 |
| CGAGAAGUCACCAAGAAGCUA | 1796 | csgsagaaGfuCfAfCfcaagaagcuaL96 | 2892 | sense | 21 |
| UAGCUUCUUGGUGACUUCUCGGU | 1797 | usAfsgcuUfcUfUfggugAfcUfucucgsgsu | 2893 | antisense | 23 |
| AGGACCGAGAAGUCACCAAGA | 1798 | asgsgaccGfaGfAfAfgucaccaagaL96 | 2894 | sense | 21 |
| UCUUGGUGACUUCUCGGUCCUUG | 1799 | usCfsuugGfuGfAfcuucUfcGfguccususg | 2895 | antisense | 23 |
| AAGGACCGAGAAGUCACCAAG | 1800 | asasggacCfgAfGfAfagucaccaagL96 | 2896 | sense | 21 |
| CUUGGUGACUUCUCGGUCCUUGU | 1801 | csUfsuggUfgAfCfuucuCfgGfuccuusgsu | 2897 | antisense | 23 |
| AAACAUGGCUUGAAUGGGAUC | 1802 | asasacauGfgCfUfUfgaaugggaucL96 | 2898 | sense | 21 |
| GAUCCCAUUCAAGCCAUGUUUAA | 1803 | gsAfsuccCfaUfUfcaagCfcAfuguuusasa | 2899 | antisense | 23 |
| AACAUGGCUUGAAUGGGAUCU | 1804 | asascaugGfcUfUfGfaaugggaucuL96 | 2900 | sense | 21 |
| AGAUCCCAUUCAAGCCAUGUUUA | 1805 | asGfsaucCfcAfUfucaaGfcCfauguususa | 2901 | antisense | 23 |
| UGUUAAACAUGGCUUGAAUGG | 1806 | usgsuuaaAfcAfUfGfgcuugaauggL96 | 2902 | sense | 21 |
| CCAUUCAAGCCAUGUUUAACAGC | 1807 | csCfsauuCfaAfGfccauGfuUfuaacasgsc | 2903 | antisense | 23 |
| CUGUUAAACAUGGCUUGAAUG | 1808 | csusguuaAfaCfAfUfggcuugaaugL96 | 2904 | sense | 21 |
| CAUUCAAGCCAUGUUUAACAGCC | 1809 | csAfsuucAfaGfCfcaugUfuUfaacagscsc | 2905 | antisense | 23 |
| GACUUGCUGCAUAUGUGGCUA | 1810 | gsascuugCfuGfCfAfuauguggcuaL96 | 2906 | sense | 21 |
| UAGCCACAUAUGCAGCAAGUCCA | 1811 | usAfsgccAfcAfUfaugcAfgCfaagucscsa | 2907 | antisense | 23 |
| ACUUGCUGCAUAUGUGGCUAA | 1812 | ascsuugcUfgCfAfUfauguggcuaaL96 | 2908 | sense | 21 |
| UUAGCCACAUAUGCAGCAAGUCC | 1813 | usUfsagcCfaCfAfuaugCfaGfcaaguscsc | 2909 | antisense | 23 |
| AGUGGACUUGCUGCAUAUGUG | 1814 | asgsuggaCfuUfGfCfugcauaugugL96 | 2910 | sense | 21 |
| CACAUAUGCAGCAAGUCCACUGU | 1815 | csAfscauAfuGfCfagcaAfgUfccacusgsu | 2911 | antisense | 23 |
| CAGUGGACUUGCUGCAUAUGU | 1816 | csasguggAfcUfUfGfcugcauauguL96 | 2912 | sense | 21 |
| ACAUAUGCAGCAAGUCCACUGUC | 1817 | asCfsauaUfgCfAfgcaaGfuCfcacugsusc | 2913 | antisense | 23 |
| UAAAUCAGUACUUCCAAAGUC | 1818 | usasaaucAfgUfAfCfuuccaaagucL96 | 2914 | sense | 21 |
| GACUUUGGAAGUACUGAUUUAGC | 1819 | gsAfscuuUfgGfAfaguaCfuGfauuuasgsc | 2915 | antisense | 23 |
| AAAUCAGUACUUCCAAAGUCU | 1820 | asasaucaGfuAfCfUfuccaaagucuL96 | 2916 | sense | 21 |
| AGACUUUGGAAGUACUGAUUUAG | 1821 | asGfsacuUfuGfGfaaguAfcUfgauuusasg | 2917 | antisense | 23 |
| AUGCUAAAUCAGUACUUCCAA | 1822 | asusgcuaAfaUfCfAfguacuuccaaL96 | 2918 | sense | 21 |
| UUGGAAGUACUGAUUUAGCAUGU | 1823 | usUfsggaAfgUfAfcugaUfuUfagcausgsu | 2919 | antisense | 23 |
| CAUGCUAAAUCAGUACUUCCA | 1824 | csasugcuAfaAfUfCfaguacuuccaL96 | 2920 | sense | 21 |
| UGGAAGUACUGAUUUAGCAUGUU | 1825 | usGfsgaaGfuAfCfugauUfuAfgcaugsusu | 2921 | antisense | 23 |
| UCCUCAAUUGAAGAAGUGGCG | 1826 | uscscucaAfuUfGfAfagaaguggcgL96 | 2922 | sense | 21 |
| CGCCACUUCUUCAAUUGAGGAGG | 1827 | csGfsccaCfuUfCfuucaAfuUfgaggasgsg | 2923 | antisense | 23 |
| CCUCAAUUGAAGAAGUGGCGG | 1828 | cscsucaaUfuGfAfAfgaaguggcggL96 | 2924 | sense | 21 |
| CCGCCACUUCUUCAAUUGAGGAG | 1829 | csCfsgccAfcUfUfcuucAfaUfugaggsasg | 2925 | antisense | 23 |
| CACCUCCUCAAUUGAAGAAGU | 1830 | csasccucCfuCfAfAfuugaagaaguL96 | 2926 | sense | 21 |
| ACUUCUUCAAUUGAGGAGGUGGC | 1831 | asCfsuucUfuCfAfauugAfgGfaggugsgsc | 2927 | antisense | 23 |
| CCACCUCCUCAAUUGAAGAAG | 1832 | cscsaccuCfcUfCfAfauugaagaagL96 | 2928 | sense | 21 |
| CUUCUUCAAUUGAGGAGGUGGCC | 1833 | csUfsucuUfcAfAfuugaGfgAfgguggscsc | 2929 | antisense | 23 |
| CAAGAUGUCCUCGAGAUACUA | 1834 | csasagauGfuCfCfUfcgagauacuaL96 | 2930 | sense | 21 |
| UAGUAUCUCGAGGACAUCUUGAA | 1835 | usAfsguaUfcUfCfgaggAfcAfucuugsasa | 2931 | antisense | 23 |
| AAGAUGUCCUCGAGAUACUAA | 1836 | asasgaugUfcCfUfCfgagauacuaaL96 | 2932 | sense | 21 |
| UUAGUAUCUCGAGGACAUCUUGA | 1837 | usUfsaguAfuCfUfcgagGfaCfaucuusgsa | 2933 | antisense | 23 |
| UGUUCAAGAUGUCCUCGAGAU | 1838 | usgsuucaAfgAfUfGfuccucgagauL96 | 2934 | sense | 21 |
| AUCUCGAGGACAUCUUGAACACC | 1839 | asUfscucGfaGfGfacauCfuUfgaacascsc | 2935 | antisense | 23 |
| GUGUUCAAGAUGUCCUCGAGA | 1840 | gsusguucAfaGfAfUfguccucgagaL96 | 2936 | sense | 21 |
| UCUCGAGGACAUCUUGAACACCU | 1841 | usCfsucgAfgGfAfcaucUfuGfaacacscsu | 2937 | antisense | 23 |
| ACAUGCUAAAUCAGUACUUCC | 1842 | ascsaugcUfaAfAfUfcaguacuuccL96 | 2938 | sense | 21 |
| GGAAGUACUGAUUUAGCAUGUUG | 1843 | gsGfsaagUfaCfUfgauuUfaGfcaugususg | 2939 | antisense | 23 |
| CAUGCUAAAUCAGUACUUCCA | 1844 | csasugcuAfaAfUfCfaguacuuccaL96 | 2940 | sense | 21 |
| UGGAAGUACUGAUUUAGCAUGUU | 1845 | usGfsgaaGfuAfCfugauUfuAfgcaugsusu | 2941 | antisense | 23 |
| AACAACAUGCUAAAUCAGUAC | 1846 | asascaacAfuGfCfUfaaaucaguacL96 | 2942 | sense | 21 |
| GUACUGAUUUAGCAUGUUGUUCA | 1847 | gsUfsacuGfaUfUfuagcAfuGfuuguuscsa | 2943 | antisense | 23 |
| GAACAACAUGCUAAAUCAGUA | 1848 | gsasacaaCfaUfGfCfuaaaucaguaL96 | 2944 | sense | 21 |
| UACUGAUUUAGCAUGUUGUUCAU | 1849 | usAfscugAfuUfUfagcaUfgUfuguucsasu | 2945 | antisense | 23 |
| GAAAGGCACUGAUGUUCUGAA | 1850 | gsasaaggCfaCfUfGfauguucugaaL96 | 2946 | sense | 21 |
| UUCAGAACAUCAGUGCCUUUCCG | 1851 | usUfscagAfaCfAfucagUfgCfcuuucscsg | 2947 | antisense | 23 |
| AAAGGCACUGAUGUUCUGAAA | 1852 | asasaggcAfcUfGfAfuguucugaaaL96 | 2948 | sense | 21 |
| UUUCAGAACAUCAGUGCCUUUCC | 1853 | usUfsucaGfaAfCfaucaGfuGfccuuuscsc | 2949 | antisense | 23 |
| UGCGGAAAGGCACUGAUGUUC | 1854 | usgscggaAfaGfGfCfacugauguucL96 | 2950 | sense | 21 |
| GAACAUCAGUGCCUUUCCGCACA | 1855 | gsAfsacaUfcAfGfugccUfuUfccgcascsa | 2951 | antisense | 23 |
| GUGCGGAAAGGCACUGAUGUU | 1856 | gsusgcggAfaAfGfGfcacugauguuL96 | 2952 | sense | 21 |
| AACAUCAGUGCCUUUCCGCACAC | 1857 | asAfscauCfaGfUfgccuUfuCfcgcacsasc | 2953 | antisense | 23 |
| GUCAGCAUGCCAAUAUGUGUG | 1858 | gsuscagcAfuGfCfCfaauaugugugL96 | 2954 | sense | 21 |
| CACACAUAUUGGCAUGCUGACCC | 1859 | csAfscacAfuAfUfuggcAfuGfcugacscsc | 2955 | antisense | 23 |
| UCAGCAUGCCAAUAUGUGUGG | 1860 | uscsagcaUfgCfCfAfauauguguggL96 | 2956 | sense | 21 |
| CCACACAUAUUGGCAUGCUGACC | 1861 | csCfsacaCfaUfAfuuggCfaUfgcugascsc | 2957 | antisense | 23 |
| GAGGGUCAGCAUGCCAAUAUG | 1862 | gsasggguCfaGfCfAfugccaauaugL96 | 2958 | sense | 21 |
| CAUAUUGGCAUGCUGACCCUCUG | 1863 | csAfsuauUfgGfCfaugcUfgAfcccucsusg | 2959 | antisense | 23 |
| AGAGGGUCAGCAUGCCAAUAU | 1864 | asgsagggUfcAfGfCfaugccaauauL96 | 2960 | sense | 21 |
| AUAUUGGCAUGCUGACCCUCUGU | 1865 | asUfsauuGfgCfAfugcuGfaCfccucusgsu | 2961 | antisense | 23 |
| GAUGCUCCGGAAUGUUGCUGA | 1866 | gsasugcuCfcGfGfAfauguugcugaL96 | 2962 | sense | 21 |
| UCAGCAACAUUCCGGAGCAUCCU | 1867 | usCfsagcAfaCfAfuuccGfgAfgcaucscsu | 2963 | antisense | 23 |
| AUGCUCCGGAAUGUUGCUGAA | 1868 | asusgcucCfgGfAfAfuguugcugaaL96 | 2964 | sense | 21 |
| UUCAGCAACAUUCCGGAGCAUCC | 1869 | usUfscagCfaAfCfauucCfgGfagcauscsc | 2965 | antisense | 23 |
| CAAGGAUGCUCCGGAAUGUUG | 1870 | csasaggaUfgCfUfCfcggaauguugL96 | 2966 | sense | 21 |
| CAACAUUCCGGAGCAUCCUUGGA | 1871 | csAfsacaUfuCfCfggagCfaUfccuugsgsa | 2967 | antisense | 23 |
| CCAAGGAUGCUCCGGAAUGUU | 1872 | cscsaaggAfuGfCfUfccggaauguuL96 | 2968 | sense | 21 |
| AACAUUCCGGAGCAUCCUUGGAU | 1873 | asAfscauUfcCfGfgagcAfuCfcuuggsasu | 2969 | antisense | 23 |
| GCGUAACAGAUUCAAACUGCC | 1874 | gscsguaaCfaGfAfUfucaaacugccL96 | 2970 | sense | 21 |
| GGCAGUUUGAAUCUGUUACGCAC | 1875 | gsGfscagUfuUfGfaaucUfgUfuacgcsasc | 2971 | antisense | 23 |
| CGUAACAGAUUCAAACUGCCG | 1876 | csgsuaacAfgAfUfUfcaaacugccgL96 | 2972 | sense | 21 |
| CGGCAGUUUGAAUCUGUUACGCA | 1877 | csGfsgcaGfuUfUfgaauCfuGfuuacgscsa | 2973 | antisense | 23 |
| AUGUGCGUAACAGAUUCAAAC | 1878 | asusgugcGfuAfAfCfagauucaaacL96 | 2974 | sense | 21 |
| GUUUGAAUCUGUUACGCACAUCA | 1879 | gsUfsuugAfaUfCfuguuAfcGfcacauscsa | 2975 | antisense | 23 |
| GAUGUGCGUAACAGAUUCAAA | 1880 | gsasugugCfgUfAfAfcagauucaaaL96 | 2976 | sense | 21 |
| UUUGAAUCUGUUACGCACAUCAU | 1881 | usUfsugaAfuCfUfguuaCfgCfacaucsasu | 2977 | antisense | 23 |
| AGAGAAGAUGGGCUACAAGGC | 1882 | asgsagaaGfaUfGfGfgcuacaaggcL96 | 2978 | sense | 21 |
| GCCUUGUAGCCCAUCUUCUCUGC | 1883 | gsCfscuuGfuAfGfcccaUfcUfucucusgsc | 2979 | antisense | 23 |
| GAGAAGAUGGGCUACAAGGCC | 1884 | gsasgaagAfuGfGfGfcuacaaggccL96 | 2980 | sense | 21 |
| GGCCUUGUAGCCCAUCUUCUCUG | 1885 | gsGfsccuUfgUfAfgcccAfuCfuucucsusg | 2981 | antisense | 23 |
| AGGCAGAGAAGAUGGGCUACA | 1886 | asgsgcagAfgAfAfGfaugggcuacaL96 | 2982 | sense | 21 |
| UGUAGCCCAUCUUCUCUGCCUGC | 1887 | usGfsuagCfcCfAfucuuCfuCfugccusgsc | 2983 | antisense | 23 |
| CAGGCAGAGAAGAUGGGCUAC | 1888 | csasggcaGfaGfAfAfgaugggcuacL96 | 2984 | sense | 21 |
| GUAGCCCAUCUUCUCUGCCUGCC | 1889 | gsUfsagcCfcAfUfcuucUfcUfgccugscsc | 2985 | antisense | 23 |

### Example 2. A Single Dose of AD-84788 Potently Inhibits Ldha Expression and Activity In Vivo

The effect of AD-84788 on the level of expression of Ldha in vivo was evaluated in C57BL/6J wild-type mice by subcutaneous administration of a single 0.1 mg/kg, 0.3 mg/kg, 1.0 mg/kg, 3.0 mg/kg, or 10 mg/kg dose of AD-84788. Forty-eight hours after administration, mice were euthanized and the livers were dissected and flash frozen in liquid nitrogen. Livers were ground and approximately 10 mg of liver powder per sample was used for RNA isolation. RNA concentration was measured, adjusted to 100 ng/μl, cDNA was prepared, and RT-PCR analysis was performed as described above.

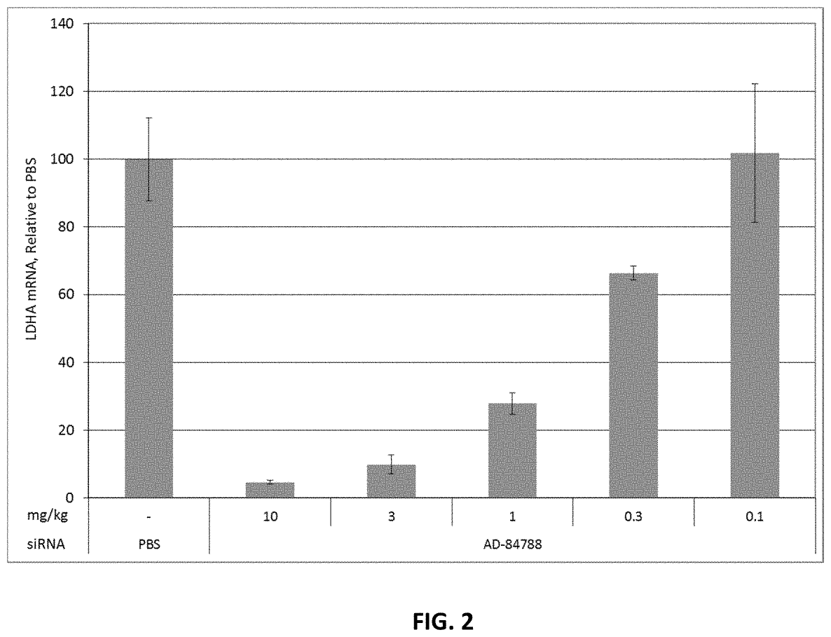

The results of these assays are depicted in FIG. 2 which demonstrates that a single 1 mg/kg, 3 mg/kg or 10 mg/kg dose of AD-84788 potently inhibits Ldha expression.

The effects of a single 0.1 mg/kg, 0.3 mg/kg, 1.0 mg/kg, 3.0 mg/kg, or 10 mg/kg subcutaneous dose of AD-84788 on hepatic Ldha enzyme activity was evaluated in Agxt deficient mice.

Agxt deficient mice have a targeted disruption of the alanine-glyoxylate amino transferase gene (Agxt) (Salido, et al. (2006) Proc. Natl. Acad. Sci. U.S.A. 103:18249). Mutant mice develop normally, but exhibit hyperoxaluria and calcium oxalate crystal formation. These Agxt knock-out mice are a recognized animal model of primary hyperoxaluria type I, a rare disease characterized by excessive hepatic oxalate production that leads to renal failure and which is casued by mutations in the AGXT gene.

Liver LDH enzyme activity was measured by the reduction of NAD to NADH in liver tissue lysates. Four weeks after administration, mice were euthanized and liver samples were collected and processed. Briefly, liver samples were weighed, homogenized in lysis buffer (25 mM HEPES, 1% Triton, 1% protease inhibitor) and homogenates were centrifuged to pellet cell debris. The supernantants were recovered, and solutions of NAD and either lactic acid or glyoxylate were added. The samples were placed into a multi-well plate and placed into a plate reader. Absorbance readings at 340 nm were collected for 20 minutes at 1 minute intervals. The data was used to calculate LDHA specific activity (nmoles of LDHA activity/min/mg protein).

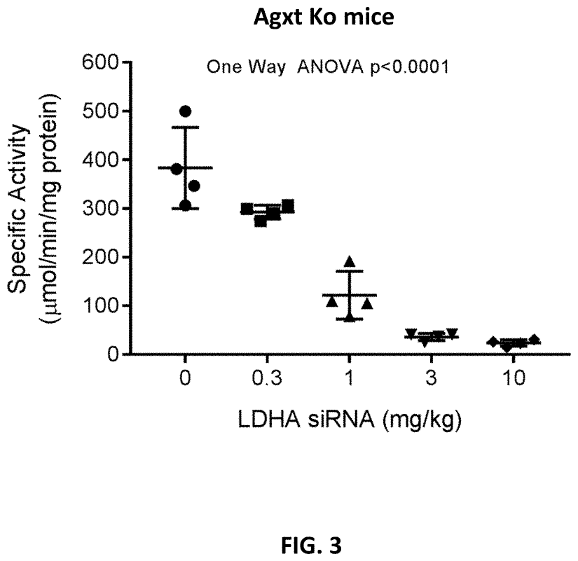

The results of these assays are depicted in FIG. 3 which demonstrates that a single 0.3 mg/kg, 1 mg/kg, 3 mg/kg or 10 mg/kg dose of AD-84788 potently inhibits Ldha enzyme activity.

### Example 3. AD-84788 Potently Reduces Endogenous LDHA Expression, LDHA Activity, and Oxalate Levels In Vivo

The effect of AD-84788 on endogenous oxalate production in vivo was evaluated in wild-type mice, Agxt deficient mice, and Grhpr knockout mice

Grhpr deficient mice have a targeted disruption of the glyoxylate reductase/hydroxypyruvate reductase (Grhpr) gene (see, e.g., Knight et al., (2011) Am J Physiol Renal Physiol 302(6): F688-F693). Mutant mice exhibit no difference in growth and development, but exhibit nephrocalcinosis including deposits of calcium oxalate in cortical and medullary tubules. Grhpr knock-out mice are an art recognized animal model of primary hyperoxaluria type II, an inherited disease characterized by excessive production of oxalate caused by mutations in the Grhpr gene.

Methods and Materials

Animals

Adult (12-14 weeks of age) male Agt deficient (Agxt Ko) mice on a C57BL/6J background, Grhpr deficient (Grhpr Ko) mice, and wild type litter mates were used for these studies. Mice were maintained in a barrier facility with a 12:12-hour light-dark cycle and an ambient temperature of 23±1° C. and had free access to food and water. All mice were placed on an ultra low oxalate diet to eliminate dietary oxalate contributions, e.g., so that urinary oxalate excretion levels represent substantially only endogenous oxalate production. All animal studies were approved by the Institutional Animal Use and Care Committee.

Metabolic Cage Urine Collections

For metabolic cage urine collections, animals were singly housed in Nalgene metabolic cages for collection of 24-hour urines, as previously described (Li, et. al. (2016) Biochimica et Biophysica Acta 1862:233). Three to four 24-hour urines were performed for each mouse before and after administration of an iRNA agent. The mean of these collections was used to characterize the urinary oxalate excretion of each animal.

LDHA iRNA Administration

The effect and durability of AD-84788 on urinary oxalate excretion was determined by administering Agxt deficient mice (n=6) a single 0.3 mg/kg, 1 mg/kg, 3 mg/kg, or 10 mg/kg dose of AD-84788 diluted in sterile 0.9% sodium chloride on Day 0. Twenty-four-hour urines were collected on weeks 1, 2, 3, 4, 6, 8, 9, and 10 post-dose. Baseline twenty-four-hour urine collections were also performed prior to the administration of AD-84788.

The effect of AD-84788 on urinary oxalate excretion was further determined by administering wild-type mice (n=6), Agxt mice (n=6) or Grhpr mice (n=6) a single 10 mg/kg dose of AD-84788 diluted in sterile 0.9% sodium chloride on Day 0. Twenty-four-hour urine samples were collected on days 7-10 post-dose. Baseline twenty-four-hour urine collections were also performed prior to the administration of AD-84788

The effect of multi-dose administration of AD-84788 on urinary oxalate excretion and was also determined Agxt mice (n=6). Agxt deficient mice were administered a 10 mg/kg dose of AD-84788 on Days 0, 11, 18, and 25. Twenty-four-hour urines were collected on Days −6, −5, −4, and −3 pre-dose. Twenty-four-hour urines were also collected on Days 7, 8, 9, and 10 post-dose; and on Days 28, 29, 30, and 31 post-dose.

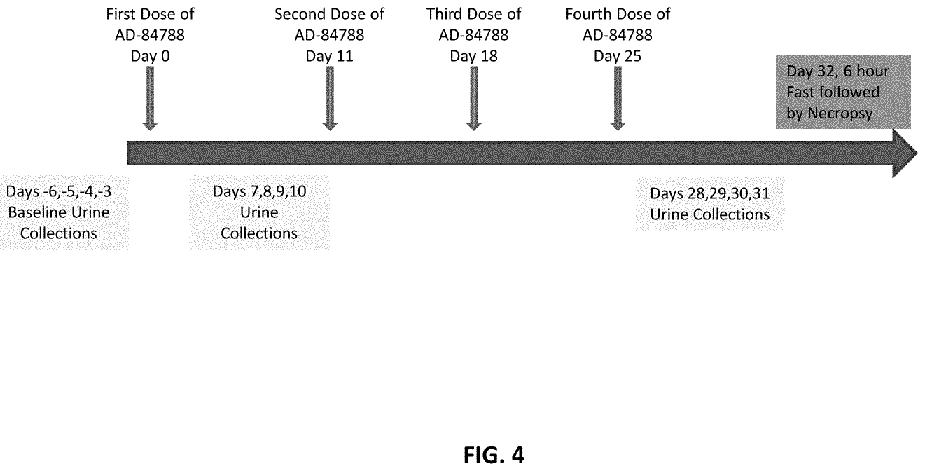

Following completion of 24-hour urine collections (Day 32 post-dose), tissue was collected to determine inhibition of LDHA protein and activity by enzymatic assays. Animals were fasted for 6 hours and anesthetized with vaporized isoflurane (Fluriso, MWI, Boise Id.) prior to tissue procurement. A schematic of this multi-dose study protocol is provided in FIG. 4.

Analytical Methods

Urinary oxalate levels were determined by ion chromatography coupled with mass spectroscopy (ICMS), as previously described (Li, et. al. (2016) Biochimica et Biophysica Acta 1862:233). Liver lactate was determined by ICMS (Knight, et. al. (2012). Anal Biochem. 421:121-124), and pyruvate and glyoxylate levels by HPLC (Knight and Holme s(2005) Am J Nephrol 25:171). Prior to lactate, pyruvate and glyoxylate measurements, tissue was extracted in trichloroacetic acid (final 10% v/v).

Liver LDH Enzyme Assay—Lactic Acid or Glyoxylate Substrates

Liver LDH enzyme activity was measured by the reduction of NAD to NADH in liver tissue lysates. Briefly, liver samples were weighed, homogenized in lysis buffer (25 mM HEPES, 1% Triton, 1% protease inhibitor) and homogenates were centrifuged to pellet cell debris. The supernantants were recovered, and solutions of NAD and either lactic acid or glyoxylate were added. The samples were placed into a multi-well plate and placed into a plate reader. Absornace readings at 340 nm were collected for 20 minutes at 1 minute intervals. The data was used to calculate LDHA specific activity (nmoles of LDHA activity/min/mg protein).

Heart and Thigh Skeletal Muscle LDH Enzyme Assay

Heart and thigh skeletal muscle LDH enzyme activity was also measured using lactic acid as a substrate. Briefly, liver samples were weighed, homogenized in lysis buffer (25 mM HEPES, 1% Triton, 1% protease inhibitor) and homogenates were centrifuged to pellet cell debris. The supernantants were recovered, and solutions of NAD and lactic acid were added. The samples were placed into a multi-well plate and placed into a plate reader. Absornace readings at 340 nm were collected for 20 minutes at 1 minute intervals. The data was used to calculate LDHA specific activity (nmoles of LDHA activity/min/mg protein).

Results

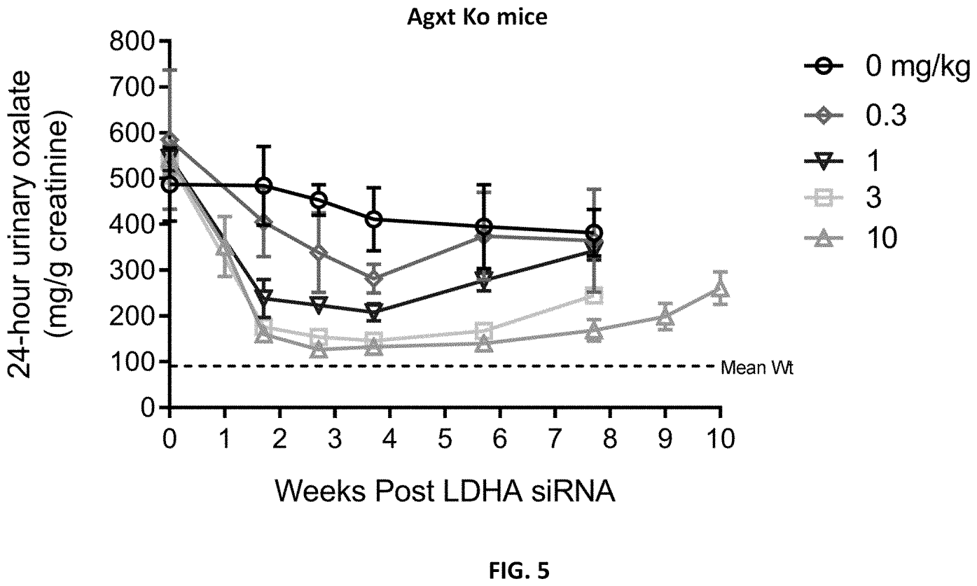

The effect and durability of LDHA inhibtion on endogenous oxalate excretion was also assessed and, as depicted in FIG. 5, compared to untreated control animals, administration of a single 0.3 mg/kg 1 mg/kg, 3 mg/kg or 10 mg/kg dose of AD-84788 decreased urinary oxalate excretion for at least 4 weeks post-dose of AD-84788.

Furthermore, as depicted in FIG. 6, four weeks after the administration of a single 10 mg/kg dose of siRNA, the level of endogenous oxalate excreted in the urine of Agxt deficient mice was significantly reduced by about 75%±3% compared to baseline, and the level of endogenous oxalate excretion in the urine of Grhpr deficient mice was reduced by about 32%±5%

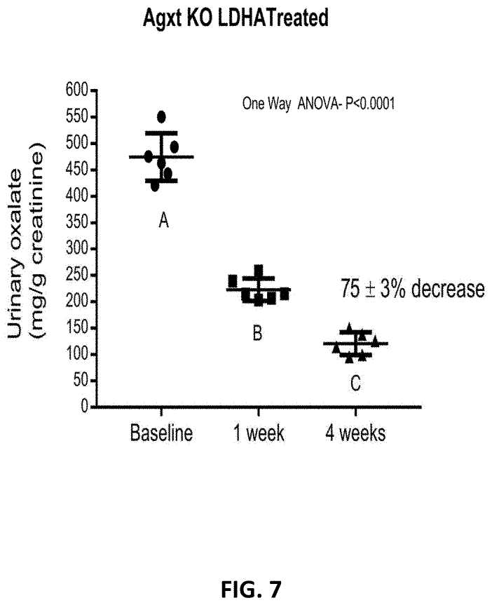

As depicted in FIG. 7, at one week following a single 10 mg/kg dose of AD-84788, the level of endogenous oxalate excreted in the urine of Agxt deficient mice was decreased. After the administration of four 10 mg/kg doses of AD-84788, endogenous oxalate levels excreted in the urine of Agxt deficient mice were unexpectedly reduced by about 75±3% from baseline levels of 120 mg/dl, demonstrating that decreasing the level of Ldha decreases the level of excreted oxalate and, thus, is useful for treating subjects having a kidney stone formation disease, disorder, or condition (e.g., a subject having a non-hyperoxaluria kidney stone formation disease, disorder, or condition).

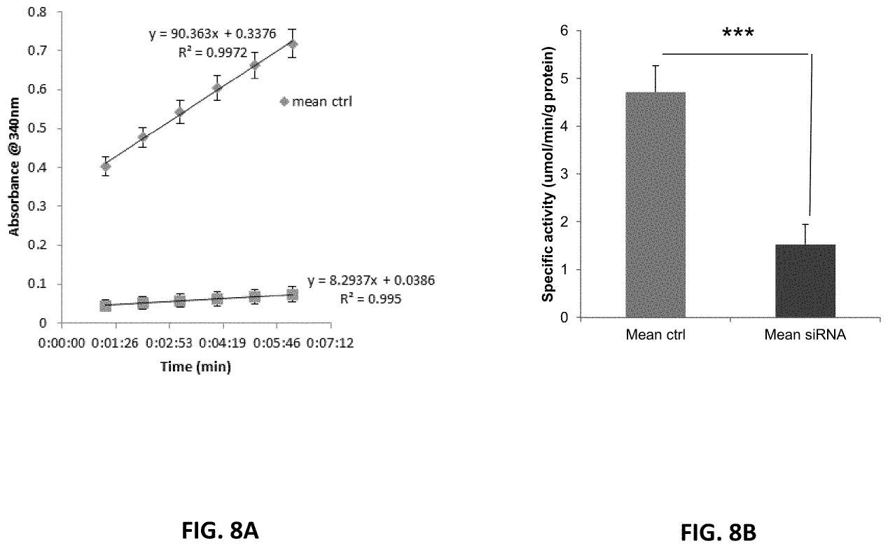

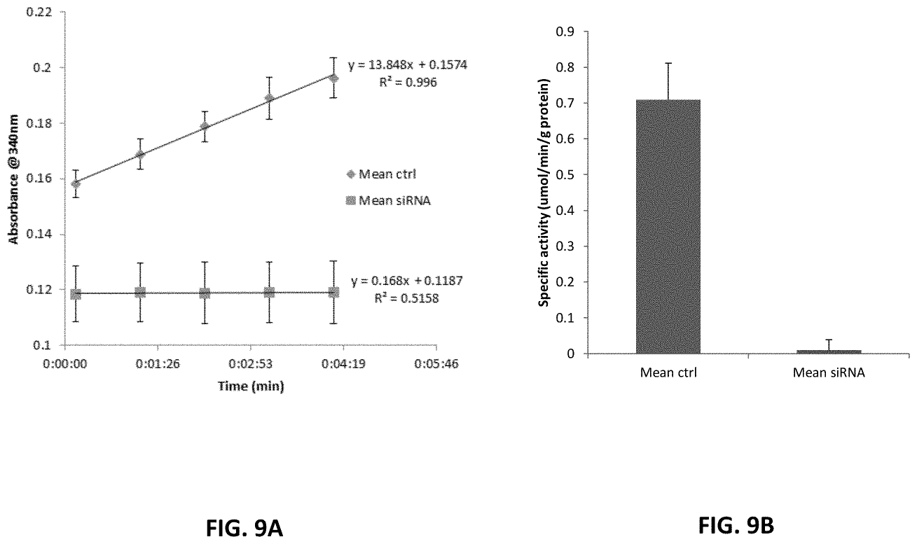

The effect of administration of four 10 mg/kg doses of AD-84788 on the levels of Ldha protein was also assessed by measuring the enzymic activity of Ldha present in liver samples from both wild-type and Agxt mice using either lactic acid or glyoxylate as a substrate. FIGS. 8A, 8B, 9A, and 9B demonstrate that, compared to untreated control animals, after the administration of four 10 mg/kg doses of AD-84788 to wild-type mice, significantly decreased liver LDH enzymatic activity as measured by the reduction of NAD to NADH using either lactic acid (FIGS. 8A and 8B) or glyoxylate (FIGS. 9A and 9B).

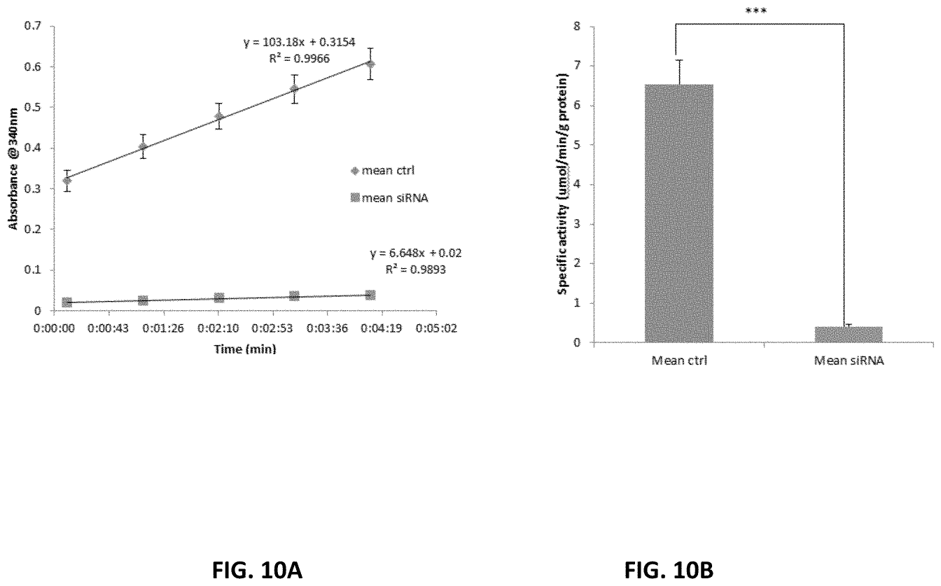

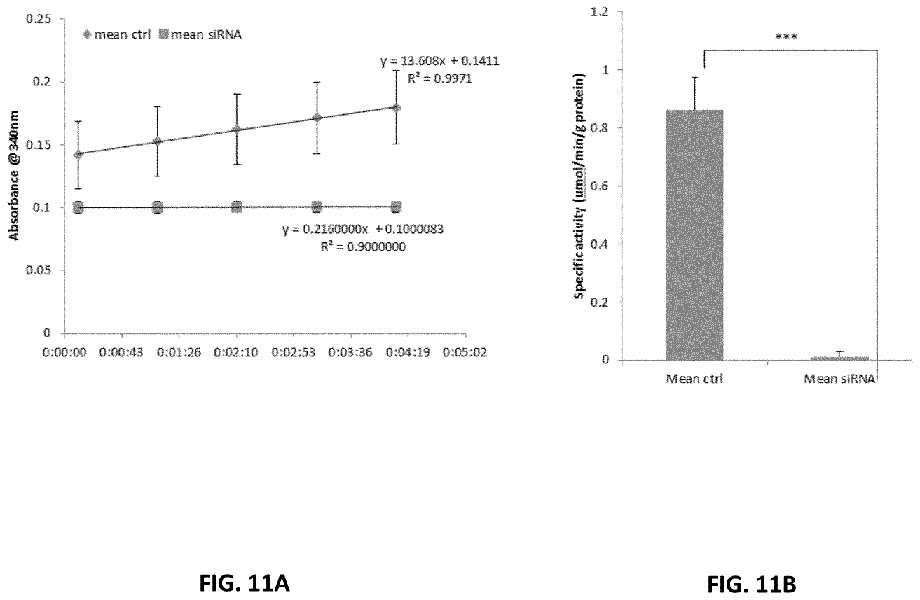

Similarly, in Agxt mice, compared to untreated control animals, after the administration of four 10 mg/kg doses of AD-84788 significantly decreased liver LDH enzymatic activity as measured by the reduction of NAD to NADH using either lactic acid (FIGS. 10A and 10B) or glyoxylate (FIGS. 11A and 11B).

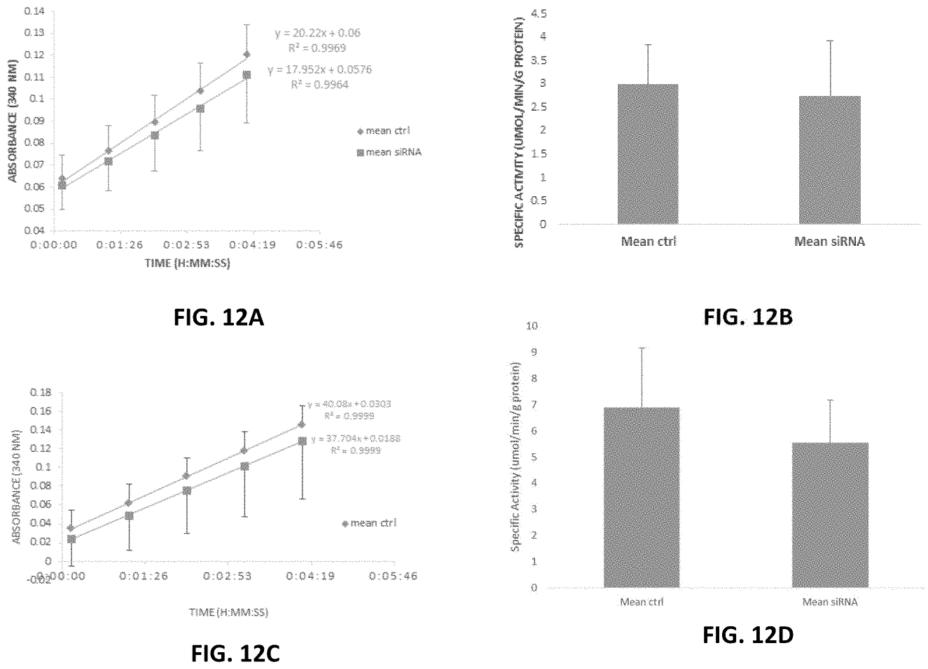

Lactate dehydrogenase is present throughout the body and the use of iRNA agents targeting LDHA may have systemic effects. However, as depicted in FIGS. 12A-12D, the reduction in LDH enzymatic activity by administration of AD-84788 (i.e., an iRNA agent conjugated to a GalNAc ligand which targets hepatocytes) is specific to the LDH present in the liver. In particular, compared to untreated control animals, administration of four 10 mg/kg doses of AD-84788 to wild-type mice does not significantly reduce either heart (FIGS. 12A and 12B) or skeletal muscle (FIGS. 12C and 12D) LDH enzymatic activity using lactic acid (FIGS. 8A and 8B) as a substrate.

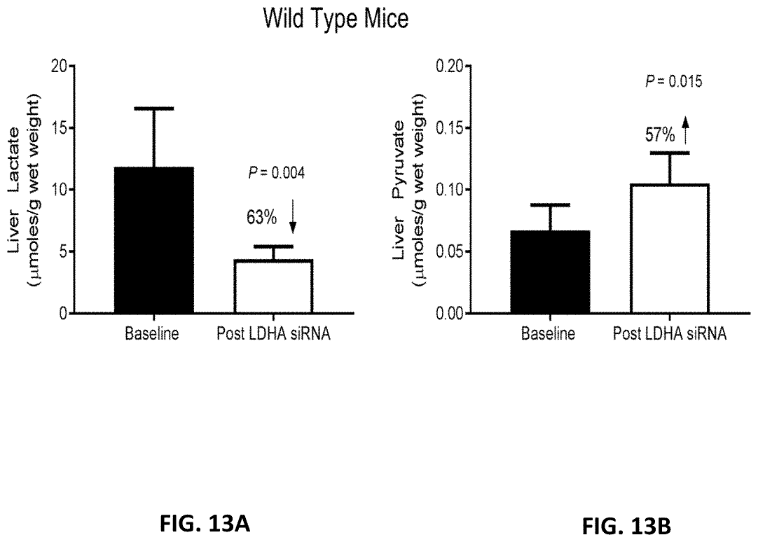

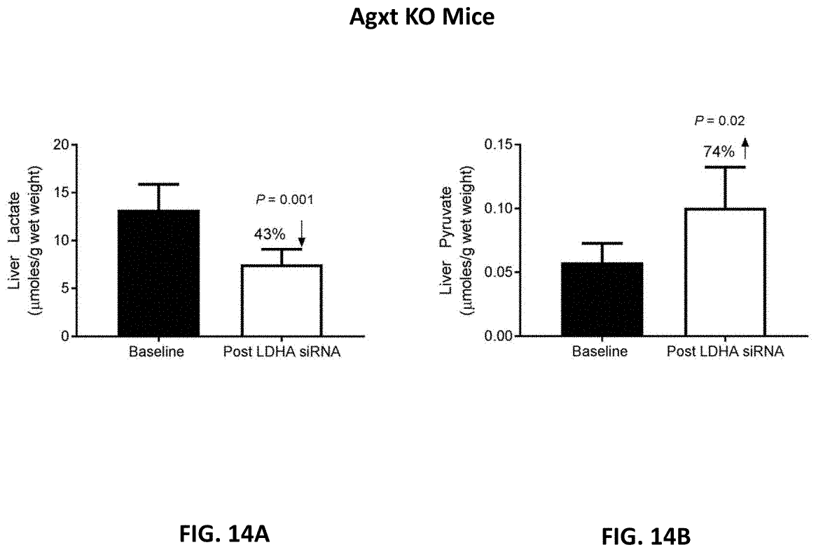

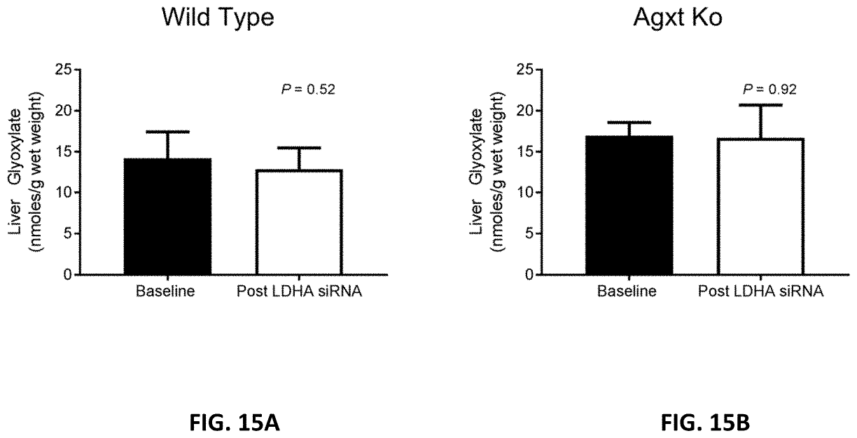

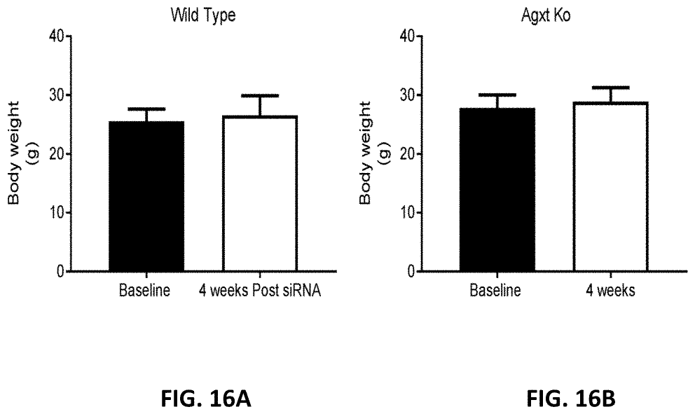

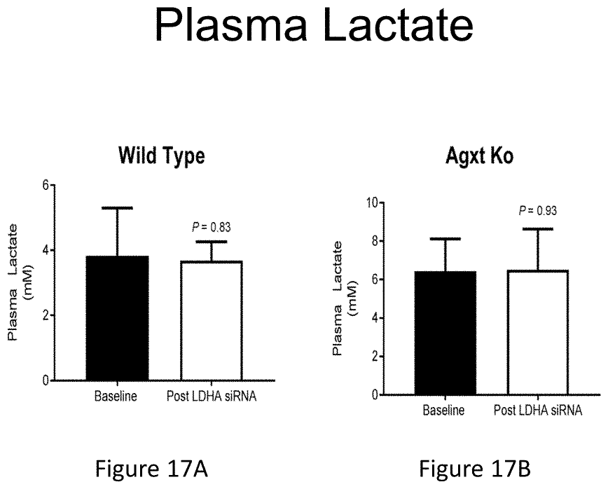

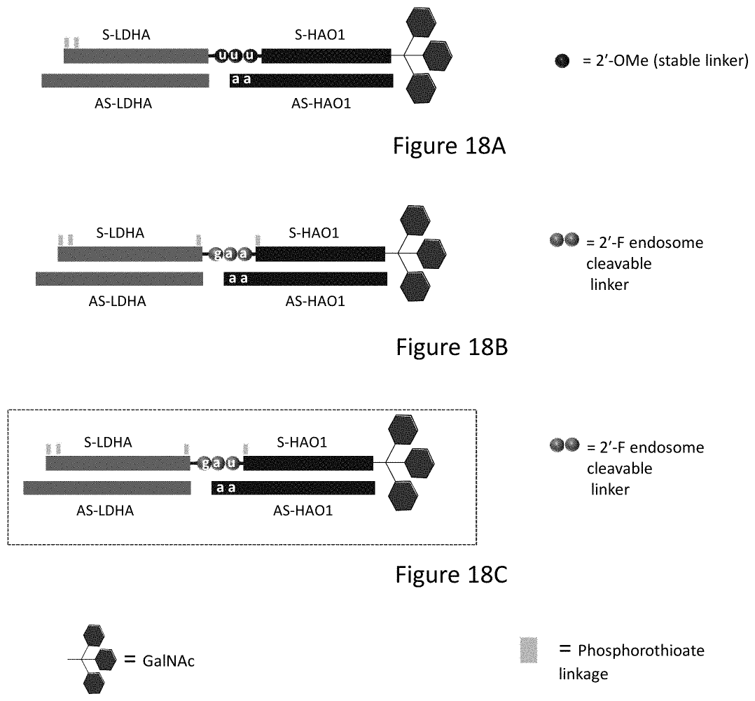

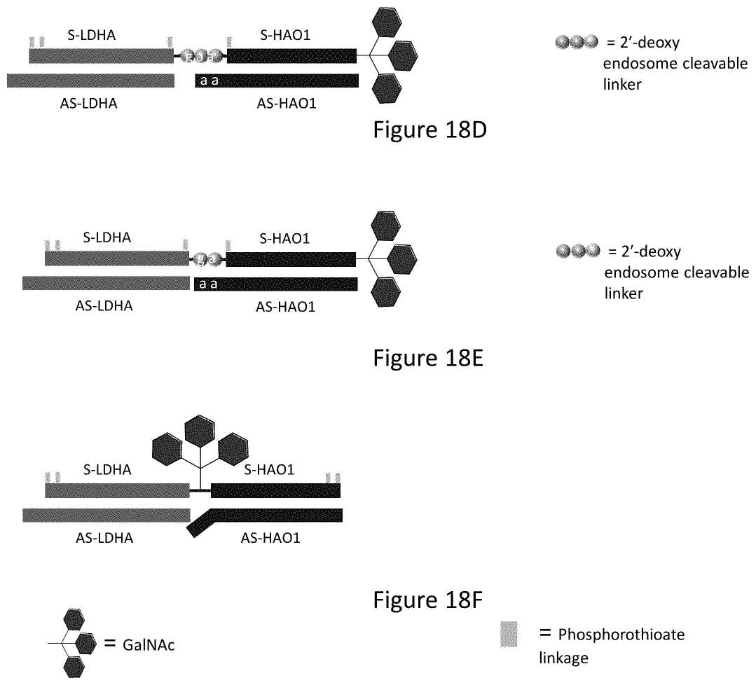

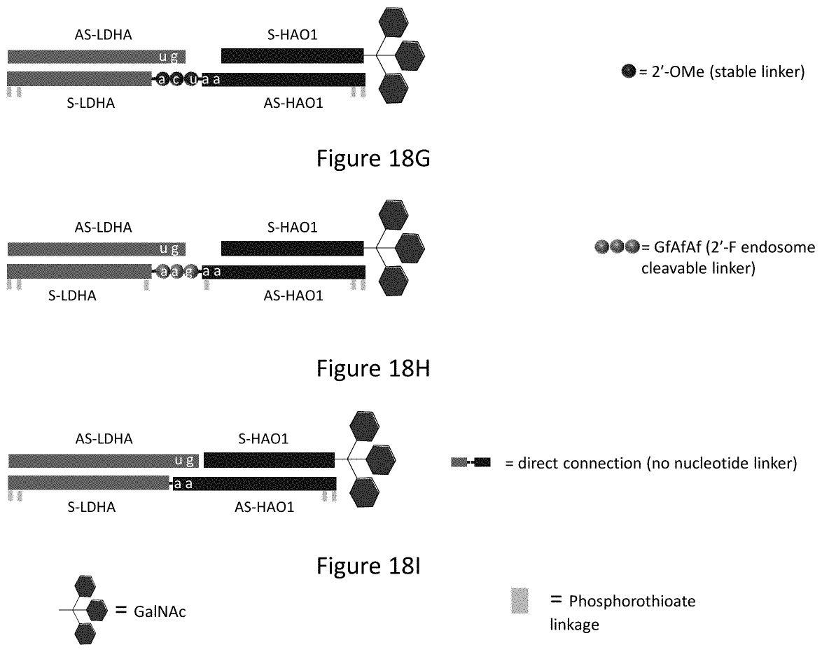

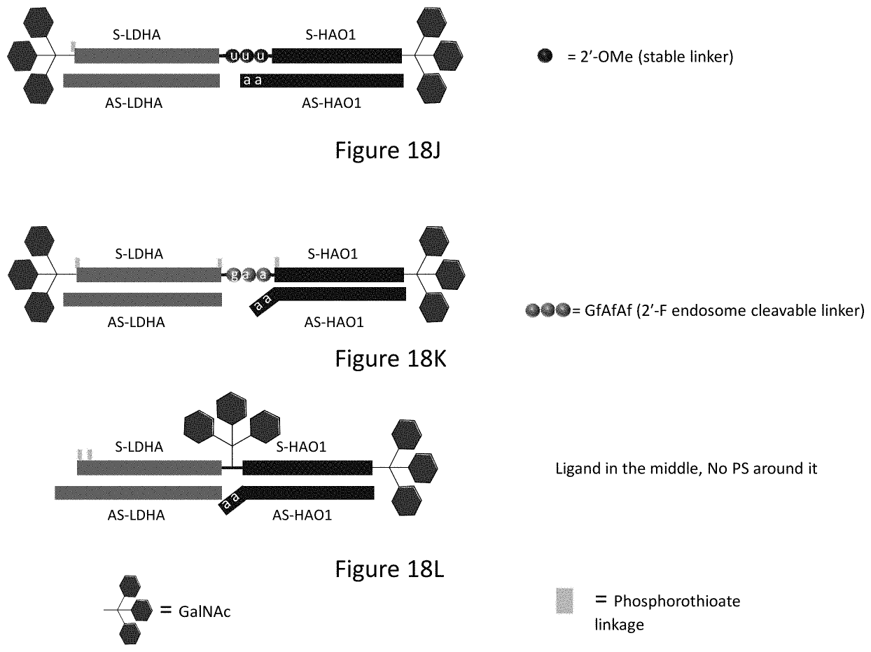

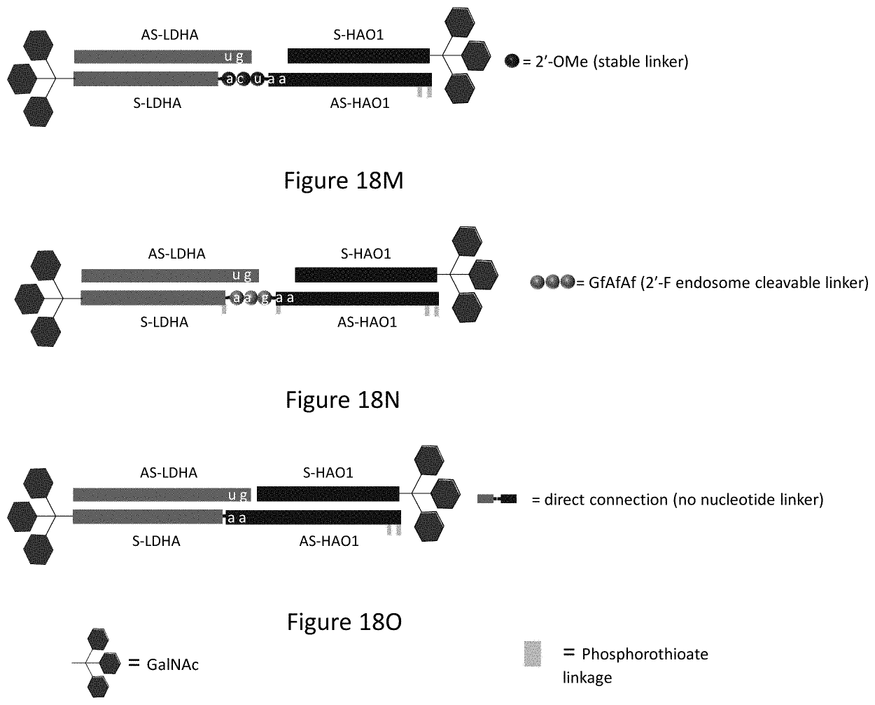

Furthermore, the reduction of Ldha levels by administration of four 10 mg/kg doses of AD-84788 to either wild-type of Agxt deficient mice did not increase liver or muscle lactate levels. In fact, in both wild-type (FIG. 13A) and Agxt deficient mice (FIG. 14A), lactate levels were significantly decreased in animals administered multiple doses of AD-84788. In addition, as depicted in FIGS. 13B and 14B, liver pyruvate levels were higher and, as depicted in FIGS. 15A and 15B, liver glyoxylate levels were unchanged in wild-type mice and Agxt deficient mice administered multiple doses of AD-84788. Further despite reduction of liver lactate levels in both the wild-type and Agxt deficient mice after the administration of four 10 mg/kg doses of AD-84788, plasma levels of lactate in both the wild-type and Agxt deficient mice were unaffected (FIGS. 17A and 17B). Notably, during the entirety of the study, the behavior and weights (see FIGS. 16A and 16B) of the treated and untreated control mice remained constant indicating that there were no significant metabolic changes in the animals, thus, demonstrating the safety of specific inhibition of liver Ldha using an iRNA agent such as AD-84788.

In summary, liver-specific knockdown of LDHA using the dsRNA agents of the invention resulted in profound oxalate lowering in both healthy and diseased animals. Additionally, substantial changes were seen in the levels of lactate, pyruvate and TCA Cycle organic acids in the livers of treated animals, consistent with the role of LDH in carbohydrate metabolism (see, e.g., FIG. 1B). However, none of the treated mice showed signs of behavioral and/or weight changes indicating that there were no significant metabolic changes in the animals. Accordingly, the data presented herein demonstrates the utility of the compositions and methods provided herein to decrease oxalate synthesis in subjects, such as subjects having a kidney stone formation disease, disorder, or condition (e.g., a subject having a non-hyperoxaluria kidney stone formation disease, disorder, or condition) and permit the determination of a suitable decrease in the level of oxalate that is beneficial to such subjects without resulting in adverse effects or safety concerns.

We claim:

1. A double stranded ribonucleic acid (dsRNA) agent for inhibiting expression of lactic acid dehydrogenase A (LDHA) in a cell, wherein the dsRNA agent comprises a sense strand and an antisense strand forming a double stranded region,
wherein the antisense strand comprises at least 19 contiguous nucleotides differing by no more than 3 nucleotides from the nucleotide sequence 5′-AUCAGAUAAAAAGGACAACAUGC-3′ (SEQ ID NO:3406),
wherein the antisense strand is 19-23 nucleotides in length,
wherein all of the nucleotides of the sense strand that comprise the double stranded region are modified nucleotides
wherein all of the nucleotides of the antisense strand are modified nucleotides, and
wherein a ligand comprising one or more N-acetylgalactosamine (GalNAc) derivatives attached through a monovalent, bivalent, or trivalent branched linker is conjugated to at least one strand of the agent.

2. The dsRNA agent of claim 1, wherein the antisense strand comprises at least 20 contiguous nucleotides differing by no more than 3 nucleotides from the nucleotide sequence
(SEQ ID NO: 3406)
5′-AUCAGAUAAAAAGGACAACAUGC-3′.

3. The dsRNA agent of claim 1, wherein the antisense strand comprises at least 21 contiguous nucleotides differing by no more than 3 nucleotides from the nucleotide sequence
(SEQ ID NO: 3406)
5′-AUCAGAUAAAAAGGACAACAUGC-3′.

4. The dsRNA agent of claim 1, wherein at least one of said modified nucleotides is selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modifice nucleotide, and a 2-O—(N-methylacetamide) modified nucleotide, and combinations thereof.

5. The dsRNA agent of claim 1, wherein the modified nucleotides are selected from the group consisting of a 2′-O-methyl modified nucleotide and a 2′-fluoro modified nucleotide.

6. The dsRNA agent of claim 1, wherein at least one strand comprises a 3′ overhang of at least 1 nucleotide.

7. The dsRNA agent of claim 1, wherein the agent further comprises at least one phosphorothioate or methylphosphonate internucleotide linkage.

8. The dsRNA agent of claim 7, wherein the sense strand comprises at least one phosphorothioate internucleotide linkage at the 3′terminus.

9. The dsRNA agent of claim 7, wherein the antisense strand comprises at least one phosphorothioate internucleotide linkage at the 3′terminus

10. The dsRNA agent of claim 1, wherein the ligand is conjugated to the 3′ end of the sense strand of the dsRNA agent.

11. The dsRNA agent of claim 1, wherein the first base pair of the double stranded region from the 5′ end of the antisense strand is an AU base pair.

12. The dsRNA agent of claim 1, wherein said double stranded region comprises 20 nucleotides.

13. The dsRNA agent of claim 1, wherein said double stranded region exhibits 100% complementarity between the sense and antisense strands.

14. The dsRNA agent of claim 1, wherein the dsRNA agent is capable of knocking down LDHA expression by at least 70% in a cell when provided as a single dose of 10 nM to the cell.

15. The dsRNA agent of claim 1, wherein the dsRNA agent is capable of knocking down LDHA expression by at least 80% in a cell when provided as a single dose of 10 nM to the cell.

16. The dsRNA agent of claim 1, wherein the antisense strand is 22 nucleotides in length.

17. A cell containing the dsRNA agent of claim 1.

18. A pharmaceutical composition for inhibiting expression of a lactic acid dehydrogenase A (LDHA) gene comprising the dsRNA agent of claim 1.

19. The pharmaceutical composition of claim 18, wherein the dsRNA agent is formulated in an unbuffered solution.

20. The pharmaceutical composition of claim 18, wherein the dsRNA agent is formulated with a buffered solution.

21. A method of inhibiting lactic acid dehydrogenase A (LDHA) expression in a cell, the method comprising contacting the cell with the dsRNA agent of claim 1, or the pharmaceutical composition of claim 18, thereby inhibiting expression of LDHA in the cell.

22. The method of claim 21, wherein the cell is within a subject.

23. The method of claim 22, wherein the subject is a human.

24. The method of claim 21, wherein the LDHA expression is inhibited by at least 30%, 40%, 50%, 60%, 70%, 80%, 90% or 95%, or to below the level of detection of LDHA expression.

25. The method of claim 23, wherein the human subject suffers from an oxalate pathway-associated disease, disorder, or condition.

26. The method of claim 25, wherein the oxalate pathway-associated disease, disorder, or condition is an oxalate-associated disease, disorder, or condition, or a lactate dehydrogenase-associated disease, disorder, or condition.

27. The method of claim 26, wherein the oxalate-associated disease, disorder, or condition is a kidney stone formation disease, disorder, or condition, or a calcium oxalate tissue deposition disease, disorder, or condition.

28. The method of claim 26, wherein the lactate dehydrogenase-associated disease, disorder, or condition is selected from the group consisting of cancer, fatty liver (steatosis), nonalcoholic steatohepatitis (NASH), cirrhosis of the liver, accumulation of fat in the liver, inflammation of the liver, hepatocellular necrosis, liver fibrosis, and nonalcoholic fatty liver disease (NAFLD).

29. The method of claim 21, wherein the cell is a liver cell.

30. A method of treating a subject having a disorder that would benefit from a reduction in LDHA expression, the method comprising
administering to the subject a therapeutically effective amount of the dsRNA agent of claim 1, or the pharmaceutical composition of claim 18, thereby treating the subject.
